(19) United States
(12) Patent Application Publication
(10) Pub. No.: US 2026/0365672 A1
(43) Pub. Date: Nov. 19, 2026

(54) K-RAS MUTANT PROTEIN INHIBITORS

(71) Applicant: Jacobio Pharmaceuticals Co., Ltd., Beijing (CN)

(72) Inventors: Hongwei YANG, Beijing (CN); Cunbo MA, Beijing (CN); Peng WANG, Beijing (CN); Panliang GAO, Beijing (CN); Huifeng HAN, Beijing (CN); Runze LI, Beijing (CN); Dan LIU, Beijing (CN); Yanping WANG, Beijing (CN); Wei LONG, Beijing (CN)

(73) Assignee: Jacobio Pharmaceuticals Co., Ltd., Beijing (CN)

(21) Appl. No.: 19/106,372

(22) PCT Filed: Aug. 25, 2023
(86) PCT No.: PCT/CN2023/114824

§ 371 Date: Feb. 25, 2025

(30) Foreign Application Priority Data
Aug. 25, 2022 (WO) PCT/CN2022/114762
Aug. 26, 2022 (WO) PCT/CN2022/115089
Sep. 22, 2022 (WO) PCT/CN2022/120586
Sep. 23, 2022 (WO) PCT/CN2022/120995
Sep. 27, 2022 (WO) PCT/CN2022/121656
Oct. 14, 2022 (WO) PCT/CN2022/125529
Oct. 17, 2022 (WO) PCT/CN2022/125684
Oct. 25, 2022 (WO) PCT/CN2022/127167
Jan. 17, 2023 (WO) PCT/CN2023/072571

(51) Int. Cl. C07D 519/00; A61K 31/551

(52) U.S. Cl. CPC C07D 519/00; A61K 31/551

(57) ABSTRACT

The invention relates to K-Ras mutant protein inhibitors of formula (I), a composition containing the same and the use thereof.

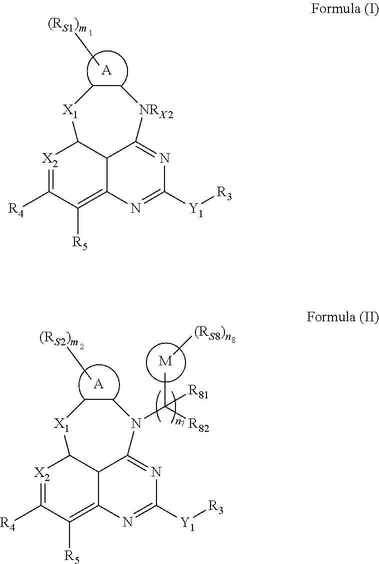

### CROSS-REFERENCES TO RELATED APPLICATIONS

This application claims the benefit of priority to PCT/CN2022/114762, filed on Aug. 25, 2022; PCT/CN2022/115089, filed on Aug. 26, 2022; PCT/CN2022/120586, filed on Sep. 22, 2022; PCT/CN2022/120995, filed on Sep. 23, 2022; PCT/CN2022/121656, filed on Sep. 27, 2022; PCT/CN2022/125529, filed on Oct. 14, 2022; PCT/CN2022/125684, filed on Oct. 17, 2022; PCT/CN2022/127167, filed on Oct. 25, 2022; and PCT/CN2023/072571, filed on Jan. 17, 2023, all of which are hereby incorporated herein by reference in their entireties.

### TECHNICAL FIELD

The invention relates to compounds that inhibit the activity of multiple forms of K-Ras protein including K-Ras wild type and K-Ras mutant types, compositions comprising the same, and the methods of using the same.

### BACKGROUND ART

Kirsten Rat Sarcoma 2 Viral Oncogene Homolog (“K-Ras”) is a small GTPase and a member of the RAS family of oncogenes. K-Ras serves as a molecular switch cycling between inactive (GDP-bound) and active (GTP-bound) states to transduce upstream cellular signals received from multiple tyrosine kinases to downstream effectors to regulate a wide variety of processes, including cellular proliferation. Aberrant expression of K-Ras accounts for up to ~20% of all cancers and oncogenic K-Ras mutations that stabilize GTP binding and lead to constitutive activation of K-Ras. K-Ras mutations at codons 12, 13, 61 and other positions of the K-Ras primary amino acid sequence are present in 88% of all pancreatic adenocarcinoma patients, 50% of all colorectal adenocarcinoma patients, and 32% lung adenocarcinoma patients. A recent publication also suggested wild type K-Ras inhibition could be a viable therapeutic strategy to treat K-Ras wild type dependent cancers.

Allele-specific K-Ras G12C inhibitors, such as sotorasib (AMG510) or adagrasib (MRTX849), are currently changing the treatment paradigm for patients with K-Ras G12C mutated non-small cell lung cancer and colorectal cancer. The success of addressing a previously elusive K-Ras allele has fueled drug discovery efforts for all K-Ras mutants. Multiple K-Ras inhibitors have the potential to address broad patient populations, including K-Ras G12C, K-Ras G12D, K-Ras G12V, K-Ras G13D, K-Ras G12R, K-Ras G12S, K-Ras G12A, K-Ras Q61H mutant and K-Ras wild type amplified cancers.

Therefore, there are unmet needs to develop new multiple K-Ras inhibitors for treating K-Ras mediated cancers.

### SUMMARY OF THE INVENTION

Provided herein is a compound of formula (I), a stereoisomer thereof, a pharmaceutically acceptable salt thereof, a pharmaceutically acceptable salt of the stereoisomer thereof, a prodrug thereof, a deuterated molecule thereof or a conjugated form thereof:

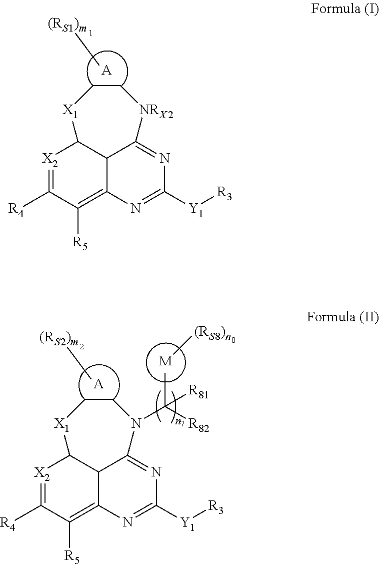

Wherein, the definition of each of variables is as below.

Also provided herein is a pharmaceutical composition comprising a therapeutically effective amount of a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof or a PROTAC molecule thereof as defined herein; and a pharmaceutically acceptable excipient.

Also provided herein is a method for treating cancer in a subject comprising administering a therapeutically effective amount of a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein to a subject in need thereof.

Also provided herein is a method for treating cancer in a subject in need thereof, the method comprising (a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and (b) if so, administering a therapeutically effective amount of a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein to the subject in need thereof.

Also provided herein is a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein for use in therapy.

Also provided herein is a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein for use as a medicament.

Also provided herein is a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein for use in a method for the treatment of cancer.

Also provided herein is a use of a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein for the treatment of cancer.

Also provided herein is a use of a compound of formula (I) or formula (II), a stereoisomer thereof, a pharmaceutically acceptable salt thereof, a pharmaceutically acceptable salt of the stereoisomer thereof, a prodrug thereof, a deuterated molecule thereof, a PROTAC molecule thereof, or a pharmaceutical composition as defined herein for the manufacture of a medicament for the treatment of cancer.

### DETAILED DESCRIPTION

Provided herein are the following disclosures:
[1]. A compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof:

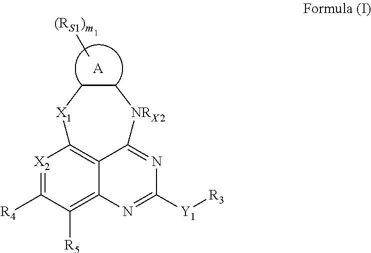

Wherein,
X₁ at each occurrence is independently selected from a bond, —C(R_{X11}) (R_{X12})—, —NR_{X13}—, —O—, —S—, —S(═O)— or —S(═O)₂—;
R_{X11} or R_{X12} is independently selected from hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, R_{X11} and R_{X12} together with the carbon atom to which they are both attached forms

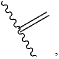

a 3-10 membered carbocyclic ring or a 3-10 heterocyclic ring; wherein, said

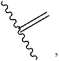

3-10 membered carbocylic ring or 3-10 heterocyclic ring is independently unsubstituted or substituted with one or more R_{SX1};
R_{X13} is selected from hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —OS(═O)R_{D}, —S(═O)OR_{C}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R_{X2} is selected from hydrogen, deuterium, —C₁₋₆alkyl, haloC₁₋₆alkyl, —C₁₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆ alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
X₂ at each occurrence is independently selected from N or CR₁;
R₁ is selected from hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1A})₂, —OR_{1A}, —SR_{1A}, —S(═O)R_{1B}, —S(═O)₂R_{1B}, —C(═O)R_{1B}, —C(═O)OR_{1A}, —OC(═O)R_{1B}, —C(═O)N(R_{1A})₂, —NR_{1A}C(═O)R_{1B}, —OC(═O)OR_{1B}, —NR_{1A}C(═O)OR_{1A}, —NR_{1A}C(═S)OR_{1A}, —OC(═O)N(R_{1A})₂, —NR_{1A}C(═O)N(R_{1A})₂, —S(═O)OR_{1A}, —OS(═O)R_{1B}, —S(═O)N(R_{1A})₂, —NR_{1A}S(═O)R_{1B}, —S(═O)₂OR_{1A}, —OS(═O)₂R_{1B}, —S(═O)₂N(R_{1A})₂, —NR_{1A}S(═O)R_{1B}, —OS(═O)₂OR_{1A}, —NR_{1A}S(═O)₂OR_{1A}, —OS(═O)₂N(R_{1A})₂, —NR_{1A}S(═O)₂N(R_{1A})₂; —P(R_{1A})₂, —P(═O)(R_{1B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1C})₂, —OR_{1C}, —SR_{1C}, —S(═O)R_{1D}, —S(═O)₂R_{1D}, —C(═O)R_{1D}, —C(═O) OR_{1D}, —OC(═O)R_{1D}, —C(═O)N(R_{1C})₂, —NR_{1C}C(═O)R_{1D}, —OC(═O)OR_{1C}, —NR_{1C}C(═O)OR_{1C}, —NR_{1C}C(═S)OR_{1C}, —OC(═O)N(R_{1C})₂, —NR_{1C}C(═O)N(R_{1C})₂, —S(═O)OR_{1C}, —OS(═O)R_{1D}, —S(═O)N(R_{1C})₂, —NR_{1C}S(═O)R_{1D}, —S(═O)₂OR_{1C}, —OS(═O)₂R_{1D}, —S(═O)N(R_{1C})₂, —NR_{1C}S(═O)₂R_{1D}, —OS(═O)₂OR_{1C}, —NR_{1C}S(═O)₂OR_{1C}, —OS(O)₂N(R_{1C})₂N(R_{1C})₂, —NR_{1C}S(═O)₂N(R_{1C})₂, —P(R_{1C})₂, —P(═O)(R_{1D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Ring A is selected from a 3-10 membered carbocyclic ring, 3-10 membered heterocyclic ring, 6-10 membered aryl ring, 5-10 membered heteroaryl ring;
R_{S1} is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1A})₂, —OR_{S1A}, —SR_{S1A}, —S(═O)R_{S1B}, —S(═O)₂R_{S1B}, —C(═O)R_{S1B}, —C(═O)OR_{S1A}, —OC(═O)R_{S1B}, —C(═O)N(R_{S1A})₂, —N_{S1A}C(═O)R_{S1B}, —OC(═O)OR_{S1A}; —N_{S1A}C(═O)OR_{S1A}, —NR_{S1A}C(═S)O_{S1A}, —OC(═O)N(R_{S1A})₂, —S(═O)OR_{S1A}, —OS(═O)R_{S1B}, —S(═O)N(R_{S1A})₂, —NR_{S1A}S(═O)R_{S1B}, —S(═O)₂OR_{S1A}, —OS(═O)₂R_{S1B}, —S(═O)₂N(R_{S1A}), —NR_{S1A}S(═O)₂R_{S1B}, —OS(═O)₂OR_{S1A}, —NR_{S1A}S(═O)₂OR_{S1A}, —OS(═O)₂N(R_{S1A})₂, —NR_{S1A}S(═O)₂N(R_{S1A})₂, —P(R_{S1A})₂, —P(═O)(R_{S1B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1C})₂, —OR_{S1C}, —SR_{S1C}, —S(═O)R_{S1D}, —S(═O)₂R_{S1D}, —C(═O)R_{S1D}, —C(═O)OR_{S1C}, —OC(═O)R_{S1D}, —C(═O)N(R_{S1C})₂, —NR_{S1C}C(═O)OR_{S1C}, —NR_{S1C}C(═S)OR_{S1C}, —OC(═O)N(R_{S1C})₂, —NR_{S1C}C(═O)R_{S1D}, —OC(═O)OR_{S1C}, —NR_{C}C(═O)N(R_{S1C})₂, —S(═O)OR_{S1C}, —OS(═O)R_{S1D}, —S(═O)N(R_{S1C})₂, —NR_{S1C}S(═O)R_{S1D}, —S(═O)₂OR_{S1C}, —OS(═O)₂R_{S1D}, —S(═O)N(R_{S1C})₂, —NR_{S1C}S(═O)₂R_{S1D}, —OS(═O)₂OR_{S1C}, —NR_{S1C}S(═O)₂OR_{S1C}, —OS(═O)₂N(R_{S1D})₂, —NR_{S1C}S(═O)₂N(R_{S1C})₂, —P(R_{S1C})₂, —P(═O)(R_{S1D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S1} together with the carbon atom to which they are both attached form

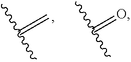

a 3-10 membered carbocyclic ring or a 3-10 heterocyclic ring; wherein, said

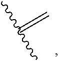

3-10 membered carbocylic ring or 3-10 heterocyclic ring is independently unsubstituted or substituted with one or more R_{S11};

Optionally, two adjacent R_{S1} together with the atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S12};

Optionally, two nonadjacent R_{S1} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S13};
m₁ is selected from 0, 1, 2, 3, 4, 5, 6, 7, 8 or 9;
Y₁ is a bond, O, S, S(═O), S(═O)₂ or NR_{Y11};
R_{Y11} is selected from hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —OS(═O)R_{D}, —S(═O)OR_{C}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R₃ is selected from

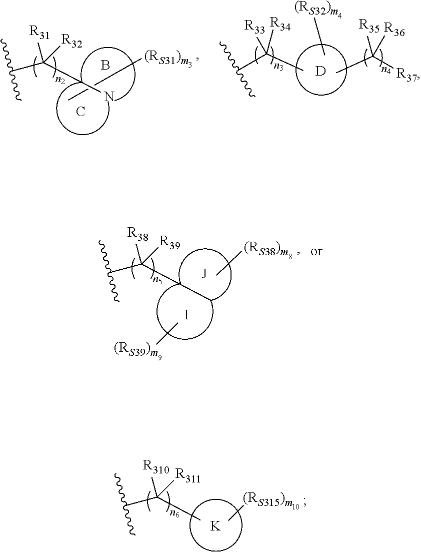

Each of R₃₁, R₃₂, R₃₃, R₃₄, R₃₅, R₃₆, R₃₈, R₃₉, R₃₁₀ and R₃₁₁ is independently selected form hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C}), —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{D})₂, —S(═O)OR_{C}, —OS(═O)R_{D}, —S(═O)N(R_{D})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, R₃₁ and R₃₂ together with the carbon atom to which they are both attached form

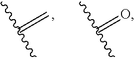

a 3-10 membered carbocyclic ring or a 3-10 heterocyclic ring; wherein, said

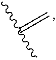

3-10 membered carbocyclic ring or 3-10 heterocyclic ring is independently unsubstituted or substituted with one or more R_{S33};

Optionally, R₃₃ and R₃₄ together with the carbon atom to which they are both attached form

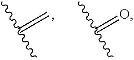

a 3-10 membered carbocyclic ring or a 3-10 heterocyclic ring; wherein, said

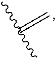

3-10 membered carbocyclic ring or 3-10 heterocyclic ring is independently unsubstituted or substituted with one or more R_{S34};

Optionally, R₃₅ and R₃₆ together with the carbon atom to which they are both attached form

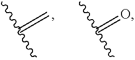

a 3-10 membered carbocyclic ring or a 3-10 heterocyclic ring; wherein, said

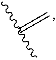

3-10 membered carbocyclic ring or 3-10 heterocyclic ring is independently unsubstituted or substituted with one or more R_{S35};

Optionally, R₃₈ and R₃₉ together with the carbon atom to which they are both attached form

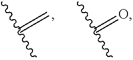

a 3-10 membered carbocyclic ring or a 3-10 heterocyclic ring; wherein, said

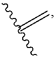

3-10 membered carbocyclic ring or 3-10 heterocyclic ring is independently unsubstituted or substituted with one or more R₃₁₀;

Optionally, R₃₁₀ and R₃₁₁ together with the carbon atom to which they are both attached form

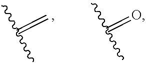

a 3-10 membered carbocyclic ring or a 3-10 heterocyclic ring; wherein, said

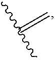

3-10 membered carbocyclic ring or 3-10 heterocyclic ring is independently unsubstituted or substituted with one or more R_{S316};
n₂ is selected from 0, 1, 2, 3, 4, 5 or 6;
n₃ is selected from 0, 1, 2, 3, 4, 5 or 6;
n₄ is selected from 0, 1, 2, 3, 4, 5 or 6;
n₅ is selected from 0, 1, 2, 3, 4, 5 or 6;
n₆ is selected from 0, 1, 2, 3, 4, 5 or 6;
Ring B is a 3-10 membered heterocyclic ring optionally further containing 1, 2, or 3 heteroatoms selected from N, O, S, S(═O) or S(═O)₂;
Ring C is a 3-10 membered heterocyclic ring optionally further containing 1, 2, or 3 heteroatoms selected from N, O, S, S(═O) or S(═O)₂;
Ring D is selected from a 3-10 membered carbocyclic ring or a 3-10 heterocyclic ring;
Ring I is a 3-10 membered carbocyclic ring or a 3-10 membered heterocyclic ring containing 1, 2, or 3 heteroatoms selected from N, O, S, S(═O) or S(═O)₂;
Ring J is a 3-10 membered carbocyclic ring or a 3-10 membered heterocyclic ring containing 1, 2, or 3 heteroatoms selected from N, O, S, S(═O) or S(═O)₂;
Ring K is selected from 3-10 membered carbocyclic ring or a 3-10 membered heterocyclic ring containing 1, 2, or 3 heteroatoms selected from N, O, S, S(═O) or S(═O)₂;
R_{S31} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31A})₂, —OR_{S31A}, —SR_{S31A}, —S(═O)R_{S31B}, —S(═O)₂R_{S31B}, —C(═O)R_{S31B}, —C(═O)OR_{S31A}, —OC(═O)R_{S31B}, —C(═O)N(R_{S31A})₂, —NR_{S31A}C(═O)R_{S31B}, —OC(═O)OR_{S31A}, —NR_{S31A}C(═O)OR_{S31A}, —NR_{S31A}C(═S)OR_{S31A}, —OC(═O)N(R_{S31A})₂, —NR_{S31A}C(═O)N(R_{S31A})₂, —S(═O)OR_{S31A}, —OS(═O)R_{S31B}, —S(═O)N(R_{S31A})₂, —NR_{S31A}S(═O)R_{S31B}, —S(═O)₂OR_{S31A}, —OS(═O)₂N(R_{S31A})₂, —NR_{S31A}S(═O)₂N(R_{S31A})₂, —P(R_{S31A})₂; —P(═O)(R_{S31B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31C})₂, —OR_{S31C}, —SR_{S31C}, —S(═O)R_{S31D}, —S(═O)₂R_{S31D}, —C(═O)R_{S31D}, —C(═O)OR_{S31C}, —OC(═O)R_{S31D}, —C(═O)N(R_{S31C})₂, —NR_{S31C}C(═O)R_{S31D}, —OC(═O)OR_{S31C}, —NR_{S31C}C(═O)OR_{S31C}, —OC(═O)N(R_{S31C})₂, —NR_{S31C}C(═O)N(R_{S31C})₂, —S(═O)OR_{S31C}, —OS(═O)R_{S31D}, —S(═O)N(R_{S31C})₂, —NR_{S31C}S(═O)R_{S31D}, —S(═O)₂OR_{S31C}, —OS(═O)₂R_{S31D}, —S(═O)₂N(R_{S31C})₂, —NR_{S31C}S(═O)₂R_{S31D}, —OS(═O)₂OR_{S31C}, —NR_{S31C}S(═O)₂OR_{S31C}, —OS(═O)₂N(R_{S31C})₂, —NR_{S31C}S(═O)₂N(R_{S31C})₂, —P(R_{S31C})₂, —P(═O)(R_{S31D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S31} together with the carbon atom to which they are both attached form

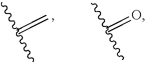

a 3-10 membered carbocyclic ring or a 3-10 heterocyclic ring; wherein, said

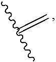

3-10 membered carbocylic ring or 3-10 heterocyclic ring is independently unsubstituted or substituted with one or more R_{S311};

Optionally, two adjacent R_{S31} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S312};

Optionally, two nonadjacent R_{S31} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S313};
m₃ is selected from 0, 1, 2, 3, 4, 5, 6, 7, 8, 9, 10, 11, or 12;
R_{S32} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32A})₂, —OR_{S32A}, —SR_{S32A}, —S(═O)R_{S32B}, —S(═O)₂R_{S32B}, —C(═O)R_{S32B}, —C(═O)₂OR_{S32A}, —OC(═O)R_{S32B}, —C(═O)N(R_{S32A})₂, —NR_{S32A}C(═O)R_{S32B}, —OC(═O)OR_{S32A}, —NR_{S32A}C(═O)OR_{S32A}, —NR_{S32A}C(═S)OR_{S32A}, —OC(═O)N(R_{S32A})₂, —NR_{S32A}C(═O)N(R_{S32A})₂, —S(═O)OR_{S32A}, —OS(═O)R_{S32B}, —S(═O)N(R_{S32A})₂, —NR_{S32A}S(═O)R_{S32B}, —S(═O)₂OR_{S32A}, —OS(═O)₂R_{S32B}, —S(═O)N(R_{S32A})₂, —NR_{S32A}S(═O)₂R_{S32B}, —OS(═O)₂OR_{S32A}, —NR_{S32A}S(═O)₂OR_{S32A}, —OS(═O)₂N(R_{S32A})₂, —NR_{S32A}S(═O)₂N(R_{S32A})₂, —P(R_{S32A})₂, —P(═O)(R_{S32B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32C})₂, —OR_{S32C}, —SR_{S32C}, —S(═O)R_{S32C}, —S(═O)₂R_{S32D}, —C(═O)R_{S32D}, —C(═O)OR_{S32C}, —OC(═O)R_{S32D}, —C(═O)N(R_{S32C})₂, —NR_{S32C}C(═O)R_{S32D}, —OC(═O)OR_{S32C}, —NR_{S32C}C(═O)OR_{S32C}, —NR_{S32C}C(═S)OR_{S32C}, —OC(═O)N(R_{S32C})₂, —NR_{S32C}C(═O)N(R_{S32C})₂, —S(═O)OR_{S32C}, —OS(═O)R_{S32C}, —S(═O)N(R_{S32C})₂, —NR_{S32C}S(═O)R_{S32D}, —S(═O)₂OR_{S32C}, —OS(═O)₂R_{S32D}, —S(═O)₂N(R_{S32C})₂, —NR_{S32C}S(═O)₂R_{S32D}, —OS(═O)₂OR_{S32C}, —NR_{S32C}S(═O)₂OR_{S32C}, —OS(═O)₂N(R_{S32C})₂, —NR_{S32C}S(═O)₂N(R_{S32C})₂, —P(R_{S32C})₂, —P(═O)(R_{S32D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S32} together with the carbon atom to which they are both attached form

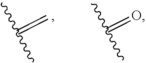

a 3-10 membered carbocyclic ring or a 3-10 heterocyclic ring; wherein, said

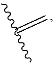

3-10 membered carbocyclic ring or 3-10 heterocyclic ring is independently unsubstituted or substituted with one or more R_{S321};

Optionally, two adjacent R_{S32} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S322};

Optionally, two nonadjacent R_{S32} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S323};
m₄ is selected from 0, 1, 2, 3, 4, 5 or 6;
R₃₇ is selected from —N(R_{37A})₂ or 3-10 membered heterocyclyl, wherein said 3-10 membered heterocyclyl is optionally independently substituted with one or more R_{S37};
R_{S38} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38A})₂, —OR_{S38A}, —SR_{S38A}, —S(═O)R_{S38B}, —S(═O)₂R_{S38B}, —C(═O)R_{S38B}, —C(═O)OR_{S38A}, —OC(═O)R_{S38B}, —C(═O)N(R_{S38A})₂, —NR_{S38A}C(═O)R_{S38B}, —OC(═O)OR_{S38A}, —NR_{S38A}C(═O)OR_{S38A}, —NR_{S38A}C(═S)OR_{S38A}, —OC(═O)N(R_{S38A})₂, —NR_{S38A}C(═O)N(R_{S38A})₂, —S(═O)OR_{S38A}, —OS(═O)R_{S32B}, —S(═O)N(R_{S38A})₂, —NR_{S38A}S(═O)R_{S38B}, —S(═O)₂OR_{S38A}, —OS(═O)₂R_{S38B}, —S(═O)₂N(R_{S38A})₂, —NR_{S38A}S(═O)₂R_{S38B}, —OS(═O)₂OR_{S38A}, —NR_{S38A}S(═O)₂OR_{S38A}, —OS(═O)₂N(R_{S38A})₂, —NR_{S38A}S(═O)₂N(R_{S38A})₂, —P(R_{S38A})₂, —P(═O)(R_{S38B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₂₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38C})₂, —OR_{S38C}, —SR_{S38C}, —S(═O)R_{S38D}, —S(═O)₂R_{S38D}, —C(═O)R_{S38C}, —C(═O)OR_{S38C}, —OC(═O)R_{S38D}, —C(═O)N(R_{S38C})₂, —NR_{S38C}C(═O)R_{S38D}, —OC(═O)OR_{S38C}, —NR_{S38C}C(═O)OR_{S38C}, —NR_{S38C}C(═S)OR_{S38C}, —OC(═O)N(R_{S38C})₂, —NR_{S38C}C(═O)N(R_{S38C})₂, —S(═O)OR_{S38C}, —OS(═O)R_{S38D}, —S(═O)N(R_{S38C})₂, —NR_{S38C}S(═O)R_{S38D}, —S(═O)₂OR_{S38C}, —OS(═O)₂R_{S38D}, —S(═O)₂N(R_{S38C})₂, —NR_{S38C}S(═O)₂R_{S38D}; —OS(═O)₂OR_{S38C}, —NR_{S38C}S(═O)₂OR_{S38C}, —OS(═O)₂N(R_{S38C})₂, —P(R_{S38C})₂, —P(═O)(R_{S38D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S38} together with the carbon atom to which they are both attached form

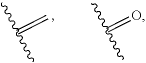

a 3-10 membered carbocyclic ring or a 3-10 membered heterocyclic ring; wherein, said

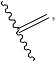

3-10 membered carbocylic ring or 3-10 heterocyclic ring is independently unsubstituted or substituted with one or more R_{S381};

Optionally, two adjacent R_{S38} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S382};

Optionally, two nonadjacent R_{S38} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S383};
m₈ is selected from 0, 1, 2, 3, 4, 5, 6, 7, 8, 9, 10, 11, or 12;
R_{S39} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S394})₂, —OR_{S39A}, —SR_{S39A}, —S(═O)R_{S39B}, —S(═O)₂R_{S39B}, —C(═O)R_{S39B}, —C(═O)₂OR_{S39A}, —OC(═O)R_{S39B}, —C(═O)N(R_{S39A})₂, —NR_{S39A}C(═O)R_{S39B}, —OC(═O)OR_{S39A}, —NR_{S39A}C(═O)OR_{S39A}, —NR_{S39A}C(═S)OR_{S39A}, —OC(═O)N(R_{S39A})₂, —NR_{S39A}C(═O)N(R_{S39A})₂, —S(═O)OR_{S39A}, —OS(═O)R_{S39B}; —S(═O)N(R_{S39A})₂, —NR_{S39A}S(═O)R_{S39B}, —S(═O)₂OR_{S39A}, —OS(═O)₂R_{S39B}, —S(═O)N(R_{S39A})₂, —NR_{S39A}S(═O)₂R_{S39B}, —OS(═O)₂OR_{S39A}, —NR_{S39A}S(═O)₂OR_{S39A}, —OS(═O)₂N(R_{S39A})₂, —NR_{S39A}S(═O)₂N(R_{S39A})₂, —P(R_{S39A})₂, —P(═O)(R_{S39B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39C})₂, —OR_{S39C}, —SR_{S39C}, —S(═O)R_{S39C}, —S(═O)₂R_{S39D}, —C(═O)R_{S39D}, —C(═O)OR_{S39C}, —OC(═O)R_{S39D}, —C(═O)N(R_{S39C})₂, —NR_{S39C}C(═O)R_{S39D}, —OC(═O)OR_{S39C}, —NR_{S39C}C(═O)OR_{S39C}, —NR_{S39C}C(═S)OR_{S39C}, —OC(═O)N(R_{S39C})₂, —NR_{S39C}C(═O)N(R_{S39C})₂, —S(═O)OR_{S39C}, —OS(═O)R_{S39C}, —S(═O)N(R_{S39C})₂, —NR_{S39C}S(═O)R_{S39D}, —S(═O)₂OR_{S39C}, —OS(═O)₂R_{S39D}, —S(═O)₂N(R_{S39C})₂, —NR_{S39C}S(═O)₂R_{S39D}, —OS(═O)₂N(R_{S39C})₂, —NR_{S39C}S(═O)₂N(R_{S39C})₂, —P(R_{S39C})₂, —OS(═O)₂OR_{S39C}, —NR_{S39C}S(═O)₂OR_{S39C}, —P(═O)(R_{S39D}))₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S39} together with the carbon atom to which they are both attached form

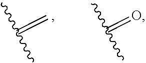

a 3-10 membered carbocyclic ring or a 3-10 heterocyclic ring; wherein, said

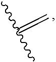

3-10 membered carbocylic ring or 3-10 heterocyclic ring is independently unsubstituted or substituted with one or more R_{S391};

Optionally, two adjacent R_{S39} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S392};

Optionally, two nonadjacent R_{S39} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂, the hydrogen on the each of carbon atoms or N atoms is independently unsubstituted or substituted with R_{S393},
m₉ is selected from 0, 1, 2, 3, 4, 5 or 6;
R_{S315} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315A})₂, —OR_{S315A}, —SR_{S315A}, —S(═O)R_{S315B}, —C(═O)₂R_{S315B}, —C(═O)OR_{S315A}, —OC(═O)R_{S315B}, —C(═O)N(R_{S315A})₂, —NR_{S315A}C(═O)R_{S315B}, —OC(═O)OR_{S315A}, —NR_{S315A}C(═O)OR_{S315A}, —NR_{S315A}C(═S)OR_{S315A}, —OC(═O)N(R_{S315A})₂, —NR_{S315A}C(═O)N(R_{S315A})₂, —S(═O)OR_{S315A}, —OS(═O)R_{S315B}, —NR_{S315A}S(═O)₂R_{S315B}, —S(═O)₂OR_{S315A}, —OS(═O)₂R_{S315B}, —S(═O)₂N(R_{S315A})₂, —OS(═O)₂OR_{S315A}, —S(═O)N(R_{S315A})₂, —NR_{S315A}S(═O)R_{S315B}, —NR_{S315A}S(═O)₂OR_{S315A}, —OS(═O)₂N(R_{S315A})₂, —NR_{S315A}S(═O)₂N(R_{S315A})₂, —P(R_{S315A})₂, —P(═O)(R_{S315B})₂, 3- 10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315C})₂, —OR_{S315C}, —SR_{S315C}, —S(═O)R_{S315C}, —S(═O)₂R_{S315D}, —C(═O)R_{S315D}, —C(═O)OR_{S315C}, —OC(═O)R_{S315D}, —C(═O)N(R_{S315C})₂, —NR_{S315C}C(═O)R_{S315D}, —OC(═O)OR_{S315C}, —NR_{S315C}C(═O)OR_{S315C}, —NR_{S315C}C(═S)OR_{S315C}, —OC(═O)N(R_{S315C})₂, —NR_{S315C}C(═O)N(R_{S315C})₂, —S(═O)OR_{S315C}, —OS(═O)R_{S315C}, —S(═O)N(R_{S315C})₂, —NR_{S315C}S(═O)R_{S315D}, —S(═O)₂OR_{S315C}, —OS(═O)₂R_{S315D}, —S(═O)₂N(R_{S315C})₂, —NR_{S315C}S(═O)₂R_{S315D}, —OS(═O)₂OR_{S315C}, —NR_{S315C}S(═O)₂OR_{S315C}, —OS(═O)₂N(R_{S315C})₂, —NR_{S315C}S(═O)₂N(R_{S315C})₂, —P(R_{S315C})₂, —P(═O)(R_{S315D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S315} together with the carbon atom to which they are both attached form

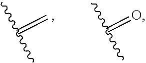

a 3-10 membered carbocyclic ring or a 3-10 heterocyclic ring; wherein, said

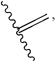

3-10 membered carbocylic ring or 3-10 heterocyclic ring is independently unsubstituted or substituted with one or more R_{S3151};

Optionally, two adjacent R_{S315} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S3152};

Optionally, two nonadjacent R_{S315} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S3153};
m₁₀ is selected from 0, 1, 2, 3, 4, 5 or 6;
R₄ is selected from 6-10 membered aryl, 5-10 membered heteroaryl,

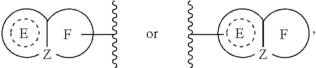

wherein said 6-10 membered aryl, 5-10 membered heteroaryl,

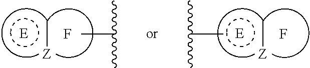

is independently unsubstituted or substituted with one or more R_{S4};
Z at each occurrence is independently selected from C or N;
Ring E at each occurrence is independently selected from a 6 membered aryl ring or a 5-6 membered heteroaryl ring and ring F at each occurrence is a 3-10 membered carbocyclic ring or a 3-10 membered heterocyclic ring when Z is selected from C;
Ring E at each occurrence is selected from a 5-6 membered heteroaryl ring and ring F at each occurrence is a 3-10 membered heterocyclic ring when Z is selected from N;
R_{S4} is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4A})₂, —OR_{S4A}, —SR_{S4A}, —S(═O)R_{S4B}, —S(═O)₂R_{S4B}, —C(═O)R_{S4B}, —C(═O)OR_{S4A}, —OC(═O)R_{S4B}, —C(═O)N(R_{S4A})₂, —NR_{S4C}(═O)R_{S4B}, —OC(═O)OR_{S4A}, —NR_{S4A}C(═O)OR_{S4A}, —NR_{S4A}C(═S)OR_{S4A}, —OC(═O)N(R_{S4A})₂, —NR_{S4A}C(═O)N(R_{S4A})₂, —S(═O)OR_{S4A}, —OS(═O)R_{S4B}, —S(═O)N(R_{S4A})₂, —NR_{S4A}S(═O)R_{S4B}, —OS(═O)₂R_{S4B}, —S(═O)₂OR_{S4A}, —S(═O)₂N(R_{S4A})₂, —NR_{S4A}S(═O)₂R_{S4B}, —OS(═O)₂OR_{S4A}, —NR_{S4A}S(═O)₂OR_{S4A}, —OS(═O)₂N(R_{S4A})₂, —NR_{S4A}S(═O)₂N(R_{S4A})₂, —P(R_{S4A})₂, —P(═O)(R_{S4B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4C})₂, —OR_{S4C}, —SR_{S4C}, —S(═O)R_{S4D}, —S(═O)₂R_{S4D}, —C(═O)R_{S4C}, —C(═O)OR_{S4D}, —OC(═O)R_{S4D}, —C(═O)N(R_{S4C})₂, —NR_{S4C}C(═O)R_{S4D}, —OC(═O)OR_{S4C}, —NR_{S4C}C(═O)OR_{S4C}, —NR_{S4C}C(═S)OR_{S4C}, —OC(═O)N(R_{S4C})₂, —NR_{S4C}C(═O)N(R_{S4C})₂, —S(═O)OR_{S4C}, —OS(═O)R_{S4D}, —S(═O)N(R_{S4C})₂, —NR_{S4C}S(═O)R_{S4D}, —S(═O)₂OR_{S4C}, —OS(═O)₂R_{S4D}, —S(═O)N(R_{S4C})₂, —NR_{S4C}S(═O)₂R_{S4D}, —OS(═O)₂OR_{S4C}, —NR_{S4C}S(═O)₂OR_{S4C}, —OS(═O)₂N(R_{S4C})₂, —NR_{S4C}S(═O)₂N(R_{S4C})₂, —P(R_{S4C})₂, —P(═O)(R_{S4D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R₅ is selected from hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5A})₂, —OR_{5A}, —SR_{5A}, —S(═O)R_{5B}, —S(═O)₂R_{5B}, —C(═O)R_{5B}, —C(═O)OR_{5A}, —OC(═O)R_{5B}, —C(═O)N(R_{5A})₂, —NR_{5A}C(═O)R_{5B}, —OC(═O)OR_{5A}, —NR_{5A}C(═O)OR_{5A}, —NR_{5A}C(═S)OR_{5A}, —OC(═O)N(R_{5A})₂, —NR_{5A}C(═O)N(R_{5A})₂, —S(═O)OR_{5A}, —OS(═O)R_{5B}, —S(═O)N(R_{5A})₂, —NR_{5A}S(═O)R_{5B}, —S(═O)₂OR_{5A}, —OS(═O)₂R_{5B}, —S(═O)₂N(R_{5A})₂, —NR_{5A}S(═O)₂R_{5B}, —OS(═O)₂OR_{5A}, —NR_{5A}S(═O)₂OR_{5A}, —OS(═O)₂N(R_{5A})₂, —NR_{5A}S(═O)₂N(R_{5A})₂, —P(R_{5A})₂, —P(═O)(R_{5B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₁₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5C})₂, —OR_{5C}, —SR_{5C}, —S(═O)R_{5D}, —S(═O)₂R_{5D}, —C(═O)R_{5D}, —C(═O)OR_{5D}, —OC(═O)R_{5D}, —C(═O)N(R_{5C})₂, —NR_{5C}C(═O)R_{5D}, —OC(═O)OR_{5C}, —NR_{5C}C(═O)OR_{5C}, —NR_{5C}C(═S)OR_{5C}, —OC(═O)N(R_{5C})₂, —NR_{5C}C(═O)N(R_{5C})₂, —S(═O)OR_{5C}, —OS(═O)R_{5D}, —S(═O)N(R_{5C})₂, —NR_{5C}S(═O)R_{5D}, —S(═O)₂OR_{5C}, —OS(═O)₂R_{5D}, —S(═O)₂N(R_{5C})₂, —NR_{5C}S(═O)₂R_{5D}, —OS(═O)₂OR_{5C}, —NR_{5C}S(═O)₂OR_{5C}, —OS(═O)₂N(R_{5C})₂, —NR_{5C}S(═O)₂N(R_{5C}), —P(R_{5C})₂, —P(═O)(R_{5D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Each of R_{1A}, R_{1C}, R_{S1A}, R_{S1C}, R_{S31A}, R_{S31C}, R_{S32A}, R_{S32C}, R_{37A}, R_{S38A}, R_{S38C}, R_{S39A}, R_{S39C}, R_{S315A}, R_{S315C}, R_{S4A}, R_{S4C}, R_{5A} and R_{5C} is independently selected from hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)OR_{C}, —OC(═O)R_{D}, —C(═O)N(R_{C})₂, —NR_{C}C(═O)R_{D}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{C}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, (two R_{1A}, two R_{1C}, two R_{S1A}, two R_{S1C}, two R_{S31A}, two R_{S31C}, two R_{S32A}, two R_{S32C}, two R_{37A}, two R_{S38A}, two R_{S38C}, two R_{S39A}, two R_{S39C}, two R_{S315A}, two R_{S315C}, two R_{S4A}, two R_{S4C}, two R_{5A} and two R_{5C}) together with the nitrogen atom to which they are both attached forms a 3-10 membered heterocyclic ring or a 5-10 membered heteroaryl ring, wherein, said 3-10 membered heterocyclic ring or 5-10 membered heteroaryl ring is independently unsubstituted or substituted with one or more R_{SS};

Each of R_{1B}, R_{1D}, R_{S1B}, R_{S1D}, R_{S31B}, R_{S31D}, R_{S32B}, R_{S32D}, R_{S38B}, R_{S38D}, R_{S39B}, R_{S39D}, R_{S315B}, R_{S315D}, R_{S4B}, R_{S4D}, R_{5B} and R_{5D} is independently selected from hydrogen, deuterium, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C}), —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Each of (R_{A}, R_{B}, R_{C} and R_{D}) is independently selected from hydrogen, deuterium, —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubsituted or substituted with one or more R_{SA};

Each of R_{SX1}, R_{S11}, R_{S12}, R_{S13}, R_{S33}, R_{S34}, R_{S35}, R_{S316}, R_{S311}, R_{S312}, R_{S313}, R_{S321}, R_{S322}, R_{S323}, R_{S37}, R_{S310}, R_{S381}, R_{S382}, R_{S383}, R_{S391}, R_{S392}, R_{S393}, R_{S3151}, R_{S3152}, R_{S3153}, R_{SS} and R_{SA} is independently selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂N(C₁₋₆alkyl)₂, —NHS(═O)₂(C₁₋₆alkyl), —N(C₁₋₆alkyl)S(═O)₂ (C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)NH₂, —OS(═O)₂NH(C₁₋₆alkyl), —OS(═O)₂N(C₁₋₆alkyl)₂, —NHS(═O)NH₂, —NHS(═O)₂NH(C₁₋₆alkyl), —N(C₁₋₆alkyl)S(═O)₂NH(C₁₋₆alkyl), —NHS(═O)₂N(C₁₋₆alkyl)₂, —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Each of heterocyclyl or heterocyclic at each occurrence independently contains 1, 2, 3 or 4 heteroatoms selected from N, O, S, S(═O) or S(═O)₂;

Each of heteroaryl at each occurrence independently contains 1, 2, 3 or 4 heteroatoms selected from N, O, or S.
[2]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of [1], wherein, the compound is selected from any one of following formulas:

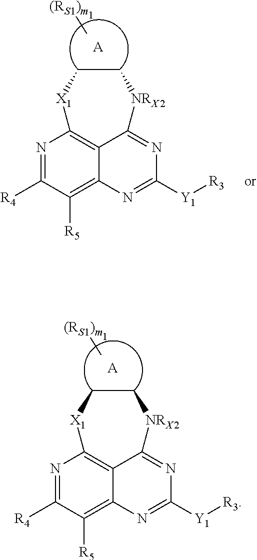

[3]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of [1] or [2], wherein:
X₁ at each occurrence is independently selected from —C(R_{X11})(R_{X12})—, —NR_{X13}—, —O— or —S—;
R_{X11} or R_{X12} is independently selected from hydrogen, deuterium, halogen or —C₁₋₆alkyl; wherein said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly;
R_{X13} is selected from hydrogen, deuterium or —C₁₋₆alkyl; wherein said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly.
[4]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [3], wherein, X₁ is selected from —O—, —S—, —NH—, —N(CH₃)—, —CH₂— or —CHF—.
[5]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [4], wherein, R_{X2} is selected from hydrogen, deuterium, or —C₁₋₆alkyl;
Preferably, R_{X2} is selected from hydrogen, deuterium, or methyl.
[6]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [5], wherein, ring A is selected from 5-6 membered carbocyclic ring or 5-6 heterocyclic ring containing 1 or 2 heteroatoms selected from N or O.
[7]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [6], wherein, the compound is selected from any one of the following formulas:

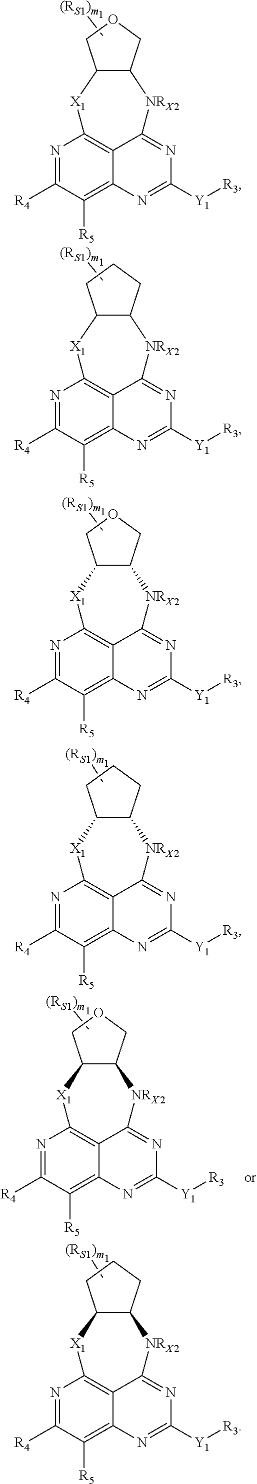

[8]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [7], wherein, m₁ is selected from 0, 1 or 2.
[9]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [8], wherein:
R_{S1} is selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly; wherein said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly;
Preferably, R_{S1} is selected from —F, —CH₃ or —OH.
[10]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [9], wherein, the compound is selected from any one of the following formulas in Table A:

TABLE A

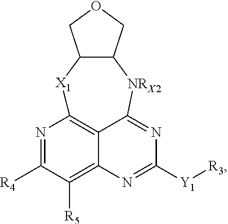

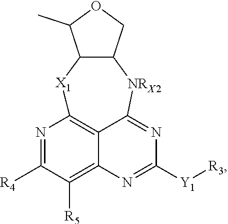

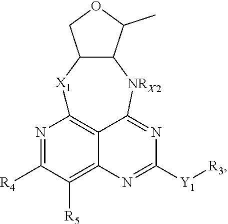

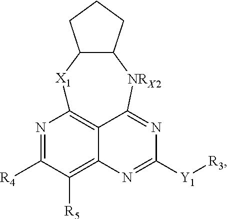

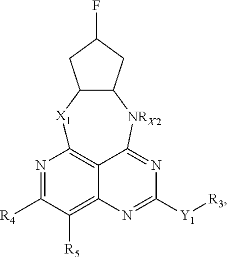

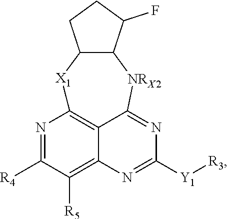

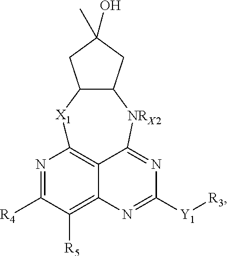

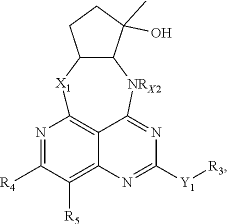

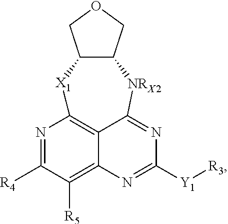

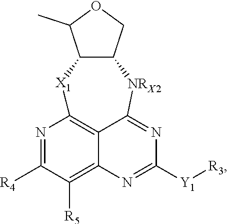

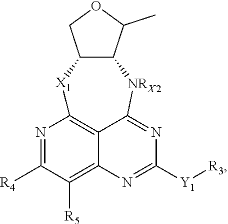

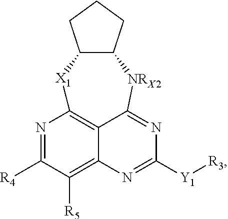

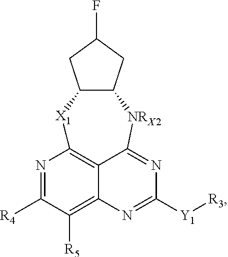

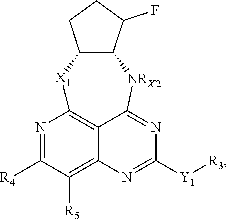

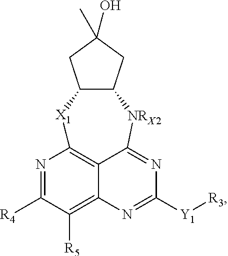

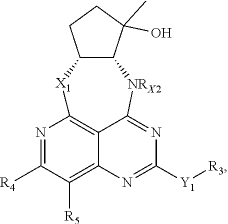

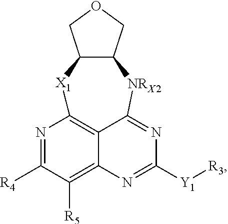

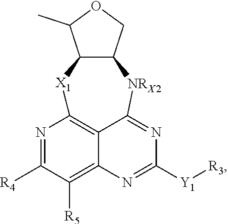

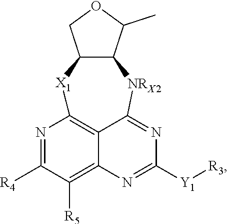

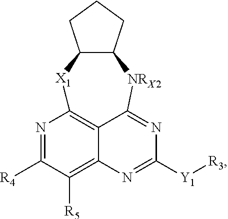

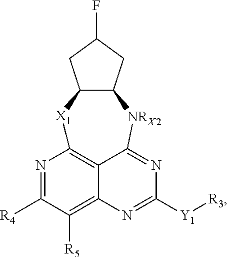

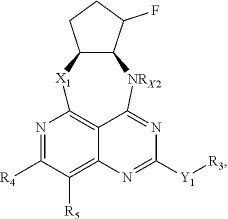

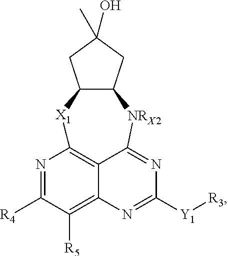

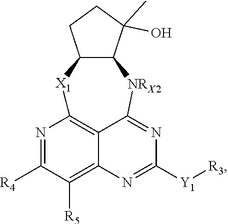

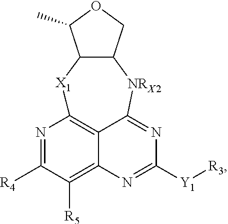

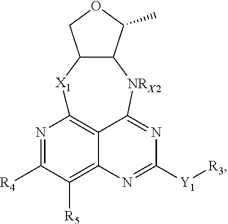

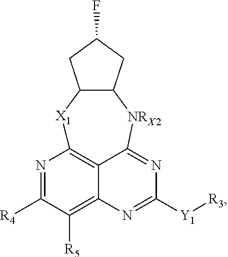

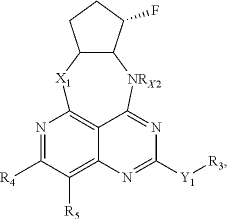

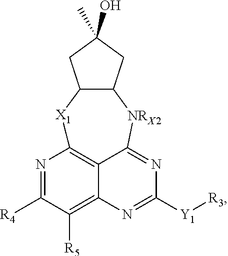

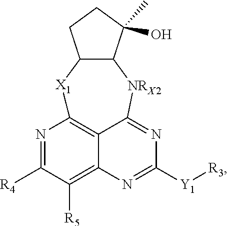

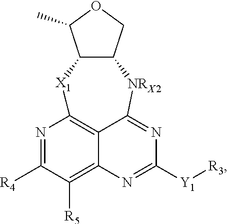

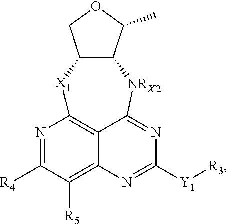

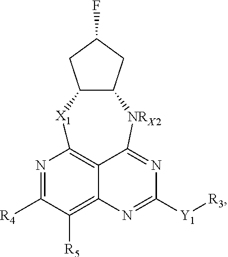

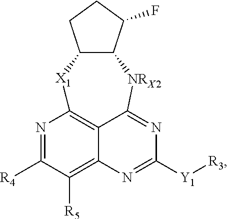

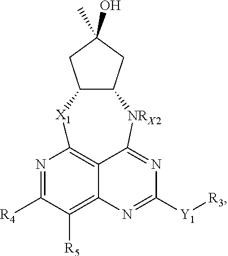

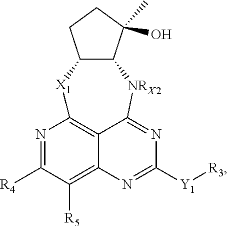

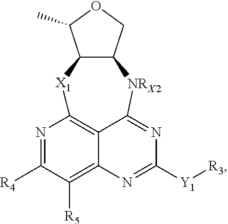

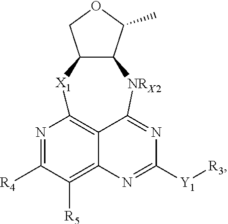

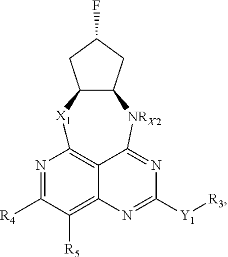

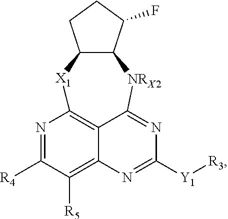

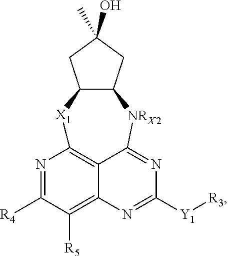

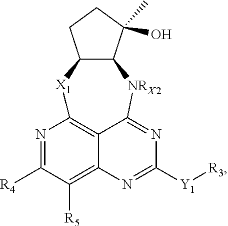

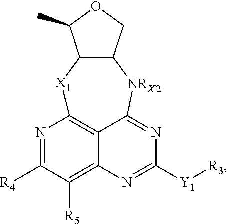

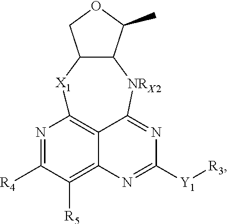

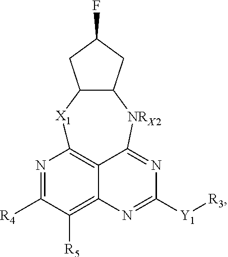

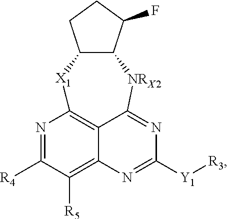

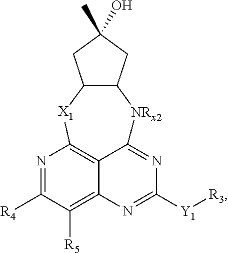

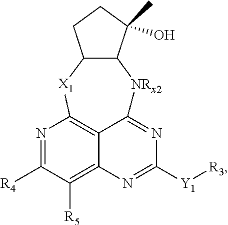

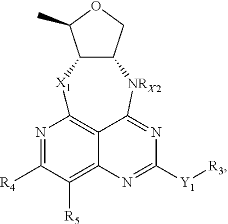

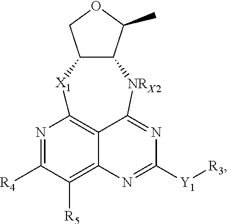

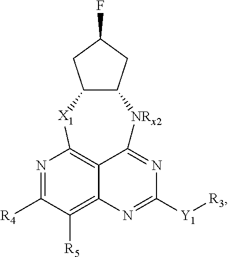

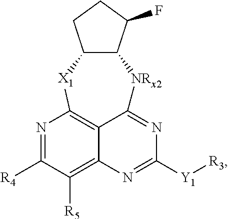

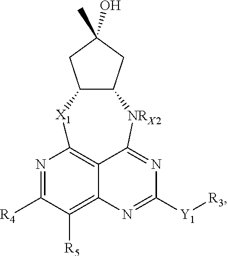

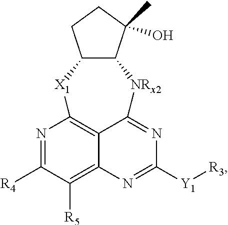

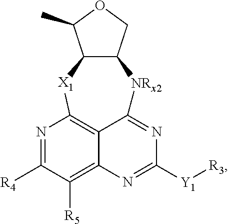

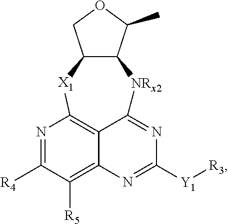

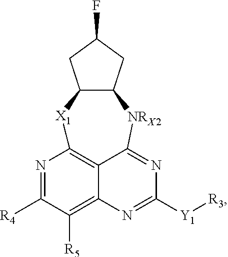

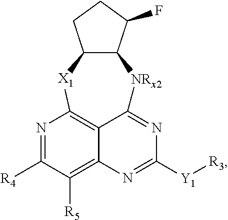

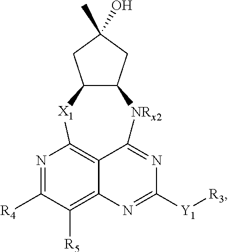

or

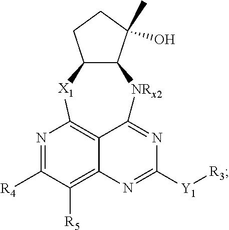

Preferably, the compound is selected from any one of the following formulas in Table B:

TABLE B

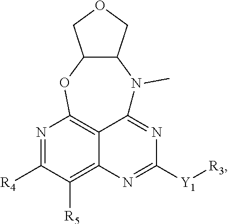

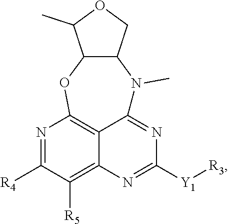

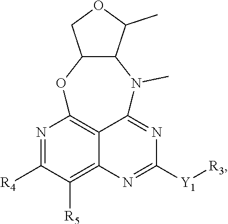

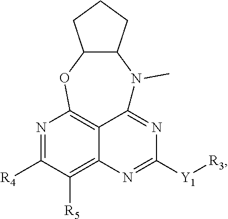

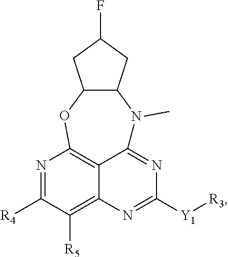

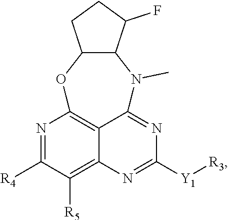

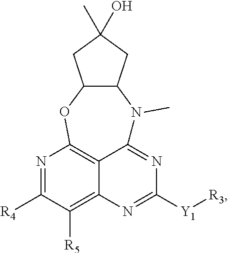

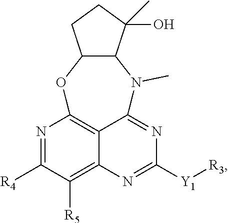

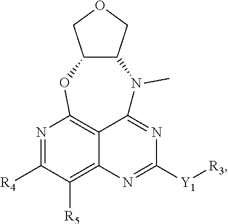

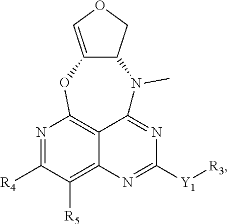

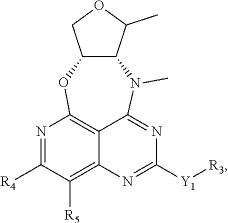

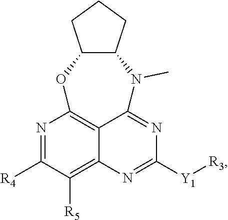

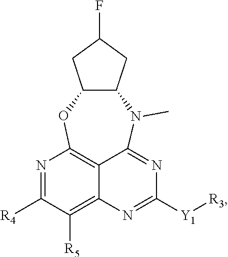

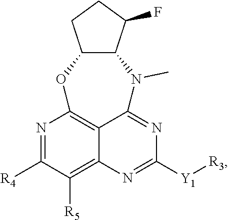

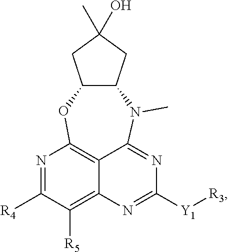

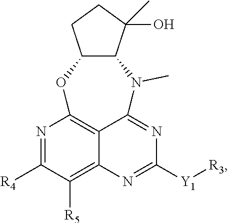

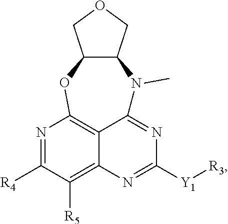

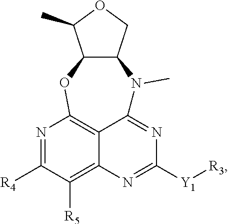

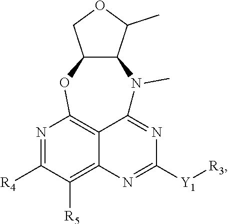

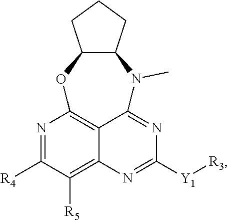

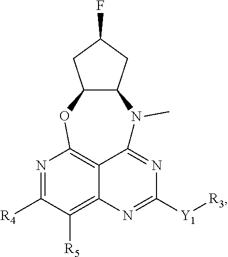

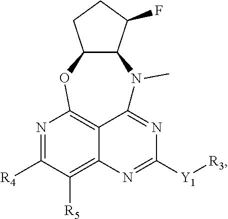

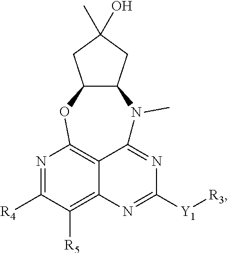

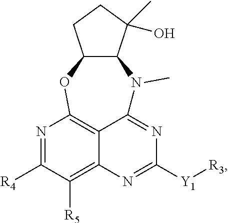

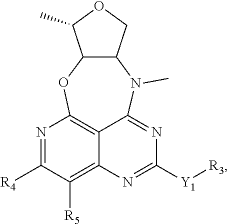

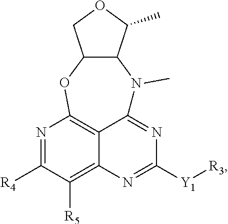

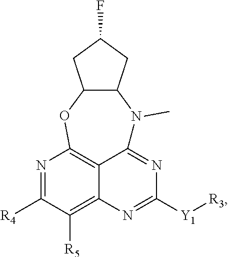

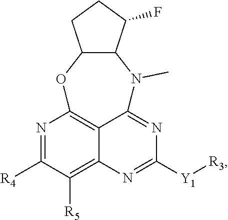

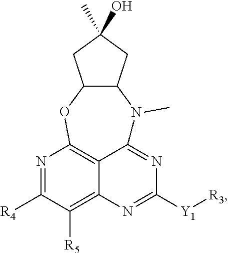

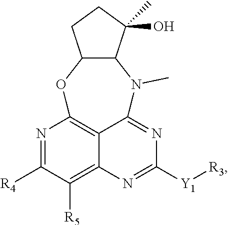

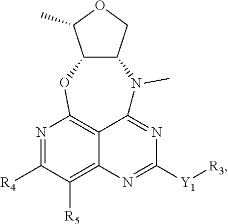

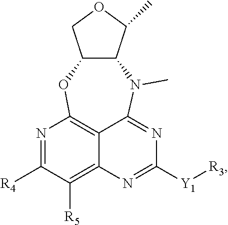

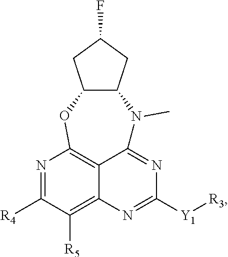

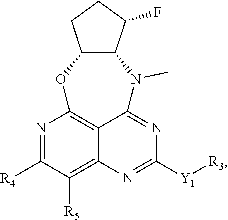

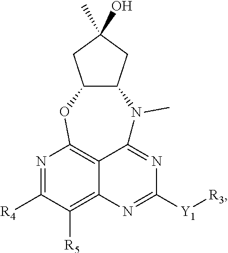

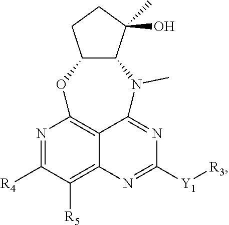

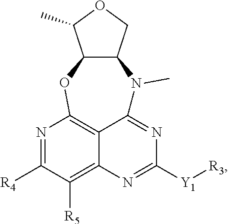

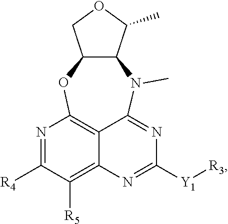

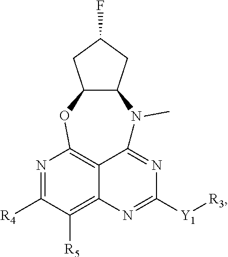

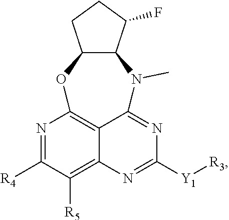

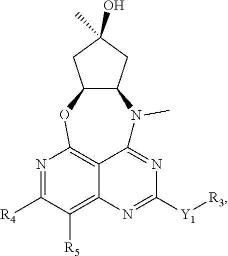

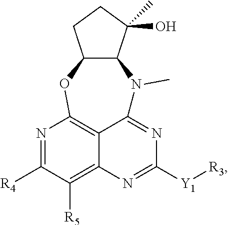

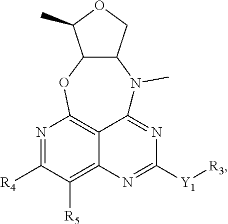

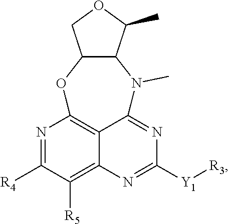

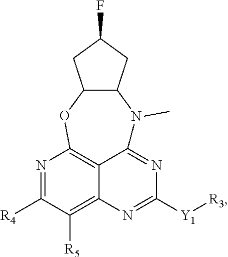

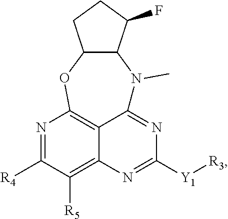

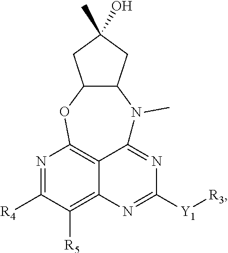

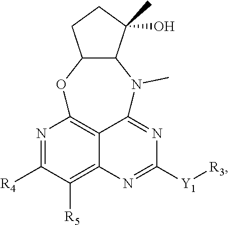

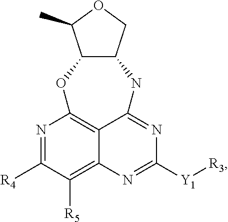

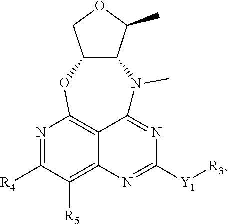

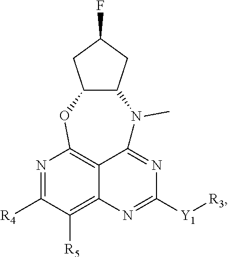

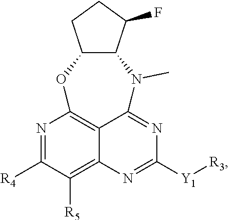

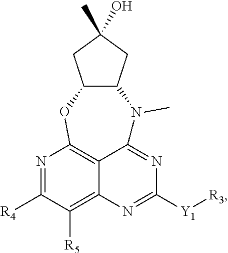

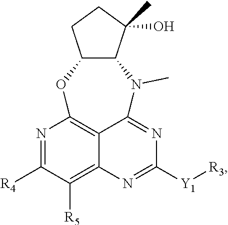

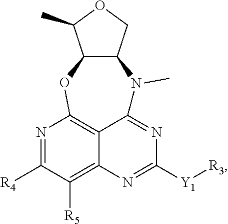

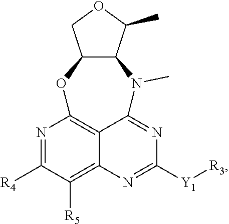

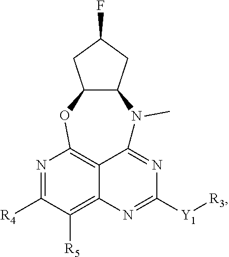

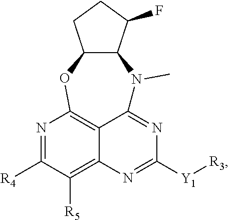

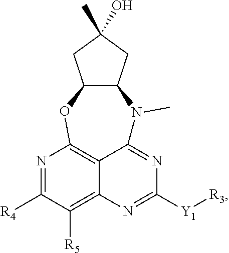

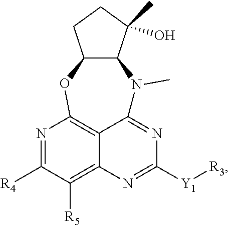

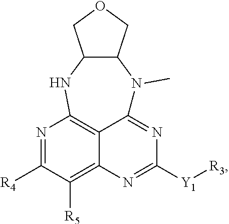

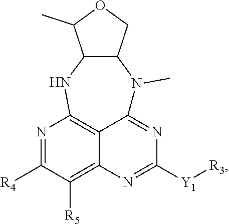

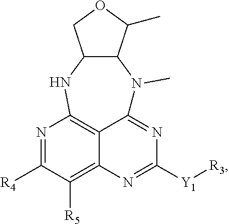

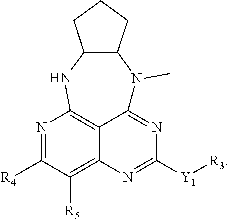

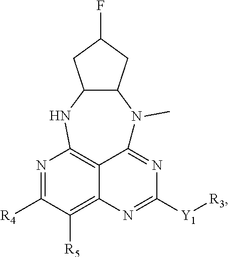

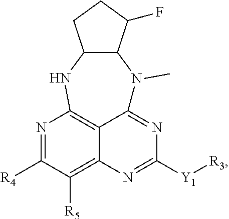

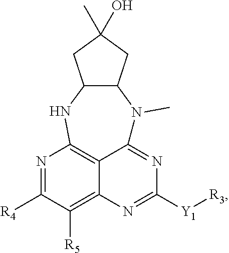

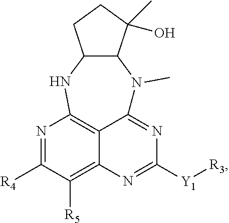

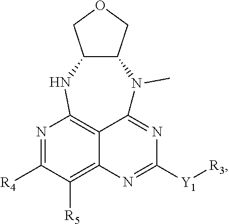

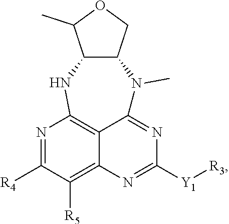

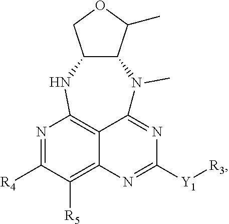

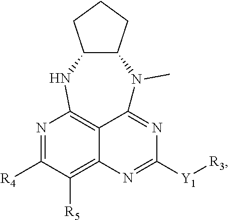

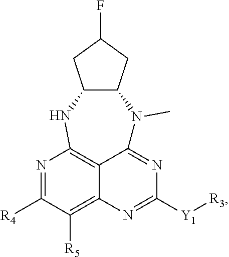

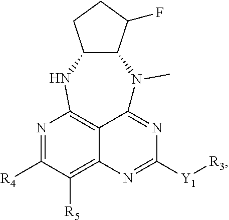

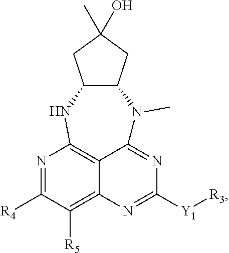

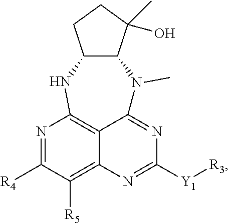

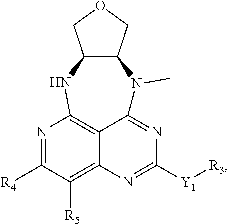

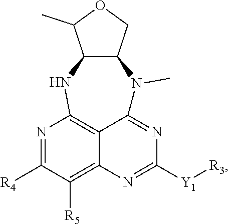

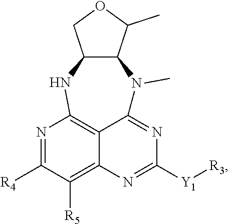

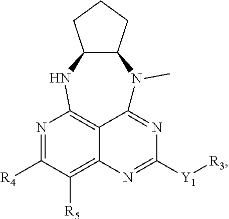

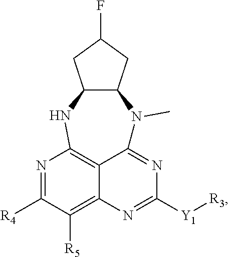

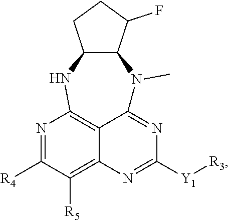

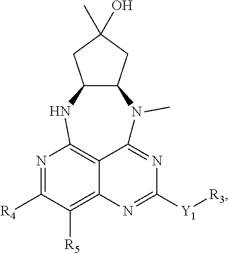

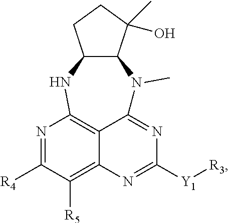

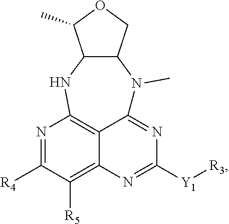

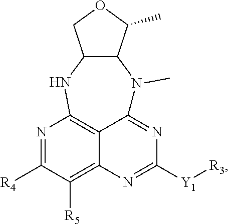

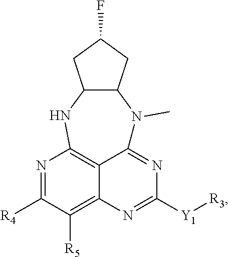

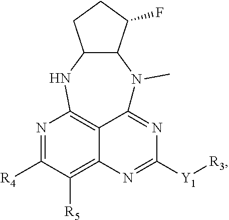

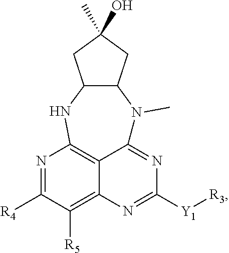

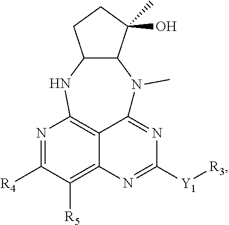

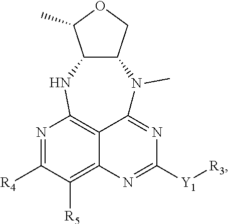

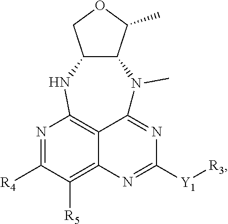

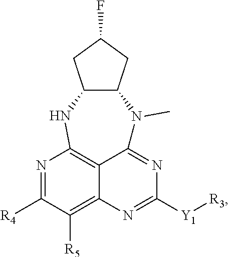

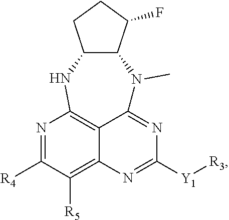

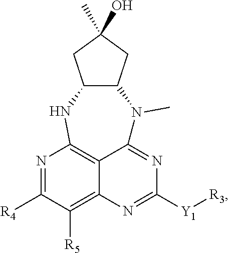

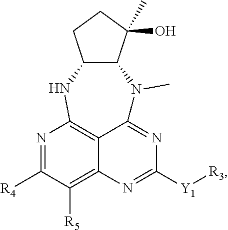

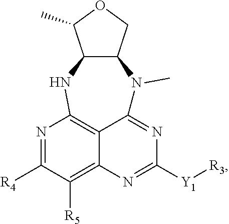

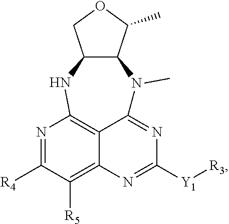

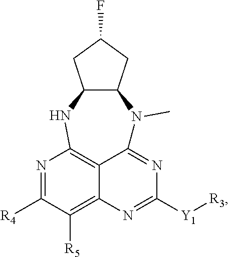

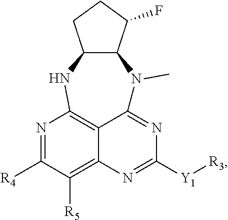

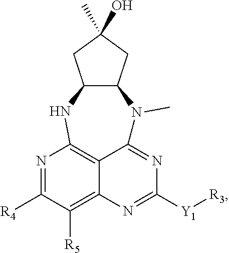

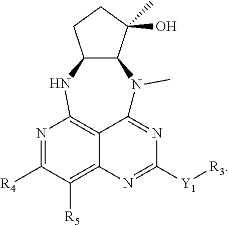

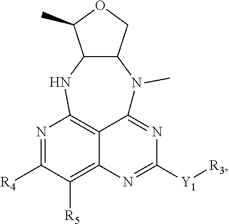

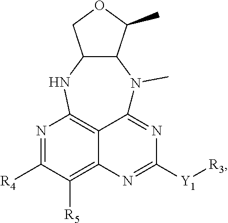

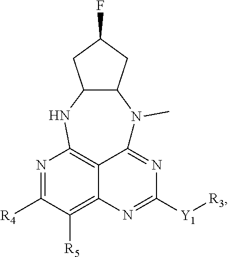

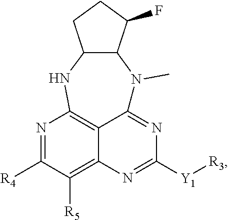

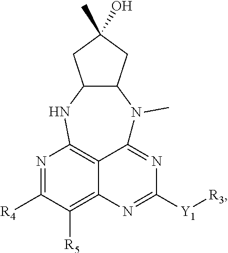

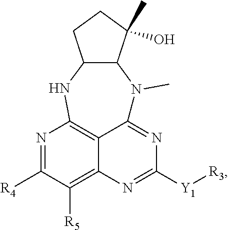

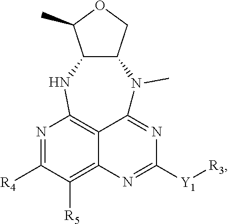

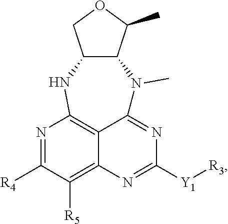

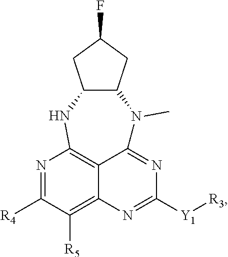

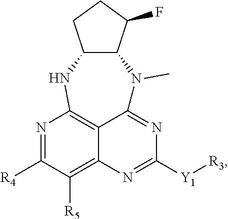

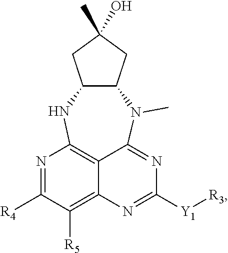

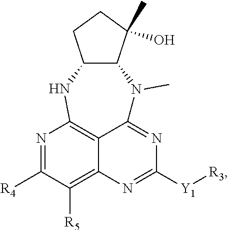

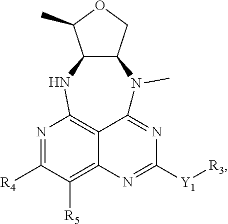

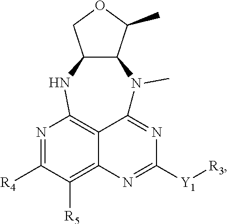

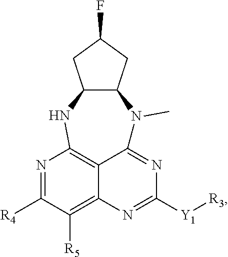

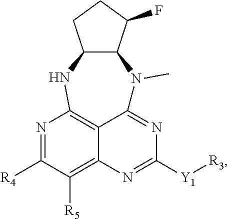

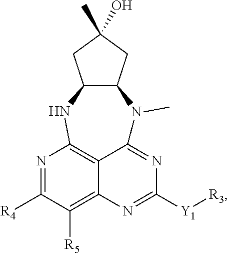

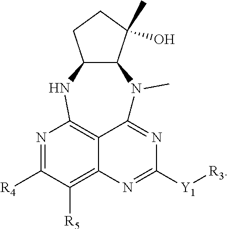

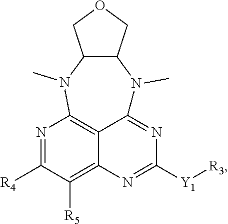

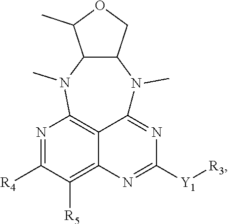

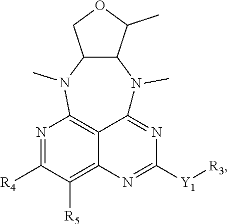

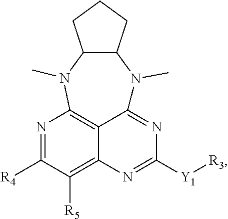

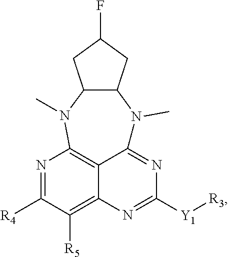

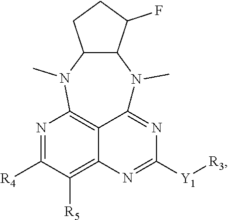

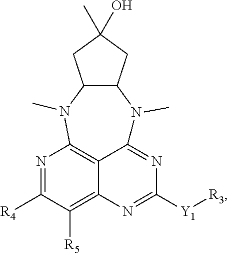

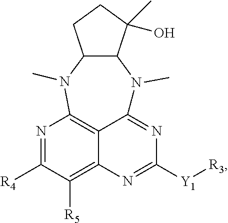

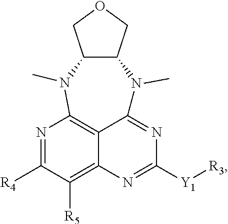

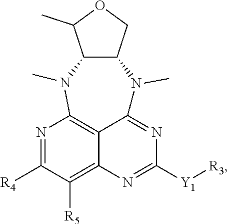

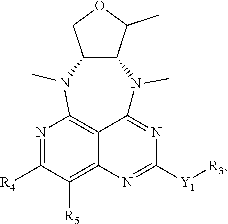

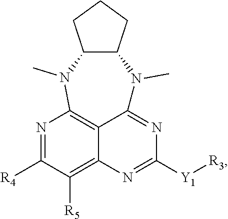

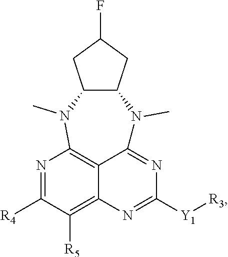

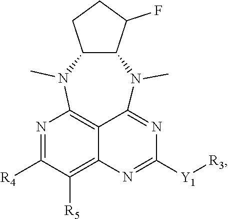

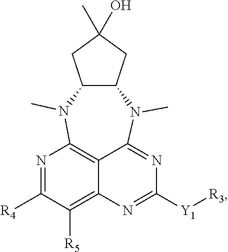

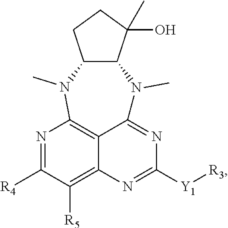

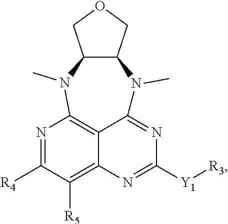

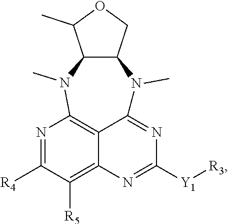

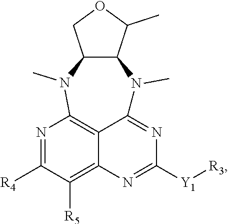

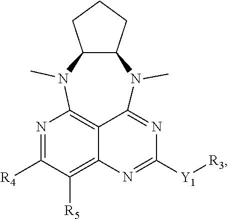

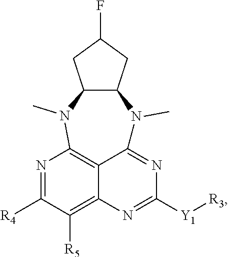

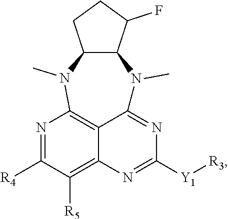

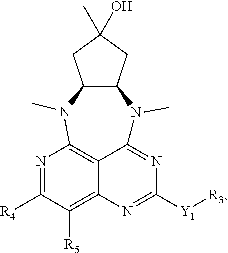

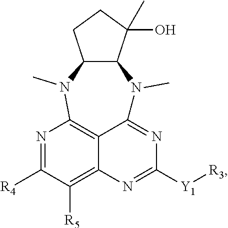

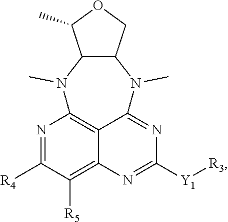

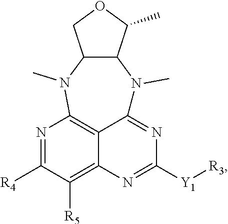

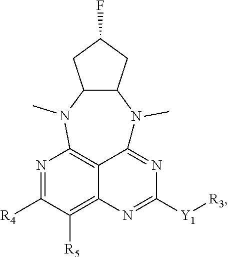

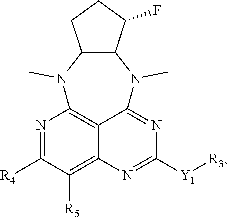

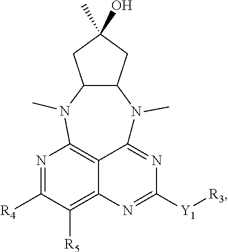

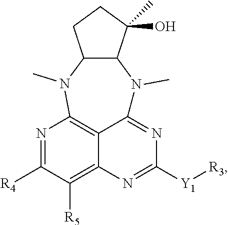

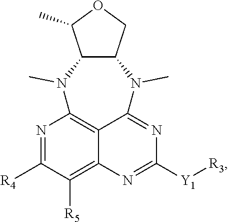

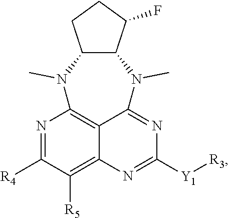

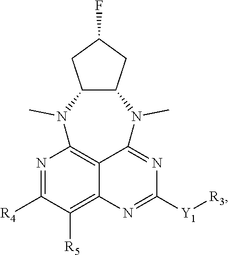

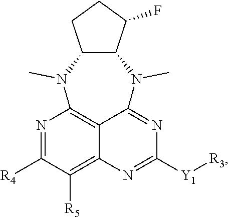

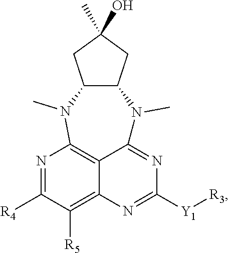

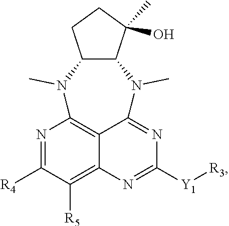

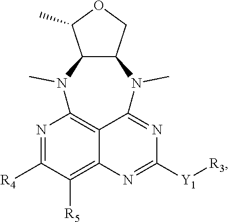

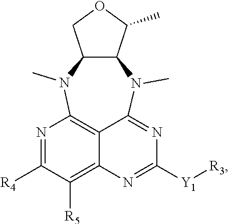

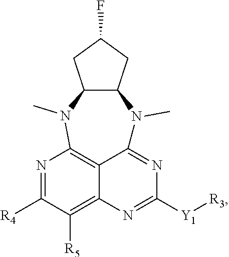

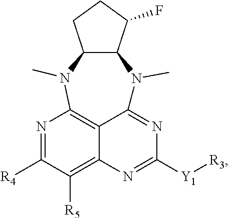

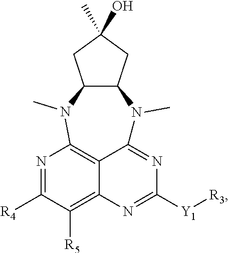

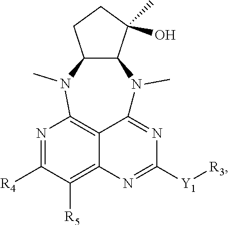

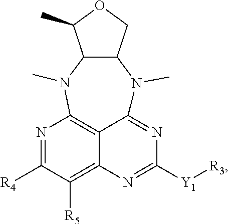

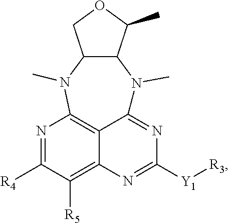

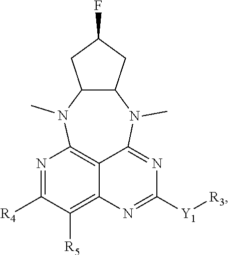

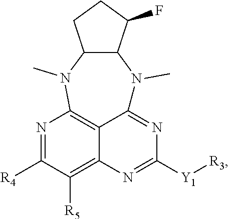

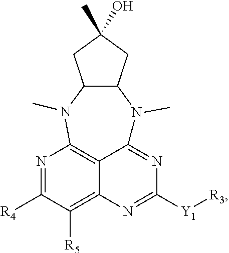

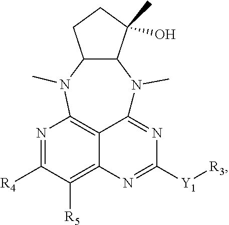

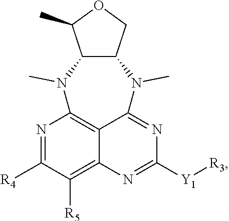

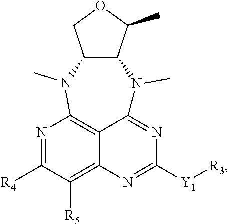

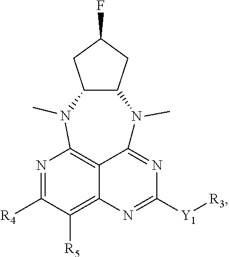

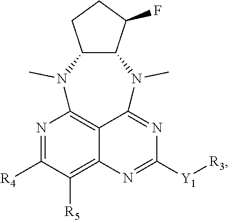

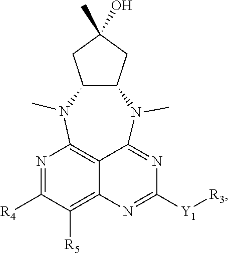

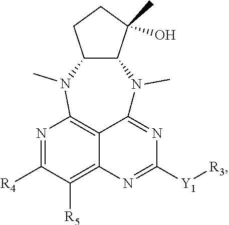

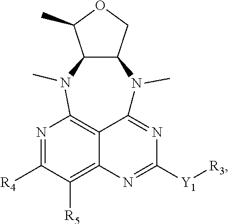

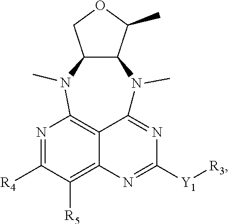

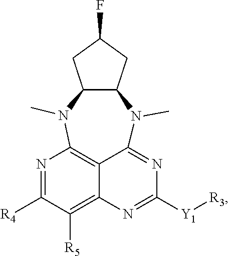

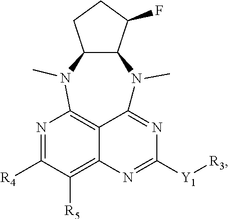

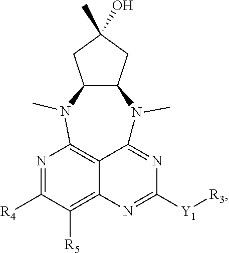

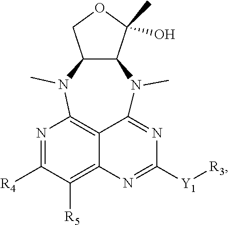

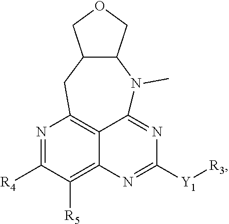

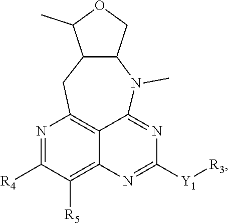

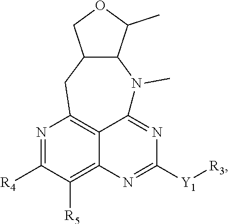

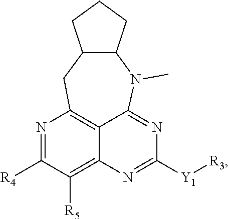

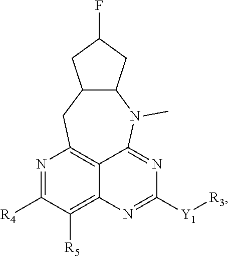

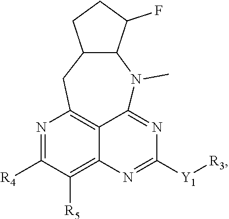

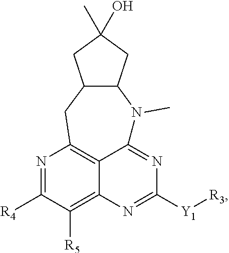

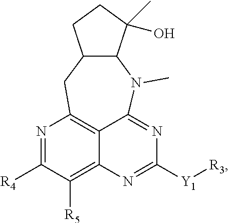

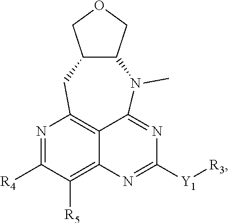

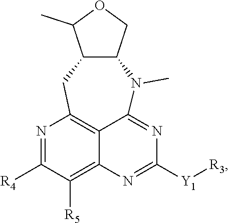

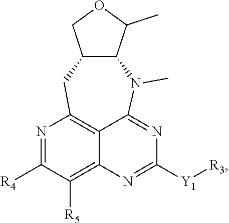

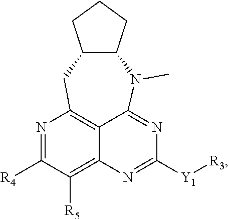

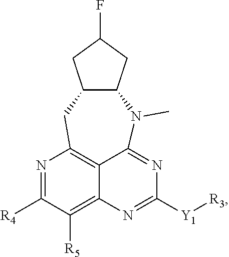

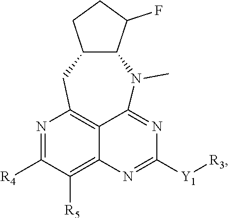

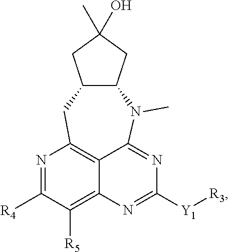

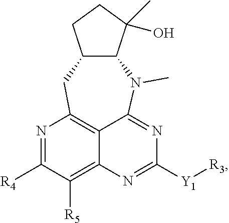

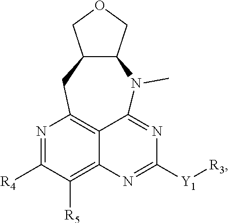

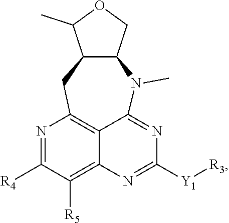

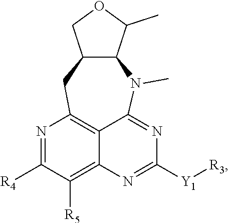

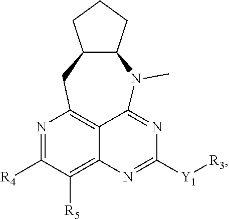

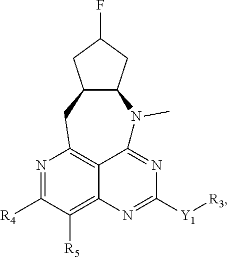

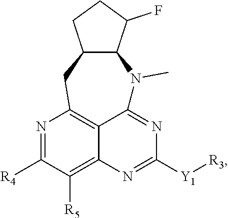

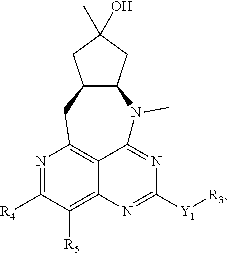

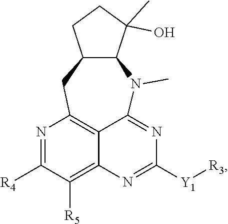

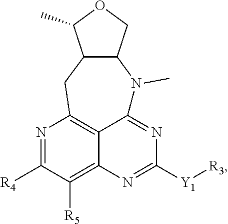

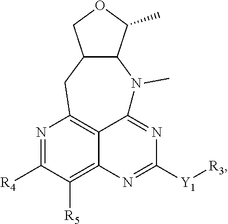

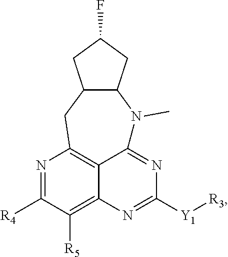

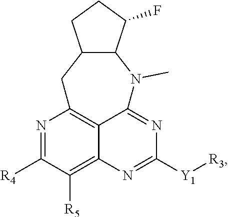

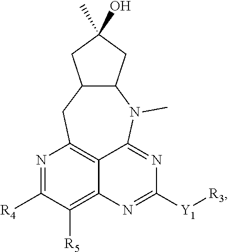

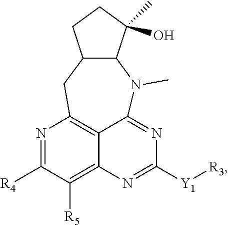

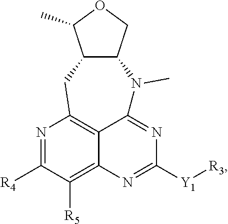

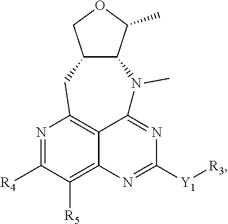

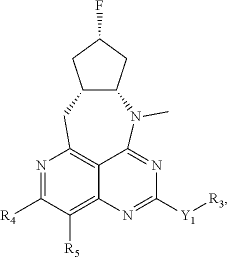

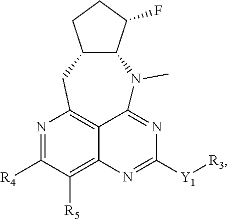

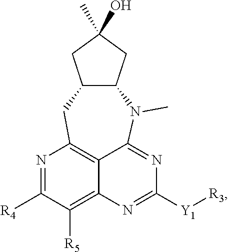

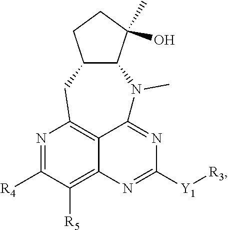

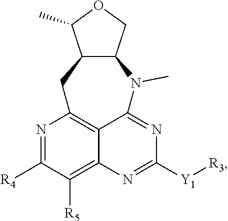

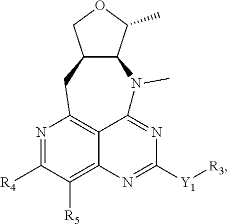

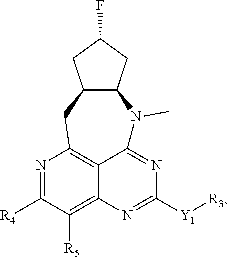

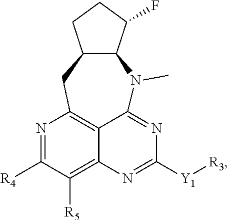

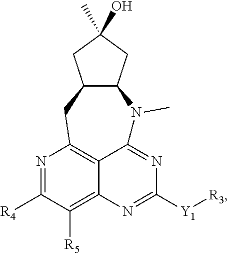

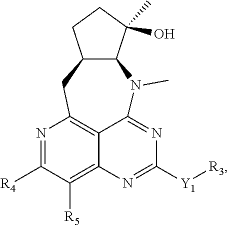

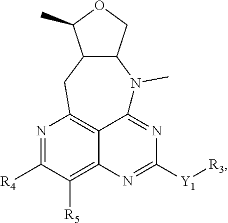

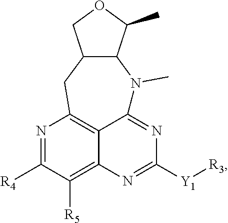

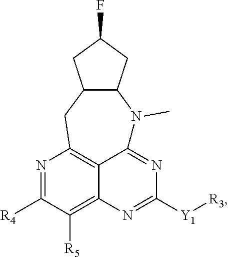

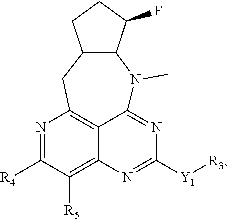

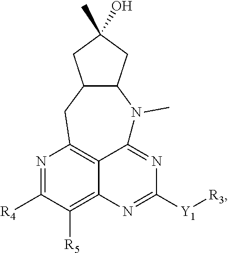

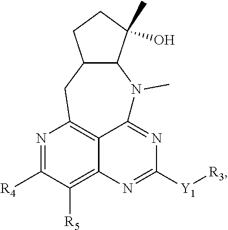

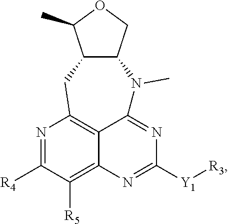

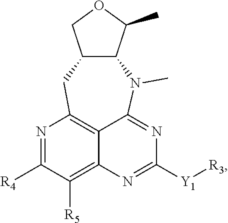

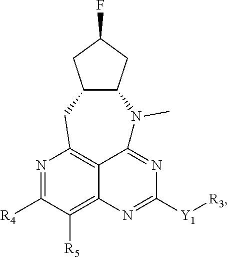

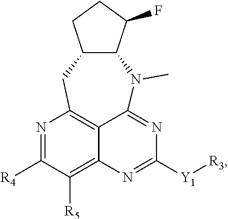

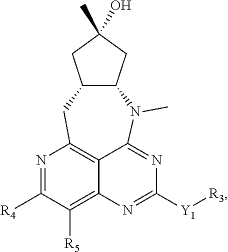

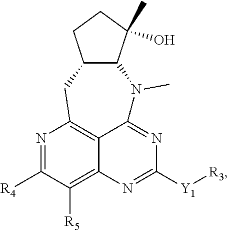

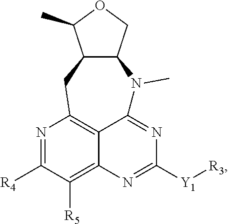

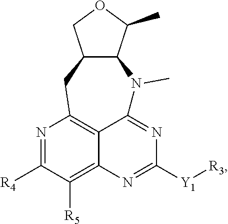

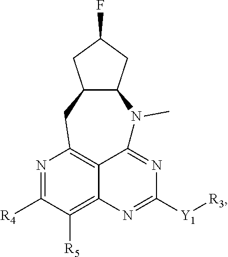

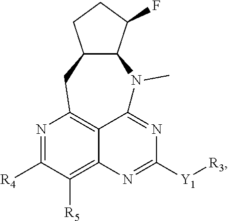

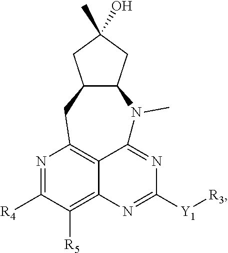

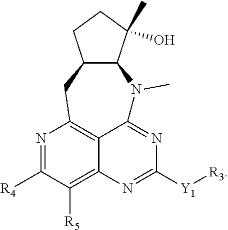

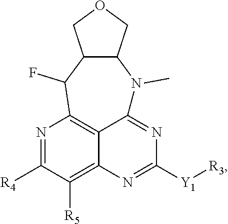

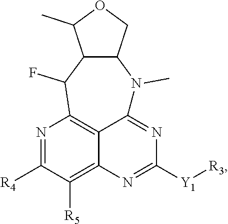

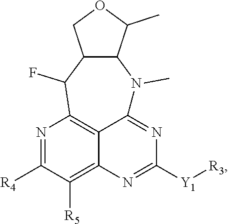

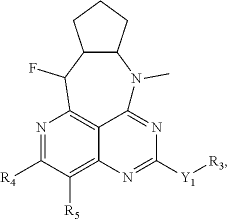

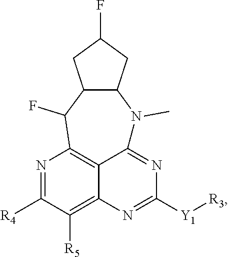

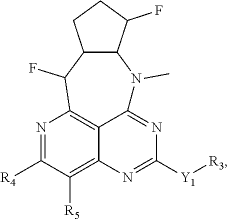

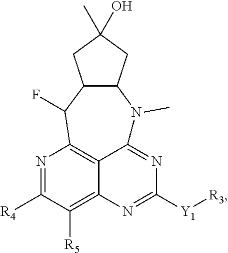

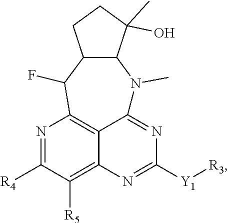

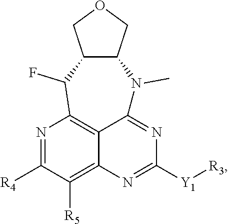

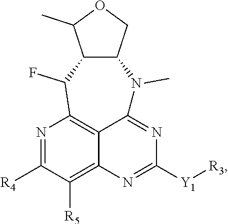

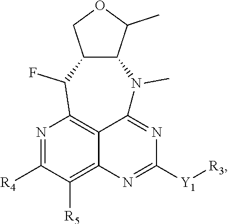

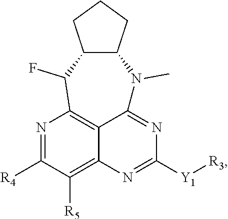

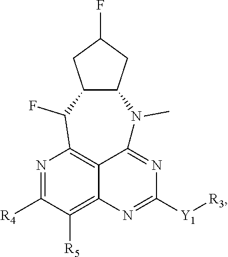

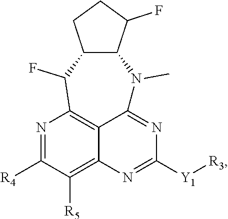

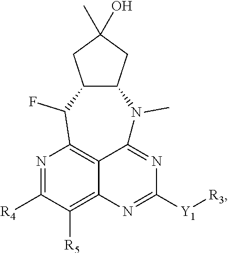

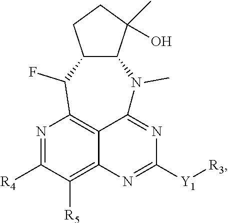

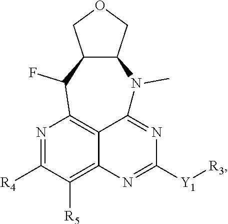

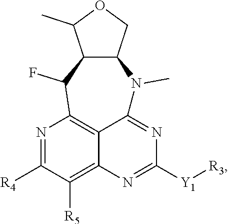

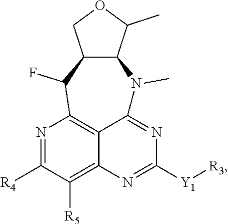

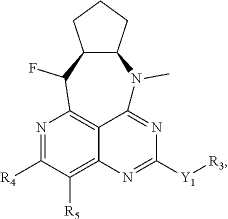

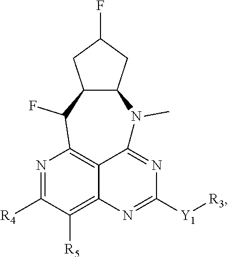

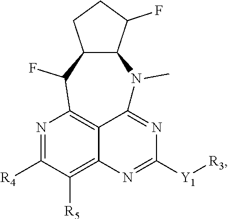

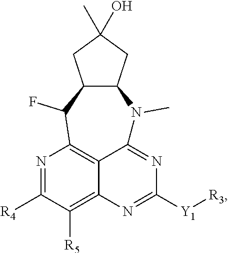

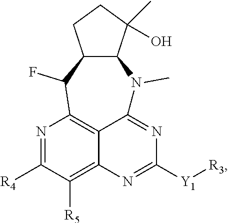

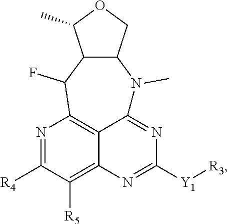

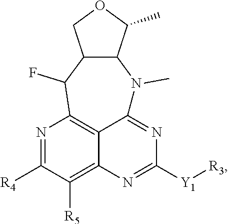

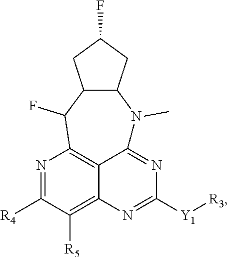

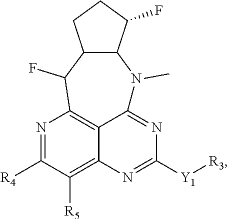

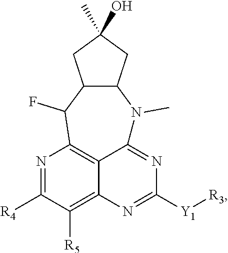

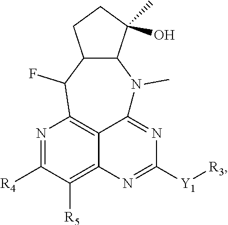

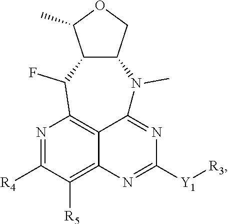

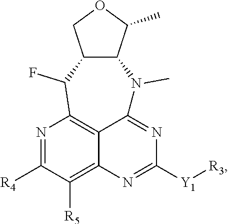

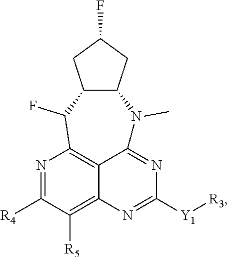

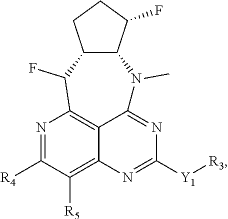

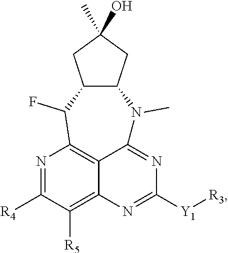

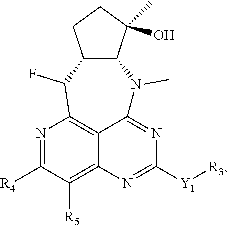

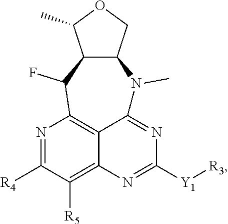

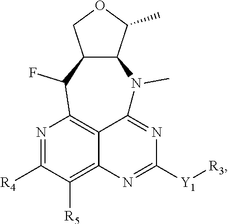

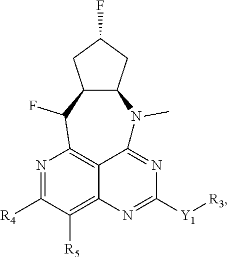

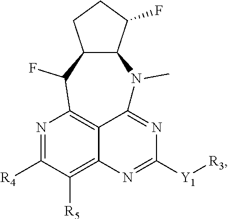

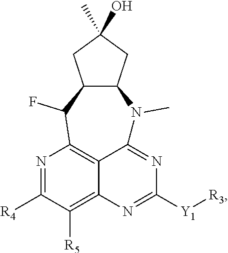

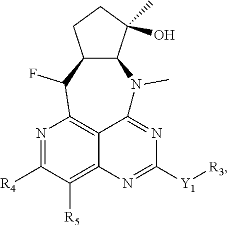

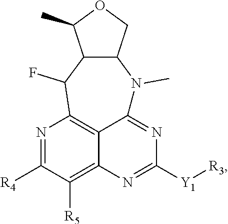

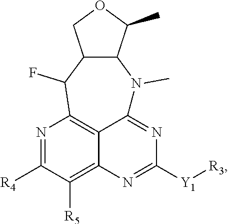

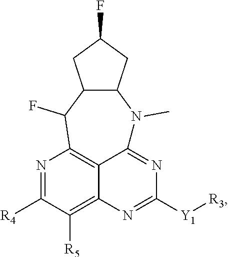

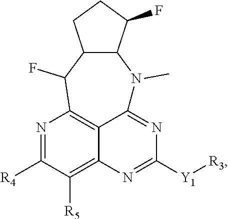

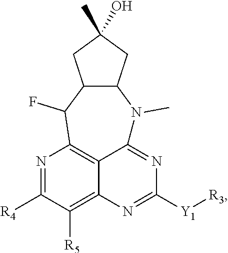

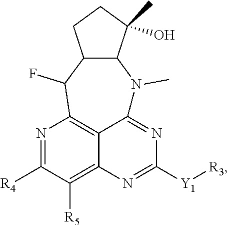

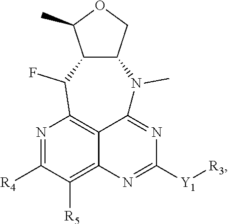

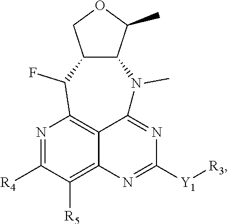

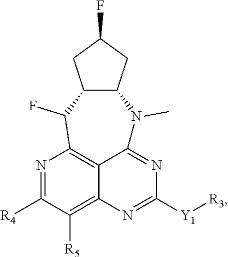

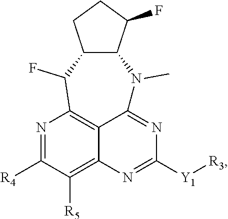

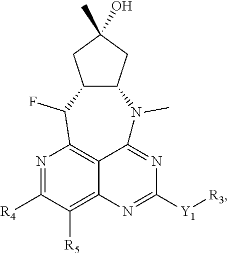

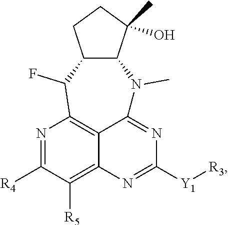

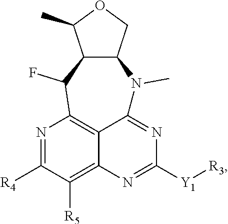

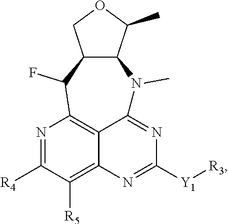

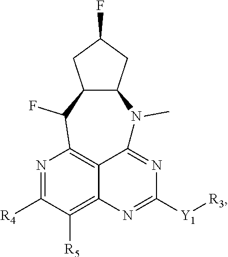

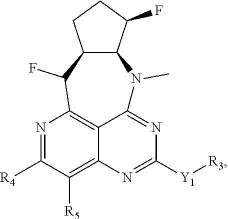

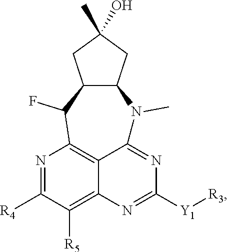

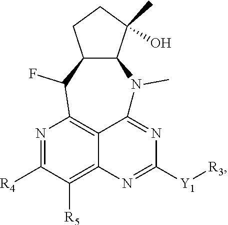

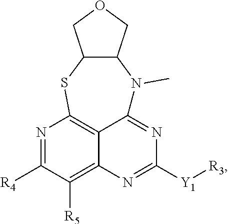

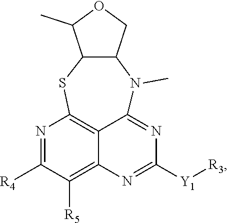

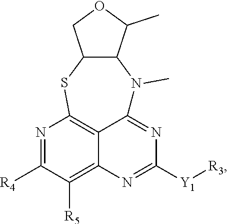

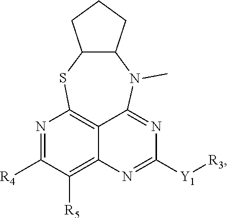

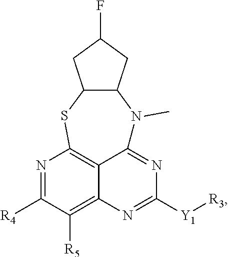

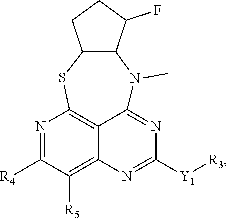

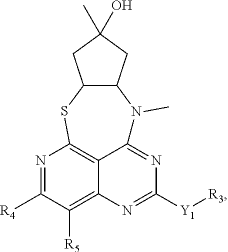

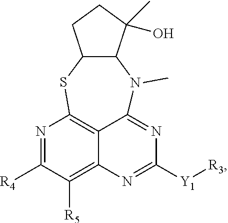

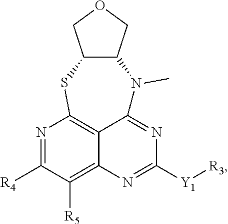

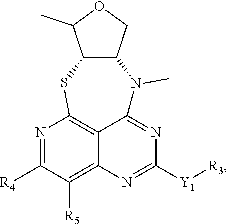

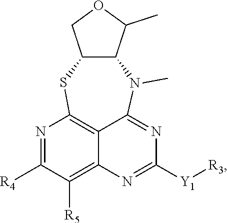

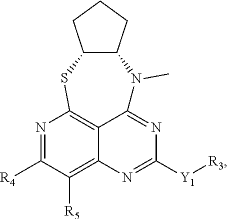

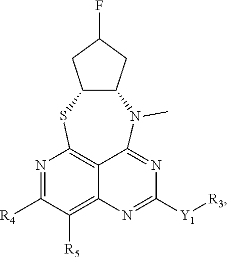

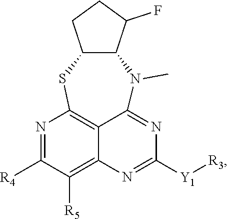

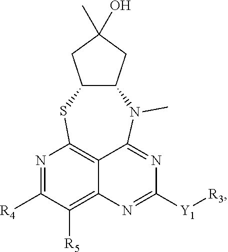

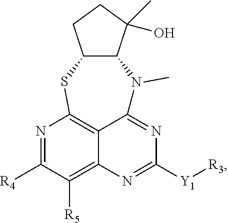

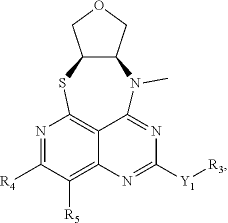

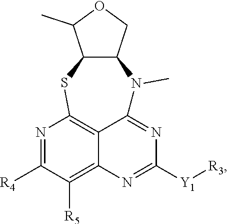

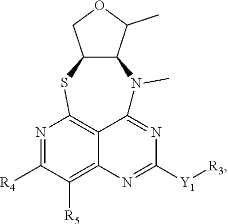

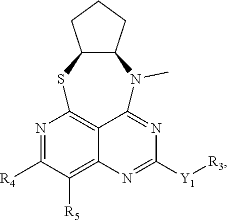

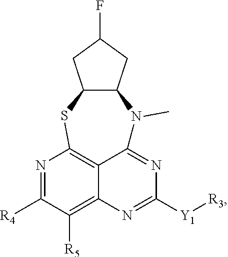

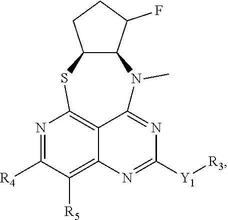

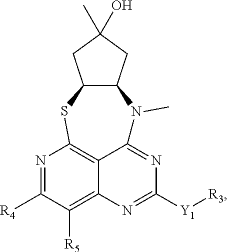

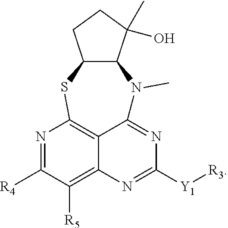

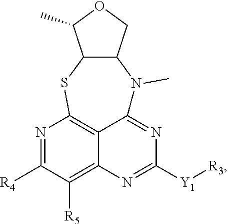

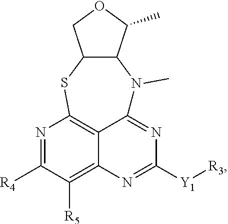

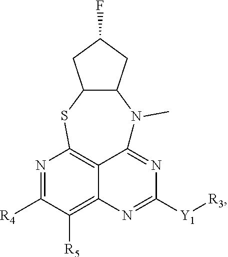

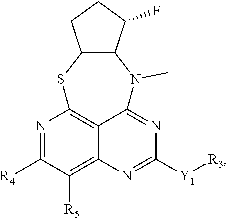

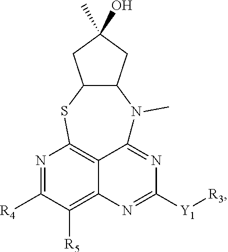

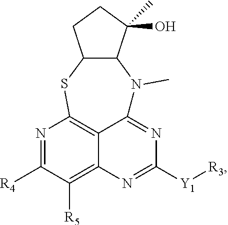

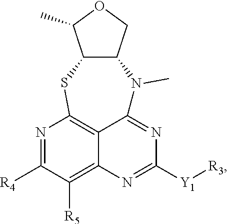

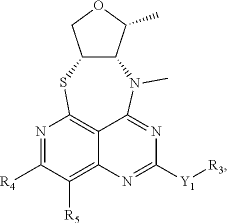

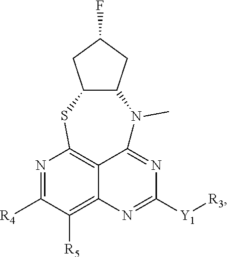

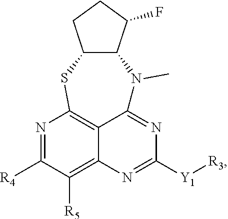

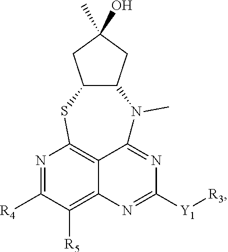

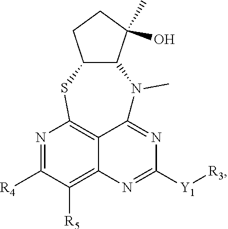

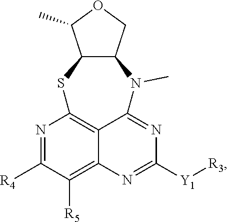

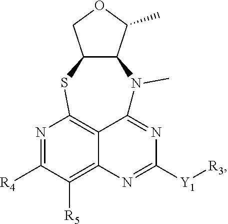

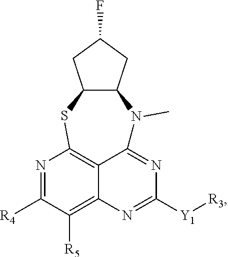

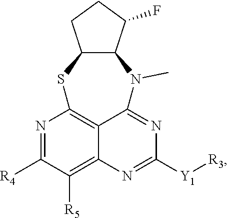

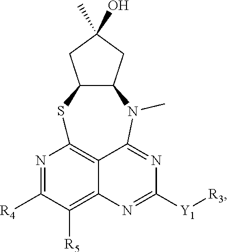

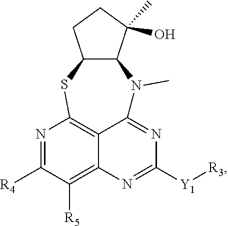

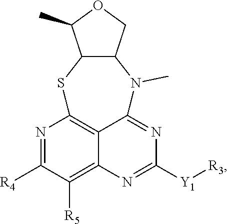

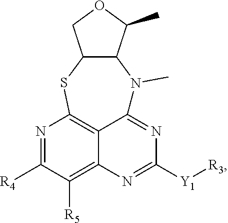

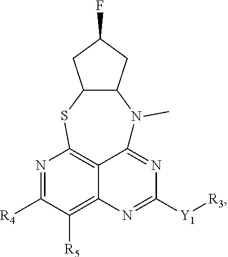

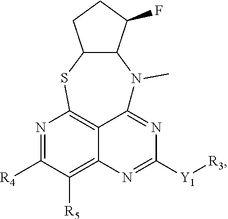

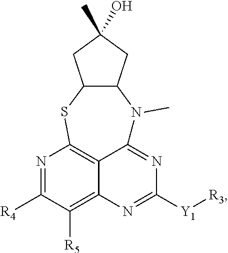

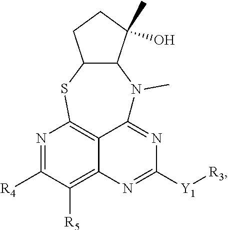

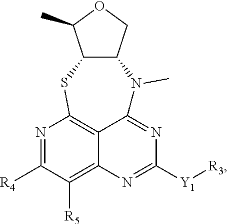

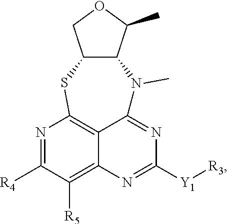

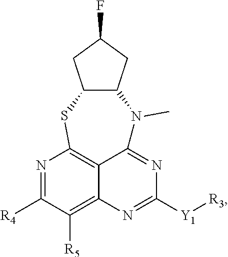

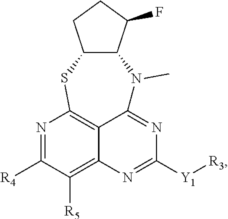

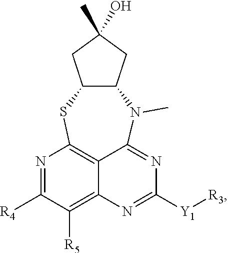

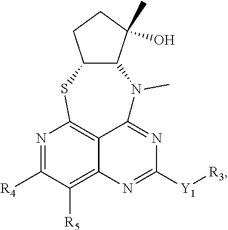

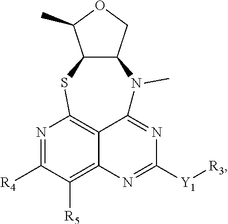

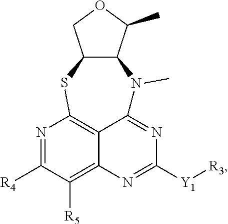

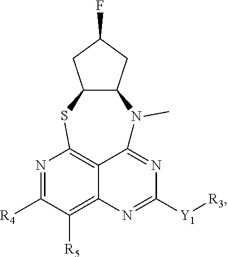

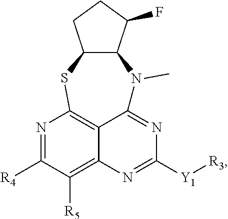

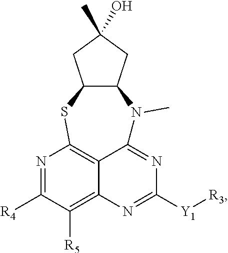

or

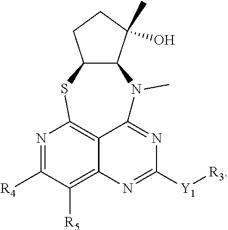

[11]. A compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof:

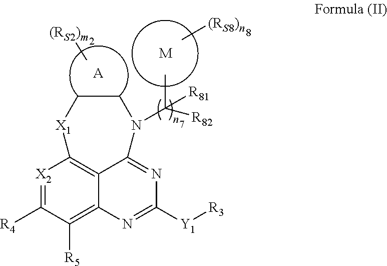

Wherein,
X₁ at each occurrence is independently selected from a bond, —C(R_{X11})(R_{X12})—, —NR_{X13}—, —O—, —S—, —S(═O)— or —S(═O)₂—;
R_{X11} or R_{X12} is independently selected from hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}), —OC(═O)N(R_{C})₂, —NR_{C}C(═O)N(R_{C})₂, —S(═O)OR_{C}, —OS(═O)R_{D}, —S(═O)N(R_{D}), —NR_{C}S(═O)R_{D}, —S(═O)₂OR_{C}, —OS(═O)R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, R_{X11} and R_{X12} together with the carbon atom to which they are both attached forms

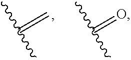

a 3-10 membered carbocyclic ring or a 3-10 heterocyclic ring; wherein, said

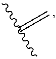

3-10 membered carbocylic ring or 3-10 heterocyclic ring is independently unsubstituted or substituted with one or more R_{SX1};
R_{X13} is selected from hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂; —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
X₂ at each occurrence is independently selected from N or CR₁;
R₁ is selected from hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{A})₂, —OR_{1A}, —SR_{1A}, —S(═O)R_{1B}, —S(═O)R_{1B}, —C(═O)R_{1B}, —C(═O)OR_{1A}, —OC(═O)R_{1B}, —C(═O)N(R_{1A})₂, —NR_{1A}C(═O)R_{1B}, —NR_{1A}C(═O)OR_{1A}, —NR_{1A}C(═S)OR_{1A}, —OC(═O)N(R_{A})₂, —NR_{1A}C(═O)N(R_{1A})₂, —S(═O)OR_{1A}, —OS(═O)R_{1B}, —OS(═O)₂R_{1B}, —S(═O)₂N(R_{1A})₂, —NR_{1A}S(═O)₂R_{1B}, —S(═O)N(R_{1A})₂, —NR_{1A}S(═O)R_{1B}, —S(═O)₂OR_{1A}, —OS(═O)₂OR_{1A}, —NR_{1A}S(═O)₂OR_{1A}, —OS(═O)₂N(R_{1A})₂, —NR_{1A}S(═O)₂N(R_{1A})₂, —P(R_{1A})₂, —P(═O)(R_{1B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1C})₂, —OR_{1C}, —SR_{1C}, —S(═O)R_{1D}, —S(═O)₂R_{1D}, —C(═O)R_{1D}, —C(═O)OR^{1D}, —OC(═O)R_{1D}, —C(═O)N(R_{1C})₂, —NR_{1C}C(═O)R_{1D}, —OC(═O)OR_{1C}, —NR_{1C}C(═O)OR_{1C}, —NR_{1C}C(═S)OR_{1C}, —OC(═O)N(R_{1C})₂, —NR_{1C}C(═O)N(R_{1C})₂, —S(═O)OR_{1C}, —OS(═O)R_{1D}, —S(═O)N(R_{1C})₂, —NR_{1C}S(═O)R_{1D}, —S(═O)₂OR_{1C}, —S(═O)₂N(R_{1C})₂, —NR_{1C}S(═O)₂R_{1D}, —OS(═O)OR_{1C}, —NR_{1C}S(═O)OR_{1C}, —OS(═O)₂N(R_{1C})₂, —NR_{1C}S(═O)₂N(R_{1C})₂, —P(R_{1C})₂, —P(═O)(R_{1D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
Ring A is selected from a 3-10 membered carbocyclic ring, a 3-10 heterocyclic ring containing 1, 2, or 3 heteroatoms selected from N, O, S, S═O or S(═O)₂, a phenyl ring, a 5-6 heteroaryl ring containing 1, 2, 3 or 4 heteroatoms selected from N, O or S;

Each of R₈₁ and R₈₂ is independently selected form hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{8A})₂, —OR_{8A}, —SR_{8A}, —S(═O)R_{8B}, —S(═O)₂R_{8B}, —C(═O)R_{8B}, —C(═O)OR_{8B}, —C(═O)N(R_{8B})₂, —S(═O)OR_{8B}, —S(═O)N(R_{8B})₂, —S(═O)₂OR_{8B}, —S(═O)₂N(R_{8B})₂, —P(═O)(R_{8B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{8C})₂, —OR_{8C}, —SR_{8C}, —S(═O)R_{8D}, —S(═O)₂R_{8D}, —C(═O)R_{8D}, —C(═O)OR_{8C}, —OC(═O)R_{8D}, —C(═O)N(R_{8C})₂, —NR_{8C}C(═O)R_{8D}, —OC(═O)OR_{8C}, —NR_{8C}C(═O)OR_{8D}, —OC(═O)N(R_{8C})₂, —NR_{8C}C(═O)N(R_{8C})₂, —S(═O)OR_{8C}, —OS(═O)R_{8D}, —S(═O)N(R_{8C})₂, —NR_{8C}S(═O)R_{8D}, —S(═O)₂OR_{8C}, —OS(═O)₂R_{8D}, —S(═O)₂N(R_{8C})₂, —NR_{8C}S(═O)₂R_{8D}, —OS(═O)OR_{8C}, —NR_{8C}S(═O)₂OR_{8C}, —OS(═O)₂NR_{8C}, —NR_{8C}S(═O)₂N(R_{8C})₂, —P(R_{8C})₂, —P(═O)(R_{8D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, R₈₁ and R₈₂ together with the carbon atom to which they are both attached form

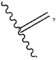

a 3-10 membered carbocylic ring 3-10 membered carbocyclic ring or a 3-10 heterocyclic ring; wherein, said

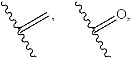

3-10 membered carbocylic ring or 3-10 heterocyclic ring is independently unsubstituted or substituted with one or more R_{S81};

Optionally, R₈₁ and R₈₂ together with the atoms to which they are respectively attached form a 3-10 membered carbocyclic ring or a 3-10 heterocyclic ring; wherein, said 3-10 membered carbocylic ring or 3-10 heterocyclic ring is independently unsubstituted or substituted with one or more R_{S82};
n₇ is selected from 0, 1, 2, 3, 4, 5 or 6;
Ring M is selected from a 6-10 membered aryl ring or 5-10 membered heteroaryl containing 1, 2, 3 or 4 heteroatoms selected from N, O or S;
R_{S8} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N, oxo, —N(R_{S8A})₂, —OR_{S8A}, —SR_{S8A}, —S(═O)R_{S8B}, —S(═O)₂R_{S8B}, C(═O)R_{S8B}, —C(═O)OR_{S8A}, —OC(═O)R_{S8B}, C(═O)N(R_{S8A})₂, —NR_{S8A}C(═O)R_{S8B}, —OC(═O)OR_{S8A}, —NR_{S8A}C(═O)OR_{S8A}, —NR_{S8A}C(═S)OR_{S8A}, —OC(═O)N(R_{S8A})₂, —NR_{S8A}C(═O)N(R_{S8A})₂, —S(═O)OR_{S8A}, —OS(═O)R_{S8B}, —S(═O)N(R_{S8A})₂, —NR_{S3A}S(═O)R_{S8B}, —S(═O)₂OR_{S8A}, —OS(═O)₂R_{S8B}, —S(═O)₂N(R_{S8A})₂, —NR_{S8A}S(═O)₂R_{S8B}, —OS(═O)₂OR_{S8A}, —NR_{S8A}S(═O)₂OR_{S8A}, —OS(═O)₂N(R_{S8A})₂, —P(R_{S8A})₂, —P(═O)(R_{S8B})₂, -10 membered cycloalkyl, -10 membered cycloalkenyl, -10 membered cycloalkynyl, -10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, -10 membered cycloalkyl, -10 membered cycloalkenyl, -10 membered cycloalkynyl, -10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N, oxo, —N(R_{S8C})₂, —OR_{S8C}, —SR_{S8C}, —S(═O)R_{S8D}, —S(═O)₂R_{S8D}, —C(═O)R_{S8D}, —C(═O)OR_{S8C}, —OC(═O)R_{S8D}, —C(═O)N(R_{S8C})₂, —NR_{S8C}C(═O)R_{S8D}, —OC(═O)OR_{S8C}, —NR_{S8C}C(═O)OR_{S8C}, —NR_{S8C}C(═S)OR_{S8C}, —OC(═O)N(R_{S8C})₂, —NR_{S8C}C(═O)N(R_{S8C})₂, —S(═O)OR_{S8C}, —OS(═O)R_{S8D}, —S(═O)N(R_{S8C})₂, —NR_{S8C}S(═O)R_{S8D}, —S(═O)OR_{S8C}, —OS(═O)₂R_{S8D}, —S(═O)₂N(R_{S8C})₂, —NR_{S8C}S(═O)₂R_{S8D}, —OS(═O)₂OR_{S8C}, —NR_{S8C}S(═O)₂OR_{S8C}, —OS(═O)₂N(R_{S8C})₂, —NR_{S8C}S(═O)₂N(R_{S8C})₂, —P(R_{S8C})₂, —P(═O)(R_{S8D})₂, -10 membered cycloalkyl, -10 membered cycloalkenyl, -10 membered cycloalkynyl, -10 membered heterocyclyl, 6-10 membered aryl or 5-10 membered heteroaryl;
n₈ is selected from 0, 1, 2, 3, 4, 5 or 6;
R_{S2} is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S2A})₂, —OR_{S2A}, —SR_{S2A}, —S(═O)R_{S2B}, —S(═O)₂R_{S2B}, —C(═O)R_{S2B}, —C(═O)OR_{S2A}, —OC(═O)R_{S2B}, —C(═O)N(R_{S2A})₂, —NR_{S2A}C(═O)R_{S2B}, —OC(═O)N(R_{S2A})₂, —NR_{S2A}C(═O)N(R_{S2A})₂, —NR_{S2A}C(═O)OR_{S2A}, —NR_{S2A}C(═S)OR_{S2A}, —OC(═O)OR_{S2A}, —S(═O)OR_{S2A}, —OS(═O)R_{S2B}, —S(═O)N(R_{S2A})₂, —S(═O)N(R_{S2A})₂, —NR_{S2A}S(═O)₂R_{S2B}, —OS(═O)₂OR_{S2A}, —NR_{S2A}S(═O)₂OR_{S2A}, —OS(═O)₂N(R_{S2A})₂, —NR_{S2A}S(═O)₂N(R_{S2A})₂, —P(R_{S2A})₂, —P(═O)(R_{S2B})₂, 3-10 membered cycloalkyl, 3-10 membered cycloalkenyl, —NR_{S2A}S(═O)R_{S2B}, —S(═O)₂OR_{S2A}, —OS(═O)₂R_{S2B}, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S2C})₂, —OR_{S2C}, —SR_{S2C}, —S(═O)R_{S2D}, —S(═O)₂R_{S2D}, —C(═O)R_{S2D}, —C(═O)OR_{S2D}, —OC(═O)R_{S2D}, —C(═O)N(R_{S2C})₂, —NR_{S2C}C(═O)R_{S2D}, —OC(═O)OR_{S2C}, —NR_{S2C}C(═O)OR_{S2C}, —NR_{S2C}C(═S)OR_{S2C}, —OC(═O)N(R_{S2C})₂, —NR_{S2C}C(═O)N(R_{S2C})₂, —S(═O)OR_{S2C}, —OS(═O)R_{S2D}, —S(═O)N(R_{S2C})₂, —NR_{S2C}S(═O)R_{S2C}, —S(═O)₂OR_{S2C}, —OS(═O)₂R_{S2D}, —S(═O)₂N(R_{S2C})₂, —NR_{S2C}S(═O)₂R_{S2D}, —OS(═O)₂OR_{S2C}, —NR_{S2C}S(═O)₂OR_{S2C}, —OS(═O)₂N(R_{S2C})₂, —NR_{S2C}S(═O)₂N(R_{S2C})₂, —P(R_{S2C})₂, —P(═O)(R_{S2D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S2} together with the carbon atom to which they are both attached form

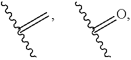

a 3-10 membered carbocyclic ring or a 3-10 heterocyclic ring; wherein, said

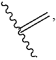

3-10 membered carbocylic ring or 3-10 heterocyclic ring is independently unsubstituted or substituted with one or more R_{S21};

Optionally, two adjacent R_{S2} together with the atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S22}:

Optionally, two nonadjacent R_{S2} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S23},
m₂ is selected from 0, 1, 2, 3, 4 or 5;
Y is a bond, O, S, S(═O), S(═O), or NR_{Y11};
R_{Y11} is selected from hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C}), —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R₃ is selected from

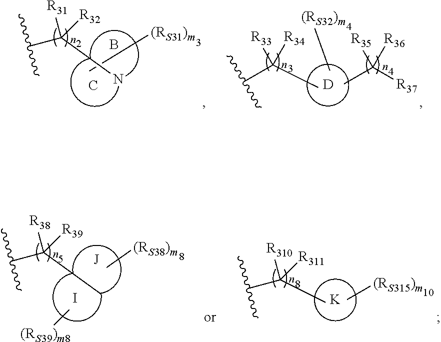

Each of R₃₁, R₃₂, R₃₃, R₃₄, R₃₅, R₃₆, R₃₈, R₃₉, R₃₁₀ and R₃₁₁ is independently selected form hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{A}, —C(═O)N(R_{A})₂, —S(═O)OR_{A}, —S(═O)N(R_{A})₂, —S(═O)₂OR_{A}, —S(═O)₂N(R_{A}), —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C}), —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, R₃₁ and R₃₂ together with the carbon atom to which they are both attached form

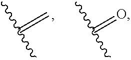

a 3-10 membered carbocylic ring 3-10 membered carbocyclic ring or a 3-10 heterocyclic ring; wherein, said

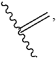

3-10 membered carbocylic ring or 3-10 heterocyclic ring is independently unsubstituted or substituted with one or more R_{S33};

Optionally, R₃₃ and R₃₄ together with the carbon atom to which they are both attached form

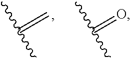

a 3-10 membered carbocylic ring 3-10 membered carbocyclic ring or a 3-10 heterocyclic ring; wherein, said

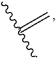

3-10 membered carbocylic ring or 3-10 heterocyclic ring is independently unsubstituted or substituted with one or more R_{S34};

Optionally, R₃₅ and R₃₆ together with the carbon atom to which they are both attached form

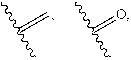

a 3-10 membered carbocylic ring 3-10 membered carbocyclic ring or a 3-10 heterocyclic ring; wherein, said

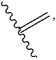

3-10 membered carbocylic ring or 3-10 heterocyclic ring is independently unsubstituted or substituted with one or more R_{S35};

Optionally, R₃₈ and R₃₉ together with the carbon atom to which they are both attached form

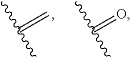

a 3-10 membered carbocylic ring 3-10 membered carbocyclic ring or a 3-10 heterocyclic ring; wherein, said

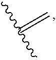

3-10 membered carbocylic ring or 3-10 heterocyclic ring is independently unsubstituted or substituted with one or more R_{S310};

Optionally, R₃₁₀ and R₃₁₁ together with the carbon atom to which they are both attached form

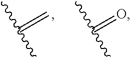

a 3-10 membered carbocylic a 3-10 membered carbocyclic ring or a 3-10 heterocyclic ring; wherein, said

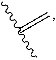

3-10 membered carbocylic ring or 3-10 heterocyclic ring is independently unsubstituted or substituted with one or more R_{S316};
n₂ is selected from 0, 1, 2, 3, 4, 5 or 6;
n₃ is selected from 0, 1, 2, 3, 4, 5 or 6;
n₄ is selected from 0, 1, 2, 3, 4, 5 or 6;
n₅ is selected from 0, 1, 2, 3, 4, 5 or 6;
n₆ is selected from 0, 1, 2, 3, 4, 5 or 6;
Ring B is a 3-10 membered heterocyclic ring optionally further containing 1, 2, or 3 heteroatoms selected from N, O, S, S(═O) or S(═O)₂;
Ring C is a 3-10 membered heterocyclic ring optionally further containing 1, 2, or 3 heteroatoms selected from N, O, S, S(═O) or S(═O)₂;
Ring D is selected from a 3-10 membered carbocyclic ring or a 3-10 membered heterocyclic ring;
Ring I is a 3-10 membered carbocyclic ring or a 3-10 membered heterocyclic ring containing 1, 2, or 3 heteroatoms selected from N, O, S, S(═O) or S(═O)₂;
Ring J is a 3-10 membered carbocyclic ring or a 3-10 membered heterocyclic ring containing 1, 2, or 3 heteroatoms selected from N, O, S, S(═O) or S(═O)₂,
Ring K is selected from 3-10 membered carbocyclic ring or a 3-10 membered heterocyclic ring containing 1, 2, or 3 heteroatoms selected from N, O, S, S(═O) or S(═O)₂;
R_{S31} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31A})₂, —OR_{S31A}, —SR_{S31A}, —S(═O)R_{S31B}, —S(═O)₂R_{S31B}, —C(═O)R_{S31B}, —C(═O)OR_{S31A}, —OC(═O)R_{S31B}, —C(═O)N(R_{S31A})₂, —NR_{S31A}C(═O)R_{S31B}, —OC(═O)OR_{S31A}, —NR_{S31A}C(═O)OR_{S31A}, —NR_{S31A}C(═S)OR_{S31A}, —OC(═O)N(R_{S31A})₂, —NR_{S31A}C(═O)N(R_{S31A})₂, —S(═O)OR_{S31A}, —OS(═O)R_{S31B}, —S(═O)N(R_{S31A})₂, —NR_{S31A}S(═O)R_{S31B}, —S(═O)₂OR_{S31A}; —OS(═O)₂R_{S31B}, —S(═O)₂N(R_{S31A})₂, —NR_{S31A}S(═O)₂R_{S31B}, —NR_{S31A}S(═O)₂OR_{S31A}, —OS(═O)₂N(R_{S31A})₂, —NR_{S31A}S(═O)₂N(R_{S31A})₂, —P(R_{S31A})₂, —P(═O)(R_{S31B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31C})₂, —OR_{S31C}, —SR_{S31C}, —S(═O)R^{S31D}, —S(═O)₂R_{S31D}, —C(═O)R_{S31D}, —C(═O)OR_{S31C}, —OC(═O)R_{S31D}, —C(═O)N(R_{S31C})₂, —NR_{S31C}C(═O)R_{S31D}, —OC(═O)OR_{S31C}, —NR_{S31C}C(═O)OR_{S31C}, —NR_{S31C}C(═S)OR_{S31C}, —OC(═O)N(R_{S31C})₂, —NR_{S31C}C(═O)N(R_{S31C})₂, —S(═O)OR_{S31C}, —OS(═O)R_{S31D}, —S(═O)N(R_{S31C})₂, —NR_{S31C}S(═O)R_{S31D}, —S(═O)₂OR_{S31C}, —OS(═O)₂R_{S31D}, —S(═O)₂N(R_{S31C})₂, —NR_{S31C}S(═O)₂R_{S31D}, —OS(═O)₂OR_{S31C}, —NR_{S31C}S(═O)₂OR_{S31C}, —OS(═O)₂N(R_{S31C})₂, —NR_{S31C}S(═O)₂N(R_{S31C})₂, —P(R_{S31C})₂, —P(═O)(R_{S31D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S31} together with the carbon atom to which they are both attached form

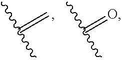

a 3-10 membered 3-10 membered carbocyclic ring or a 3-10 membered heterocyclic ring; wherein, said

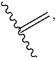

3-10 membered carbocylic ring or 3-10 heterocyclic ring is independently unsubstituted or substituted with one or more R_{S311};

Optionally, two adjacent R_{S31} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S312};

Optionally, two nonadjacent R_{S31} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S313};
m₃ is selected from 0, 1, 2, 3, 4, 5, 6, 7, 8, 9, 10, 11, or 12;
R_{S32} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32A})₂, —OR_{S32A}, —SR_{S32A}, —S(═O)R_{S32B}, —S(═O)₂R_{S32B}, —C(═O)R_{S32B}, —C(═O)OR_{S32A}, —OC(═O)R_{S32B}, —C(═O)N(R_{S32A})₂, —NR_{S32A}C(═O)R_{S32B}, —OC(═O)OR_{S32A}, —NR_{S32A}C(═O)OR_{S32A}, —NR_{S32A}C(═S)OR_{S32A}, —OC(═O)N(R_{S32A})₂, —NR_{S32A}C(═O)N(R_{S32A})₂, —S(═O)OR_{S32A}, —OS(═O)R_{S32B}, —S(═O)N(R_{S32A})₂, —NR_{S32A}S(═O)R_{S32B}, —S(═O)₂OR_{S32A}, —OS(═O)₂R_{S32B}, —S(═O)N(R_{S32A})₂, —NR_{S32A}S(═O)₂R_{S32B}, —OS(═O)OR_{S32A}, —NR_{S32A}S(═O)₂OR_{S32A}, —OS(═O)₂N(R_{S32A})₂, —NR_{S32A}S(═O)₂N(R_{S32A})₂, —P(R_{S32A})₂, —P(═O)(R_{S32B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32C})₂, —OR_{S32C}, —SR_{S32D}, —S(═O)R_{S32D}, —S(═O)₂R_{S32D}, —C(═O)R_{S32D}, —C(═O)OR_{S32D}, —OC(═O)R_{S32D}, —C(═O)N(R_{S32C})₂, —NR_{S32C}C(═O)R_{S32D}, —OC(═O)OR_{S32C}, —NR_{S32D}C(═O)OR_{S32C}, —NR_{S32C}C(═S)OR_{S32C}, —OC(═O)N(R_{S32C})₂, —NR_{S32C}C(═O)N(R_{S32C})₂, —S(═O)OR_{S32C}, —OS(═O)R_{S32C}, —S(═O)N(R_{S32C})₂, —NR_{S32C}S(═O)R_{S32D}, —S(═O)₂OR_{S32C}, —OS(═O)₂R_{S32D}, —S(═O)₂N(R_{S32C})₂, —NR_{S32C}S(═O)₂R_{S32D}, —OS(═O)₂OR_{S32C}, —NR_{S32C}S(═O)₂OR_{S32C}, —OS(═O)₂N(R_{S32C})₂, —NR_{S32C}S(═O)₂N(R_{S32C})₂, —P(R_{S32C})₂, —P(═O)(R_{S32D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S32} together with the carbon atom to which they are both attached form

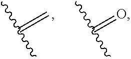

a 3-10 membered carbocylic ring 3-10 membered carbocyclic ring or a 3-10 heterocyclic ring; wherein, said

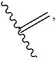

3-10 membered carbocylic ring or 3-10 heterocyclic ring is independently unsubstituted or substituted with one or more R_{S321};

Optionally, two adjacent R_{S32} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S322};

Optionally, two nonadjacent R_{S32} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S323};
m₄ is selected from 0, 1, 2, 3, 4, 5 or 6;
R₃₇ is selected from —N(R_{37A})₂ or 3-10 membered heterocyclyl, wherein said 3-10 membered heterocyclyl is optionally independently substituted with one or more R_{S37};
R_{S38} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38A})₂, —OR_{S38A}, —SR_{S38A}, —S(═O)R_{S38B}, —C(═O)R_{S38B}, —C(═O)OR_{S38A}, —OC(═O)R_{S38B}, —C(═O)N(R_{S38A})₂, —NR_{S38A}C(═O)R_{S38B}, —OC(═O)OR_{S38A}, —NR_{S38A} C(═O)OR_{S38A}, —NR_{S38A}C(═S)OR_{S38A}, —OC(═O)N(R_{S38A})₂, —NR_{S38A}C(═O)N(R_{S38A})₂, —S(═O)OR_{S38A}, —OS(═O)R_{S38B}, —S(═O)N(R_{S38A})₂, —NR_{S38A}S(═O)R_{S38B}, —S(═O)₂OR_{S38A}, —OS(═O)₂R_{S38B}, —S(═O)₂N(R_{S38A})₂, —NR_{S38A}S(═O)₂R_{S38B}, —OS(═O)₂OR_{S38A}, —NR_{S38A}S(═O)₂OR_{S38A}, —OS(═O)₂N(R_{S38A})₂, —NR_{S38A}S(═O)₂N(R_{S38A})₂, —P(R_{S38A})₂, —P(═O)(R_{S38B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38C})₂, —OR_{S38C}, —SR_{S38C}, —S(═O)R_{S38C}, —S(═O)₂R_{S38D}, —C(═O)R_{S38D}; —C(═O)OR_{S38C}, —NR_{S38C}C(═O)R_{S38D}, —OC(═O)R_{S38D}, —C(═O)N(R_{S38C})₂, —OC(═O)OR_{S38C}, —NR_{S38C}C(═O)OR_{S38C}, —OC(═O)N(R_{S38C})₂, —NR_{S38C}C(═O)N(R_{S38C})₂, —S(═O)OR_{S38C}, —OS(═O)R_{S38D}, —NR_{S38C}C(═S)OR_{S38C}, —S(═O)N(R_{S38C})₂, —NR_{S38C}S(═O)R_{S38D}, —S(═O)₂OR_{S38C}, —OS(═O)₂R_{S38D}, —S(═O)₂N(R_{S38C})₂, —NR_{S38C}S(═O)₂R_{S38D}, —NR_{S38C}S(═O)₂OR_{S38C}, —OS(═O)₂N(R_{S38C})₂, —NR_{S38C}S(═O)₂N(R_{S38C})₂, —P(R_{S38C})₂, —OS(═O)₂OR_{S38C}, —P(═O)(R_{S38D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S38} together with the carbon atom to which they are both attached form

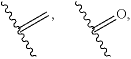

a 3-10 membered carbocylic ring 3-10 membered carbocyclic ring or a 3-10 heterocyclic ring; wherein, said

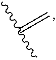

3-10 membered carbocylic ring or 3-10 heterocyclic ring is independently unsubstituted or substituted with one or more R_{S381};

Optionally, two adjacent R_{S38} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S382};

Optionally, two nonadjacent R_{S38} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S═O or S(═O)₂; the hydrogen on the each of carbon atoms or N atoms is independently unsubstituted or substituted with R_{S383};
m₈ is selected from 0, 1, 2, 3, 4, 5, 6, 7, 8, 9, 10, 11, or 12;
R_{S39} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39A})₂, —OR_{S39A}, —SR_{S39A}, —S(═O)R_{S39B}, —S(═O)₂R_{S39B}, —C(═O)R_{S39B}, —C(═O)OR_{S39A}, —OC(═O)R_{S39B}, —C(═O)N(R_{S39A})₂, —NR_{S39A}C(═O)R_{S39B}, —OC(═O)OR_{S39A}, —NR_{S39A}C(═O)OR_{S39A}, —NR_{S39A}C(═S)OR_{S39A}, —OC(═O)N(R_{S39A})₂, —NR_{S39A}C(═O)N(R_{S39A})₂, —S(═O)OR_{S39A}, —OS(═O)R_{S39B}, —S(═O)N(R_{S39A})₂, —NR_{S39A}S(═O)R_{S39B}, —S(═O)₂OR_{S39A}, —OS(═O)₂R_{S39B}, —S(═O)₂N(R_{S39A})₂, —NR_{S39A}S(═O)₂R_{S39B}, —OS(═O)₂OR_{S39A}, —NR_{S39A}S(═O)₂OR_{S39A}, —OS(═O)₂N(R_{S39A})₂, —NR_{S39A}S(═O)₂N(R_{S39A})₂, —P(R_{S39A})₂, —P(═O)(R_{S39B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39C})₂, —OR_{S39C}, —SR_{S39C}, —S(═O)R_{S39C}, —S(═O)₂R_{S39D}, —C(═O)R_{S39D}, —C(═O)OR_{S39C}, —NR_{S39C}C(═O)R_{S39D}, —OC(═O)R_{S39D}, —C(═O)N(R_{S39C})₂, —OC(═O)OR_{S39C}, —NR_{S39C}C(═O)OR_{S39C}, —NR_{S39C}C(═S)OR_{S39C}, —OC(═O)N(R_{S39C})₂, —NR_{S39C}C(═O)N(R_{S39C})₂, —S(═O)OR_{S39C}, —OS(═O)R_{S39C}, —S(═O)N(R_{S39C})₂, —NR_{S39C}S(═O)R_{S39D}, —S(═O)₂OR_{S39C}, —OS(═O)₂R_{S39D}, —S(═O)₂N(R_{S39C})₂, —NR_{S39C}S(═O)₂R_{S39D}, —OS(═O)₂OR_{S39C}, —OS(═O)₂N(R_{S39C})₂, —NR_{S39C}S(═O)₂N(R_{S39C})₂, —P(R_{S39C})₂, —P(═O)(R_{S39D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S39} together with the carbon atom to which they are both attached form

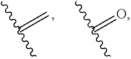

a 3-10 membered carbocylic ring 3-10 membered carbocyclic ring or a 3-10 heterocyclic ring; wherein, said

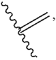

3-10 membered carbocylic ring or 3-10 heterocyclic ring is independently unsubstituted or substituted with one or more R_{S391};

Optionally, two adjacent R_{S39} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S392};

Optionally, two nonadjacent R_{S39} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S—O or S(═O)₂, the hydrogen on the each of carbon atoms or N atoms is independently unsubstituted or substituted with R_{S393};
m₉ is selected from 0, 1, 2, 3, 4, 5 or 6;
R_{S315} is selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315A})₂, —OR_{S315A}, —SR_{S315A}, —S(═O)R_{S315B}, —S(═O)R_{S315B}, —C(═O)R_{S315B}, —C(═O)OR_{S315A}, —OC(═O)R_{S315B}, —C(═O)N(R_{S315A})₂, —NR_{S315A}C(═O)R_{S315B}, —OC(═O)OR_{S315A}, —NR_{S315A}C(═O)OR_{S315A}, —NR_{S315A}C(═S)OR_{S315A}, —OC(═O)N(R_{S315A})₂, —NR_{S315A}C(═O)N(R_{S315A})₂, —S(═O)OR_{S315A}, —OS(═O)R_{S315B}, —S(═O)N(R_{S315A})₂, —NR_{S315A}S(═O)R_{S315B}, —S(═O)₂OR_{S315A}, —OS(═O)₂R_{S315B}, —S(═O)₂N(R_{S315A})₂, —NR_{S315A}S(═O)₂R_{S315B}, —OS(═O)₂OR_{S315A}, —NR_{S315A}S(═O)₂OR_{S315A}, —OS(═O)₂N(R_{S315A})₂, —NR_{S315A}S(═O)₂N(R_{S315A})₂, —P(R_{S315A})₂, —P(═O)(R_{S315B})₂, 3- 10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S25C})₂, —OR_{S315C}, —SR_{S315C}, —S(═O)R_{S315C}, —S(═O)₂R_{S315D}, —C(═O)R_{S315D}, —C(═O)OR_{S315C}, —OC(═O)R_{S315D}, —C(═O)N(R_{S315C})₂, —NR_{S315C}C(═O)R_{S315D}, —OC(═O)OR_{S315C}, —OC(═O)N(R_{S315C})₂, —NR_{S315C}C(═O)N(R_{S315C})₂, —NR_{S315C}C(═O)OR_{S315C}, —S(═O)OR_{S315C}, —OS(═O)R_{S315C}, —S(═O)N(R_{S315C})₂, —NR_{S315C}S(═O)R_{S315D}, —S(═O)₂OR_{S315C}, —OS(═O)₂R_{S315D}, —S(═O)₂N(R_{S315C})₂, —NR_{S315C}S(═O)₂R_{S315D}, —OS(═O)₂OR_{S315C}, —NR_{S315C}S(═O)₂OR_{S315C}, —OS(═O)₂N(R_{S315C})₂, —NR_{S315C}S(═O)₂N(R_{S315C})₂, —P(R_{S315C})₂, —P(═O)(R_{S315D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, two R_{S315} together with the carbon atom to which they are both attached form

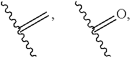

a 3-10 membered carbocyclic ring or a 3-10 heterocyclic ring; wherein, said

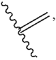

3-10 membered carbocyclo ring or 3-10 heterocyclic ring is independently unsubstituted or substituted with one or more R_{S3151};

Optionally, two adjacent R_{S315} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S3152}.

Optionally, two nonadjacent R_{S315} are connected together to form a bridge containing 0, 1, 2, 3, 4, 5 or 6 carbon atoms, wherein, each of the carbon atoms in the bridge is independently not replaced or replaced by 1 or 2 heteroatoms selected from N, O, S, S—O or S(═O)₂; the hydrogen on the each of carbon atoms or N atoms is independently unsubstituted or substituted with R_{S3153};
m₁₀ is selected from 0, 1, 2, 3, 4, 5 or 6;
R₄ is selected from 6-10 membered aryl, 5-10 membered heteroaryl,

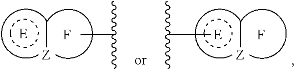

wherein said 6-10 membered aryl, 5-10 membered heteroaryl,

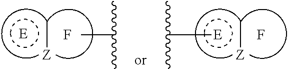

is independently unsubstituted or substituted with one or more R_{S4};
Z at each occurrence is independently selected from C or N;
Ring E at each occurrence is independently selected from a 6 membered aryl ring or a 5-6 membered heteroaryl ring and ring F at each occurrence is a 3-10 membered carbocyclic ring or a 3-10 membered heterocyclic ring when Z is selected from C;
Ring E at each occurrence is selected from a 5-6 membered heteroaryl ring and ring F at each occurrence is a 3-10 membered heterocyclic ring when Z is selected from N;
R_{S4} is independently selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4A})₂, —OR_{S4A}, —SR_{S4A}, —S(═O)R_{S4B}, —S(═O)₂R_{S4B}, —C(═O)R_{S4B}, —C(═O)OR_{S4A}, —OC(═O)R_{S4B}, —C(═O)N(R_{S4A})₂, —NR_{S4A}C(═O)R_{S4B}, —OC(═O)OR_{S4A}, —NR_{S4A}C(═O)OR_{S4A}, —NR_{S4A}C(═S)OR_{S4A}, —OC(═O)N(R_{S4A})₂, —NR_{S4A}C(═O)N(R_{S4A})₂, —S(═O)OR_{S4A}, —OS(═O)R_{S4B}, —S(═O)N(R_{S4A})₂, —NR_{S4A}S(═O)R_{S4B}B, —S(═O)₂OR_{S4A}, —OS(═O)₂R_{S4B}, —S(═O)₂N(R_{S4A})₂, —NR_{S4A}S(═O)₂R_{S4B}, —OS(═O)₂OR_{S4A}, —NR_{S4A}S(═O)₂OR_{S4A}, —OS(═O)₂N(R_{S4A})₂, —NR_{S4A}S(═O)₂N(R_{S4A})₂, —P(R_{S4A})₂, —P(═O)(R_{S4B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4C})₂, —OR_{S4C}, —SR_{S4C}, —S(═O)R_{S4D}, —S(═O)₂R_{S4D}, —C(═O)R_{S4D}, —C(═O)OR_{S4D}, —OC(═O)R_{S4D}, —C(═O)N(R_{S4C})₂, —NR_{S4C}C(═O)R_{S4D}, —OC(═O)OR_{S4C}, —NR_{S4C}C(═O)OR_{S4C}, —NR_{S4C}C(═S)OR_{S4C}, —OC(═O)N(R_{S4C})₂, —NR_{S4C}C(═O)N(R_{S4D}), —S(═O)OR_{S4C}, —OS(═O)R_{S4D}, —S(═O)N(R_{S4C})₂, —NR_{S4C}S(═O)R_{S4D}, —S(═O)₂OR_{S4D}, —OS(═O)₂R_{S4D}, —S(═O)₂N(R_{S4C})₂, —NR_{S4C}S(═O)₂R_{S4D}, —OS(═O)₂OR_{S4C}, —NR_{S4C}S(═O)₂OR_{S4C}, —OS(═O)₂N(R_{S4C}), —NR_{S4C}S(═O)₂N(R_{S4C})₂, —P(R_{S4C})₂, —P(═O)(R_{S4D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;
R₅ is selected from hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5A})₂, —OR_{5A}, —SR_{5A}, —S(═O)R_{5B}, —S(═O)₂R_{5B}, —C(═O)R_{5B}, —C(═O)OR_{5A}, —OC(═O)R_{5B}, —C(═O)N(R_{5A})₂, —NR_{5A}C(═O)R_{5B}, —OC(═O)OR_{5A}, —NR_{5A}C(═O)OR_{5A}, —NR_{5A}C(═S)OR_{5A}, —OC(═O)N(R_{5A})₂, —NR_{5A}C(═O)N(R_{5A}), —S(═O)OR_{5A}, —OS(═O)R_{5B}, —S(═O)N(R_{5A})₂, —NR_{5A}S(═O)R_{5B}, —S(═O)₂OR_{5A}, —OS(═O)₂R_{5B}, —S(═O)₂N(R_{5A})₂, —NR_{5A}S(═O)₂R_{5B}, —OS(═O)₂OR_{5A}, —NR_{5A}S(═O)₂OR_{5A}, —OS(═O)₂N(R_{5A})₂, —NR_{5A}S(═O)₂N(R_{5A})₂, —P(R_{5A})₂, —P(═O)(R_{5B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5C})₂, —OR_{5C}, —SR_{5C}, —S(═O)R_{5D}, —S(═O)₂R_{5D}, —C(═O)R_{5D}, —C(═O)OR_{5D}, —OC(═O)R_{5D}, —C(═O)N(R_{5C})₂, —NR_{5C}C(═O)R_{5D}, —OC(═O)OR_{5C}, —NR_{5C}C(═O)OR_{5C}, —NR_{5C}C(═S)OR_{5C}, —OC(═O)N(R_{5C})₂, —NR_{5C}C(═O)N(R_{5C})₂, —S(═O)OR_{5C}, —OS(═O)R_{5D}, —S(═O)N(R_{5C})₂, —NR_{5C}S(═O)R_{5D}, —S(═O)₂OR_{5C}, —OS(═O)₂R_{5D}, —S(═O)₂N(R_{5C})₂, —NR_{5C}S(═O)₂R_{5D}, —OS(═O)₂OR_{5C}, —NR_{5C}S(═O)₂OR_{5C}, —OS(═O)₂N(R_{5C})₂, —NR_{5C}S(═O)₂N(R_{5C})₂, —P(R_{5C})₂, —P(═O)(R_{5D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Each of R_{1A}, R_{1C}, R_{S2A}, R_{S2C}, R_{S31A}, R_{S31C}, R_{S32A}, R_{S32C}, R_{37A}, R_{S38A}, R_{S38C}, R_{S39A}, R_{S39C}, R_{S315A}, R_{S315C}, R_{S4A}, R_{S4C}, R_{5A}, R_{5C}, R_{8A}, R_{8C}, R_{S8A} and R_{S8C} is independently selected from hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, ~NR_{C}C(═O)R_{D}, —OC(═O)OR_{C}, —NR_{C}C(═O)OR_{D}, —OC(═O)N(R_{C})₂, —NR_{C}C(═O)N(R_{D}), —S(═O)OR_{C}, —OS(═O)R_{D}, —S(═O)N(R_{C})₂, —NR_{C}S(═O)R_{D}, —S(═O)₂OR_{D}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Optionally, (two R_{1A}, two R_{1C}, two R_{S2A}, two R_{S2C}, two R_{S31A}, two R_{S31C}, two R_{S32A}, two R_{S32C}, two R_{37A}, two R_{S38A}, two R_{S38C}, two R_{S39A}, two R_{S39C}, two R_{S315A}, two R_{S315C}, two R_{S4A}, two R_{S4C}, two R_{5A}, two R_{5C}, two R_{8A}, two R_{8C}, two R_{S8B} and two R_{S8C}) together with the nitrogen atom to which they are both attached forms a 3-10 membered heterocyclic ring or a 5-10 membered heteroaryl ring, wherein, said 3-10 membered heterocyclic ring or 5-10 membered heteroaryl ring is independently unsubstituted or substituted with one or more R_{SS};

Each of R_{1B}, R_{1D}, R_{S2B}, R_{S2D}, R_{S31B}, R_{S31D}, R_{S32B}, R_{S32D}, R_{S38B}, R_{S38D}, R_{S39B}, R_{S315B}, R_{S315D}, R_{S39B}, R_{S4B}, R_{S4D}, R_{5B}, R_{5D}, R_{8B}, R_{8D}, R_{S8B} and R_{S8D} is independently selected from hydrogen, deuterium, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)N(R_{C})₂, —OS(═O)R_{D}, —NR_{C}C(═O)OR_{D}, —OC(═O)N(R_{C})₂, —S(═O)OR_{C}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Each of (R_{A}, R_{B}, R_{C} and R_{D}) is independently selected from hydrogen, deuterium, —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more R_{SA};

Each of R_{SX1}, R_{S21}, R_{S22}, R_{S23}, R_{S33}, R_{S34}, R_{S35}, R_{S37}, R_{S310}, R_{S316}, R_{S311}, R_{S312}, R_{S313}, R_{S321}, R_{S322}, R_{S323}, R_{S381}, R_{S382}, R_{S383}, R_{S391}, R_{S392}, R_{S393}, R_{S3151}, R_{S3152}, R_{S3153}, R_{S81}, R_{S82}, R_{SS} and R_{SA} is independently selected from deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆ alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂ (OC₁₋₆alkyl), —OS(═O)₂ (C₁₋₆alkyl), —S(═O)₂NH₂, —S(═O)NH(C₁₋₆alkyl), —S(═O)₂N(C₁₋₆alkyl)₂, —NHS(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O))₂N(C₁₋₆alkyl)₂, ~N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl, wherein, said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂ (C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)NH(C₁₋₃alkyl), —S(═O)N(C₁₋₃alkyl)₂, —NHS(═O)₂(C₁₋₃alkyl), —OS(═O)₂O(C₁₋₃alkyl), —NHS(═O)₂O(C₁₋₃alkyl), —N(C₁₋₃alkyl)S(═O)₂ (C₁₋₃alkyl), —N(C₁₋₃alkyl)S(═O)₂O(C₁₋₃alkyl), —OS(═O)₂NH₂, —OS(═O)₂NH(C₁₋₃alkyl), —N(C₁₋₃alkyl)S(═O)NH₂, —OS(═O)₂N(C₁₋₃alkyl)₂, —NHS(═O)₂NH(C₁₋₃alkyl), —NHS(═O)₂N(C₁₋₃alkyl)₂, —NHS(═O)₂NH₂, —N(C₁₋₃alkyl)S(═O)₂NH(C₁₋₃alkyl), —N(C₁₋₆alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-10 membered cycloalkyl, 3-10 membered cycloalkenyl, 3-10 membered cycloalkynyl, 3-10 membered heterocyclyl, 6-10 membered aryl or 5-10 membered heteroaryl;

Each of heterocyclyl or heterocyclic at each occurrence independently contains 1, 2, 3 or 4 heteroatoms selected from N, O, S, S(═O) or S(═O)₂;

Each of heteroaryl at each occurrence independently contains 1, 2, 3 or 4 heteroatoms selected from N, O, or S.
[12]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of [11], wherein, the compound is selected from any one of following formulas:

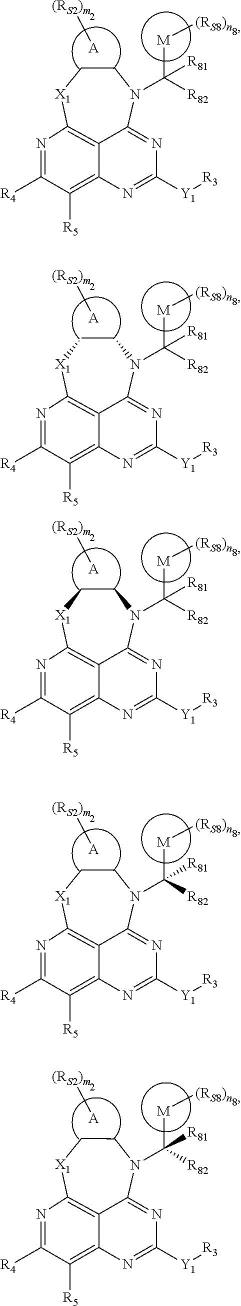

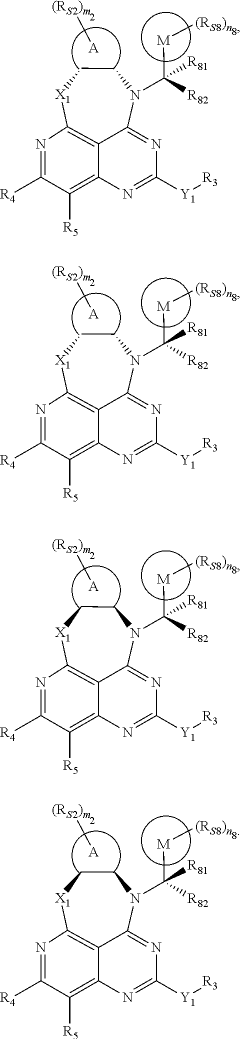

[13]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of [12], wherein, R₈₁ and R₈₂ is selected from hydrogen, deuterium, —C₁₋₆alkyl, or 3-6 membered cycloalkyl;

Preferably, R₈₁ is selected from hydrogen or deuterium; R₈₂ is selected from methyl or cyclopropyl.

Ring M is selected from a 5 membered heteroaryl containing 1, 2, 3 or 4 heteroatoms selected from N, O or S or 6 membered heteroaryl containing 1 or 2 heteroatoms selected from N.

Preferably, ring M is 6 membered heteroaryl containing 1 or 2 heteroatoms selected from N, and said 6 membered heteroaryl is substituted with one —NH₂ or —OH and further optionally substituted with one or more R_{S8}.

In some embodiments, the moiety of

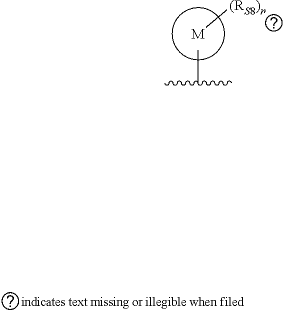

is selected from

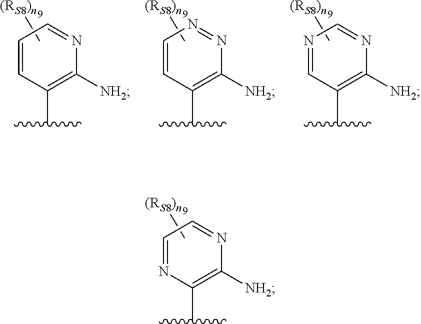

Wherein, n₉ is selected from 0, 1, 2, 3, or 4;
R_{S8} is selected from deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly.

Preferably, R_{S8} is selected from —NHCH₃.

D₈ is selected from 0 or 1, or n₉ is selected from 0 or 1.

In some embodiments, the moiety of

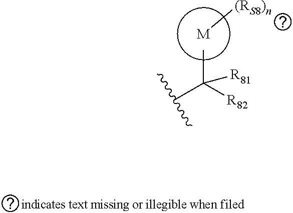

is selected from

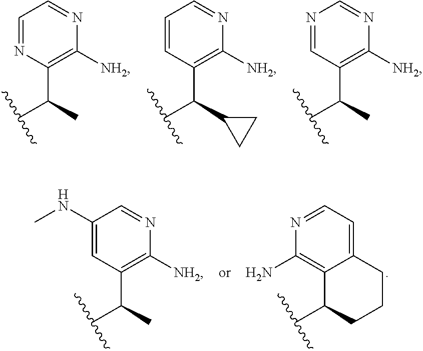

[14]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [13], wherein, Y₁ is selected from O.
[15]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], wherein, each of R₃₁, R₃₂, R₃₃, R₃₄, R₃₅ and R₃₆ is independently selected form hydrogen or deuterium.
[16]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [15], wherein, n₂ is selected from 1.
[17]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [16], wherein, n₃ is selected from 1 and n₄ is selected from 1.
[18]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [17], wherein:
R₃ is selected from

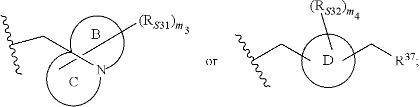

Ring B or ring C is a 4-6 membered heterocyclic ring optionally further containing 1 or 2 heteroatoms selected from N or O;
Ring D is a 3 membered carbocyclic ring;
R₃₇ is selected from —N(R_{37A})₂ or a 6 membered heterocyclyl, wherein said 6 membered heterocyclyl is optionally independently substituted with 1, 2 or 3 R_{S37};
R_{37A} is selected from hydrogen, deuterium, or —C₁₋₆alkyl.
[19]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [18], wherein, the moiety of

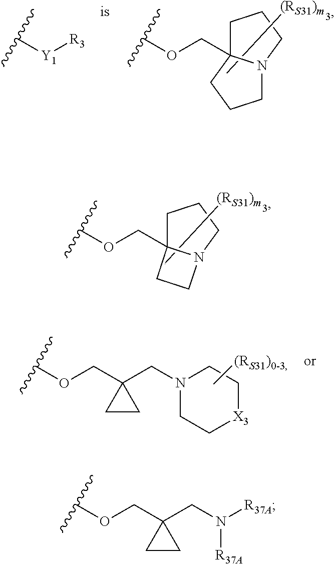

Wherein, X₃ is selected from CH₂, NH, O, S, S(═O) or S(═O)₂, when X₃ is selected from CH₂ or NH, the hydrogen in CH₂ or NH is optionally substituted with one or more R_{S37};
R_{37A} is selected from hydrogen, deuterium, or —C₁₋₃alkyl.
[20]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [19], wherein:

The moiety of

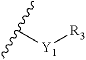

is selected from

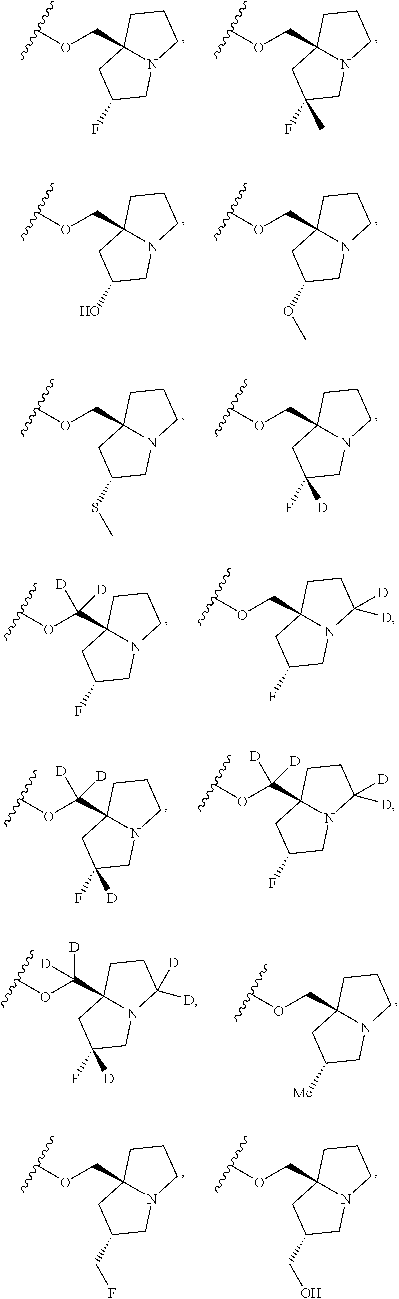

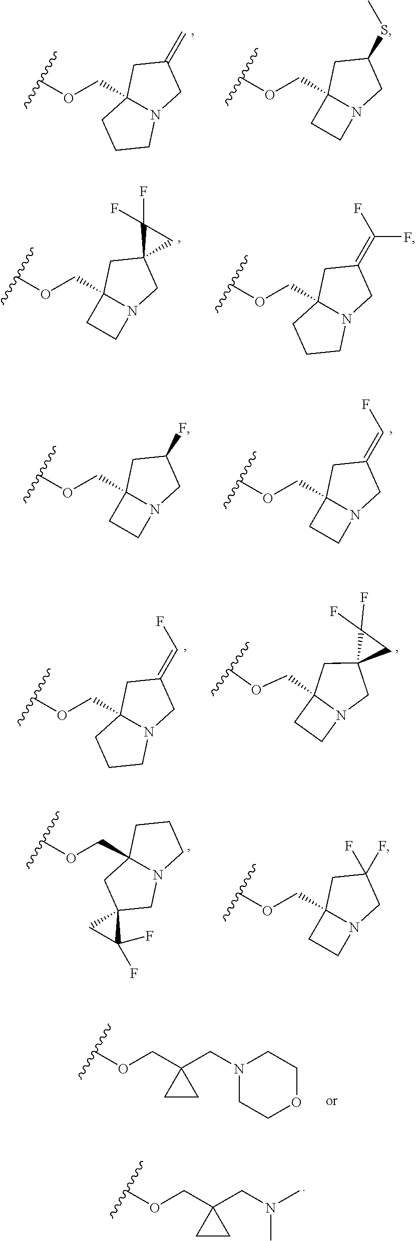

[21]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [20], wherein:

The moiety of

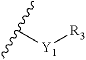

is selected from

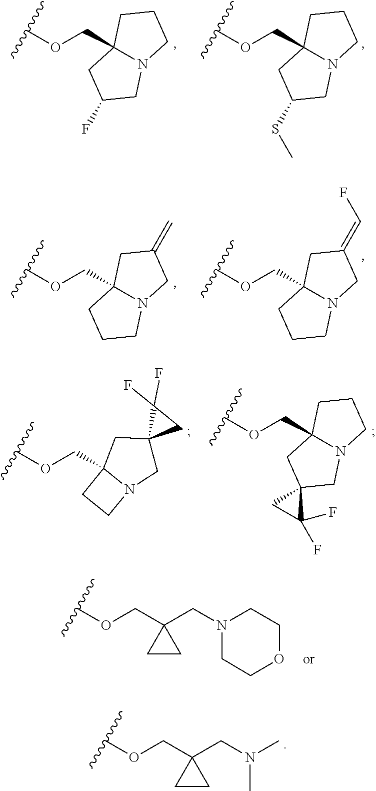

[22]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [21], wherein:
the moiety of

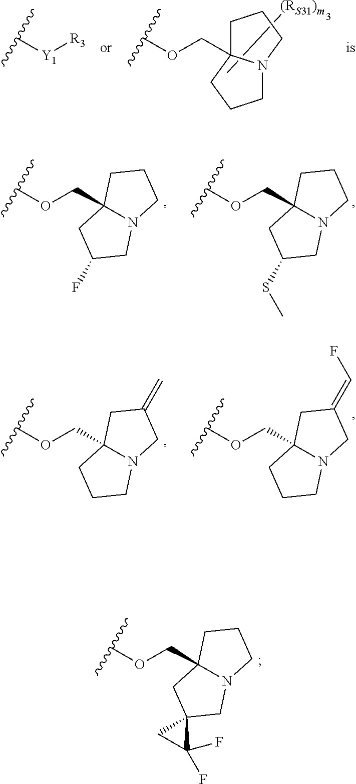

the moiety of

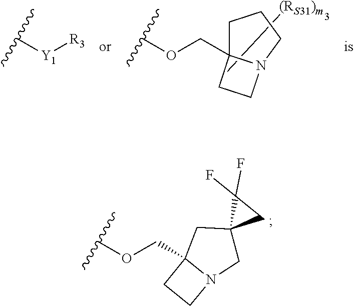

the moiety of

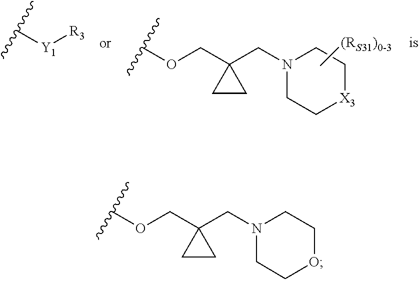

the moiety of

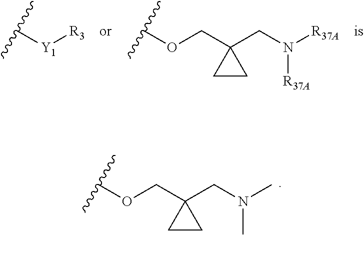

[23]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], wherein, each of R₃₈ and R₃₉ is independently selected form hydrogen or deuterium.
[24]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23], wherein, n₅ is selected from 1.
[25]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [23] and [24], wherein,
Ring I is a 4-6 membered cycloalkyl ring.
[26]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [25], wherein:
Ring J is a 4-6 membered heterocyclic ring containing 1 or 2 heteroatoms selected from N, O or S.
[27]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [26], wherein:

The moiety of

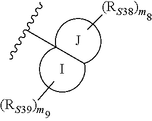

is selected from

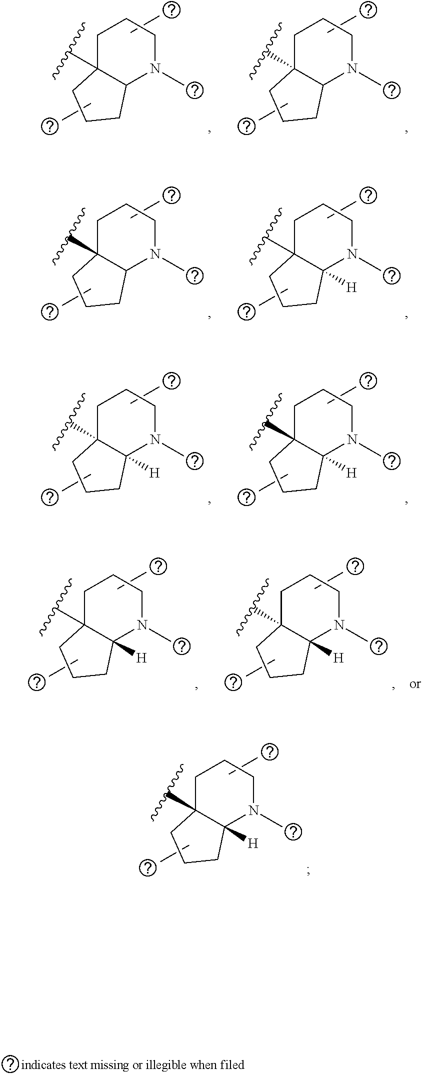

Wherein, R_{S381} is selected from hydrogen or R_{S38};
m₈₁ is selected from 0, 1, 2, 3, 4, 5, 6, 7, or 8.
[28]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [27], wherein:
R_{S381} is selected from hydrogen, deuterium, —C₁₋₆alkyl or 3-6 membered cycloalkyl wherein, said —C₁₋₆alkyl or 3-6 membered cycloalkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly.
[29]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [28], wherein, R_{S381} is selected from hydrogen, deuterium, —CH₃, —CH₂CH₃ or cyclopropyl.
[30]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [29], wherein, m₈₁ is selected from 0.
[31]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [30], wherein:
m₉ is selected from 0, 1 or 2.
[32]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [31], wherein:
m₉ is selected from 0.
[33]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [32], wherein:
m₉ is selected from 1.
[34]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [33], wherein:

The moiety of

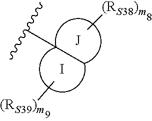

is selected from

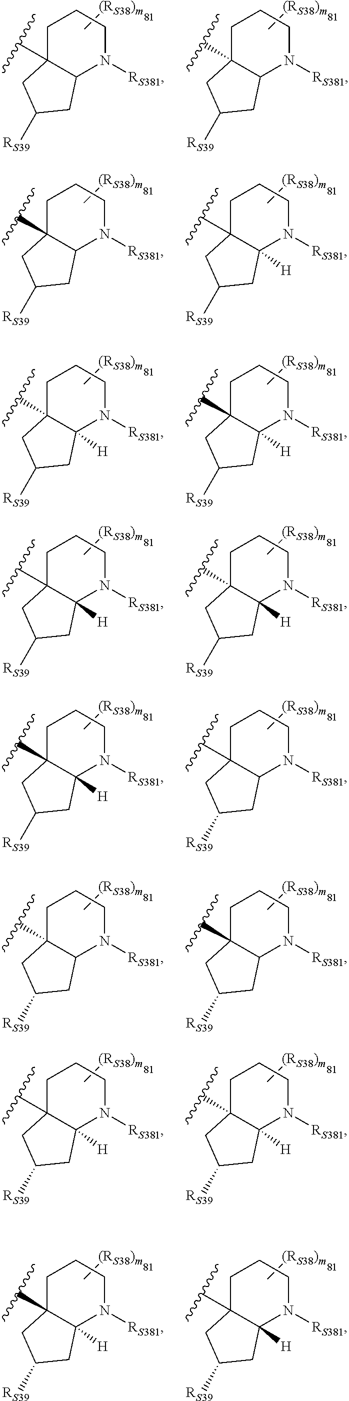

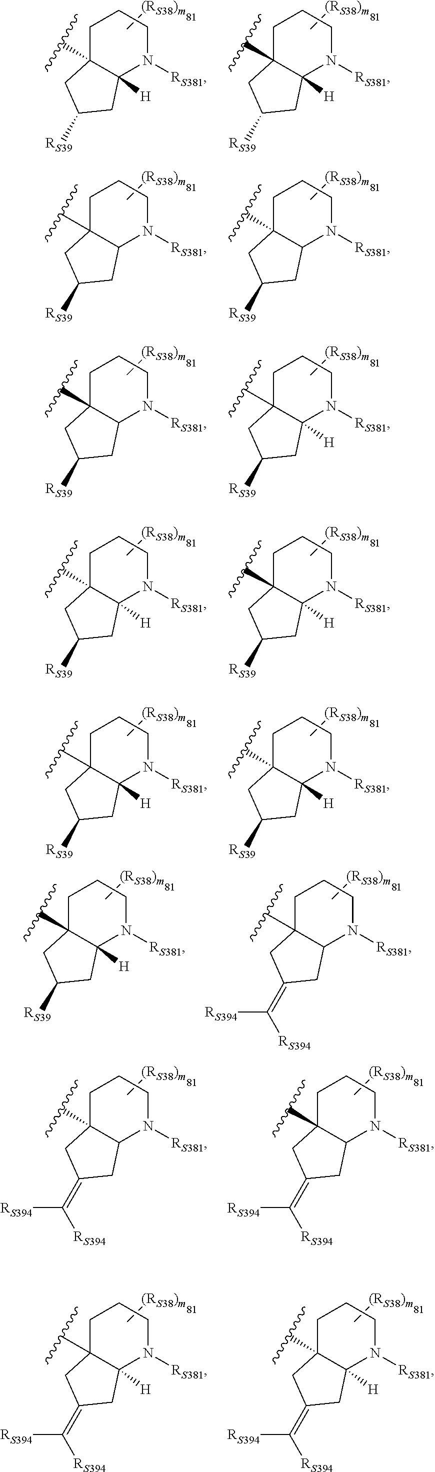

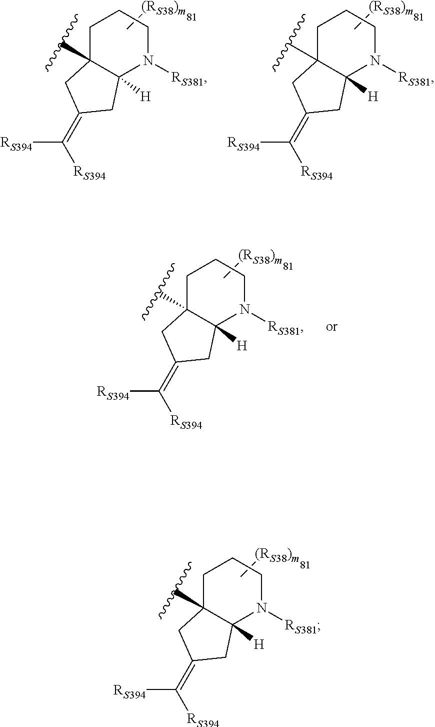

Wherein, R_{S394} is selected from hydrogen or R_{S391}.
[35]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [34], wherein:
R_{S39} is selected from halogen;
Preferably, R_{S39} is selected from —F.
[36]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [35], wherein:
R_{S391} is selected from hydrogen, deuterium, halogen, or —C₁₋₆alkyl, wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly;
Preferably, R_{S391} is selected from hydrogen, deuterium, —F, or —CH₃.
[37]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [36], wherein:

The moiety of

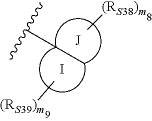

is selected from

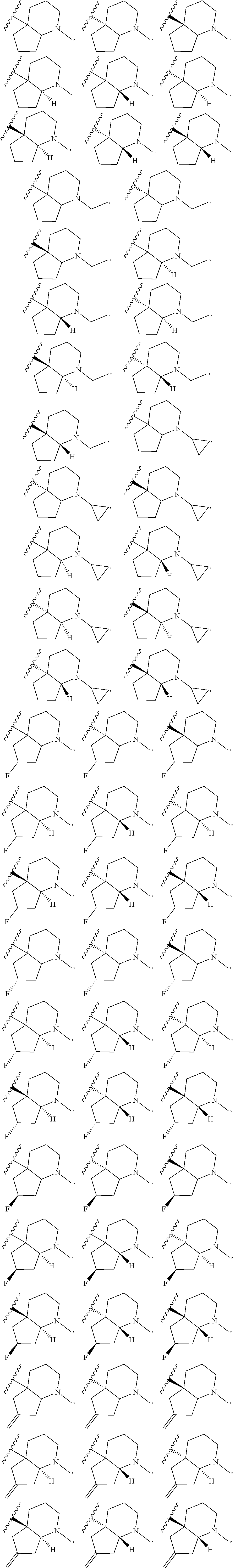

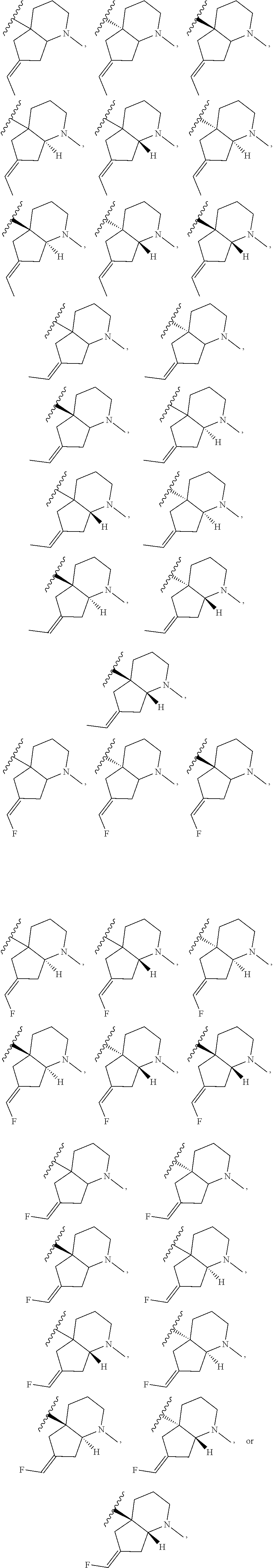

[38]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [23] to [37], wherein:

The moiety of

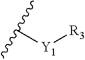

is selected from

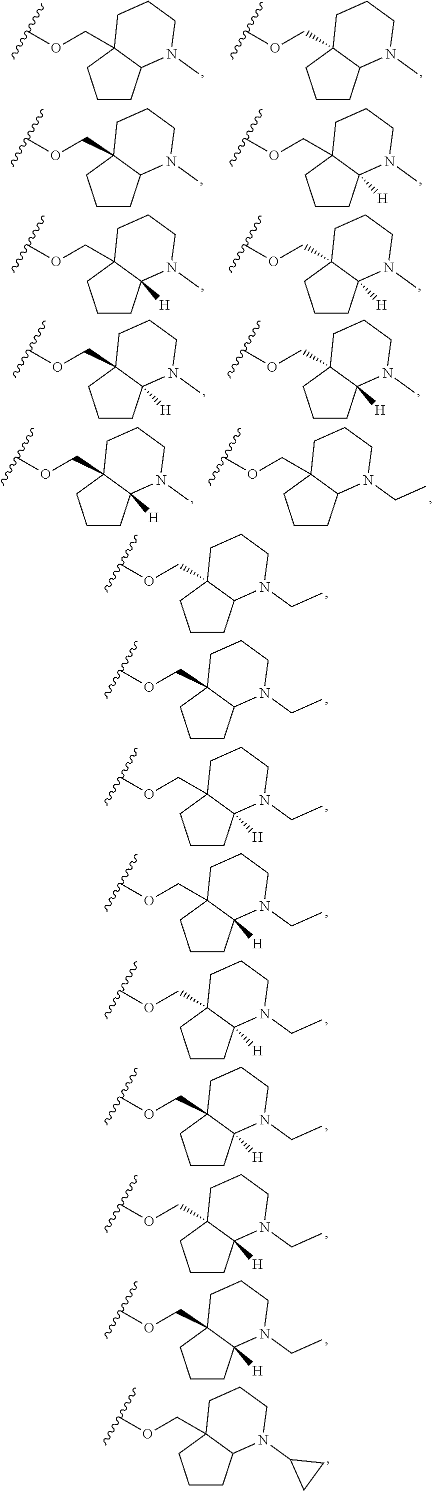

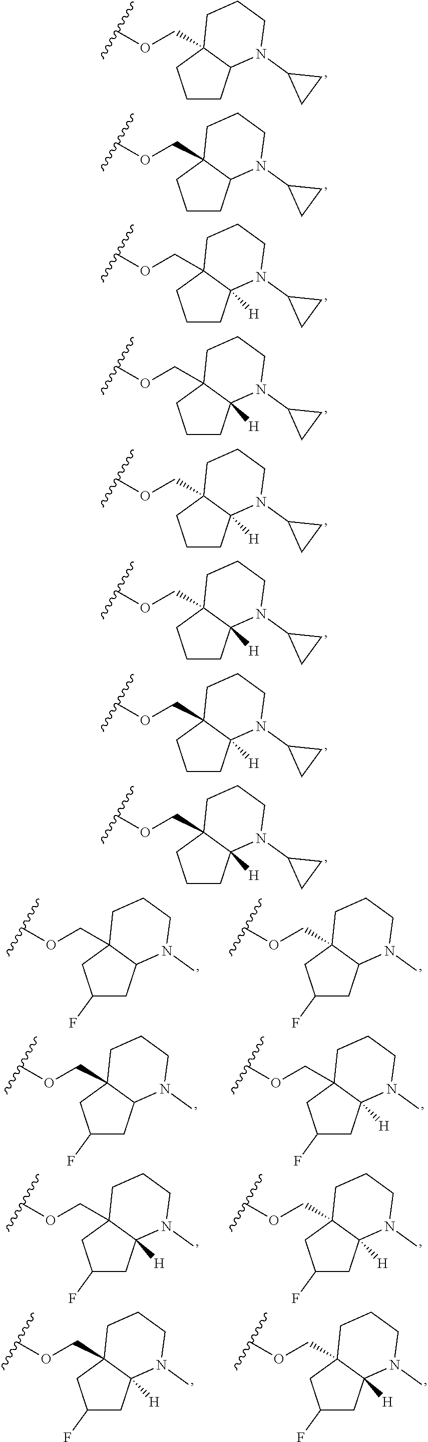

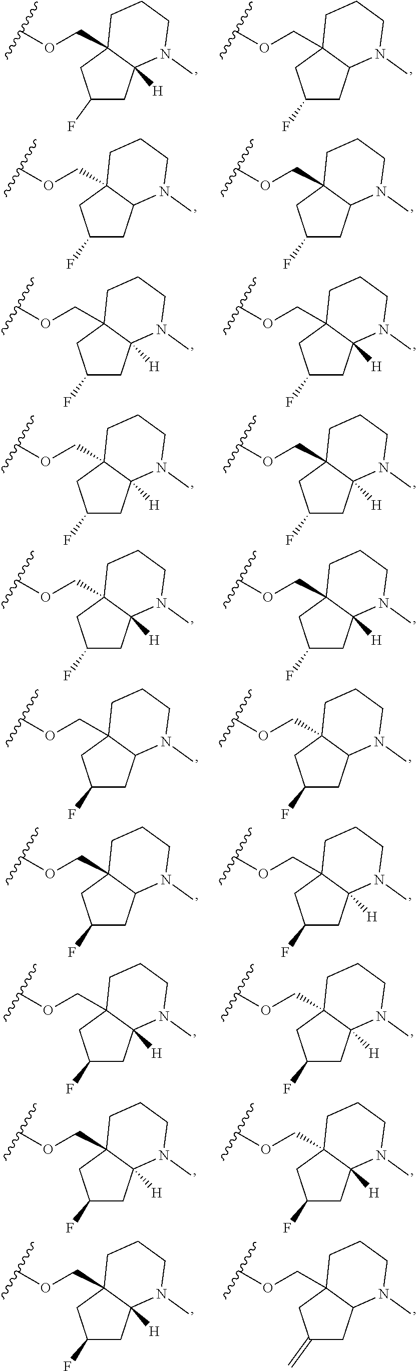

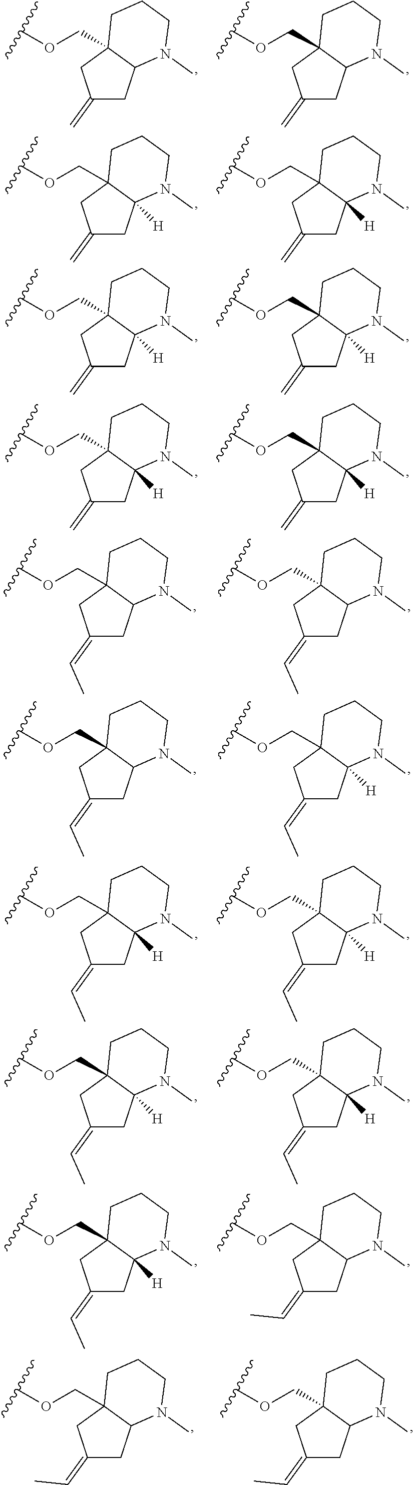

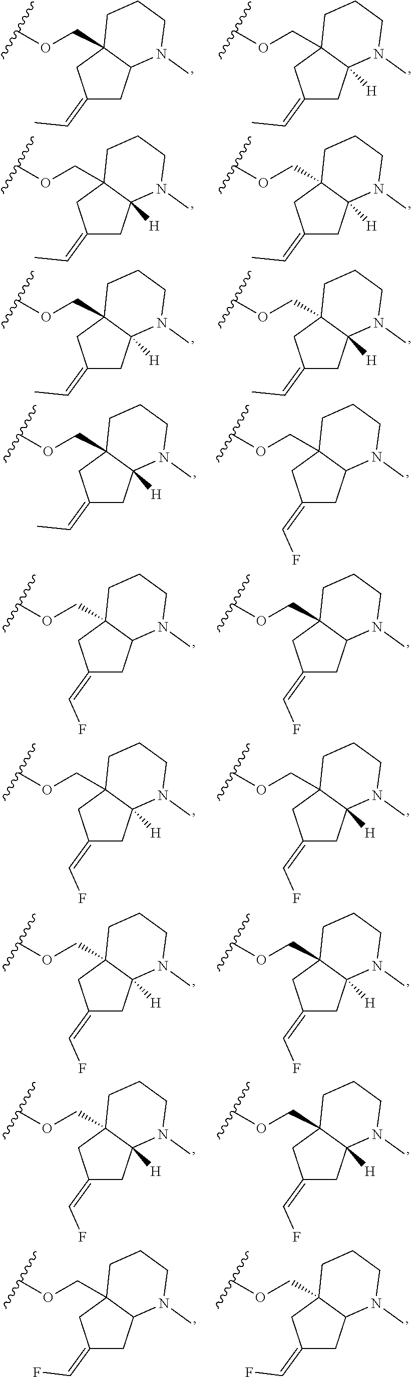

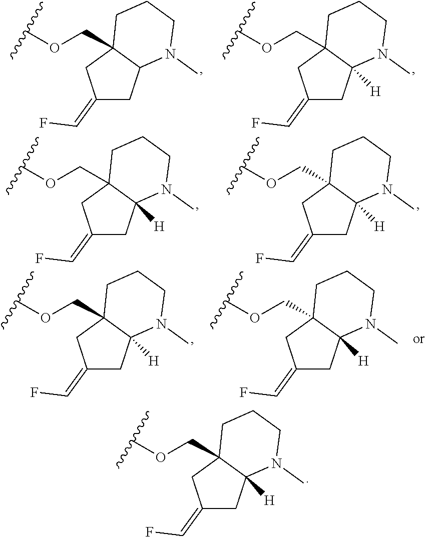

[39]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], wherein:
Ring B is a 4-6 membered heterocyclic ring containing the fused N atom.
[40]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39], wherein:
Ring C is a 4-6 membered heterocyclic ring containing the fused N atom.
[41]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [39] and [40], wherein:

The moiety of

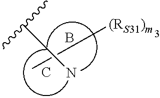

is selected from

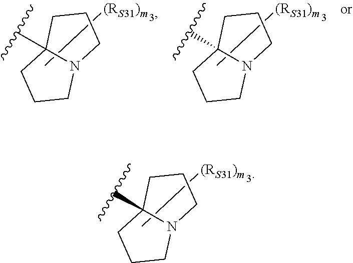

[42]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [41], wherein, m₃ is selected from 0, 1, 2, 3, or 4.
[43]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [42], wherein, R_{S31} is selected from deuterium or —F;

Optionally, two R_{S31} together with the carbon atom to which they are both attached form

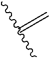

or cyclopropyl; wherein, said

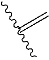

or cyclopropyl is independently unsubstituted or substituted with 1, 2 or 3 R_{S311}; or

Optionally, two adjacent R_{S31} together with the carbon atoms to which they are respectively attached form a 5-10 membered heterocyclic ring containing 1 or 2 heteroatoms selected from N or O, a phenyl ring or a 5-10 membered heteroaryl ring containing 1 or 2 heteroatoms selected from N, O or S, wherein, each of rings is independently unsubstituted or substituted with 1, 2 or 3 R_{S312}.
[44]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [43], wherein:

The moiety of

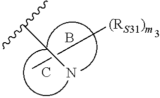

is selected from

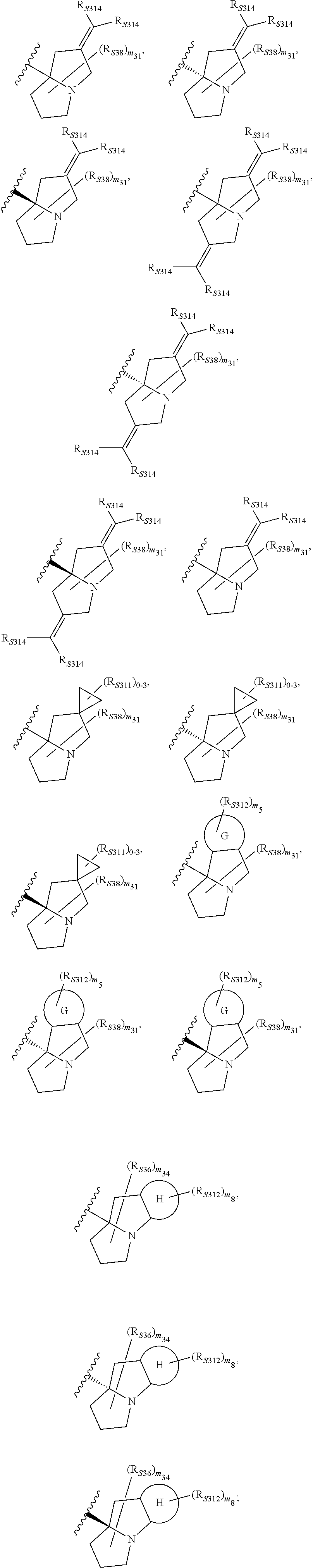

### Wherein,

Ring G is selected from a 5-6 membered heterocyclic ring containing 1 or 2 heteroatoms selected from N or O, a phenyl ring, a 5-6 membered heteroaryl containing 1, 2 or 3 heteroatoms selected from N, O, or S;
Ring H is selected from a 5-10 membered heterocyclic ring containing 1 or 2 heteroatoms selected from N or O, a phenyl ring, a 5-10 membered heteroaryl containing 1 or 2 heteroatoms selected from N, O, or S;
The definition of R_{S36} is same as R_{S31};
R_{S314} is selected from hydrogen or R_{S311};
m₃₁ is selected from 0, 1, 2, 3, 4, 5, 6, 7, 8, 9 or 10;
m₃₂ is selected from 0, 1, 2, 3, 4, 5, 6, 7 or 8;
m₃₃ is selected from 0, 1, 2, 3, 4, 5, 6, 7, 8, 9, or 10;
m₃₄ is selected from 0, 1, 2, 3, 4, 5, 6, 7, 8, 9, 10, 11 or 12;
m₅ is selected from 0, 1, 2, 3, 4, 5, or 6;
m₆ is selected from 0, 1, 2, 3, 4, 5, or 6.
[45]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [44], wherein:

The moiety of

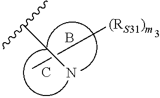

is selected from

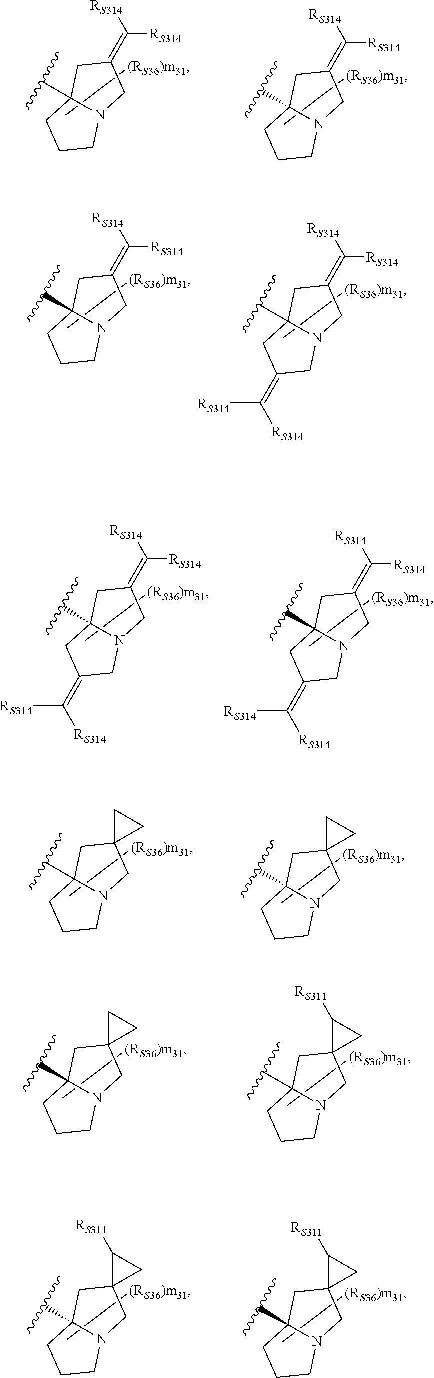

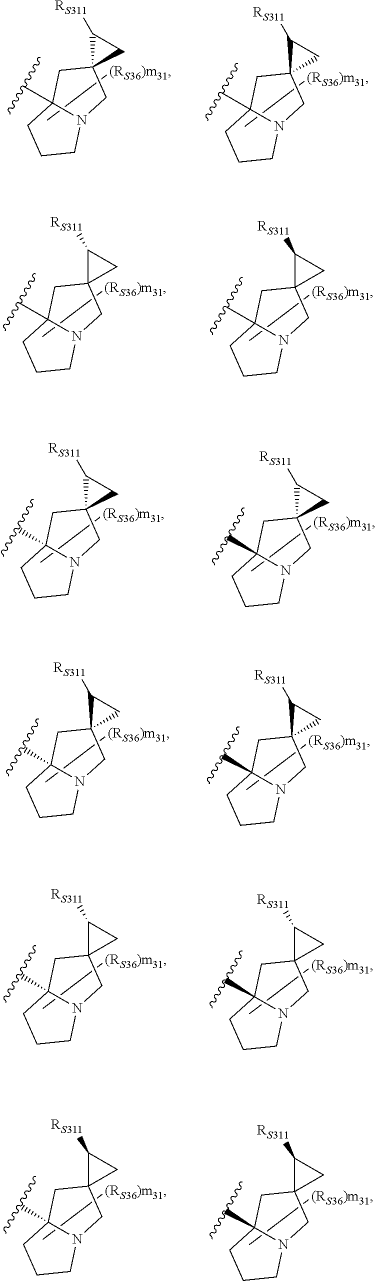

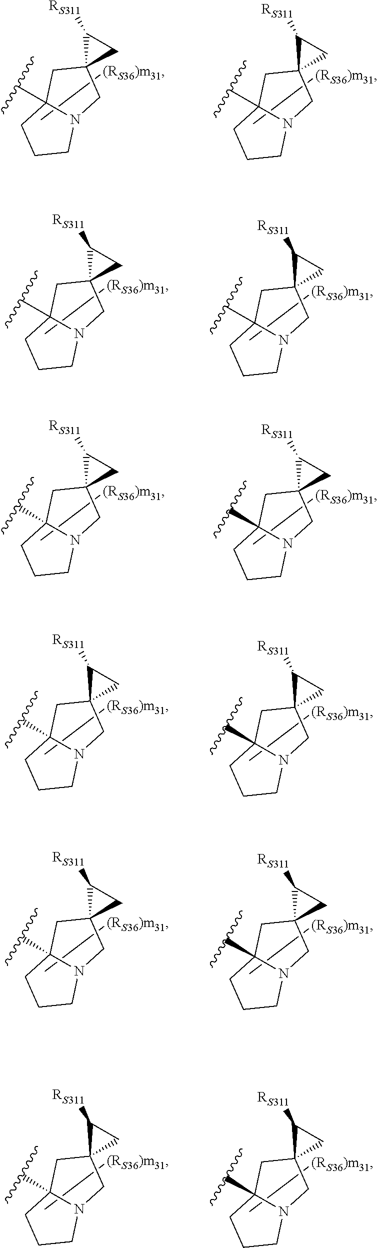

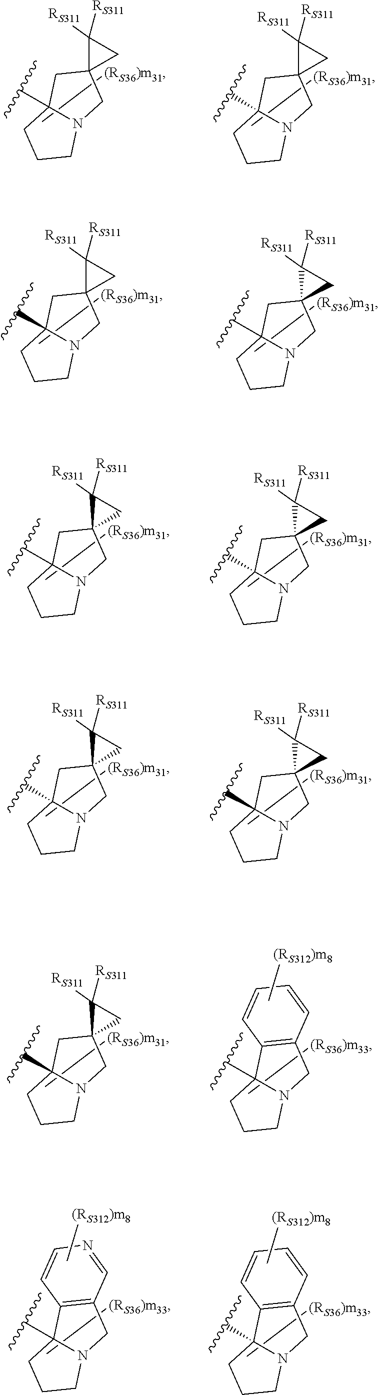

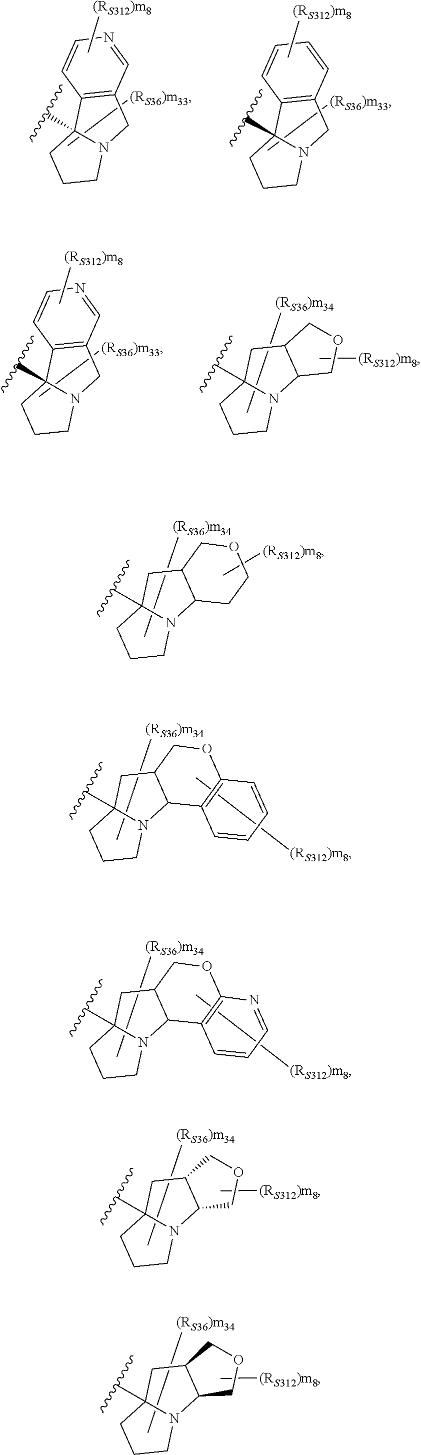

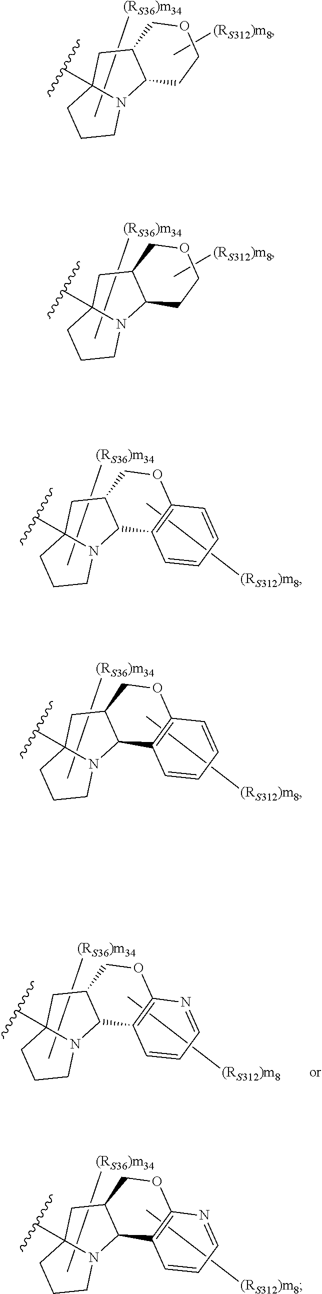

### Wherein,

m₃₁ is selected from 0, 1, 2, 3, 4, 5, 6, 7, 8, 9 or 10;
m₃₂ is selected from 0, 1, 2, 3, 4, 5, 6, 7 or 8;
m₃₃ is selected from 0, 1, 2, 3, 4, 5, 6, 7, 8, 9, or 10;
m₃₄ is selected from 0, 1, 2, 3, 4, 5, 6, 7, 8, 9, 10, 11 or 12;
m₅ is selected from 0, 1, 2, 3, 4, 5, or 6;
m₆ is selected from 0, 1, 2, 3, 4, 5, or 6.
[46]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [45], wherein:
R_{S311} is independently selected from deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly;
R_{S312} is independently selected from deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly;
R_{S314} is independently selected from hydrogen, deuterium, halogen, —C₁₋₆alkyl, wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly.
[47]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [46], wherein:
R_{S311} is independently selected from deuterium, —F or —OCH₃;
R_{S312} is independently selected from deuterium, —F, —OCH₃, or —CH₂OCH₃;
R_{S314} is independently selected from hydrogen, deuterium, —F, —CH₃, —CH₂OCH₃, —CH₂CH₂CH₃, —CH(CH₃)₂, —CHF₂, —CH₂CH(CH₃)₂.
[48]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [49], wherein:
R_{S36} is selected from deuterium or —F.
[49]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [48], wherein:
m₃₁ is selected from 0 or 1;
m₃₂ is selected from 0 or 1;
m₃₃ is selected from 0 or 1;
m₃₄ is selected from 0 or 1;
m₅ is selected from 0 or 1;
m₆ is selected from 0 or 1.
[50]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [49], wherein:

The moiety of

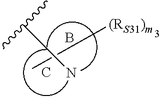

is selected from

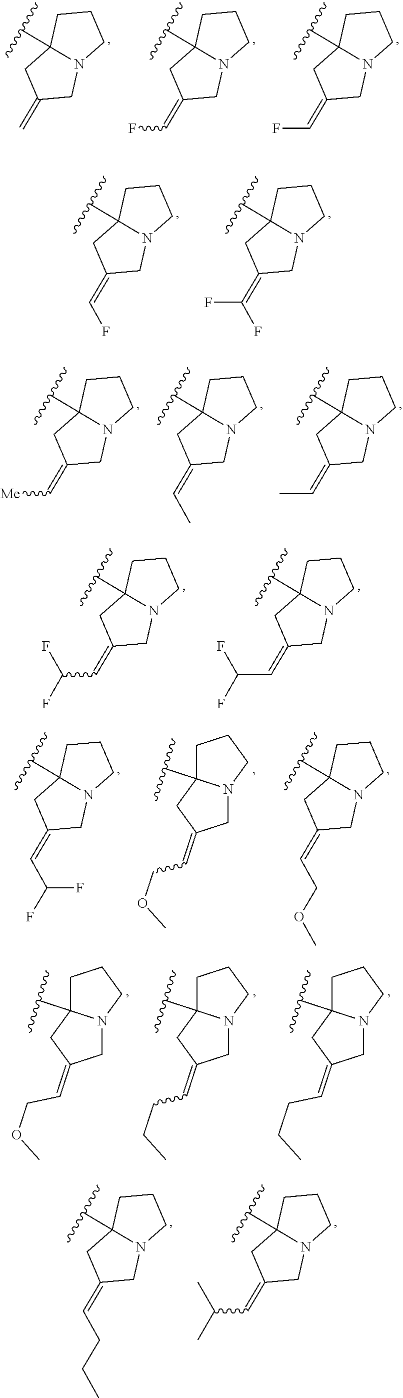

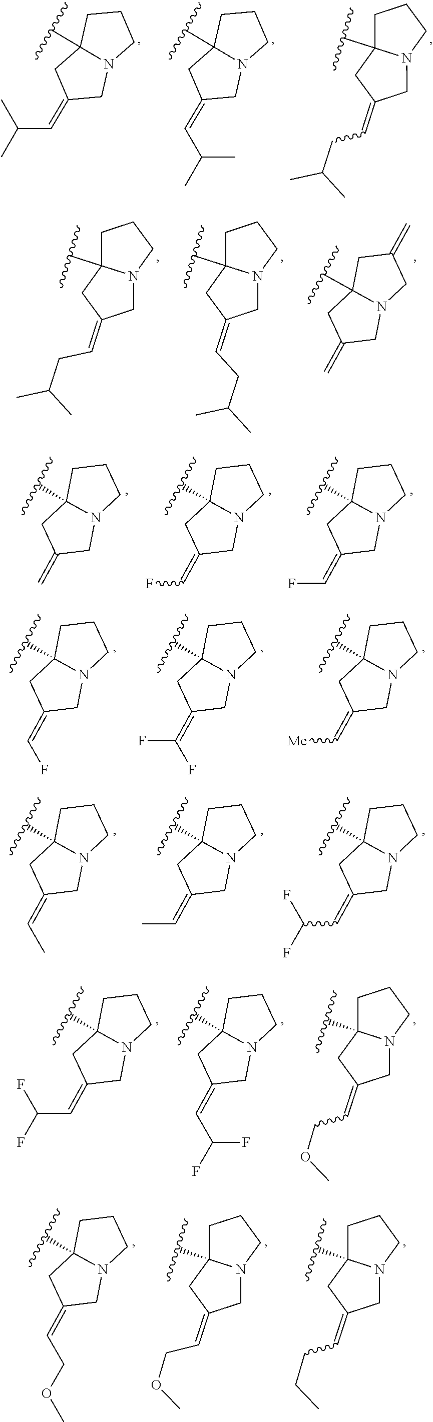

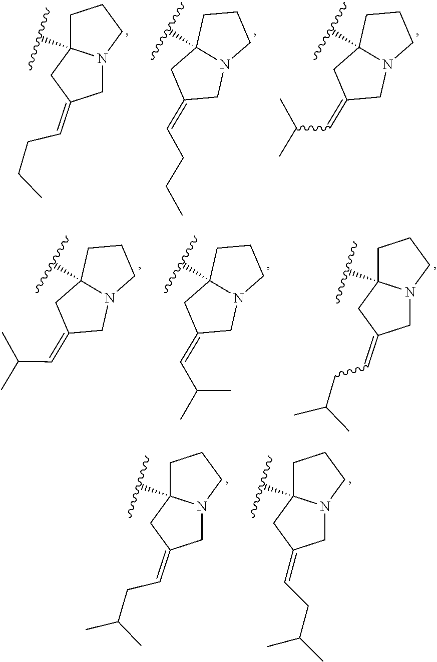

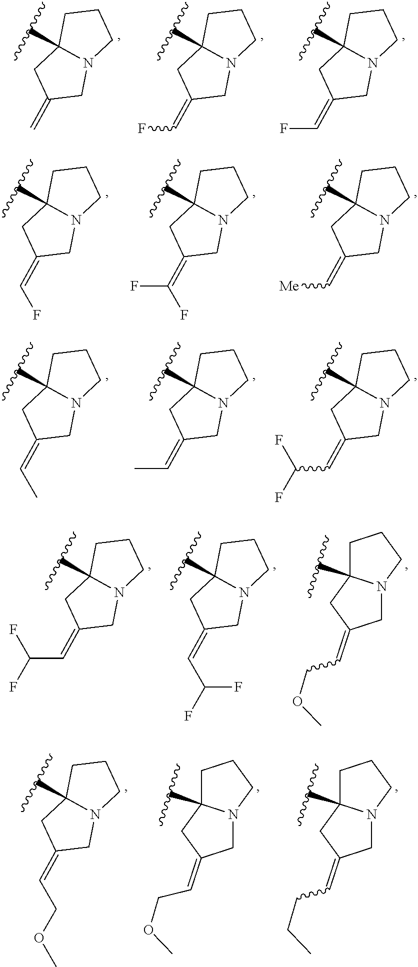

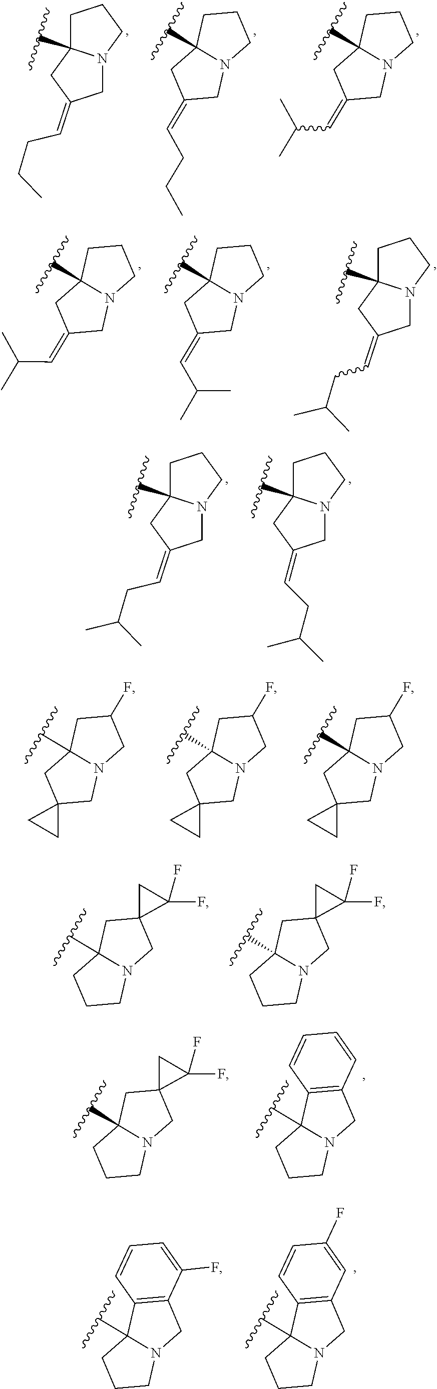

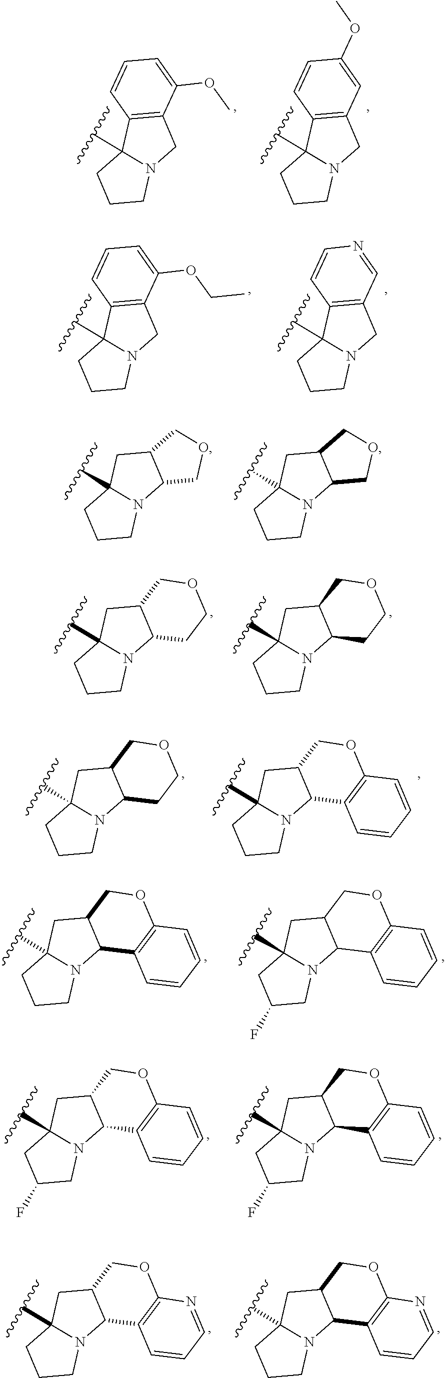

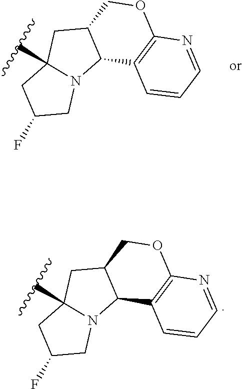 [SMILES: *[C@]12C[C@@H](F)CN1[C@H]1c3cccnc3OC[C@H]1C2 ; *[C@]12C[C@@H](F)CN1[C@@H]1c3cccnc3OC[C@@H]1C2]

[51]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14] and [39] to [50], wherein:

The moiety of

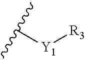

is selected from

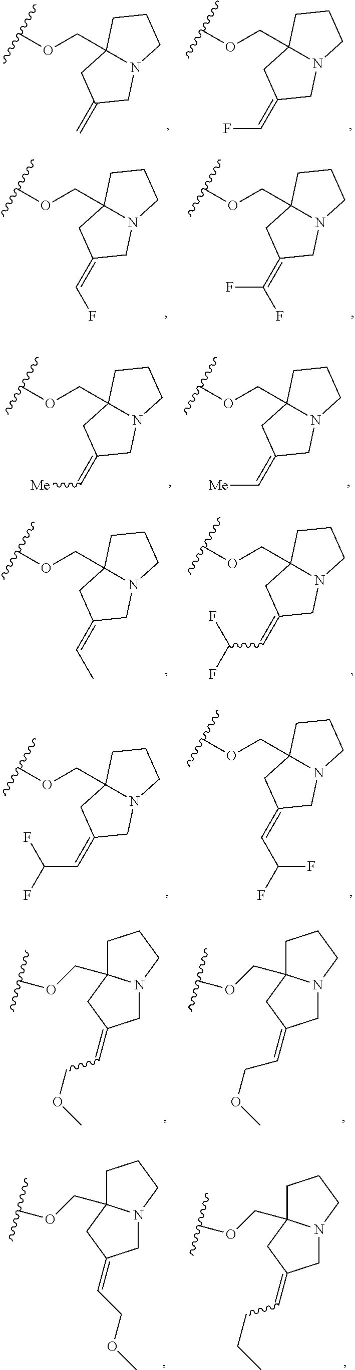

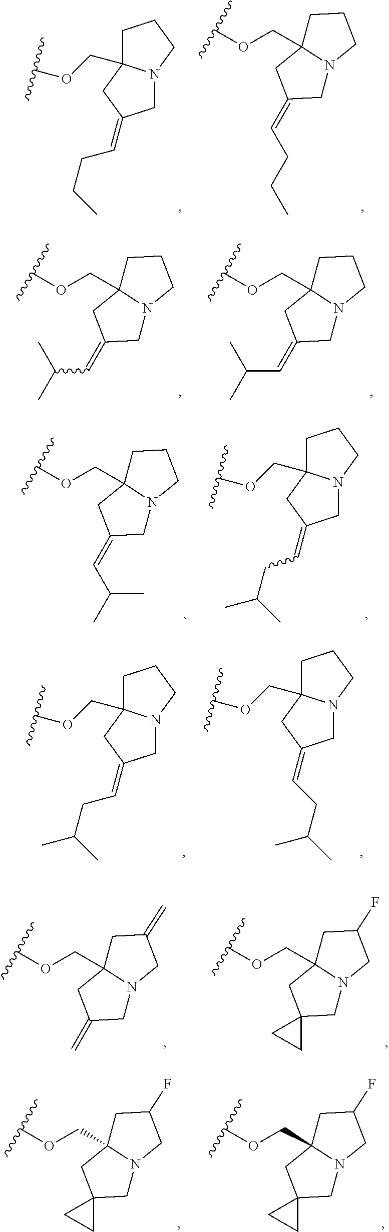

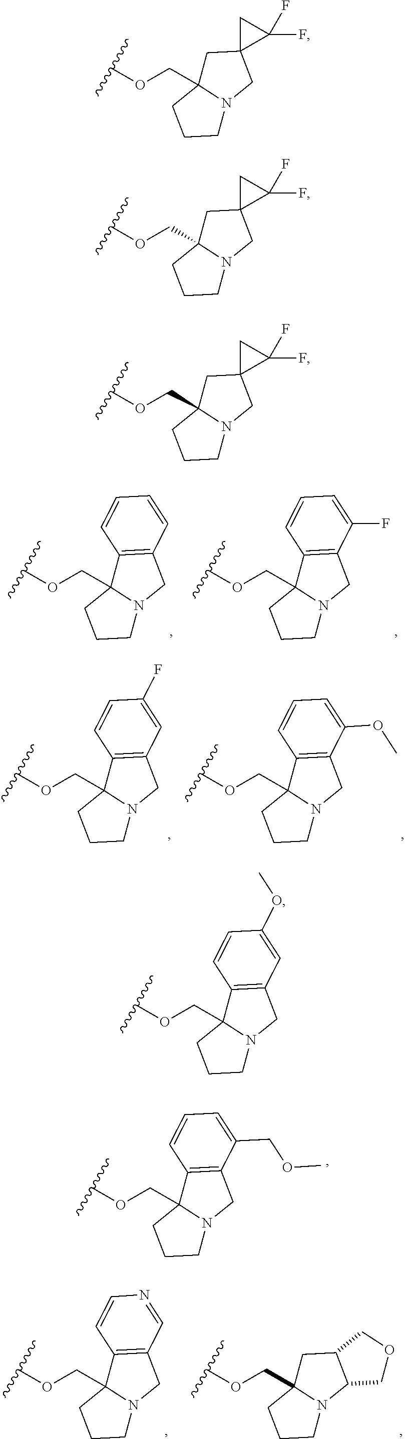

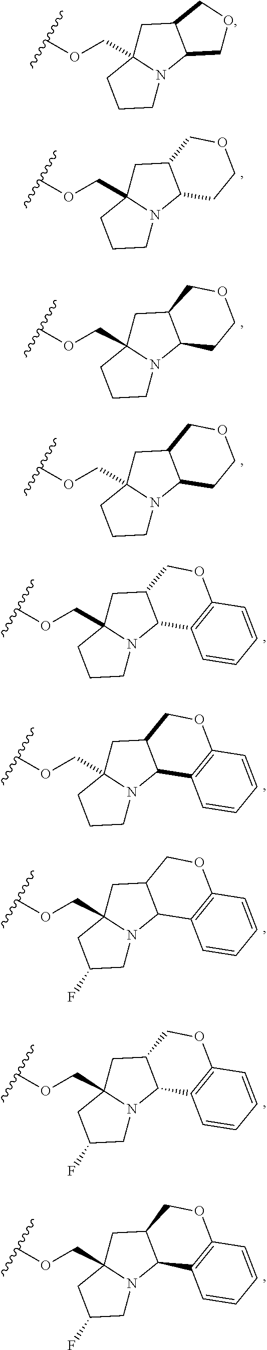

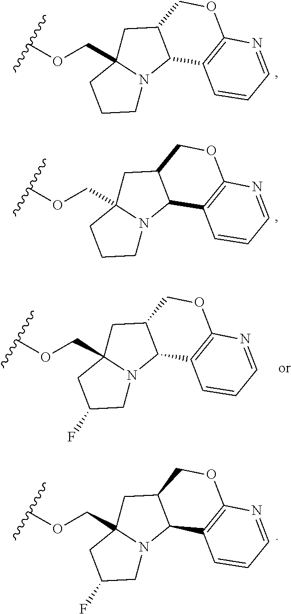

[52]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], wherein, each of R₃₁₀ and R₃₁₁ is independently selected form hydrogen or deuterium.
[53]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], or wherein, n₆ is selected from 1 or 2.
[54]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] or [53], wherein:
Ring K is a 4-10 membered heterocyclic ring containing 1 or 2 heteroatoms selected from N or O atom.
[55]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [54], wherein:

The moiety of

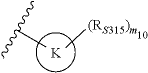

is selected from

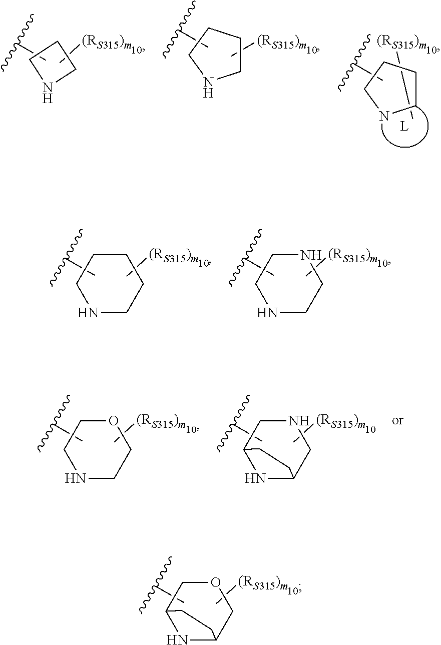

Wherein, ring L is selected from a 4-6 membered heterocyclic ring optionally further containing 1 or 2 heteroatoms selected from N or O.
[56]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [55], wherein:

The moiety of

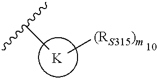

is selected from

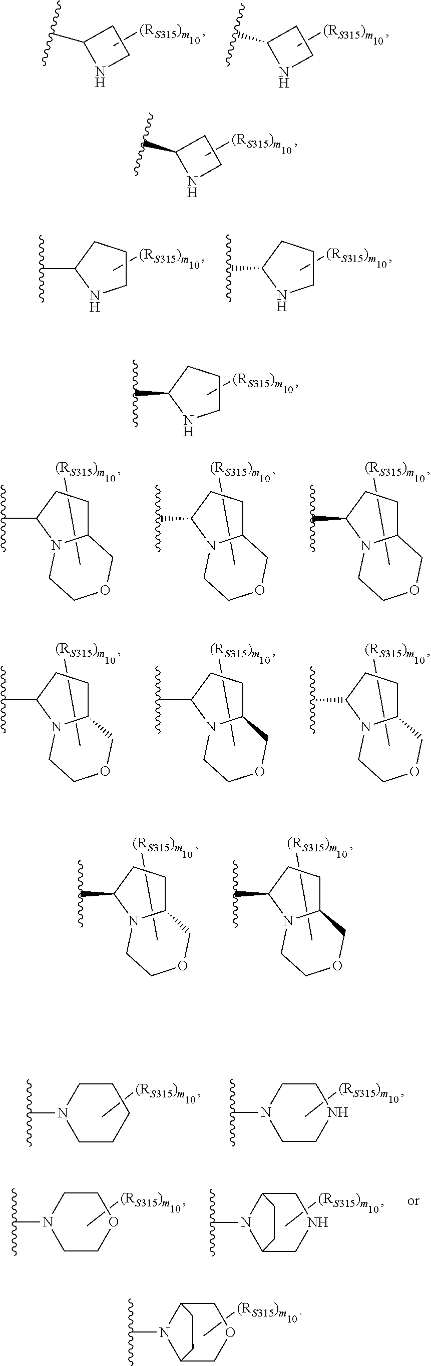

[57]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [56], wherein:
R_{S315} is independently selected from deuterium, halogen, —C₁₋₆alkyl, haloC₁₋₆alkyl, haloC₁₋₆alkoxy, —OH, —OC₁₋₆alkyl, —CN, —NH₂, —NH(C₁₋₆alkyl) or —N(C₁₋₆alkyl)₂; wherein, said —C₁₋₆alkyl is independently unsubstituted or substituted with 1, 2, or 3 substituents selected from deuterium, halogen, haloC₁₋₆alkyl, haloC₁₋₆alkoxy, —CN, —NH₂, —NH(C₁₋₆alkyl), —N(C₁₋₆alkyl)₂, —OH or —OC₁₋₆alkly.
[58]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [57], wherein, R_{S315} is independently selected from —F or —CH₃.
[59]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [58], wherein, mic is selected from 0, 1, 2, or 3.
[60]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [59], wherein:

The moiety of

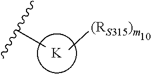

is selected from

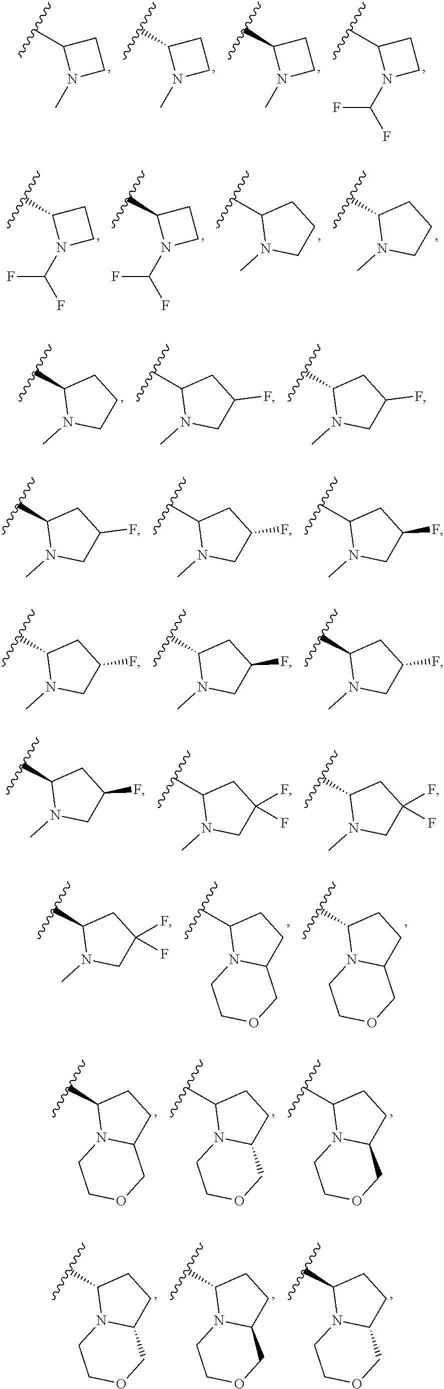

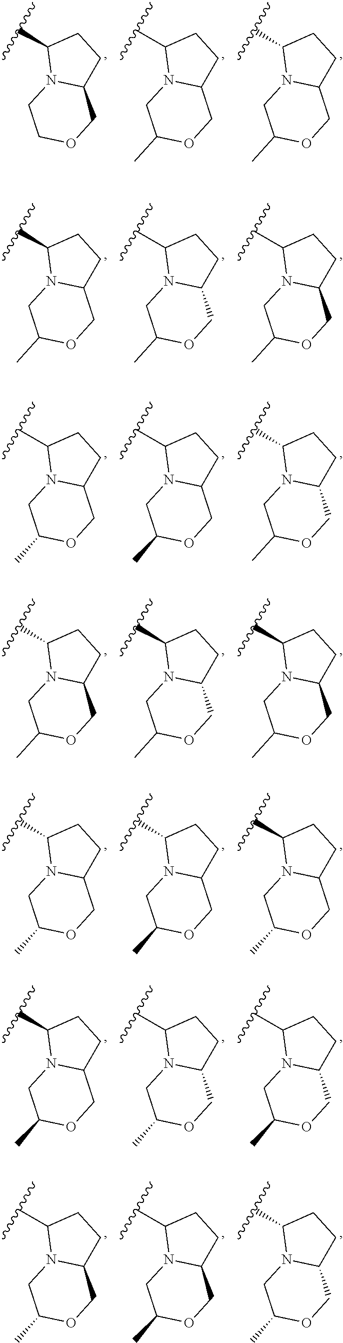

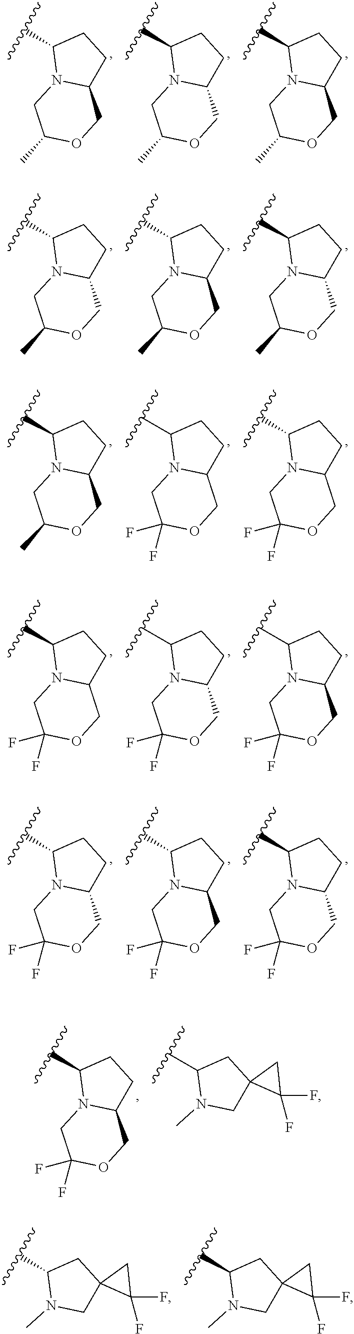

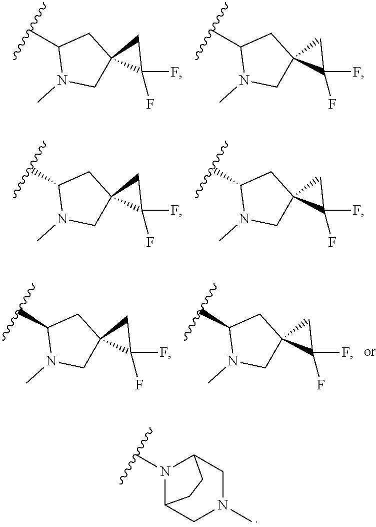

[61]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], [52] to [60], wherein:

The moiety of

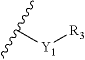

is selected from

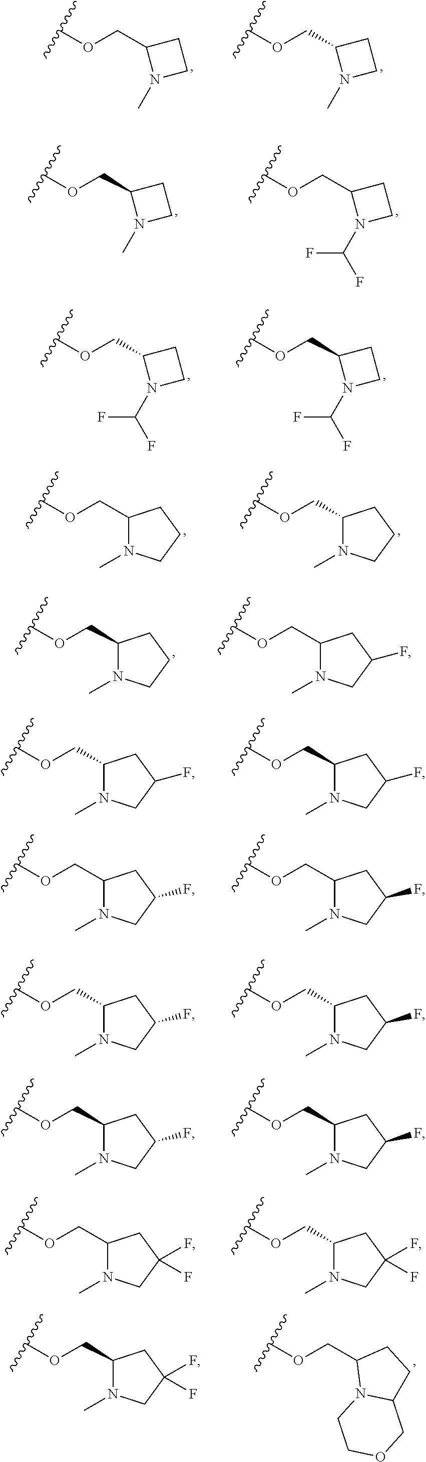

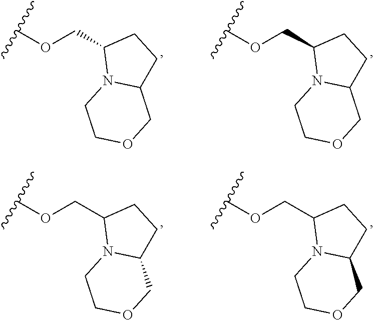 [SMILES: *OC[C@@H]1CCC2COCCN21 ; *OC[C@H]1CCC2COCCN21 ; *OCC1CC[C@@H]2COCCN12 ; *OCC1CC[C@H]2COCCN12]

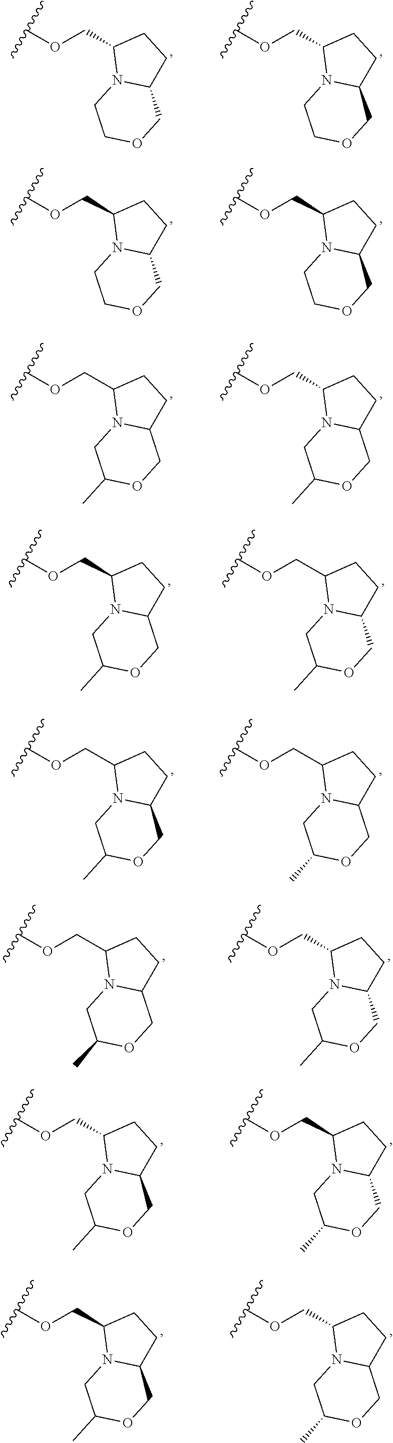

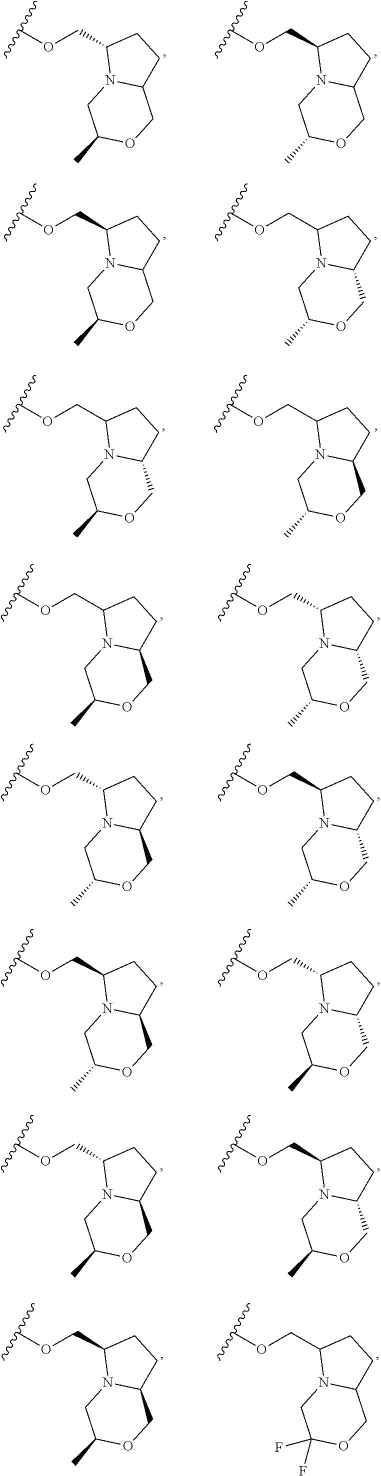

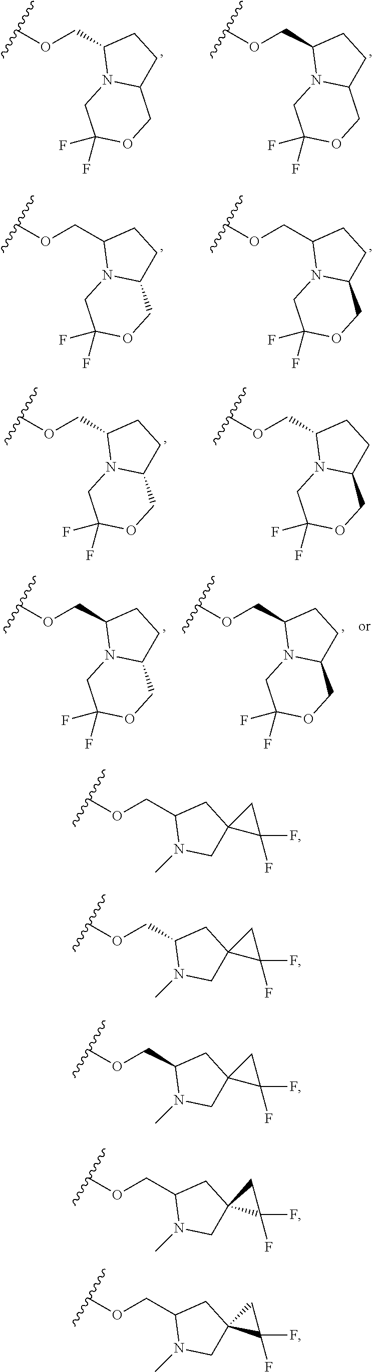

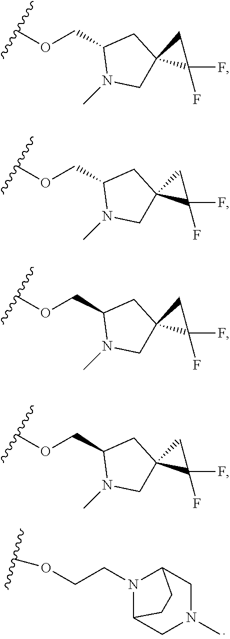 [SMILES: *OC[C@@H]1C[C@]2(CN1C)CC2(F)F ; *OC[C@@H]1C[C@@]2(CN1C)CC2(F)F ; *OC[C@H]1C[C@]2(CN1C)CC2(F)F ; *OC[C@H]1C[C@@]2(CN1C)CC2(F)F ; *OCCN1C2CCC1CN(C)C2]

[62]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [14], wherein,

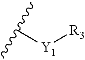

is selected from following Table C:

TABLE C

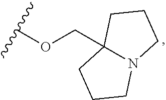 [SMILES: *OCC12CCCN1CCC2]

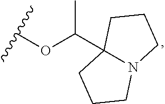 [SMILES: *OC(C)C12CCCN1CCC2]

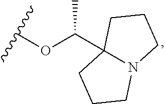 [SMILES: *O[C@H](C)C12CCCN1CCC2]

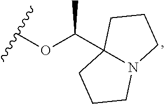 [SMILES: *O[C@@H](C)C12CCCN1CCC2]

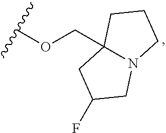 [SMILES: *OCC12CCCN1CC(F)C2]

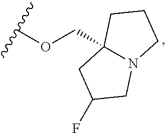 [SMILES: *OC[C@]12CCCN1CC(F)C2]

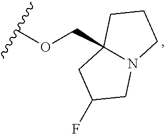 [SMILES: *OC[C@@]12CCCN1CC(F)C2]

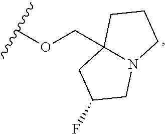 [SMILES: *OCC12CCCN1C[C@H](F)C2]

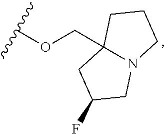 [SMILES: *OCC12CCCN1C[C@@H](F)C2]

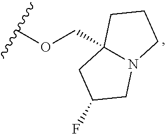 [SMILES: *OC[C@]12CCCN1C[C@H](F)C2]

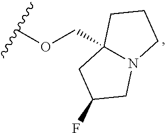 [SMILES: *OC[C@]12CCCN1C[C@@H](F)C2]

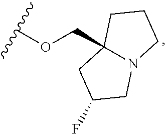 [SMILES: *OC[C@@]12CCCN1C[C@H](F)C2]

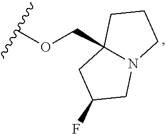 [SMILES: *OC[C@@]12CCCN1C[C@@H](F)C2]

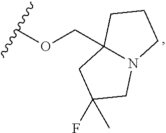 [SMILES: *OCC12CCCN1CC(C)(F)C2]

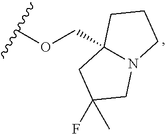 [SMILES: *OC[C@]12CCCN1CC(C)(F)C2]

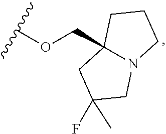 [SMILES: *OC[C@@]12CCCN1CC(C)(F)C2]

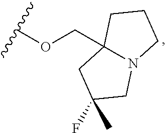 [SMILES: *OCC12CCCN1C[C@](C)(F)C2]

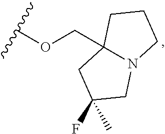 [SMILES: *OCC12CCCN1C[C@@](C)(F)C2]

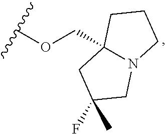 [SMILES: *OC[C@]12CCCN1C[C@](C)(F)C2]

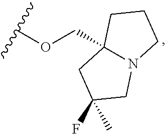 [SMILES: *OC[C@]12CCCN1C[C@@](C)(F)C2]

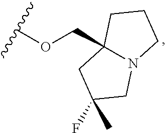 [SMILES: *OC[C@@]12CCCN1C[C@](C)(F)C2]

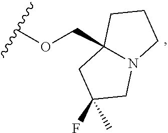 [SMILES: *OC[C@@]12CCCN1C[C@@](C)(F)C2]

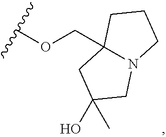 [SMILES: *OCC12CCCN1CC(C)(O)C2]

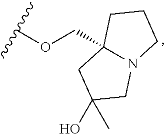 [SMILES: *OC[C@]12CCCN1CC(C)(O)C2]

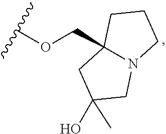 [SMILES: *OC[C@@]12CCCN1CC(C)(O)C2]

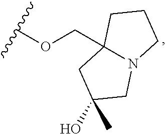 [SMILES: *OCC12CCCN1C[C@](C)(O)C2]

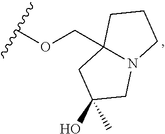 [SMILES: *OCC12CCCN1C[C@@](C)(O)C2]

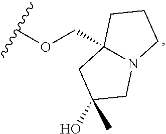 [SMILES: *OC[C@]12CCCN1C[C@](C)(O)C2]

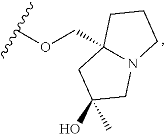 [SMILES: *OC[C@]12CCCN1C[C@@](C)(O)C2]

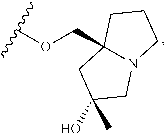 [SMILES: *OC[C@@]12CCCN1C[C@](C)(O)C2]

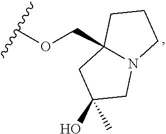 [SMILES: *OC[C@@]12CCCN1C[C@@](C)(O)C2]

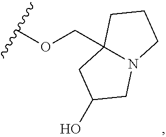 [SMILES: *OCC12CCCN1CC(O)C2]

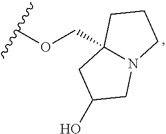 [SMILES: *OC[C@]12CCCN1CC(O)C2]

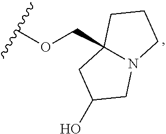 [SMILES: *OC[C@@]12CCCN1CC(O)C2]

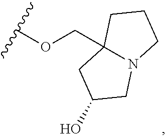 [SMILES: *OCC12CCCN1C[C@H](O)C2]

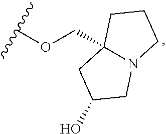 [SMILES: *OC[C@]12CCCN1C[C@H](O)C2]

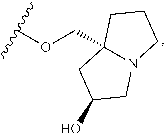 [SMILES: *OC[C@]12CCCN1C[C@@H](O)C2]

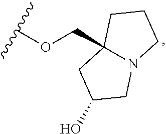 [SMILES: *OC[C@@]12CCCN1C[C@H](O)C2]

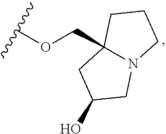 [SMILES: *OC[C@@]12CCCN1C[C@@H](O)C2]

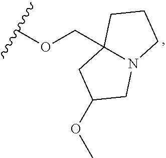 [SMILES: *OCC12CCCN1CC(OC)C2]

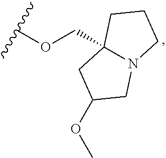 [SMILES: *OC[C@]12CCCN1CC(OC)C2]

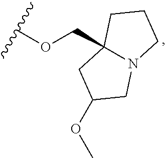 [SMILES: *OC[C@@]12CCCN1CC(OC)C2]

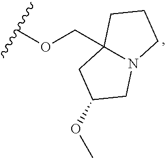 [SMILES: *OCC12CCCN1C[C@H](OC)C2]

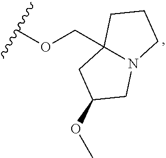 [SMILES: *OCC12CCCN1C[C@@H](OC)C2]

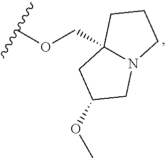 [SMILES: *OC[C@]12CCCN1C[C@H](OC)C2]

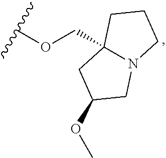 [SMILES: *OC[C@]12CCCN1C[C@@H](OC)C2]

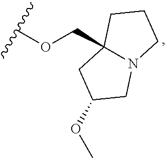 [SMILES: *OC[C@@]12CCCN1C[C@H](OC)C2]

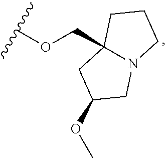 [SMILES: *OC[C@@]12CCCN1C[C@@H](OC)C2]

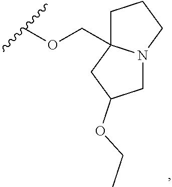 [SMILES: *OCC12CCCN1CC(OCC)C2]

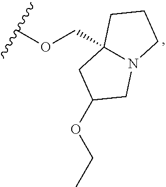 [SMILES: *OC[C@]12CCCN1CC(OCC)C2]

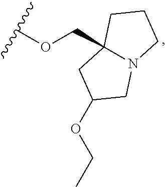 [SMILES: *OC[C@@]12CCCN1CC(OCC)C2]

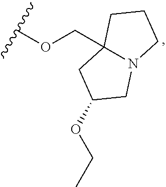 [SMILES: *OCC12CCCN1C[C@H](OCC)C2]

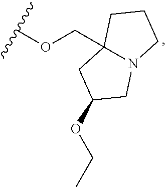 [SMILES: *OCC12CCCN1C[C@@H](OCC)C2]

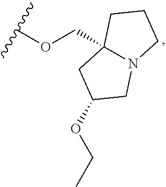 [SMILES: *OC[C@]12CCCN1C[C@H](OCC)C2]

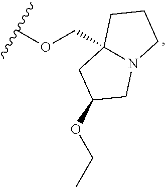 [SMILES: *OC[C@]12CCCN1C[C@@H](OCC)C2]

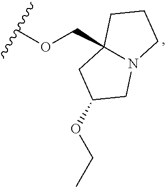 [SMILES: *OC[C@@]12CCCN1C[C@H](OCC)C2]

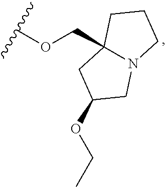 [SMILES: *OC[C@@]12CCCN1C[C@@H](OCC)C2]

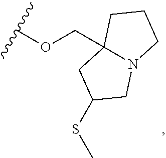 [SMILES: *OCC12CCCN1CC(SC)C2]

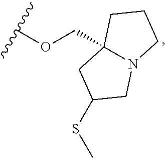 [SMILES: *OC[C@]12CCCN1CC(SC)C2]

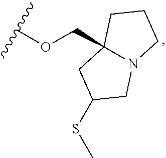 [SMILES: *OC[C@@]12CCCN1CC(SC)C2]

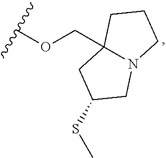 [SMILES: *OCC12CCCN1C[C@H](SC)C2]

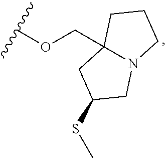 [SMILES: *OCC12CCCN1C[C@@H](SC)C2]

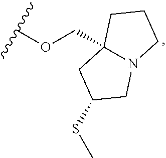 [SMILES: *OC[C@]12CCCN1C[C@H](SC)C2]

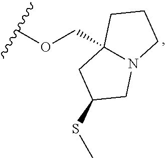 [SMILES: *OC[C@]12CCCN1C[C@@H](SC)C2]

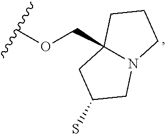 [SMILES: *OC[C@@]12CCCN1C[C@H](S)C2]

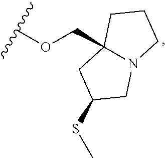 [SMILES: *OC[C@@]12CCCN1C[C@@H](SC)C2]

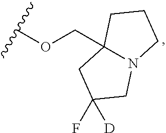 [SMILES: *OCC12CCCN1CC([2H])(F)C2]

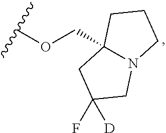 [SMILES: *OC[C@]12CCCN1CC([2H])(F)C2]

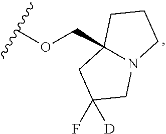 [SMILES: *OC[C@@]12CCCN1CC([2H])(F)C2]

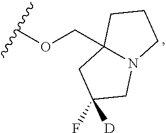 [SMILES: *OCC12CCCN1C[C@]([2H])(F)C2]

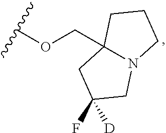 [SMILES: *OCC12CCCN1C[C@@]([2H])(F)C2]

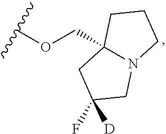 [SMILES: *OC[C@]12CCCN1C[C@]([2H])(F)C2]

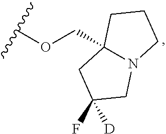 [SMILES: *OC[C@]12CCCN1C[C@@]([2H])(F)C2]

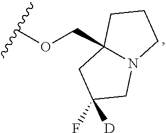 [SMILES: *OC[C@@]12CCCN1C[C@]([2H])(F)C2]

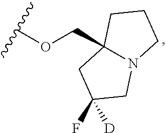 [SMILES: *OC[C@@]12CCCN1C[C@@]([2H])(F)C2]

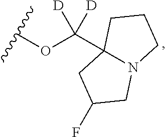 [SMILES: *OC([2H])([2H])C12CCCN1CC(F)C2]

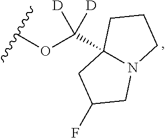 [SMILES: *OC([2H])([2H])[C@]12CCCN1CC(F)C2]

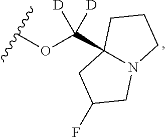 [SMILES: *OC([2H])([2H])[C@@]12CCCN1CC(F)C2]

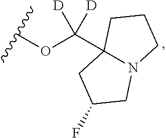 [SMILES: *OC([2H])([2H])C12CCCN1C[C@H](F)C2]

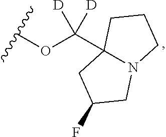 [SMILES: *OC([2H])([2H])C12CCCN1C[C@@H](F)C2]

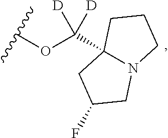 [SMILES: *OC([2H])([2H])[C@]12CCCN1C[C@H](F)C2]

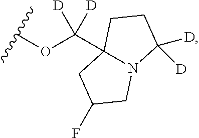

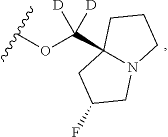 [SMILES: *OC([2H])([2H])[C@@]12CCCN1C[C@H](F)C2]

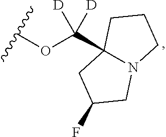 [SMILES: *OC([2H])([2H])[C@@]12CCCN1C[C@@H](F)C2]

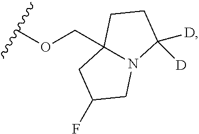

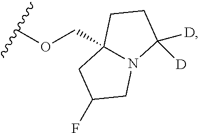

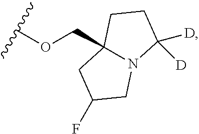

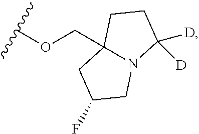

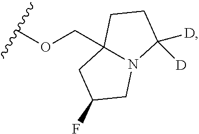

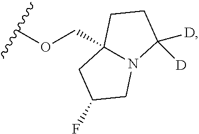

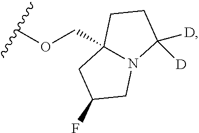

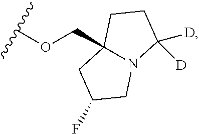

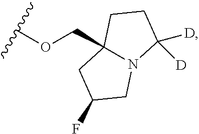

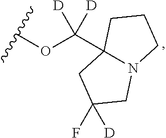 [SMILES: *OC([2H])([2H])C12CCCN1CC([2H])(F)C2]

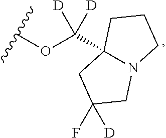 [SMILES: *OC([2H])([2H])[C@]12CCCN1CC([2H])(F)C2]

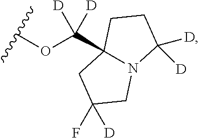

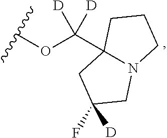 [SMILES: *OC([2H])([2H])C12CCCN1C[C@]([2H])(F)C2]

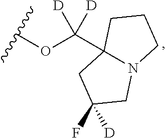 [SMILES: *OC([2H])([2H])C12CCCN1C[C@@]([2H])(F)C2]

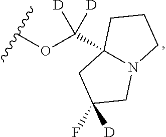 [SMILES: *OC([2H])([2H])[C@]12CCCN1C[C@]([2H])(F)C2]

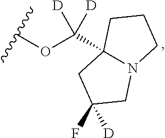 [SMILES: *OC([2H])([2H])[C@]12CCCN1C[C@@]([2H])(F)C2]

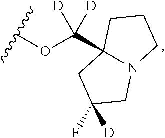 [SMILES: *OC([2H])([2H])[C@@]12CCCN1C[C@]([2H])(F)C2]

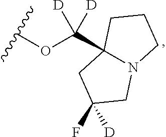 [SMILES: *OC([2H])([2H])[C@@]12CCCN1C[C@@]([2H])(F)C2]

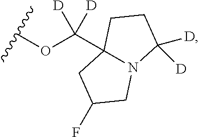

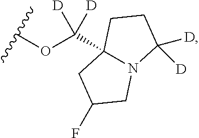

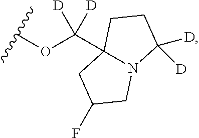

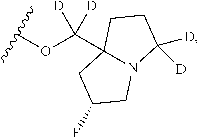

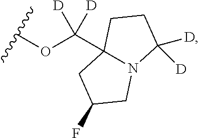

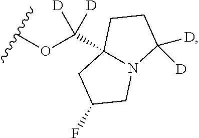

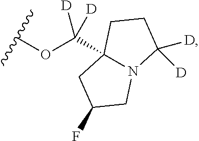

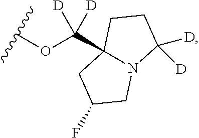

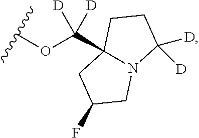

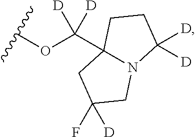

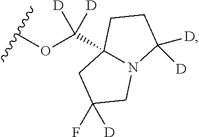

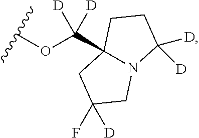

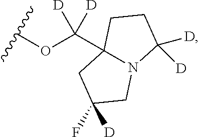

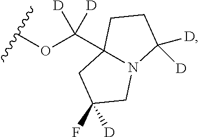

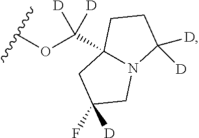

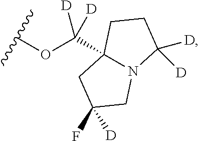

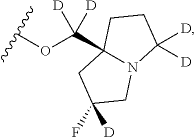

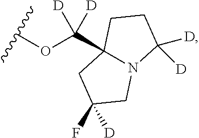

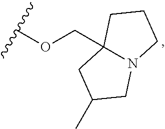 [SMILES: *OCC12CCCN1CC(C)C2]

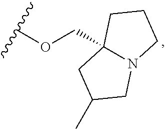 [SMILES: *OC[C@]12CCCN1CC(C)C2]

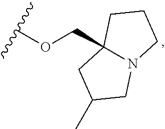 [SMILES: *OC[C@@]12CCCN1CC(C)C2]

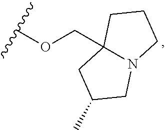 [SMILES: *OCC12CCCN1C[C@H](C)C2]

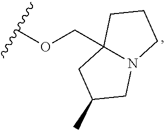 [SMILES: *OCC12CCCN1C[C@@H](C)C2]

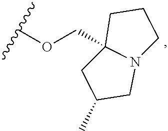 [SMILES: *OC[C@]12CCCN1C[C@H](C)C2]

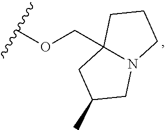 [SMILES: *OCC12CCCN1C[C@@H](C)C2]

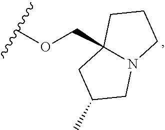 [SMILES: *OC[C@@]12CCCN1C[C@H](C)C2]

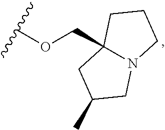 [SMILES: *OC[C@@]12CCCN1C[C@@H](C)C2]

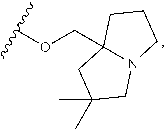 [SMILES: *OCC12CCCN1CC(C)(C)C2]

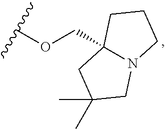 [SMILES: *OC[C@]12CCCN1CC(C)(C)C2]

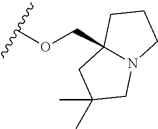 [SMILES: *OC[C@@]12CCCN1CC(C)(C)C2]

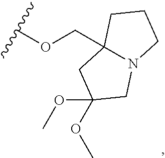 [SMILES: *OCC12CCCN1CC(OC)(OC)C2]

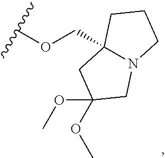 [SMILES: *OC[C@]12CCCN1CC(OC)(OC)C2]

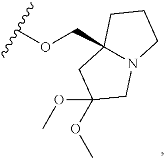 [SMILES: *OC[C@@]12CCCN1CC(OC)(OC)C2]

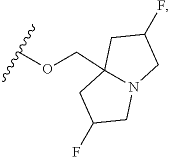 [SMILES: *OCC12CC(F)CN1CC(F)C2]

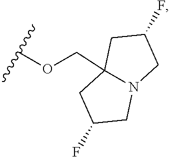 [SMILES: *OCC12C[C@H](F)CN1C[C@H](F)C2]

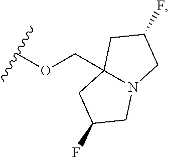 [SMILES: *OCC12C[C@H](F)CN1C[C@@H](F)C2]

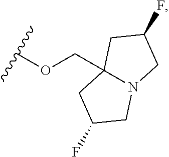 [SMILES: *OCC12C[C@@H](F)CN1C[C@H](F)C2]

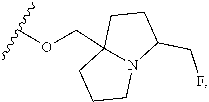 [SMILES: *OCC12CCCN1C(CF)CC2]

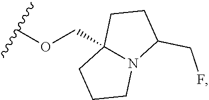 [SMILES: *OC[C@@]12CCCN1C(CF)CC2]

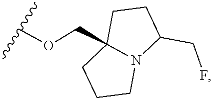 [SMILES: *OC[C@]12CCCN1C(CF)CC2]

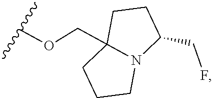 [SMILES: *OCC12CCCN1[C@@H](CF)CC2]

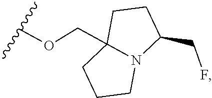 [SMILES: *OCC12CCCN1[C@H](CF)CC2]

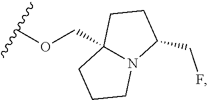 [SMILES: *OC[C@@]12CCCN1[C@@H](CF)CC2]

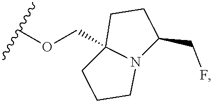 [SMILES: *OC[C@@]12CCCN1[C@H](CF)CC2]

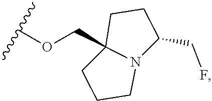 [SMILES: *OC[C@]12CCCN1[C@@H](CF)CC2]

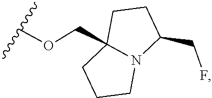 [SMILES: *OC[C@]12CCCN1[C@H](CF)CC2]

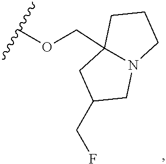 [SMILES: *OCC12CCCN1CC(CF)C2]

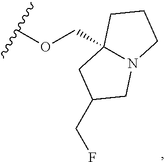 [SMILES: *OC[C@]12CCCN1CC(CF)C2]

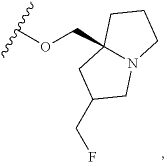 [SMILES: *OC[C@@]12CCCN1CC(CF)C2]

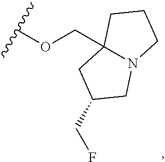 [SMILES: *OCC12CCCN1C[C@H](CF)C2]

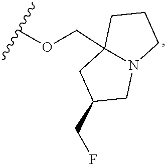 [SMILES: *OCC12CCCN1C[C@@H](CF)C2]

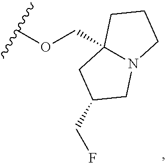 [SMILES: *OC[C@]12CCCN1C[C@H](CF)C2]

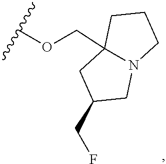 [SMILES: *OCC12CCCN1C[C@@H](CF)C2]

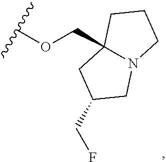 [SMILES: *OC[C@@]12CCCN1C[C@H](CF)C2]

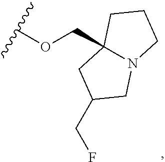 [SMILES: *OC[C@@]12CCCN1CC(CF)C2]

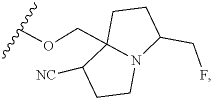 [SMILES: *OCC12CCC(CF)N1CCC2C#N]

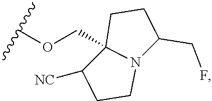 [SMILES: *OC[C@]12CCC(CF)N1CCC2C#N]

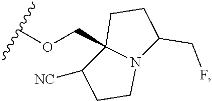 [SMILES: *OC[C@@]12CCC(CF)N1CCC2C#N]

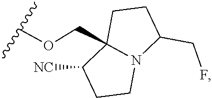 [SMILES: *OC[C@@]12CCC(CF)N1CC[C@@H]2C#N]

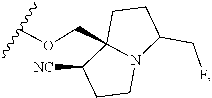 [SMILES: *OC[C@@]12CCC(CF)N1CC[C@H]2C#N]

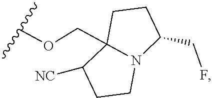 [SMILES: *OCC12CC[C@H](CF)N1CCC2C#N]

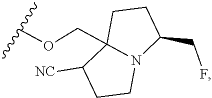 [SMILES: *OCC12CC[C@@H](CF)N1CCC2C#N]

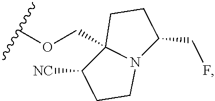 [SMILES: *OC[C@]12CC[C@H](CF)N1CC[C@@H]2C#N]

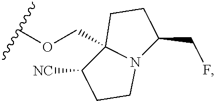 [SMILES: *OC[C@]12CC[C@@H](CF)N1CC[C@@H]2C#N]

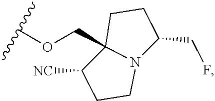 [SMILES: *OC[C@@]12CC[C@H](CF)N1CC[C@@H]2C#N]

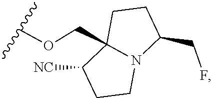 [SMILES: *OC[C@@]12CC[C@@H](CF)N1CC[C@@H]2C#N]

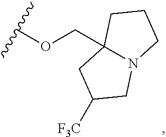 [SMILES: *OCC12CCCN1CC(C(F)(F)F)C2]

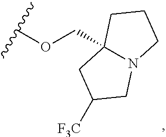 [SMILES: *OC[C@]12CCCN1CC(C(F)(F)F)C2]

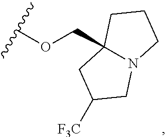 [SMILES: *OC[C@@]12CCCN1CC(C(F)(F)F)C2]

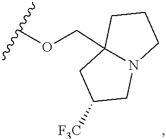 [SMILES: *OCC12CCCN1C[C@H](C(F)(F)F)C2]

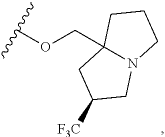 [SMILES: *OCC12CCCN1C[C@@H](C(F)(F)F)C2]

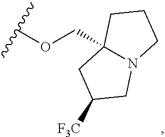 [SMILES: *OC[C@]12CCCN1C[C@@H](C(F)(F)F)C2]

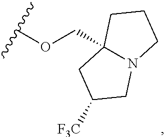 [SMILES: *OC[C@]12CCCN1C[C@H](C(F)(F)F)C2]

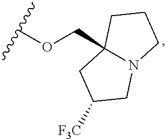 [SMILES: *OC[C@@]12CCCN1C[C@H](C(F)(F)F)C2]

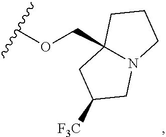 [SMILES: *OC[C@@]12CCCN1C[C@@H](C(F)(F)F)C2]

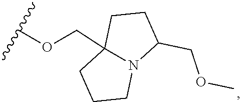 [SMILES: *OCC12CCCN1C(COC)CC2]

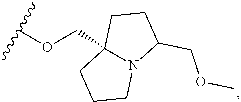 [SMILES: *OC[C@@]12CCCN1C(COC)CC2]

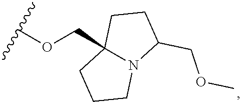 [SMILES: *OC[C@]12CCCN1C(COC)CC2]

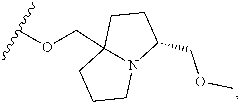 [SMILES: *OCC12CCCN1[C@@H](COC)CC2]

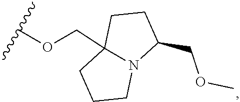 [SMILES: *OCC12CCCN1[C@H](COC)CC2]

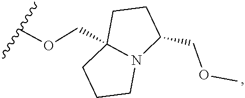 [SMILES: *OC[C@@]12CCCN1[C@@H](COC)CC2]

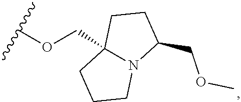 [SMILES: *OC[C@@]12CCCN1[C@H](COC)CC2]

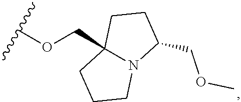 [SMILES: *OC[C@]12CCCN1[C@@H](COC)CC2]

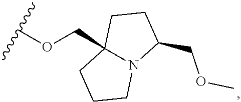 [SMILES: *OC[C@]12CCCN1[C@H](COC)CC2]

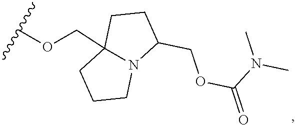 [SMILES: *OCC12CCCN1C(COC(=O)N(C)C)CC2]

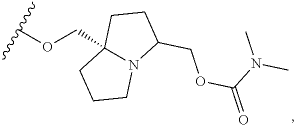 [SMILES: *OC[C@@]12CCCN1C(COC(=O)N(C)C)CC2]

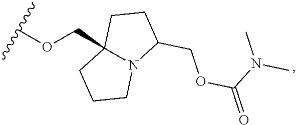 [SMILES: *OC[C@]12CCCN1C(COC(=O)N(C)C)CC2]

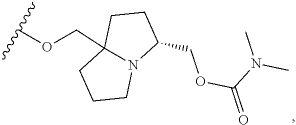 [SMILES: *OCC12CCCN1[C@@H](COC(=O)N(C)C)CC2]

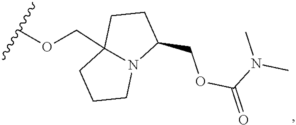 [SMILES: *OCC12CCCN1[C@H](COC(=O)N(C)C)CC2]

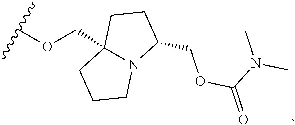 [SMILES: *OC[C@@]12CCCN1[C@@H](COC(=O)N(C)C)CC2]

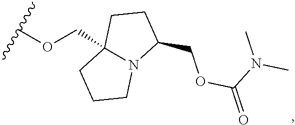 [SMILES: *OC[C@@]12CCCN1[C@H](COC(=O)N(C)C)CC2]

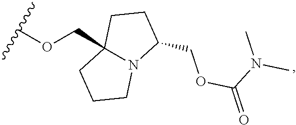 [SMILES: *OC[C@]12CCCN1[C@@H](COC(=O)N(C)C)CC2]

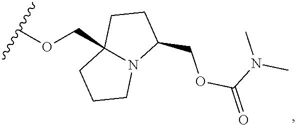 [SMILES: *OC[C@]12CCCN1[C@H](COC(=O)N(C)C)CC2]

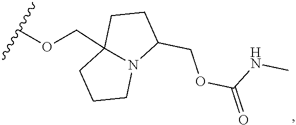 [SMILES: *OCC12CCCN1C(COC(=O)NC)CC2]

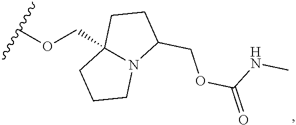 [SMILES: *OC[C@@]12CCCN1C(COC(=O)NC)CC2]

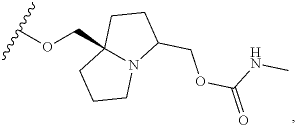 [SMILES: *OC[C@]12CCCN1C(COC(=O)NC)CC2]

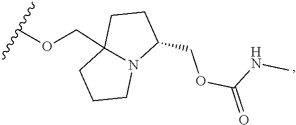 [SMILES: *OCC12CCCN1[C@@H](COC(=O)NC)CC2]

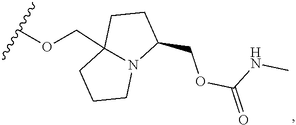 [SMILES: *OCC12CCCN1[C@H](COC(=O)NC)CC2]

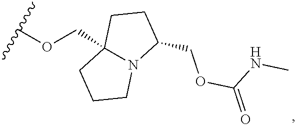 [SMILES: *OC[C@@]12CCCN1[C@@H](COC(=O)NC)CC2]

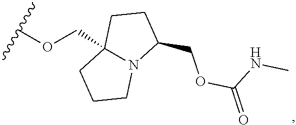 [SMILES: *OC[C@@]12CCCN1[C@H](COC(=O)NC)CC2]

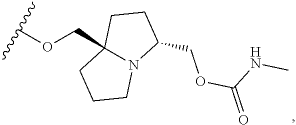 [SMILES: *OC[C@]12CCCN1[C@@H](COC(=O)NC)CC2]

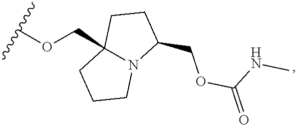 [SMILES: *OC[C@]12CCCN1[C@H](COC(=O)NC)CC2]

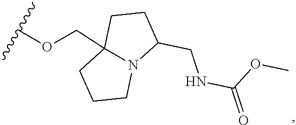 [SMILES: *OCC12CCCN1C(CNC(=O)OC)CC2]

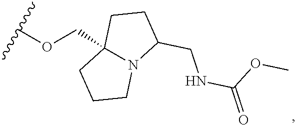 [SMILES: *OC[C@@]12CCCN1C(CNC(=O)OC)CC2]

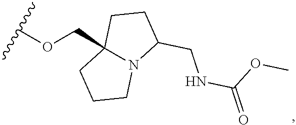 [SMILES: *OC[C@]12CCCN1C(CNC(=O)OC)CC2]

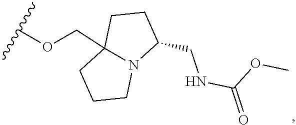 [SMILES: *OCC12CCCN1[C@@H](CNC(=O)OC)CC2]

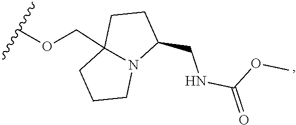 [SMILES: *OCC12CCCN1[C@H](CNC(=O)OC)CC2]

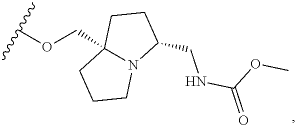 [SMILES: *OC[C@@]12CCCN1[C@@H](CNC(=O)OC)CC2]

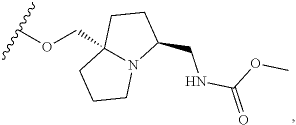 [SMILES: *OC[C@@]12CCCN1[C@H](CNC(=O)OC)CC2]

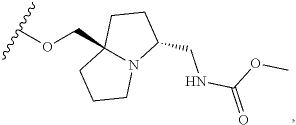 [SMILES: *OC[C@]12CCCN1[C@@H](CNC(=O)OC)CC2]

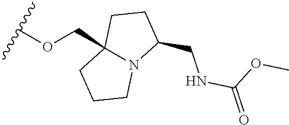 [SMILES: *OC[C@]12CCCN1[C@H](CNC(=O)OC)CC2]

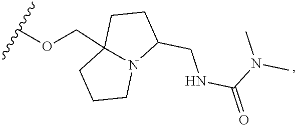 [SMILES: *OCC12CCCN1C(CNC(=O)N(C)C)CC2]

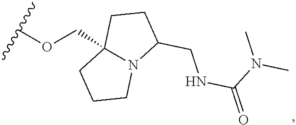 [SMILES: *OC[C@@]12CCCN1C(CNC(=O)N(C)C)CC2]

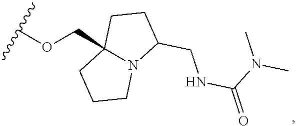 [SMILES: *OC[C@]12CCCN1C(CNC(=O)N(C)C)CC2]

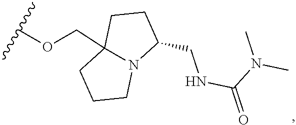 [SMILES: *OCC12CCCN1[C@@H](CNC(=O)N(C)C)CC2]

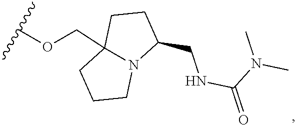 [SMILES: *OCC12CCCN1[C@H](CNC(=O)N(C)C)CC2]

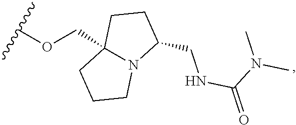 [SMILES: *OC[C@@]12CCCN1[C@@H](CNC(=O)N(C)C)CC2]

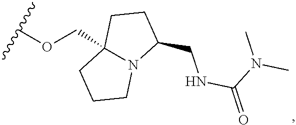 [SMILES: *OC[C@@]12CCCN1[C@H](CNC(=O)N(C)C)CC2]

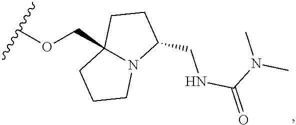 [SMILES: *OC[C@]12CCCN1[C@@H](CNC(=O)N(C)C)CC2]

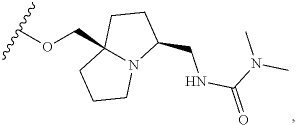 [SMILES: *OC[C@]12CCCN1[C@H](CNC(=O)N(C)C)CC2]

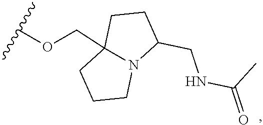 [SMILES: *OCC12CCCN1C(CNC(C)=O)CC2]

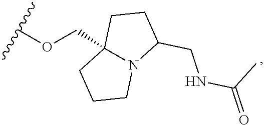 [SMILES: *OC[C@@]12CCCN1C(CNC(C)=O)CC2]

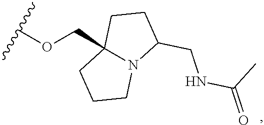 [SMILES: *OC[C@]12CCCN1C(CNC(C)=O)CC2]

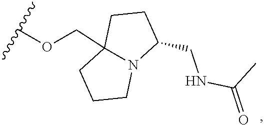 [SMILES: *OCC12CCCN1[C@@H](CNC(C)=O)CC2]

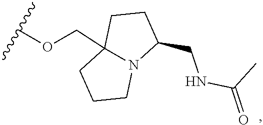 [SMILES: *OCC12CCCN1[C@H](CNC(C)=O)CC2]

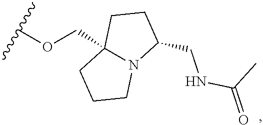 [SMILES: *OC[C@@]12CCCN1[C@@H](CNC(C)=O)CC2]

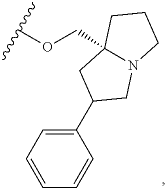 [SMILES: *OC[C@]12CCCN1CC(c1ccccc1)C2]

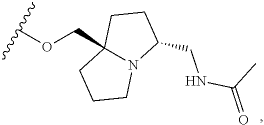 [SMILES: *OC[C@]12CCCN1[C@@H](CNC(C)=O)CC2]

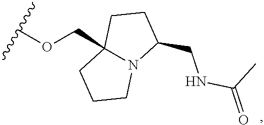 [SMILES: *OC[C@]12CCCN1[C@H](CNC(C)=O)CC2]

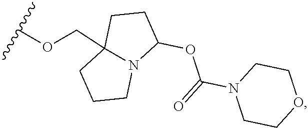 [SMILES: *OCC12CCCN1C(OC(=O)N1CCOCC1)CC2]

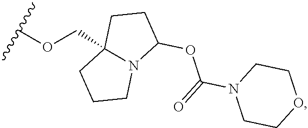 [SMILES: *OC[C@@]12CCCN1C(OC(=O)N1CCOCC1)CC2]

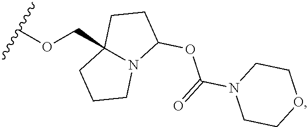 [SMILES: *OC[C@]12CCCN1C(OC(=O)N1CCOCC1)CC2]

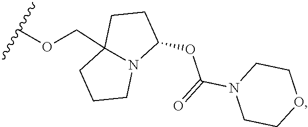 [SMILES: *OCC12CCCN1[C@@H](OC(=O)N1CCOCC1)CC2]

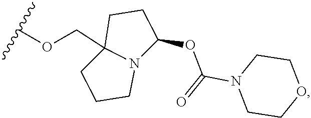 [SMILES: *OCC12CCCN1[C@H](OC(=O)N1CCOCC1)CC2]

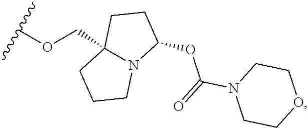 [SMILES: *OC[C@@]12CCCN1[C@@H](OC(=O)N1CCOCC1)CC2]

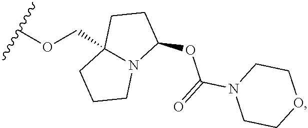 [SMILES: *OC[C@@]12CCCN1[C@H](OC(=O)N1CCOCC1)CC2]

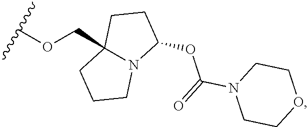 [SMILES: *OC[C@]12CCCN1[C@@H](OC(=O)N1CCOCC1)CC2]

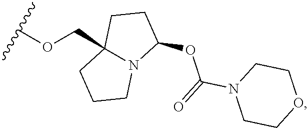 [SMILES: *OC[C@]12CCCN1[C@H](OC(=O)N1CCOCC1)CC2]

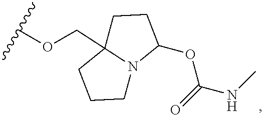 [SMILES: *OCC12CCCN1C(OC(=O)NC)CC2]

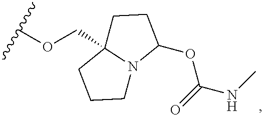 [SMILES: *OC[C@@]12CCCN1C(OC(=O)NC)CC2]

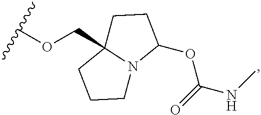 [SMILES: *OC[C@]12CCCN1C(OC(=O)NC)CC2]

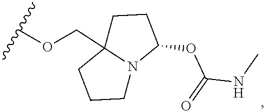 [SMILES: *OCC12CCCN1[C@@H](OC(=O)NC)CC2]

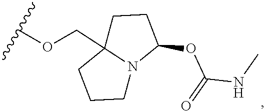 [SMILES: *OCC12CCCN1[C@H](OC(=O)NC)CC2]

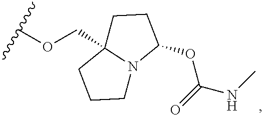 [SMILES: *OC[C@@]12CCCN1[C@@H](OC(=O)NC)CC2]

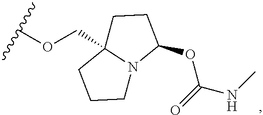 [SMILES: *OC[C@@]12CCCN1[C@H](OC(=O)NC)CC2]

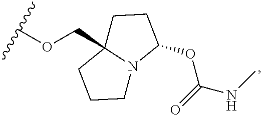 [SMILES: *OC[C@]12CCCN1[C@@H](OC(=O)NC)CC2]

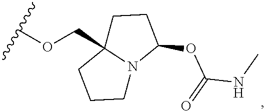 [SMILES: *OC[C@]12CCCN1[C@H](OC(=O)NC)CC2]

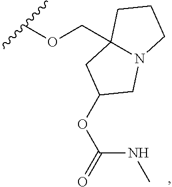 [SMILES: *OCC12CCCN1CC(OC(=O)NC)C2]

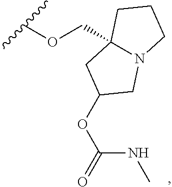 [SMILES: *OC[C@]12CCCN1CC(OC(=O)NC)C2]

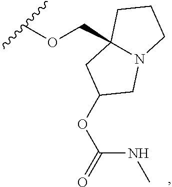 [SMILES: *OC[C@@]12CCCN1CC(OC(=O)NC)C2]

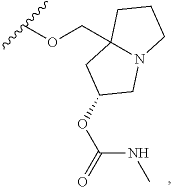 [SMILES: *OCC12CCCN1C[C@H](OC(=O)NC)C2]

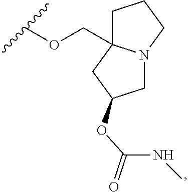 [SMILES: *OCC12CCCN1C[C@@H](OC(=O)NC)C2]

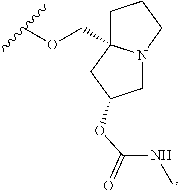 [SMILES: *OC[C@]12CCCN1C[C@H](OC(=O)NC)C2]

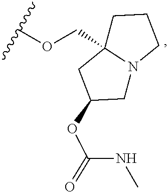 [SMILES: *OC[C@]12CCCN1C[C@@H](OC(=O)NC)C2]

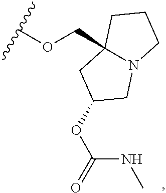 [SMILES: *OC[C@@]12CCCN1C[C@H](OC(=O)NC)C2]

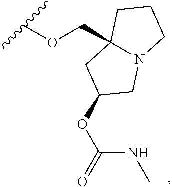 [SMILES: *OC[C@@]12CCCN1C[C@@H](OC(=O)NC)C2]

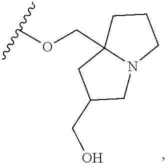 [SMILES: *OCC12CCCN1CC(CO)C2]

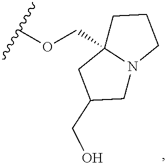 [SMILES: *OC[C@]12CCCN1CC(CO)C2]

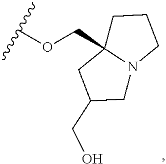 [SMILES: *OC[C@@]12CCCN1CC(CO)C2]

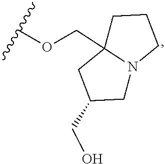 [SMILES: *OCC12CCCN1C[C@H](CO)C2]

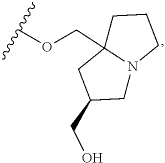 [SMILES: *OCC12CCCN1C[C@@H](CO)C2]

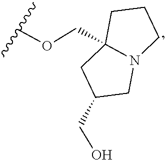 [SMILES: *OC[C@]12CCCN1C[C@H](CO)C2]

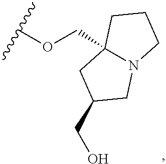 [SMILES: *OC[C@]12CCCN1C[C@@H](CO)C2]

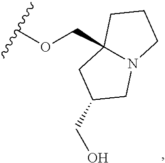 [SMILES: *OC[C@@]12CCCN1C[C@H](CO)C2]

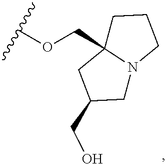 [SMILES: *OC[C@@]12CCCN1C[C@@H](CO)C2]

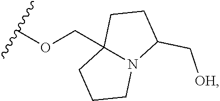 [SMILES: *OCC12CCCN1C(CO)CC2]

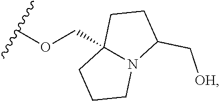 [SMILES: *OC[C@@]12CCCN1C(CO)CC2]

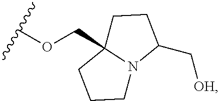 [SMILES: *OC[C@]12CCCN1C(CO)CC2]

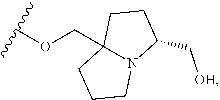 [SMILES: *OCC12CCCN1[C@@H](CO)CC2]

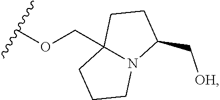 [SMILES: *OCC12CCCN1[C@H](CO)CC2]

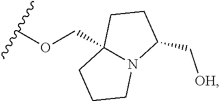 [SMILES: *OC[C@@]12CCCN1[C@@H](CO)CC2]

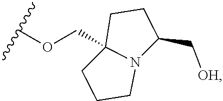 [SMILES: *OC[C@@]12CCCN1[C@H](CO)CC2]

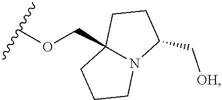 [SMILES: *OC[C@]12CCCN1[C@@H](CO)CC2]

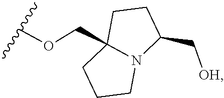 [SMILES: *OC[C@]12CCCN1[C@H](CO)CC2]

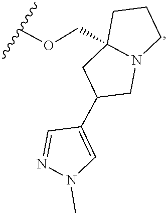 [SMILES: *OC[C@]12CCCN1CC(c1cnn(C)c1)C2]

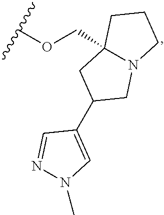 [SMILES: *OC[C@]12CCCN1CC(c1cnn(C)c1)C2]

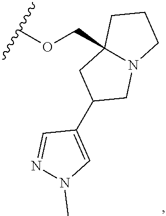 [SMILES: *OC[C@@]12CCCN1CC(c1cnn(C)c1)C2]

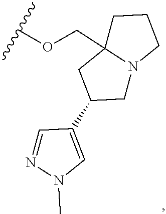 [SMILES: *OCC12CCCN1C[C@H](c1cnn(C)c1)C2]

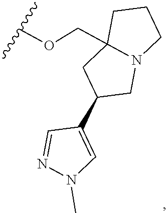 [SMILES: *OCC12CCCN1C[C@@H](c1cnn(C)c1)C2]

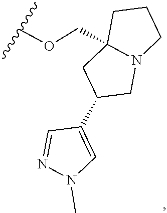 [SMILES: *OC[C@]12CCCN1C[C@H](c1cnn(C)c1)C2]

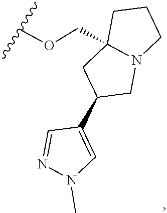 [SMILES: *OC[C@]12CCCN1C[C@@H](c1cnn(C)c1)C2]

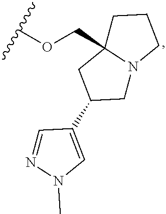 [SMILES: *OC[C@@]12CCCN1C[C@H](c1cnn(C)c1)C2]

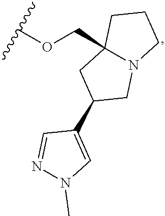 [SMILES: *OC[C@@]12CCCN1C[C@@H](c1cnn(C)c1)C2]

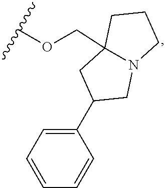 [SMILES: *OCC12CCCN1CC(c1ccccc1)C2]

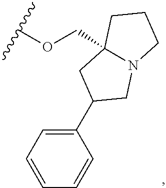 [SMILES: *OC[C@]12CCCN1CC(c1ccccc1)C2]

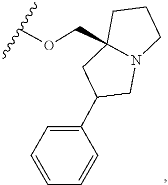 [SMILES: *OC[C@@]12CCCN1CC(c1ccccc1)C2]

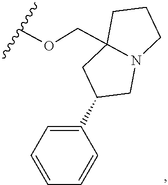 [SMILES: *OCC12CCCN1C[C@H](c1ccccc1)C2]

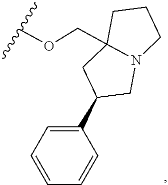 [SMILES: *OCC12CCCN1C[C@@H](c1ccccc1)C2]

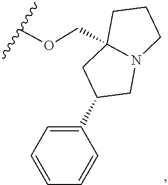 [SMILES: *OC[C@]12CCCN1C[C@H](c1ccccc1)C2]

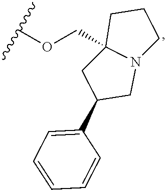 [SMILES: *OC[C@]12CCCN1C[C@@H](c1ccccc1)C2]

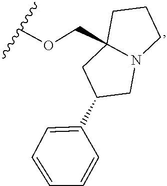 [SMILES: *OC[C@@]12CCCN1C[C@H](c1ccccc1)C2]

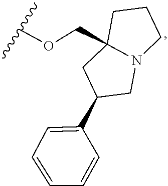 [SMILES: *OC[C@@]12CCCN1C[C@@H](c1ccccc1)C2]

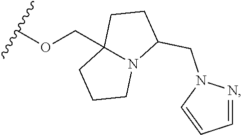 [SMILES: *OCC12CCCN1C(Cn1cccn1)CC2]

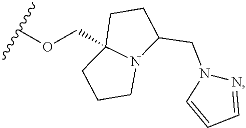 [SMILES: *OC[C@@]12CCCN1C(Cn1cccn1)CC2]

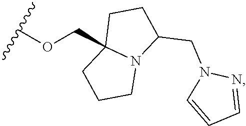 [SMILES: *OC[C@]12CCCN1C(Cn1cccn1)CC2]

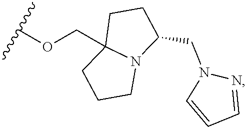 [SMILES: *OCC12CCCN1[C@@H](Cn1cccn1)CC2]

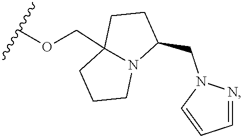 [SMILES: *OCC12CCCN1[C@H](Cn1cccn1)CC2]

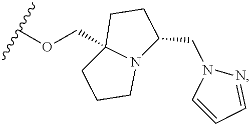 [SMILES: *OC[C@@]12CCCN1[C@@H](Cn1cccn1)CC2]

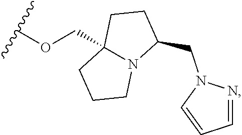 [SMILES: *OC[C@@]12CCCN1[C@H](Cn1cccn1)CC2]

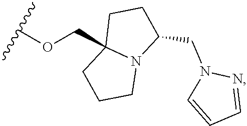 [SMILES: *OC[C@]12CCCN1[C@@H](Cn1cccn1)CC2]

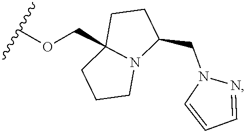 [SMILES: *OC[C@]12CCCN1[C@H](Cn1cccn1)CC2]

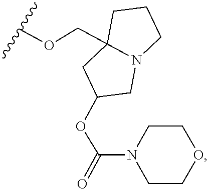 [SMILES: *OCC12CCCN1CC(OC(=O)N1CCOCC1)C2]

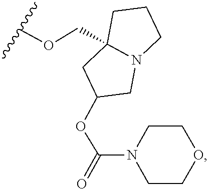 [SMILES: *OC[C@]12CCCN1CC(OC(=O)N1CCOCC1)C2]

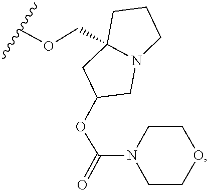 [SMILES: *OC[C@]12CCCN1CC(OC(=O)N1CCOCC1)C2]

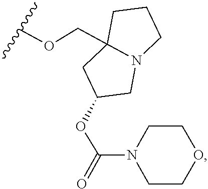 [SMILES: *OCC12CCCN1C[C@H](OC(=O)N1CCOCC1)C2]

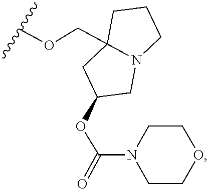 [SMILES: *OCC12CCCN1C[C@@H](OC(=O)N1CCOCC1)C2]

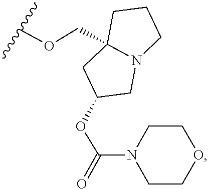 [SMILES: *OC[C@]12CCCN1C[C@H](OC(=O)N1CCOCC1)C2]

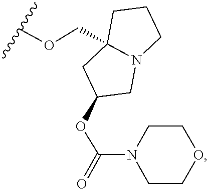 [SMILES: *OC[C@]12CCCN1C[C@@H](OC(=O)N1CCOCC1)C2]

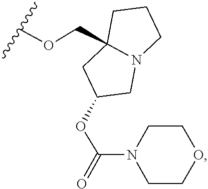 [SMILES: *OC[C@@]12CCCN1C[C@H](OC(=O)N1CCOCC1)C2]

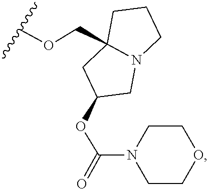 [SMILES: *OC[C@@]12CCCN1C[C@@H](OC(=O)N1CCOCC1)C2]

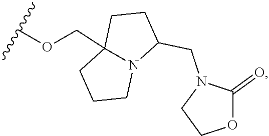 [SMILES: *OCC12CCCN1C(CN1CCOC1=O)CC2]

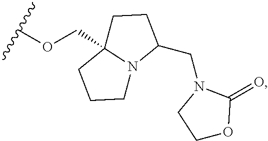 [SMILES: *OC[C@@]12CCCN1C(CN1CCOC1=O)CC2]

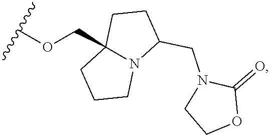 [SMILES: *OC[C@]12CCCN1C(CN1CCOC1=O)CC2]

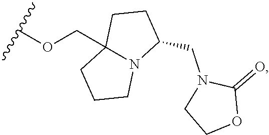 [SMILES: *OCC12CCCN1[C@@H](CN1CCOC1=O)CC2]

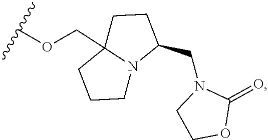 [SMILES: *OCC12CCCN1[C@H](CN1CCOC1=O)CC2]

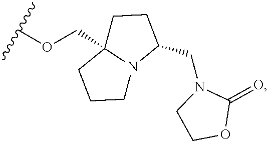 [SMILES: *OC[C@@]12CCCN1[C@@H](CN1CCOC1=O)CC2]

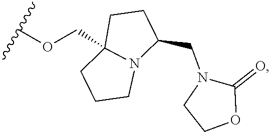 [SMILES: *OC[C@@]12CCCN1[C@H](CN1CCOC1=O)CC2]

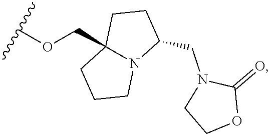 [SMILES: *OC[C@]12CCCN1[C@@H](CN1CCOC1=O)CC2]

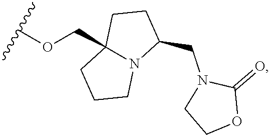 [SMILES: *OC[C@]12CCCN1[C@H](CN1CCOC1=O)CC2]

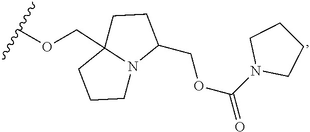 [SMILES: *OCC12CCCN1C(COC(=O)N1CCCC1)CC2]

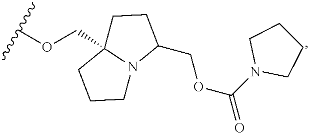 [SMILES: *OC[C@@]12CCCN1C(COC(=O)N1CCCC1)CC2]

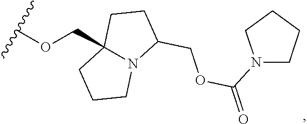 [SMILES: *OC[C@]12CCCN1C(COC(=O)N1CCCC1)CC2]

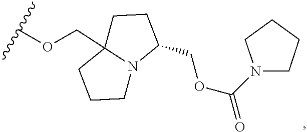 [SMILES: *OCC12CCCN1[C@@H](COC(=O)N1CCCC1)CC2]

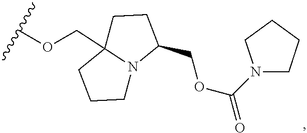 [SMILES: *OCC12CCCN1[C@H](COC(=O)N1CCCC1)CC2]

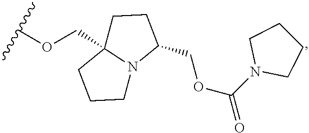 [SMILES: *OC[C@@]12CCCN1[C@@H](COC(=O)N1CCCC1)CC2]

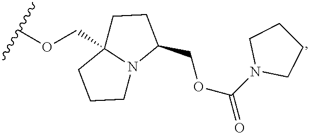 [SMILES: *OC[C@@]12CCCN1[C@H](COC(=O)N1CCCC1)CC2]

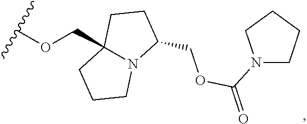 [SMILES: *OC[C@]12CCCN1[C@@H](COC(=O)N1CCCC1)CC2]

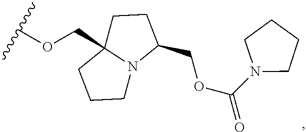 [SMILES: *OC[C@]12CCCN1[C@H](COC(=O)N1CCCC1)CC2]

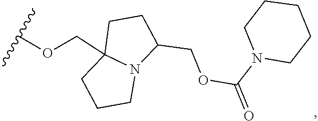 [SMILES: *OCC12CCCN1C(COC(=O)N1CCCCC1)CC2]

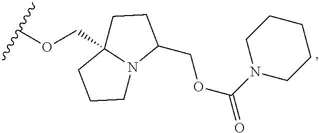 [SMILES: *OC[C@@]12CCCN1C(COC(=O)N1CCCCC1)CC2]

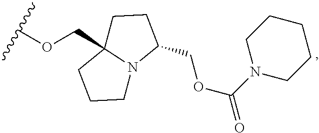 [SMILES: *OC[C@]12CCCN1[C@@H](COC(=O)N1CCCCC1)CC2]

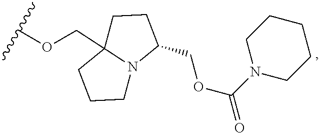 [SMILES: *OCC12CCCN1[C@@H](COC(=O)N1CCCCC1)CC2]

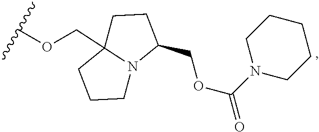 [SMILES: *OCC12CCCN1[C@H](COC(=O)N1CCCCC1)CC2]

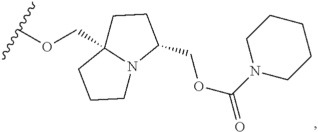 [SMILES: *OC[C@@]12CCCN1[C@@H](COC(=O)N1CCCCC1)CC2]

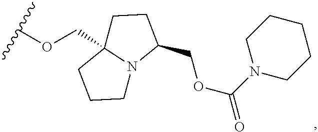 [SMILES: *OC[C@@]12CCCN1[C@H](COC(=O)N1CCCCC1)CC2]

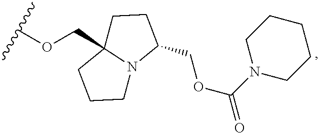 [SMILES: *OC[C@]12CCCN1[C@@H](COC(=O)N1CCCCC1)CC2]

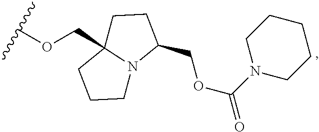 [SMILES: *OC[C@]12CCCN1[C@H](COC(=O)N1CCCCC1)CC2]

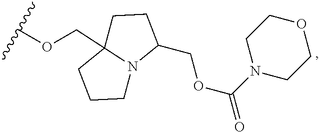 [SMILES: *OCC12CCCN1C(COC(=O)N1CCOCC1)CC2]

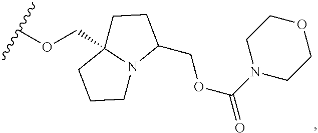 [SMILES: *OC[C@@]12CCCN1C(COC(=O)N1CCOCC1)CC2]

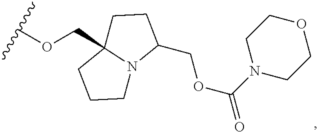 [SMILES: *OC[C@]12CCCN1C(COC(=O)N1CCOCC1)CC2]

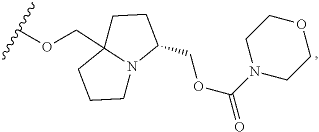 [SMILES: *OCC12CCCN1[C@@H](COC(=O)N1CCOCC1)CC2]

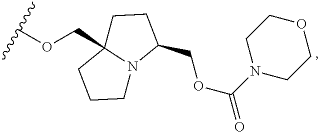 [SMILES: *OC[C@]12CCCN1[C@H](COC(=O)N1CCOCC1)CC2]

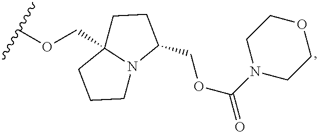 [SMILES: *OC[C@@]12CCCN1[C@@H](COC(=O)N1CCOCC1)CC2]

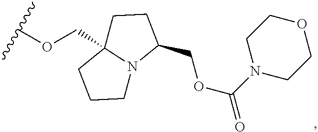 [SMILES: *OC[C@@]12CCCN1[C@H](COC(=O)N1CCOCC1)CC2]

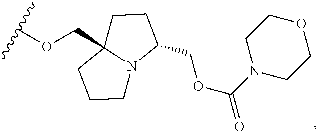 [SMILES: *OC[C@]12CCCN1[C@@H](COC(=O)N1CCOCC1)CC2]

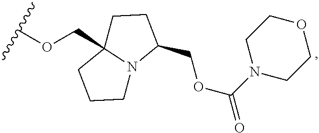 [SMILES: *OC[C@]12CCCN1[C@H](COC(=O)N1CCOCC1)CC2]

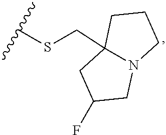 [SMILES: *SCC12CCCN1CC(F)C2]

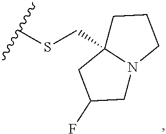 [SMILES: *SC[C@]12CCCN1CC(F)C2]

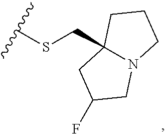 [SMILES: *SC[C@@]12CCCN1CC(F)C2]

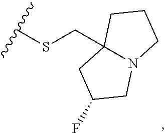 [SMILES: *SCC12CCCN1C[C@H](F)C2]

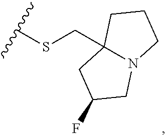 [SMILES: *SCC12CCCN1C[C@@H](F)C2]

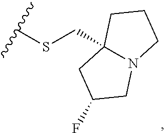 [SMILES: *SC[C@]12CCCN1C[C@H](F)C2]

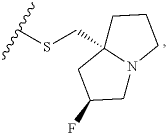 [SMILES: *SC[C@]12CCCN1C[C@@H](F)C2]

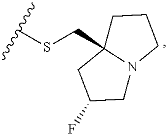 [SMILES: *SC[C@@]12CCCN1C[C@H](F)C2]

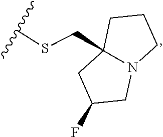 [SMILES: *SC[C@@]12CCCN1C[C@@H](F)C2]

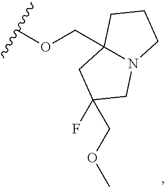 [SMILES: *OCC12CCCN1CC(F)(COC)C2]

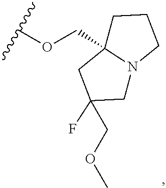 [SMILES: *OC[C@]12CCCN1CC(F)(COC)C2]

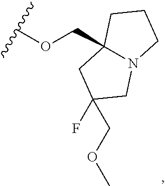 [SMILES: *OC[C@@]12CCCN1CC(F)(COC)C2]

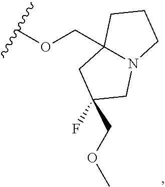 [SMILES: *OCC12CCCN1C[C@@](F)(COC)C2]

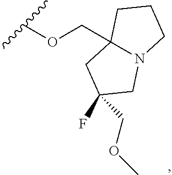 [SMILES: *OCC12CCCN1C[C@](F)(COC)C2]

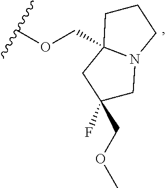 [SMILES: *OC[C@]12CCCN1C[C@@](F)(COC)C2]

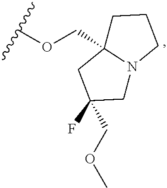 [SMILES: *OC[C@]12CCCN1C[C@](F)(COC)C2]

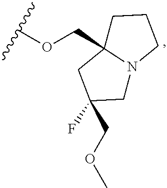 [SMILES: *OC[C@@]12CCCN1C[C@@](F)(COC)C2]

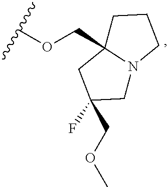 [SMILES: *OC[C@@]12CCCN1C[C@@](F)(COC)C2]

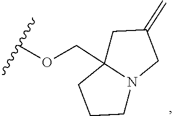 [SMILES: *OCC12CCCN1CC(=C)C2]

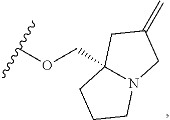 [SMILES: *OC[C@@]12CCCN1CC(=C)C2]

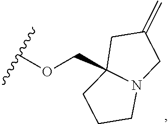 [SMILES: *OC[C@]12CCCN1CC(=C)C2]

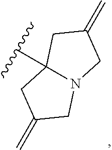 [SMILES: *C12CC(=C)CN1CC(=C)C2]

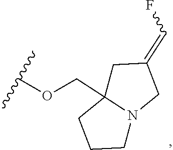 [SMILES: *OCC12CCCN1CC(=CF)C2]

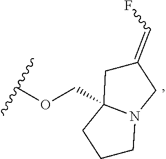 [SMILES: *OC[C@@]12CCCN1CC(=CF)C2]

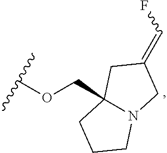 [SMILES: *OC[C@]12CCCN1CC(=CF)C2]

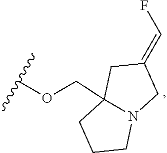 [SMILES: *OCC12CCCN1C/C(=C/F)C2]

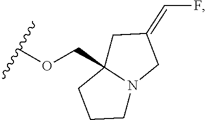 [SMILES: *OC[C@]12CCCN1C/C(=C\F)C2]

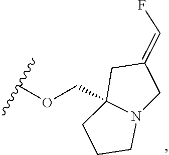 [SMILES: *OC[C@@]12CCCN1C/C(=C/F)C2]

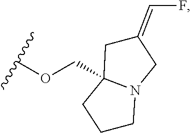 [SMILES: *OC[C@@]12CCCN1C/C(=C\F)C2]

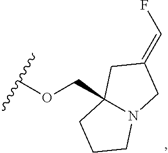 [SMILES: *OC[C@]12CCCN1C/C(=C/F)C2]

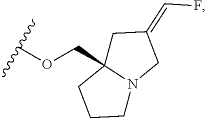 [SMILES: *OC[C@]12CCCN1C/C(=C\F)C2]

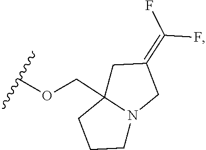 [SMILES: *OCC12CCCN1CC(=C(F)F)C2]

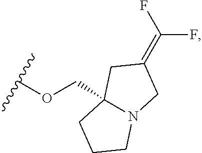 [SMILES: *OC[C@@]12CCCN1CC(=C(F)F)C2]

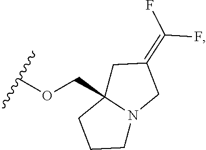 [SMILES: *OC[C@]12CCCN1CC(=C(F)F)C2]

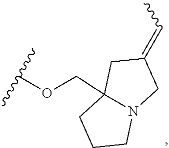

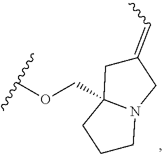

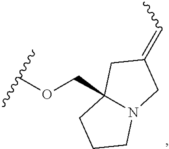

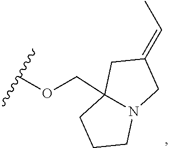 [SMILES: *OCC12CCCN1C/C(=C/C)C2]

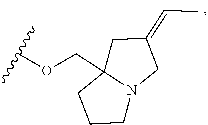 [SMILES: *OCC12CCCN1C/C(=C\C)C2]

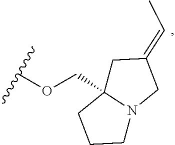 [SMILES: *OC[C@@]12CCCN1C/C(=C/C)C2]

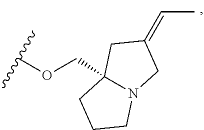 [SMILES: *OC[C@@]12CCCN1C/C(=C\C)C2]

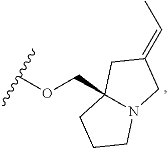 [SMILES: *OC[C@]12CCCN1C/C(=C/C)C2]

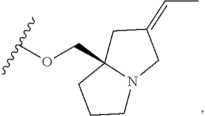 [SMILES: *OC[C@]12CCCN1C/C(=C\C)C2]

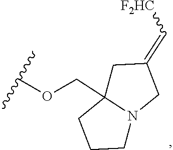 [SMILES: *OCC12CCCN1CC(=CC(F)F)C2]

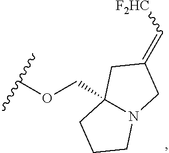 [SMILES: *OC[C@@]12CCCN1CC(=CC(F)F)C2]

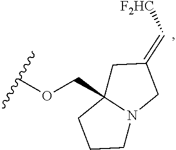

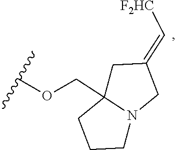 [SMILES: *OCC12CCCN1C/C(=C/C(F)F)C2]

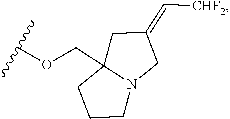 [SMILES: *OCC12CCCN1C/C(=C\C(F)F)C2]

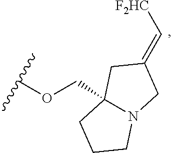 [SMILES: *OC[C@@]12CCCN1C/C(=C/C(F)F)C2]

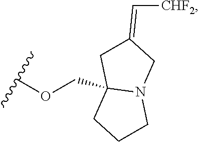 [SMILES: *OC[C@@]12CCCN1C/C(=C\C(F)F)C2]

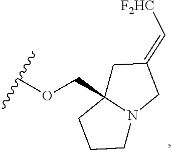 [SMILES: *OC[C@]12CCCN1C/C(=C/C(F)F)C2]

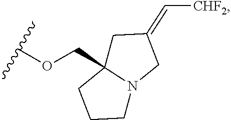 [SMILES: *OC[C@]12CCCN1C/C(=C\C(F)F)C2]

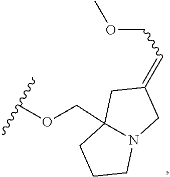 [SMILES: *OCC12CCCN1CC(=CCOC)C2]

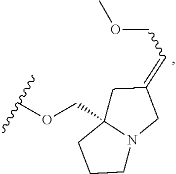 [SMILES: *OC[C@@]12CCCN1CC(=CCOC)C2]

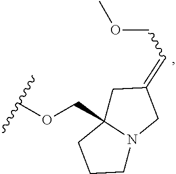 [SMILES: *OC[C@]12CCCN1CC(=CCOC)C2]

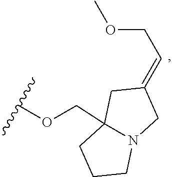 [SMILES: *OCC12CCCN1C/C(=C/COC)C2]

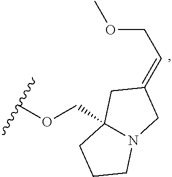 [SMILES: *OC[C@@]12CCCN1C/C(=C/COC)C2]

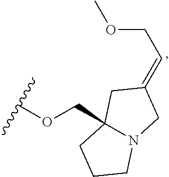 [SMILES: *OC[C@]12CCCN1C/C(=C/COC)C2]

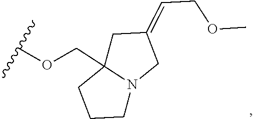 [SMILES: *OCC12CCCN1C/C(=C\COC)C2]

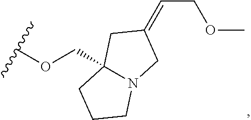 [SMILES: *OC[C@@]12CCCN1C/C(=C\COC)C2]

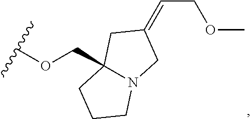 [SMILES: *OC[C@]12CCCN1C/C(=C\COC)C2]

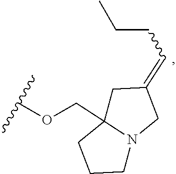 [SMILES: *OCC12CCCN1CC(=CCCC)C2]

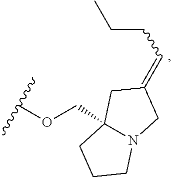 [SMILES: *OC[C@@]12CCCN1CC(=CCCC)C2]

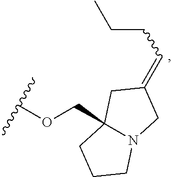 [SMILES: *OC[C@]12CCCN1CC(=CCCC)C2]

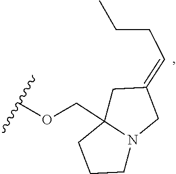 [SMILES: *OCC12CCCN1C/C(=C/CCC)C2]

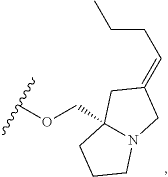 [SMILES: *OC[C@@]12CCCN1C/C(=C/CCC)C2]

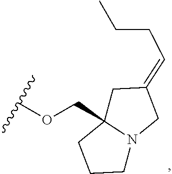 [SMILES: *OC[C@]12CCCN1C/C(=C/CCC)C2]

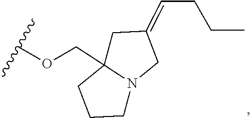 [SMILES: *OCC12CCCN1C/C(=C\CCC)C2]

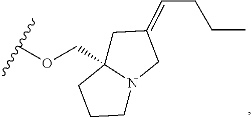 [SMILES: *OC[C@@]12CCCN1C/C(=C\CCC)C2]

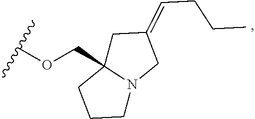 [SMILES: *OC[C@]12CCCN1C/C(=C\CCC)C2]

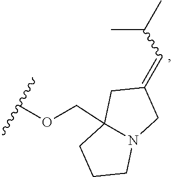 [SMILES: *OCC12CCCN1CC(=CC(C)C)C2]

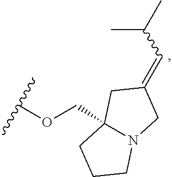 [SMILES: *OC[C@@]12CCCN1CC(=CC(C)C)C2]

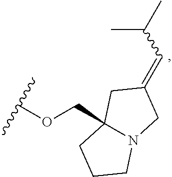 [SMILES: *OC[C@]12CCCN1CC(=CC(C)C)C2]

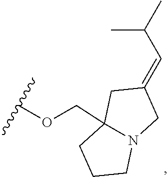 [SMILES: *OCC12CCCN1C/C(=C/C(C)C)C2]

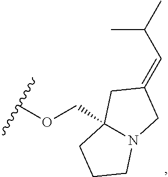 [SMILES: *OC[C@@]12CCCN1C/C(=C/C(C)C)C2]

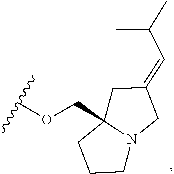 [SMILES: *OC[C@]12CCCN1C/C(=C/C(C)C)C2]

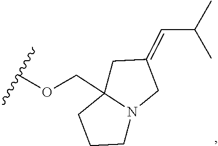 [SMILES: *OCC12CCCN1C/C(=C\C(C)C)C2]

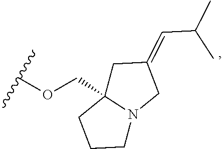 [SMILES: *OC[C@@]12CCCN1C/C(=C\C(C)C)C2]

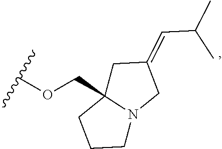 [SMILES: *OC[C@]12CCCN1C/C(=C\C(C)C)C2]

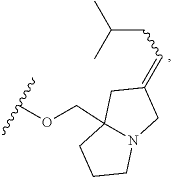 [SMILES: *OCC12CCCN1CC(=CCC(C)C)C2]

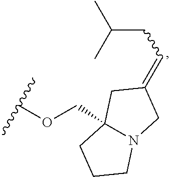 [SMILES: *OC[C@@]12CCCN1CC(=CCC(C)C)C2]

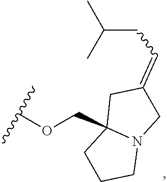 [SMILES: *OC[C@]12CCCN1CC(=CCC(C)C)C2]

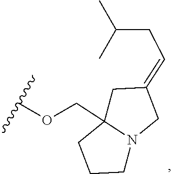 [SMILES: *OCC12CCCN1C/C(=C/CC(C)C)C2]

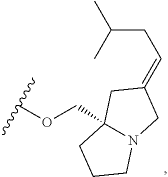 [SMILES: *OC[C@@]12CCCN1C/C(=C/CC(C)C)C2]

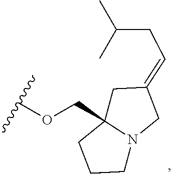 [SMILES: *OC[C@]12CCCN1C/C(=C/CC(C)C)C2]

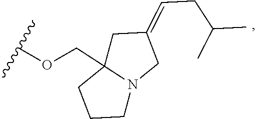 [SMILES: *OCC12CCCN1C/C(=C\CC(C)C)C2]

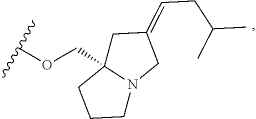 [SMILES: *OC[C@@]12CCCN1C/C(=C\CC(C)C)C2]

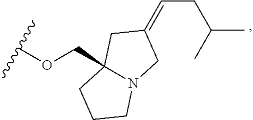 [SMILES: *OC[C@]12CCCN1C/C(=C\CC(C)C)C2]

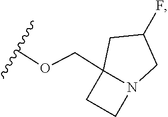 [SMILES: *OCC12CCN1CC(F)C2]

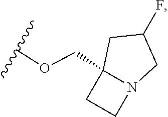 [SMILES: *OC[C@@]12CCN1CC(F)C2]

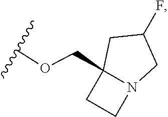 [SMILES: *OC[C@]12CCN1CC(F)C2]

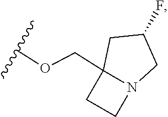 [SMILES: *OCC12CCN1C[C@@H](F)C2]

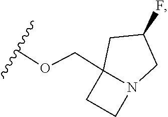 [SMILES: *OCC12CCN1C[C@H](F)C2]

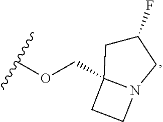 [SMILES: *OC[C@@]12CCN1C[C@@H](F)C2]

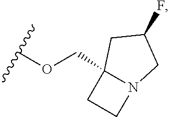 [SMILES: *OC[C@@]12CCN1C[C@H](F)C2]

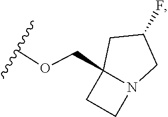 [SMILES: *OC[C@]12CCN1C[C@@H](F)C2]

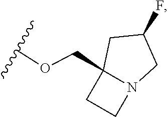 [SMILES: *OC[C@]12CCN1C[C@H](F)C2]

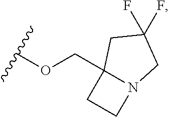 [SMILES: *OCC12CCN1CC(F)(F)C2]

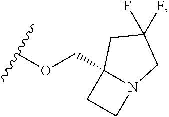 [SMILES: *OC[C@@]12CCN1CC(F)(F)C2]

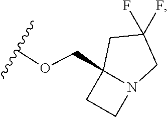 [SMILES: *OC[C@]12CCN1CC(F)(F)C2]

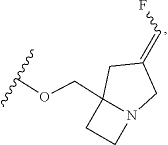 [SMILES: *OCC12CCN1CC(=CF)C2]

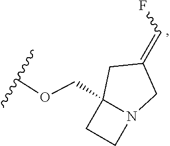 [SMILES: *OC[C@@]12CCN1CC(=CF)C2]

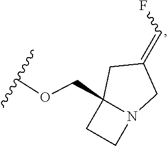 [SMILES: *OC[C@]12CCN1CC(=CF)C2]

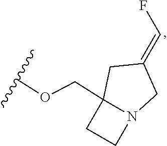 [SMILES: *OCC12CCN1C/C(=C/F)C2]

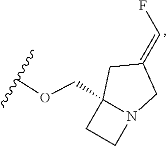 [SMILES: *OC[C@@]12CCN1C/C(=C/F)C2]

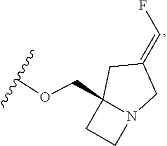 [SMILES: *OC[C@]12CCN1C/C(=C/F)C2]

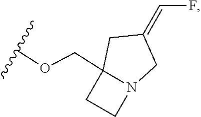 [SMILES: *OCC12CCN1C/C(=C\F)C2]

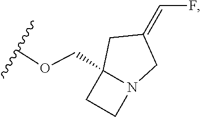 [SMILES: *OC[C@@]12CCN1C/C(=C\F)C2]

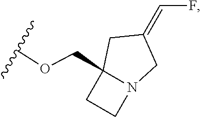 [SMILES: *OC[C@]12CCN1C/C(=C\F)C2]

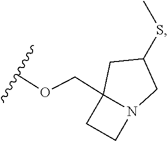 [SMILES: *OCC12CCN1CC(SC)C2]

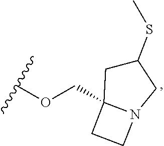 [SMILES: *OC[C@@]12CCN1CC(SC)C2]

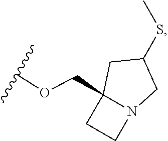 [SMILES: *OC[C@]12CCN1CC(SC)C2]

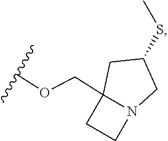 [SMILES: *OCC12CCN1C[C@@H](SC)C2]

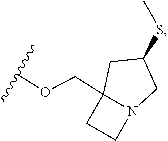 [SMILES: *OCC12CCN1C[C@H](SC)C2]

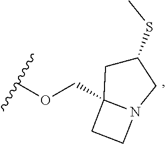 [SMILES: *OC[C@@]12CCN1C[C@@H](SC)C2]

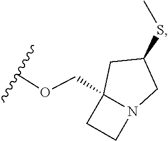 [SMILES: *OC[C@@]12CCN1C[C@H](SC)C2]

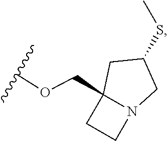 [SMILES: *OC[C@]12CCN1C[C@@H](SC)C2]

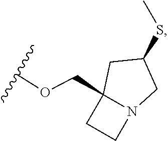 [SMILES: *OC[C@]12CCN1C[C@H](SC)C2]

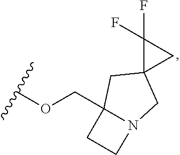 [SMILES: *OCC12CCN1CC1(C2)CC1(F)F]

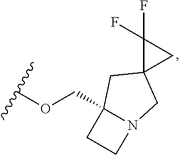 [SMILES: *OC[C@@]12CCN1CC1(CC1(F)F)C2]

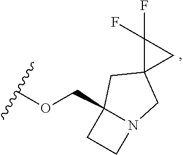 [SMILES: *OC[C@]12CCN1CC1(CC1(F)F)C2]

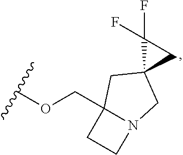 [SMILES: *OCC12CCN1C[C@@]1(C2)CC1(F)F]

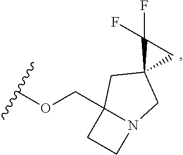 [SMILES: *OCC12CCN1C[C@]1(C2)CC1(F)F]

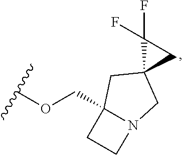 [SMILES: *OC[C@@]12CCN1C[C@]1(CC1(F)F)C2]

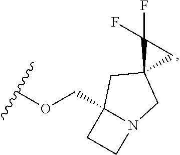 [SMILES: *OC[C@@]12CCN1C[C@@]1(CC1(F)F)C2]

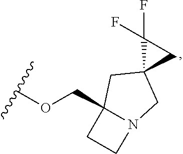 [SMILES: *OC[C@]12CCN1C[C@]1(CC1(F)F)C2]

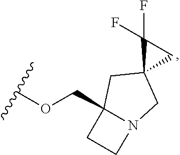 [SMILES: *OC[C@]12CCN1C[C@@]1(CC1(F)F)C2]

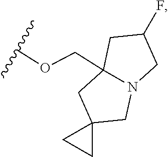 [SMILES: *OCC12CC(F)CN1CC1(CC1)C2]

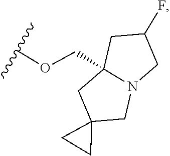 [SMILES: *OC[C@]12CC(F)CN1CC1(CC1)C2]

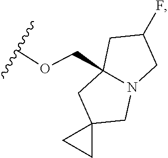 [SMILES: *OC[C@@]12CC(F)CN1CC1(CC1)C2]

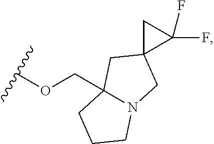 [SMILES: *OCC12CCCN1CC1(C2)CC1(F)F]

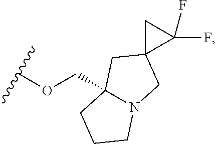 [SMILES: *OC[C@@]12CCCN1CC1(CC1(F)F)C2]

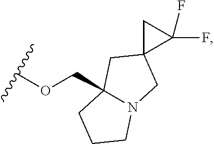 [SMILES: *OC[C@]12CCCN1CC1(CC1(F)F)C2]

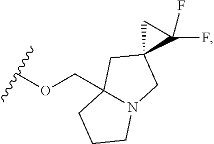 [SMILES: *OCC12CCCN1C[C@]1(C2)CC1(F)F]

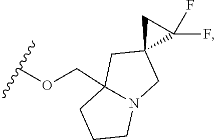 [SMILES: *OCC12CCCN1C[C@@]1(C2)CC1(F)F]

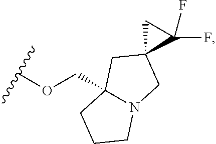 [SMILES: *OC[C@@]12CCCN1C[C@@]1(CC1(F)F)C2]

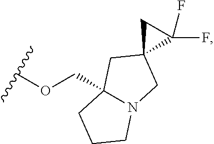 [SMILES: *OC[C@@]12CCCN1C[C@]1(CC1(F)F)C2]

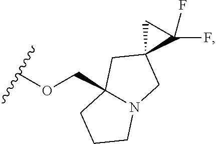 [SMILES: *OC[C@]12CCCN1C[C@@]1(CC1(F)F)C2]

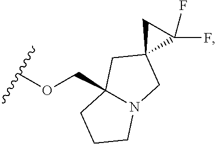 [SMILES: *OC[C@]12CCCN1C[C@]1(CC1(F)F)C2]

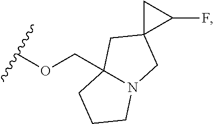 [SMILES: *OCC12CCCN1CC1(CC1F)C2]

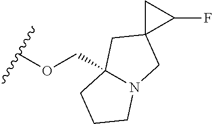 [SMILES: *OC[C@@]12CCCN1CC1(CC1F)C2]

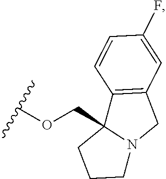 [SMILES: *OC[C@]12CCCN1Cc1cc(F)ccc12]

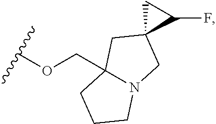

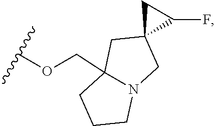

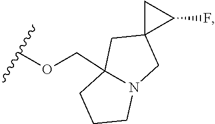 [SMILES: *OCC12CCCN1CC1(C[C@@H]1F)C2]

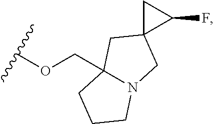 [SMILES: *OCC12CCCN1CC1(C[C@H]1F)C2]

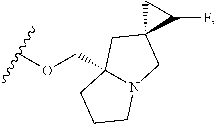

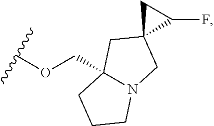

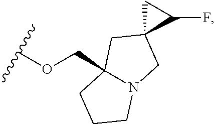

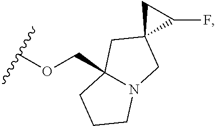

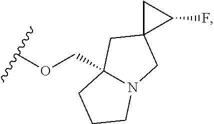 [SMILES: *OC[C@@]12CCCN1CC1(C[C@@H]1F)C2]

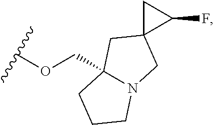 [SMILES: *OC[C@@]12CCCN1CC1(C[C@H]1F)C2]

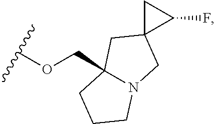 [SMILES: *OC[C@]12CCCN1CC1(C[C@@H]1F)C2]

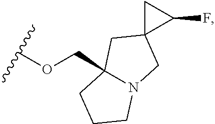 [SMILES: *OC[C@]12CCCN1CC1(C[C@H]1F)C2]

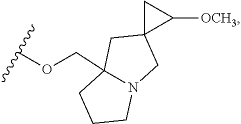 [SMILES: *OCC12CCCN1CC1(CC1OC)C2]

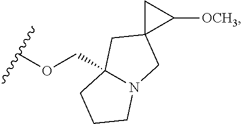 [SMILES: *OC[C@@]12CCCN1CC1(CC1OC)C2]

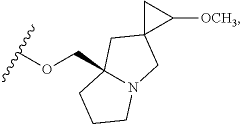 [SMILES: *OC[C@]12CCCN1CC1(CC1OC)C2]

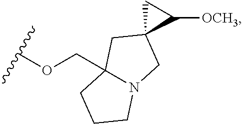

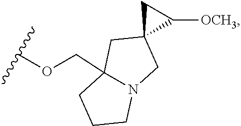

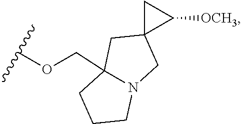 [SMILES: *OCC12CCCN1CC1(C[C@@H]1OC)C2]

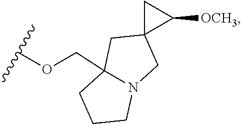 [SMILES: *OCC12CCCN1CC1(C[C@H]1OC)C2]

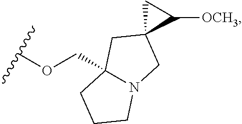

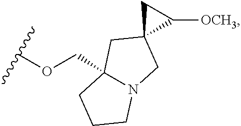

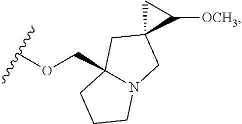

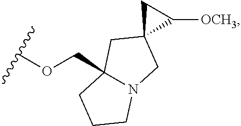

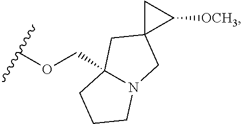 [SMILES: *OC[C@@]12CCCN1CC1(C[C@@H]1OC)C2]

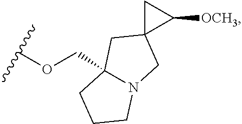 [SMILES: *OC[C@@]12CCCN1CC1(C[C@H]1OC)C2]

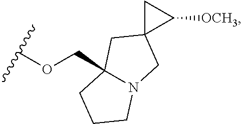 [SMILES: *OC[C@]12CCCN1CC1(C[C@@H]1OC)C2]

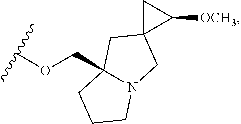 [SMILES: *OC[C@]12CCCN1CC1(C[C@H]1OC)C2]

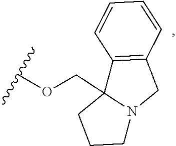 [SMILES: *OCC12CCCN1Cc1ccccc12]

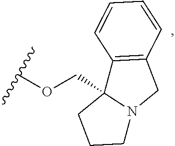 [SMILES: *OC[C@@]12CCCN1Cc1ccccc12]

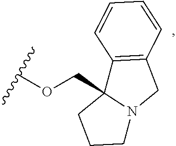 [SMILES: *OC[C@]12CCCN1Cc1ccccc12]

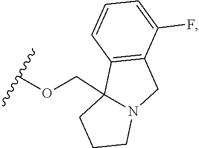 [SMILES: *OCC12CCCN1Cc1c(F)cccc12]

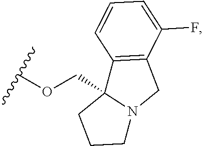 [SMILES: *OC[C@@]12CCCN1Cc1c(F)cccc12]

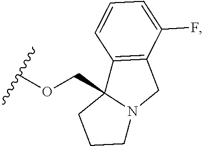 [SMILES: *OC[C@]12CCCN1Cc1c(F)cccc12]

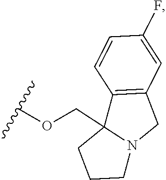 [SMILES: *OCC12CCCN1Cc1cc(F)ccc12]

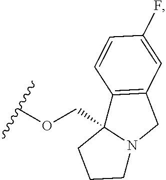 [SMILES: *OC[C@@]12CCCN1Cc1cc(F)ccc12]

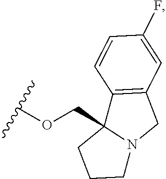 [SMILES: *OC[C@]12CCCN1Cc1cc(F)ccc12]

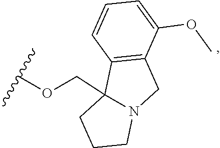 [SMILES: *OCC12CCCN1Cc1c(OC)cccc12]

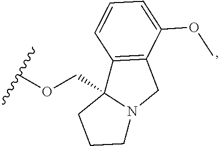 [SMILES: *OC[C@@]12CCCN1Cc1c(OC)cccc12]

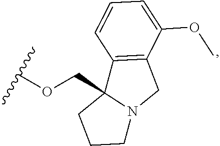 [SMILES: *OC[C@]12CCCN1Cc1c(OC)cccc12]

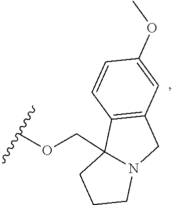 [SMILES: *OCC12CCCN1Cc1cc(OC)ccc12]

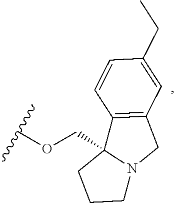 [SMILES: *OC[C@@]12CCCN1Cc1cc(CC)ccc12]

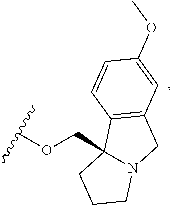 [SMILES: *OC[C@]12CCCN1Cc1cc(OC)ccc12]

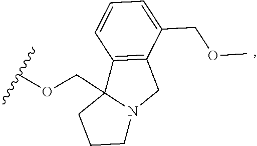 [SMILES: *OCC12CCCN1Cc1c(COC)cccc12]

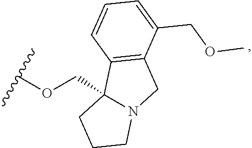 [SMILES: *OC[C@@]12CCCN1Cc1c(COC)cccc12]

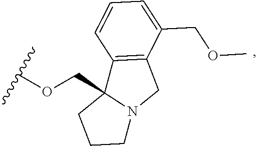 [SMILES: *OC[C@]12CCCN1Cc1c(COC)cccc12]

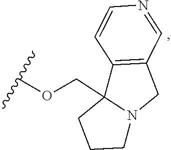 [SMILES: *OCC12CCCN1Cc1cnccc12]

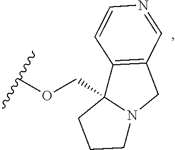 [SMILES: *OC[C@@]12CCCN1Cc1cnccc12]

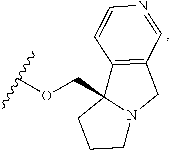 [SMILES: *OC[C@]12CCCN1Cc1cnccc12]

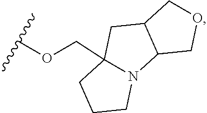 [SMILES: *OCC12CCCN1C1COCC1C2]

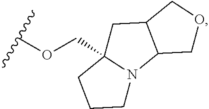 [SMILES: *OC[C@@]12CCCN1C1COCC1C2]

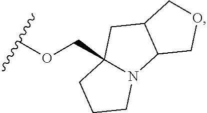 [SMILES: *OC[C@]12CCCN1C1COCC1C2]

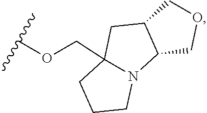 [SMILES: *OCC12CCCN1[C@H]1COC[C@H]1C2]

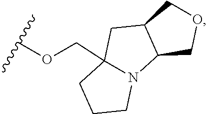 [SMILES: *OCC12CCCN1[C@@H]1COC[C@@H]1C2]

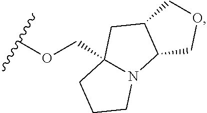 [SMILES: *OC[C@@]12CCCN1[C@H]1COC[C@H]1C2]

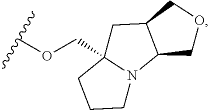 [SMILES: *OC[C@@]12CCCN1[C@@H]1COC[C@@H]1C2]

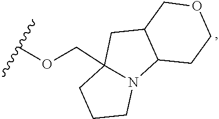 [SMILES: *OCC12CCCN1C1CCOCC1C2]

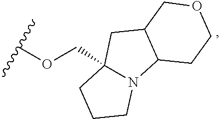 [SMILES: *OC[C@@]12CCCN1C1CCOCC1C2]

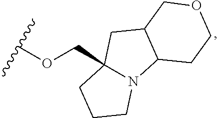 [SMILES: *OC[C@]12CCCN1C1CCOCC1C2]

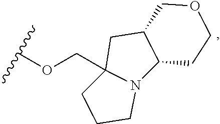 [SMILES: *OCC12CCCN1[C@H]1CCOC[C@H]1C2]

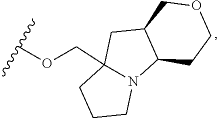 [SMILES: *OCC12CCCN1[C@@H]1CCOC[C@@H]1C2]

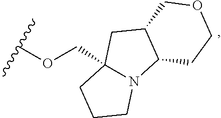 [SMILES: *OC[C@@]12CCCN1[C@H]1CCOC[C@H]1C2]

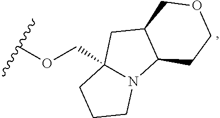 [SMILES: *OC[C@@]12CCCN1[C@@H]1CCOC[C@@H]1C2]

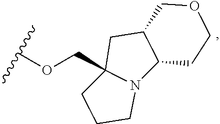 [SMILES: *OC[C@]12CCCN1[C@H]1CCOC[C@H]1C2]

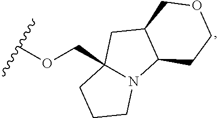 [SMILES: *OC[C@]12CCCN1[C@@H]1CCOC[C@@H]1C2]

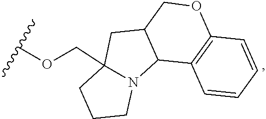 [SMILES: *OCC12CCCN1C1c3ccccc3OCC1C2]

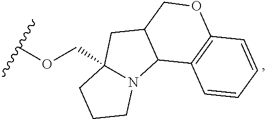 [SMILES: *OC[C@@]12CCCN1C1c3ccccc3OCC1C2]

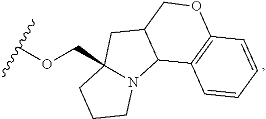 [SMILES: *OC[C@]12CCCN1C1c3ccccc3OCC1C2]

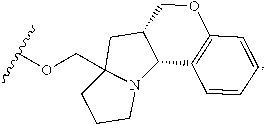 [SMILES: *OCC12CCCN1[C@H]1c3ccccc3OC[C@H]1C2]

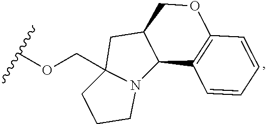 [SMILES: *OCC12CCCN1[C@@H]1c3ccccc3OC[C@@H]1C2]

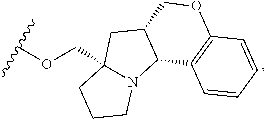 [SMILES: *OC[C@@]12CCCN1[C@H]1c3ccccc3OC[C@H]1C2]

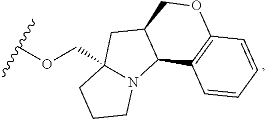 [SMILES: *OC[C@@]12CCCN1[C@@H]1c3ccccc3OC[C@@H]1C2]

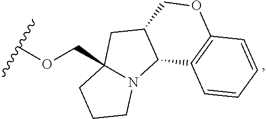 [SMILES: *OC[C@]12CCCN1[C@H]1c3ccccc3OC[C@H]1C2]

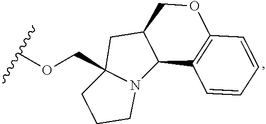 [SMILES: *OC[C@]12CCCN1[C@@H]1c3ccccc3OC[C@@H]1C2]

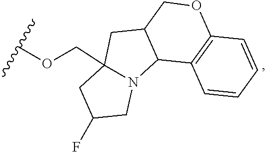 [SMILES: *OCC12CC(F)CN1C1c3ccccc3OCC1C2]

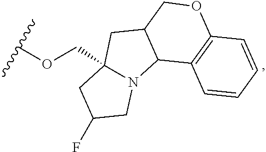 [SMILES: *OC[C@@]12CC(F)CN1C1c3ccccc3OCC1C2]

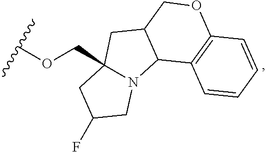 [SMILES: *OC[C@]12CC(F)CN1C1c3ccccc3OCC1C2]

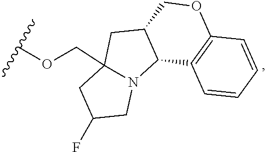 [SMILES: *OCC12CC(F)CN1[C@H]1c3ccccc3OC[C@H]1C2]

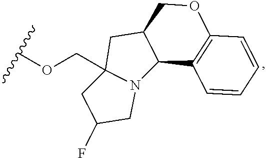 [SMILES: *OCC12CC(F)CN1[C@@H]1c3ccccc3OC[C@@H]1C2]

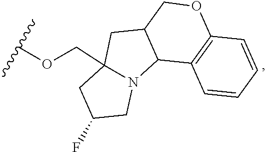 [SMILES: *OCC12CC3COc4ccccc4C3N1C[C@H](F)C2]

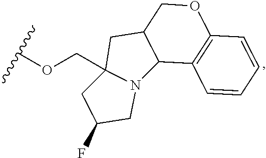 [SMILES: *OCC12CC3COc4ccccc4C3N1C[C@@H](F)C2]

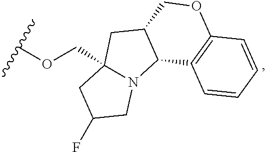 [SMILES: *OC[C@@]12CC(F)CN1[C@H]1c3ccccc3OC[C@H]1C2]

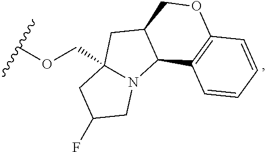 [SMILES: *OC[C@@]12CC(F)CN1[C@@H]1c3ccccc3OC[C@@H]1C2]

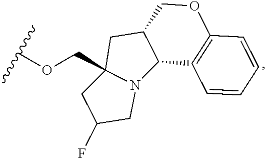 [SMILES: *OC[C@]12CC(F)CN1[C@H]1c3ccccc3OC[C@H]1C2]

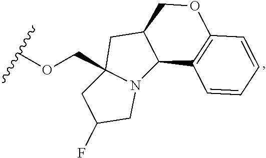 [SMILES: *OC[C@]12CC(F)CN1[C@@H]1c3ccccc3OC[C@@H]1C2]

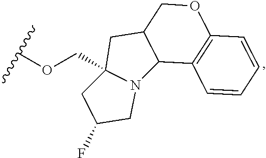 [SMILES: *OC[C@]12CC3COc4ccccc4C3N1C[C@H](F)C2]

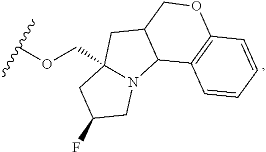 [SMILES: *OC[C@]12CC3COc4ccccc4C3N1C[C@@H](F)C2]

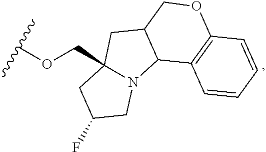 [SMILES: *OC[C@@]12CC3COc4ccccc4C3N1C[C@H](F)C2]

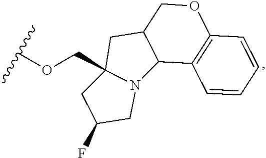 [SMILES: *OC[C@@]12CC3COc4ccccc4C3N1C[C@@H](F)C2]

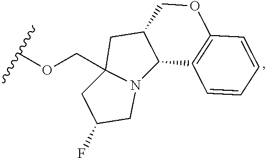 [SMILES: *OCC12C[C@@H](F)CN1[C@H]1c3ccccc3OC[C@H]1C2]

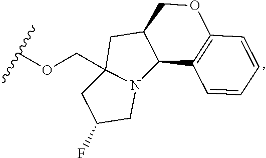 [SMILES: *OCC12C[C@@H](F)CN1[C@@H]1c3ccccc3OC[C@@H]1C2]

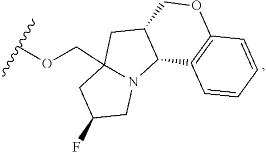 [SMILES: *OCC12C[C@H](F)CN1[C@H]1c3ccccc3OC[C@H]1C2]

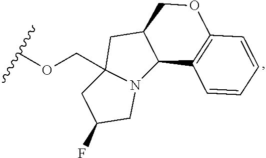 [SMILES: *OCC12C[C@H](F)CN1[C@@H]1c3ccccc3OC[C@@H]1C2]

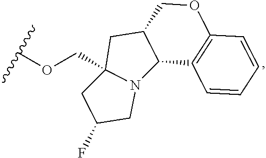 [SMILES: *OC[C@@]12C[C@@H](F)CN1[C@H]1c3ccccc3OC[C@H]1C2]

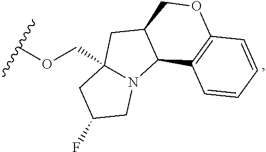 [SMILES: *OC[C@@]12C[C@@H](F)CN1[C@@H]1c3ccccc3OC[C@@H]1C2]

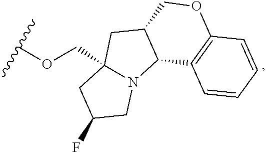 [SMILES: *OC[C@@]12C[C@H](F)CN1[C@H]1c3ccccc3OC[C@H]1C2]

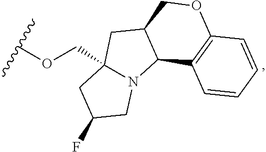 [SMILES: *OC[C@@]12C[C@H](F)CN1[C@@H]1c3ccccc3OC[C@@H]1C2]

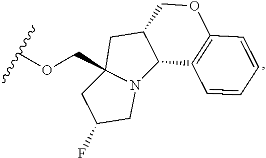 [SMILES: *OC[C@]12C[C@@H](F)CN1[C@H]1c3ccccc3OC[C@H]1C2]

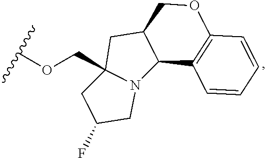 [SMILES: *OC[C@]12C[C@@H](F)CN1[C@@H]1c3ccccc3OC[C@@H]1C2]

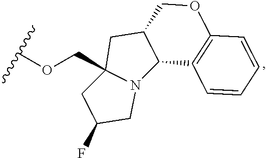 [SMILES: *OC[C@]12C[C@H](F)CN1[C@H]1c3ccccc3OC[C@H]1C2]

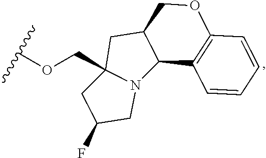 [SMILES: *OC[C@]12C[C@H](F)CN1[C@@H]1c3ccccc3OC[C@@H]1C2]

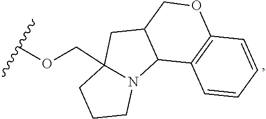 [SMILES: *OCC12CCCN1C1c3ccccc3OCC1C2]

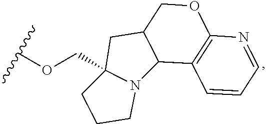 [SMILES: *OC[C@@]12CCCN1C1c3cccnc3OCC1C2]

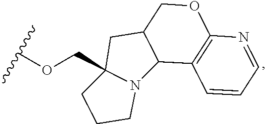 [SMILES: *OC[C@]12CCCN1C1c3cccnc3OCC1C2]

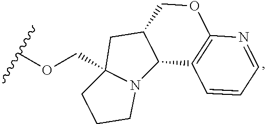 [SMILES: *OC[C@@]12CCCN1[C@H]1c3cccnc3OC[C@H]1C2]

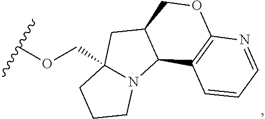 [SMILES: *OC[C@@]12CCCN1[C@@H]1c3cccnc3OC[C@@H]1C2]

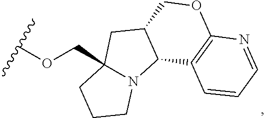 [SMILES: *OC[C@]12CCCN1[C@H]1c3cccnc3OC[C@H]1C2]

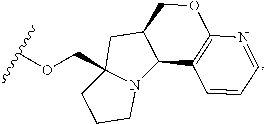 [SMILES: *OC[C@]12CCCN1[C@@H]1c3cccnc3OC[C@@H]1C2]

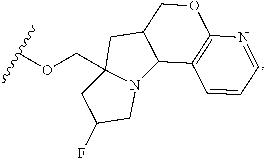 [SMILES: *OCC12CC(F)CN1C1c3cccnc3OCC1C2]

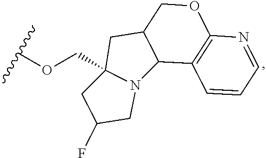 [SMILES: *OC[C@@]12CC(F)CN1C1c3cccnc3OCC1C2]

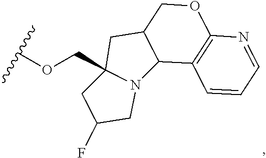 [SMILES: *OC[C@]12CC(F)CN1C1c3cccnc3OCC1C2]

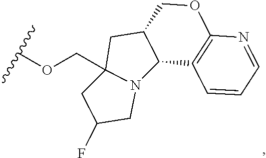 [SMILES: *OCC12CC(F)CN1[C@H]1c3cccnc3OC[C@H]1C2]

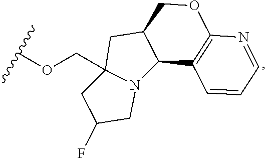 [SMILES: *OCC12CC(F)CN1[C@@H]1c3cccnc3OC[C@@H]1C2]

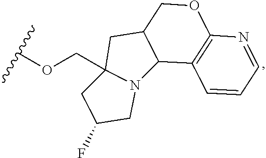 [SMILES: *OCC12CC3COc4ncccc4C3N1C[C@H](F)C2]

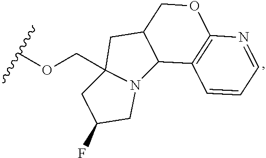 [SMILES: *OCC12CC3COc4ncccc4C3N1C[C@@H](F)C2]

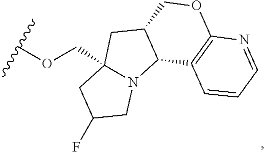 [SMILES: *OC[C@@]12CC(F)CN1[C@H]1c3cccnc3OC[C@H]1C2]

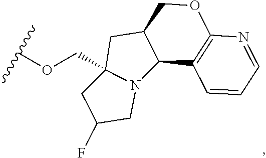 [SMILES: *OC[C@@]12CC(F)CN1[C@@H]1c3cccnc3OC[C@@H]1C2]

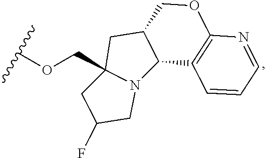 [SMILES: *OC[C@]12CC(F)CN1[C@H]1c3cccnc3OC[C@H]1C2]

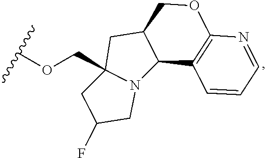 [SMILES: *OC[C@]12CC(F)CN1[C@@H]1c3cccnc3OC[C@@H]1C2]

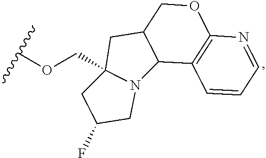 [SMILES: *OC[C@]12CC3COc4ncccc4C3N1C[C@H](F)C2]

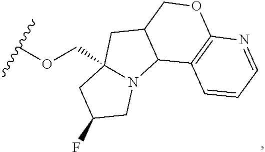 [SMILES: *OC[C@]12CC3COc4ncccc4C3N1C[C@@H](F)C2]

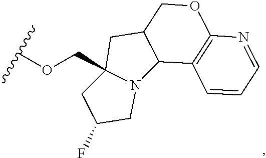 [SMILES: *OC[C@@]12CC3COc4ncccc4C3N1C[C@H](F)C2]

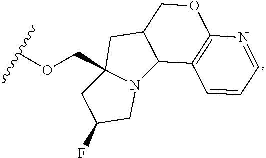 [SMILES: *OC[C@@]12CC3COc4ncccc4C3N1C[C@@H](F)C2]

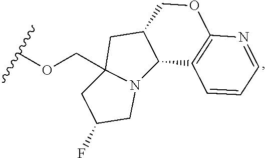 [SMILES: *OCC12C[C@@H](F)CN1[C@H]1c3cccnc3OC[C@H]1C2]

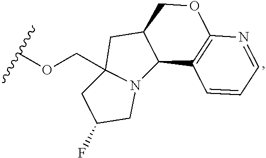 [SMILES: *OCC12C[C@@H](F)CN1[C@@H]1c3cccnc3OC[C@@H]1C2]

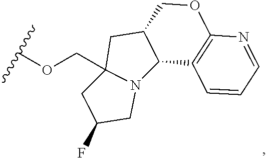 [SMILES: *OCC12C[C@H](F)CN1[C@H]1c3cccnc3OC[C@H]1C2]

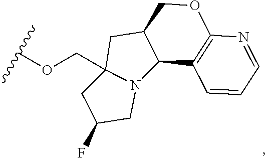 [SMILES: *OCC12C[C@H](F)CN1[C@@H]1c3cccnc3OC[C@@H]1C2]

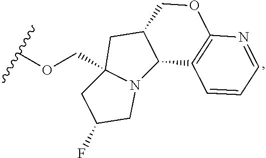 [SMILES: *OC[C@@]12C[C@@H](F)CN1[C@H]1c3cccnc3OC[C@H]1C2]

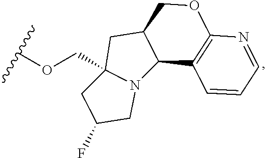 [SMILES: *OC[C@@]12C[C@@H](F)CN1[C@@H]1c3cccnc3OC[C@@H]1C2]

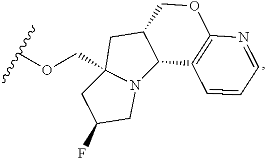 [SMILES: *OC[C@@]12C[C@H](F)CN1[C@H]1c3cccnc3OC[C@H]1C2]

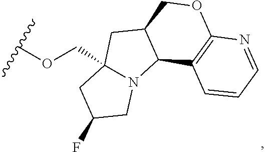 [SMILES: *OC[C@@]12C[C@H](F)CN1[C@@H]1c3cccnc3OC[C@@H]1C2]

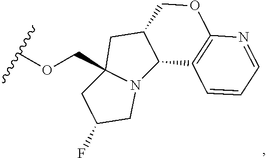 [SMILES: *OC[C@]12C[C@@H](F)CN1[C@H]1c3cccnc3OC[C@H]1C2]

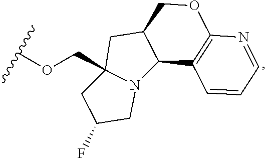 [SMILES: *OC[C@]12C[C@@H](F)CN1[C@@H]1c3cccnc3OC[C@@H]1C2]

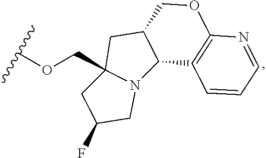 [SMILES: *OC[C@]12C[C@H](F)CN1[C@H]1c3cccnc3OC[C@H]1C2]

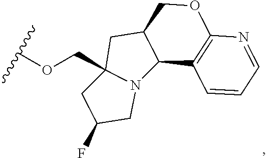 [SMILES: *OC[C@]12C[C@H](F)CN1[C@@H]1c3cccnc3OC[C@@H]1C2]

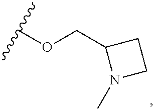 [SMILES: *OCC1CCN1C]

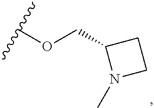 [SMILES: *OC[C@@H]1CCN1C]

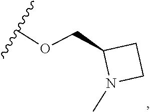 [SMILES: *OC[C@H]1CCN1C]

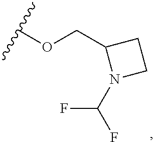 [SMILES: *OCC1CCN1C(F)F]

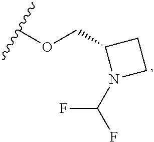 [SMILES: *OC[C@@H]1CCN1C(F)F]

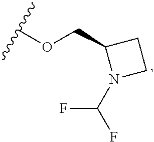 [SMILES: *OC[C@H]1CCN1C(F)F]

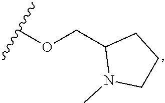 [SMILES: *OCC1CCCN1C]

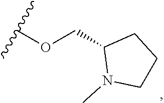 [SMILES: *OC[C@@H]1CCCN1C]

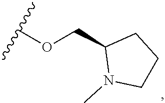 [SMILES: *OC[C@H]1CCCN1C]

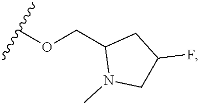 [SMILES: *OCC1CC(F)CN1C]

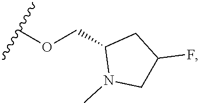 [SMILES: *OC[C@@H]1CC(F)CN1C]

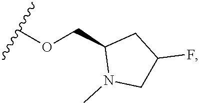 [SMILES: *OC[C@H]1CC(F)CN1C]

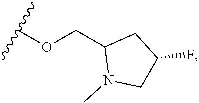 [SMILES: *OCC1C[C@H](F)CN1C]

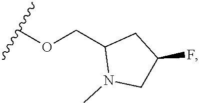 [SMILES: *OCC1C[C@@H](F)CN1C]

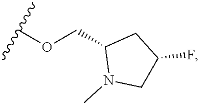 [SMILES: *OC[C@@H]1C[C@H](F)CN1C]

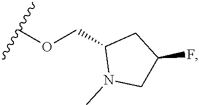 [SMILES: *OC[C@@H]1C[C@@H](F)CN1C]

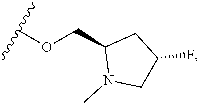 [SMILES: *OC[C@H]1C[C@H](F)CN1C]

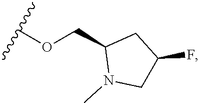 [SMILES: *OC[C@H]1C[C@@H](F)CN1C]

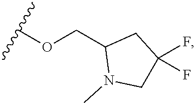 [SMILES: *OCC1CC(F)(F)CN1C]

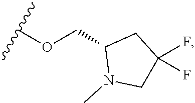 [SMILES: *OC[C@@H]1CC(F)(F)CN1C]

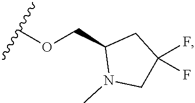 [SMILES: *OC[C@H]1CC(F)(F)CN1C]

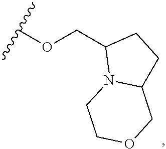 [SMILES: *OCC1CCC2COCCN12]

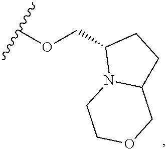 [SMILES: *OC[C@@H]1CCC2COCCN21]

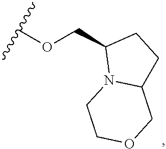 [SMILES: *OC[C@H]1CCC2COCCN21]

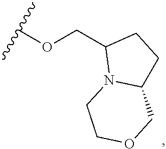 [SMILES: *OCC1CC[C@@H]2COCCN12]

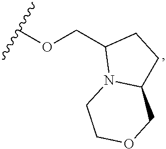 [SMILES: *OCC1CC[C@H]2COCCN12]

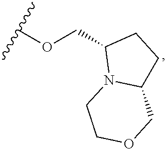 [SMILES: *OC[C@@H]1CC[C@@H]2COCCN21]

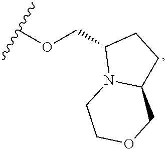 [SMILES: *OC[C@@H]1CC[C@H]2COCCN12]

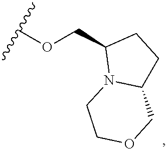 [SMILES: *OC[C@H]1CC[C@@H]2COCCN12]

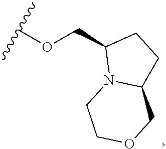 [SMILES: *OC[C@H]1CC[C@H]2COCCN12]

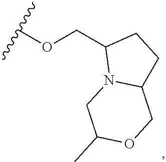 [SMILES: *OCC1CCC2COC(C)CN12]

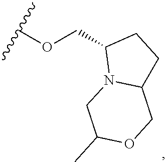 [SMILES: *OC[C@@H]1CCC2COC(C)CN21]

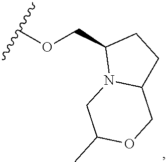 [SMILES: *OC[C@H]1CCC2COC(C)CN21]

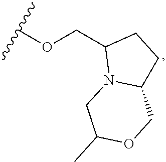

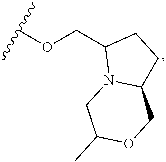

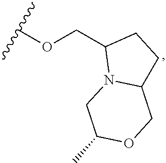 [SMILES: *OCC1CCC2CO[C@H](C)CN12]

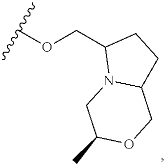 [SMILES: *OCC1CCC2CO[C@@H](C)CN12]

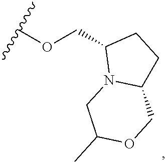

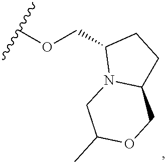

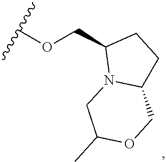

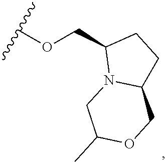

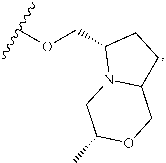 [SMILES: *OC[C@@H]1CCC2CO[C@H](C)CN21]

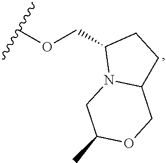 [SMILES: *OC[C@@H]1CCC2CO[C@@H](C)CN21]

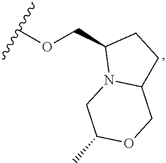 [SMILES: *OC[C@H]1CCC2CO[C@H](C)CN21]

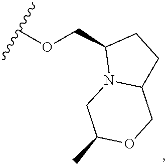 [SMILES: *OC[C@H]1CCC2CO[C@@H](C)CN21]

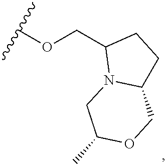 [SMILES: *OCC1CC[C@@H]2CO[C@H](C)CN12]

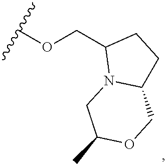 [SMILES: *OCC1CC[C@@H]2CO[C@@H](C)CN12]

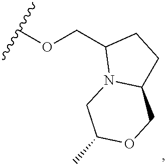 [SMILES: *OCC1CC[C@H]2CO[C@H](C)CN12]

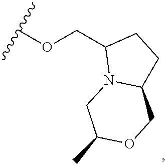 [SMILES: *OCC1CC[C@H]2CO[C@@H](C)CN12]

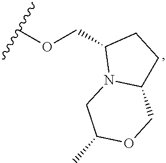 [SMILES: *OC[C@@H]1CC[C@@H]2CO[C@H](C)CN21]

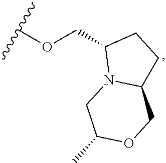 [SMILES: *OC[C@@H]1CC[C@H]2CO[C@H](C)CN12]

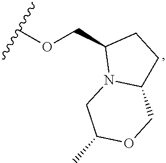 [SMILES: *OC[C@H]1CC[C@@H]2CO[C@H](C)CN12]

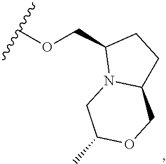 [SMILES: *OC[C@H]1CC[C@H]2CO[C@H](C)CN12]

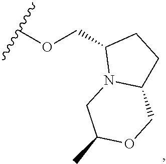 [SMILES: *OC[C@@H]1CC[C@@H]2CO[C@@H](C)CN21]

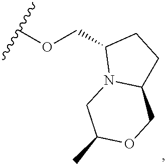 [SMILES: *OC[C@@H]1CC[C@H]2CO[C@@H](C)CN12]

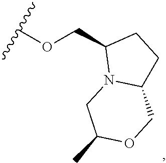 [SMILES: *OC[C@H]1CC[C@@H]2CO[C@@H](C)CN12]

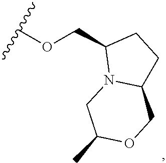 [SMILES: *OC[C@H]1CC[C@H]2CO[C@@H](C)CN12]

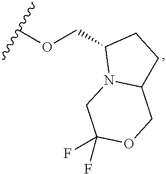 [SMILES: *OC[C@@H]1CCC2COC(F)(F)CN21]

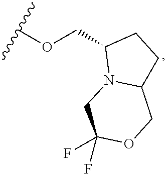

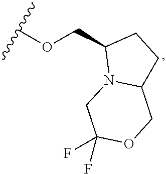 [SMILES: *OC[C@H]1CCC2COC(F)(F)CN21]

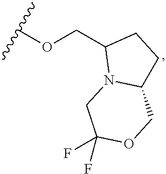 [SMILES: *OCC1CC[C@@H]2COC(F)(F)CN12]

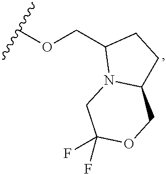 [SMILES: *OCC1CC[C@H]2COC(F)(F)CN12]

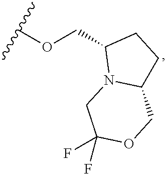 [SMILES: *OC[C@@H]1CC[C@@H]2COC(F)(F)CN21]

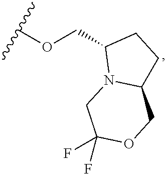 [SMILES: *OC[C@@H]1CC[C@H]2COC(F)(F)CN12]

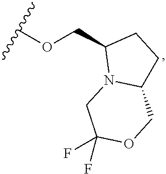 [SMILES: *OC[C@H]1CC[C@@H]2COC(F)(F)CN12]

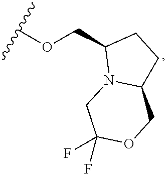 [SMILES: *OC[C@H]1CC[C@H]2COC(F)(F)CN12]

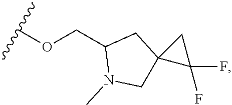 [SMILES: *OCC1CC2(CN1C)CC2(F)F]

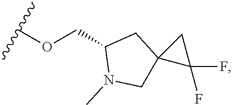 [SMILES: *OC[C@@H]1CC2(CN1C)CC2(F)F]

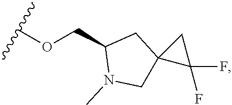 [SMILES: *OC[C@H]1CC2(CN1C)CC2(F)F]

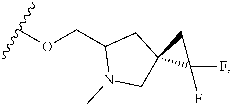 [SMILES: *OCC1C[C@]2(CN1C)CC2(F)F]

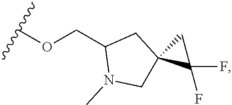 [SMILES: *OCC1C[C@@]2(CN1C)CC2(F)F]

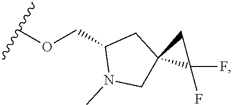 [SMILES: *OC[C@@H]1C[C@]2(CN1C)CC2(F)F]

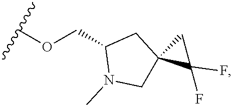 [SMILES: *OC[C@@H]1C[C@@]2(CN1C)CC2(F)F]

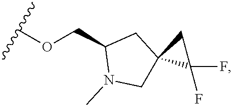 [SMILES: *OC[C@H]1C[C@]2(CN1C)CC2(F)F]

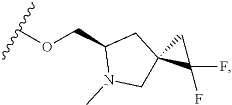 [SMILES: *OC[C@H]1C[C@@]2(CN1C)CC2(F)F]

or

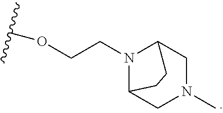 [SMILES: *OCCN1C2CCC1CN(C)C2]

[63]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [62], wherein, R₄ is selected from phenyl, pyridyl, naphthyl, quinolyl, isoquinolyl, indazolyl, benzothiaphenyl or benzothiozolyl, said phenyl, pyridyl, naphthyl, quinolyl, isoquinolyl, indazolyl, benzothiaphenyl or benzothiozolyl is unsubstituted or substituted with 1, 2, 3, 4, 5 or 6 R_{S4}.
[64]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [63], wherein, R₄ is selected from

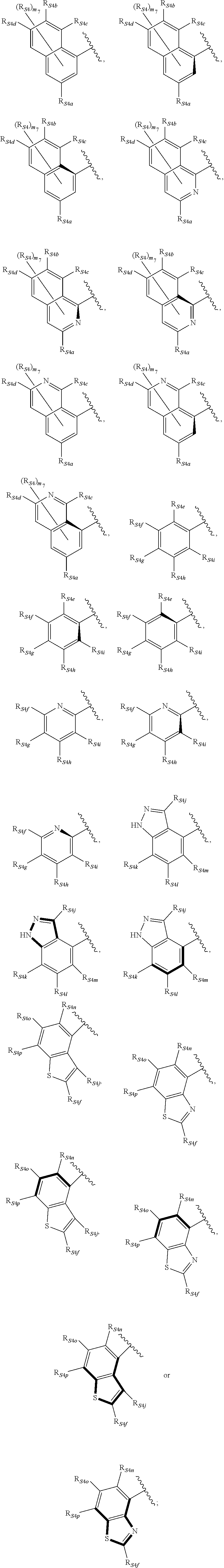

m₇ is selected from 0, 1, 2 or, 3;
R_{S4a} is selected from —OH or —NH₂;
R_{S4b} is selected from hydrogen, deuterium, halogen;
R_{S4c} is selected from hydrogen, deuterium, —C₁₋₃alkyl, —C₂₋₃alkenyl or —C₂₋₃alkynyl;
R_{S4d} is selected from hydrogen, deuterium or halogen;
R_{S4e} is selected from hydrogen, deuterium, halogen, —C₁₋₃alkyl or haloC₁₋₃alkyl;
R_{S4f} is selected from —OH or —NH₂;
R_{S4g} is selected from hydrogen, deuterium, halogen, —C₁₋₃alkyl or haloC₁₋₃alkyl;
R_{S4h} is selected from hydrogen, deuterium, halogen, —C₁₋₃alkyl or haloC₁₋₃alkyl;
R_{S4i} is selected from hydrogen, deuterium, halogen, —C₁₋₃alkyl or haloC₁₋₃alkyl;
R_{S4j} is selected from hydrogen, deuterium, halogen, —CN, —C₁₋₃alkyl, haloC₁₋₃alkyl or —OhaloC₁₋₃alkyl;
R_{S4k} is selected from hydrogen, deuterium, halogen, —CN, —C₁₋₃alkyl, haloC₁₋₃alkyl or —OhaloC₁₋₃alkyl;
R_{S4l} is selected from hydrogen, deuterium, halogen, —CN, —C₁₋₃alkyl, haloC₁₋₃alkyl or —OhaloC₁₋₃alkyl;
R_{S4m} is selected from hydrogen, deuterium, halogen, —CN, —C₁₋₃alkyl, haloC₁₋₃alkyl or —OhaloC₁₋₃alkyl;
R_{S4n} is selected from hydrogen, deuterium, halogen, —C₁₋₃alkyl or haloC₁₋₃alkyl;
R_{S4o} is selected from hydrogen, deuterium, halogen, —C₁₋₃alkyl or haloC₁₋₃alkyl;
R_{S4p} is selected from hydrogen, deuterium, halogen, —C₁₋₃alkyl or haloC₁₋₃alkyl.
[65]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [64], wherein:
m₇ is selected from 0;
R_{S4a} is selected from —OH or —NH₂;
R_{S4b} is selected from —F;
R_{S4c} is selected from ethyl, ethenyl or ethynyl;
R_{S4d} is selected from hydrogen, or —F;
R_{S4e} is selected from —F;
R_{S4f} is selected from —NH₂;
R_{S4g} is selected from hydrogen, —F, or methyl;
R_{S4h} is selected from hydrogen, —F or methyl;
R_{S4i} is selected from —I or —CF₃;
R_{S4j} is selected from —CN;
R_{S4k} is selected from hydrogen;
R_{S4l} is selected from methyl;
R_{S4m} is selected from —CF₃, —OCF₂Cl, —OCF₃ or —CF₂H;
R_{S4n} is selected from hydrogen, —F, or methyl;
R_{S4o} is selected from hydrogen, —F or methyl;
R_{S4p} p is selected from hydrogen, —F, or methyl.
[66]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [65], wherein, R₄ is selected from the following Table D:

TABLE D

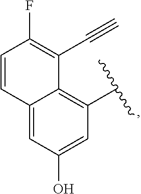 [SMILES: *c1cc(O)cc2ccc(F)c(C#C)c12]

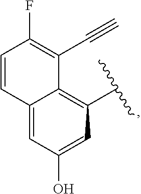

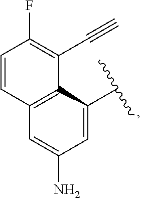

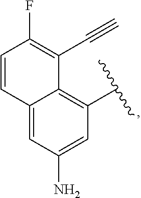 [SMILES: *c1cc(N)cc2ccc(F)c(C#C)c12]

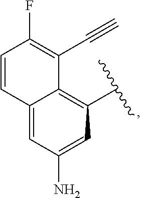

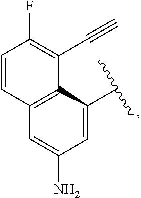

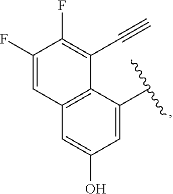 [SMILES: *c1cc(O)cc2cc(F)c(F)c(C#C)c12]

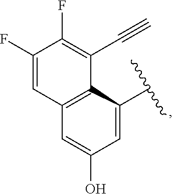

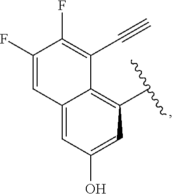

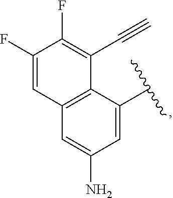 [SMILES: *c1cc(N)cc2cc(F)c(F)c(C#C)c12]

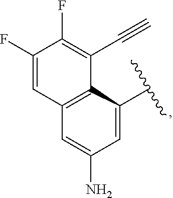

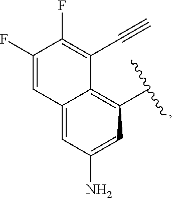

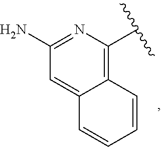 [SMILES: *c1nc(N)cc2ccccc12]

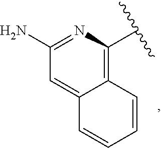

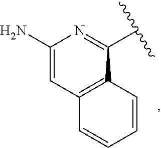

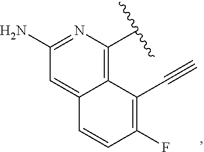 [SMILES: *c1nc(N)cc2ccc(F)c(C#C)c12]

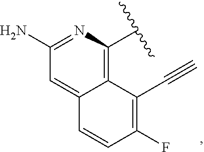

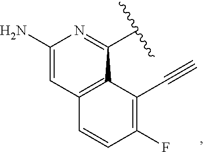

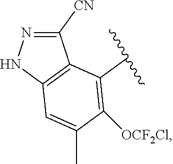

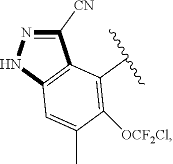

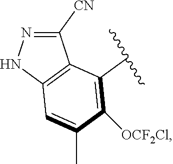

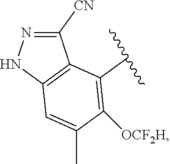

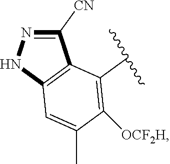

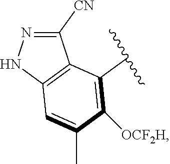

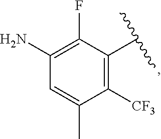 [SMILES: *c1c(F)c(N)cc(C)c1C(F)(F)F]

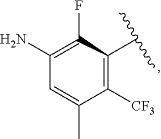

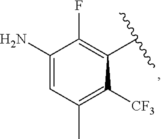

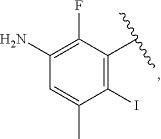 [SMILES: *c1c(F)c(N)cc(C)c1I ; ","]

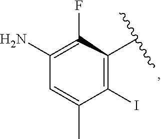

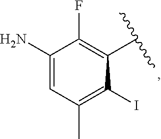

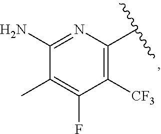 [SMILES: *c1nc(N)c(C)c(F)c1C(F)(F)F ; ","]

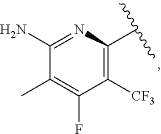

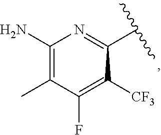

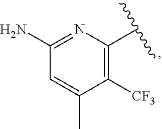 [SMILES: *c1nc(N)cc(C)c1C(F)(F)F]

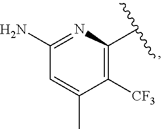

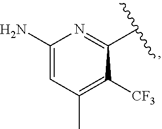

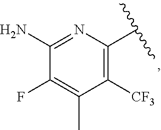 [SMILES: *c1nc(N)c(F)c(C)c1C(F)(F)F ; ","]

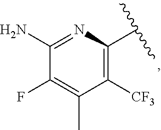

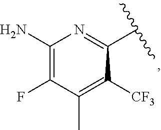

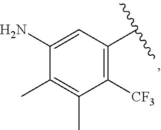 [SMILES: *c1cc(N)c(C)c(C)c1C(F)(F)F ; ","]

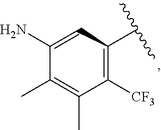

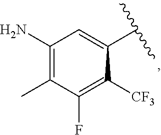

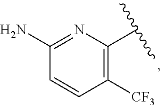 [SMILES: *c1nc(N)ccc1C(F)(F)F ; ","]

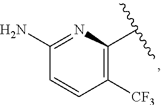

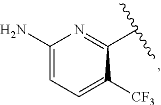

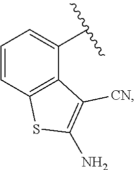 [SMILES: *c1cccc2sc(N)c(C#N)c12]

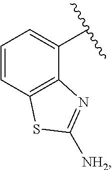 [SMILES: *c1cccc2sc(N)nc12]

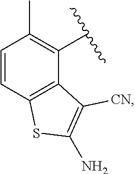 [SMILES: *c1c(C)ccc2sc(N)c(C#N)c12]

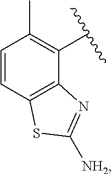 [SMILES: *c1c(C)ccc2sc(N)nc12]

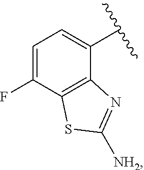 [SMILES: *c1ccc(F)c2sc(N)nc12]

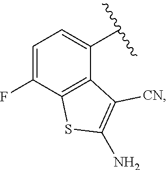 [SMILES: *c1ccc(F)c2sc(N)c(C#N)c12]

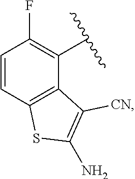 [SMILES: *c1c(F)ccc2sc(N)c(C#N)c12]

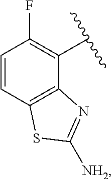 [SMILES: *c1c(F)ccc2sc(N)nc12]

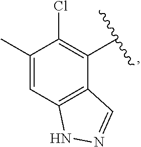 [SMILES: *c1c(Cl)c(C)cc2[nH]ncc12]

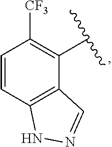 [SMILES: *c1c(C(F)(F)F)ccc2[nH]ncc12]

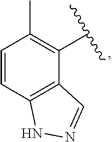 [SMILES: *c1c(C)ccc2[nH]ncc12]

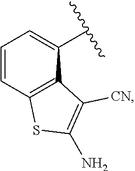

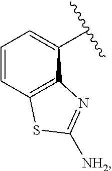

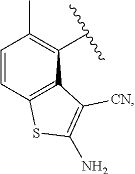

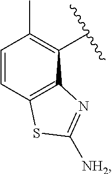

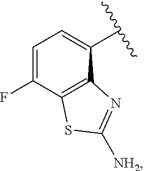

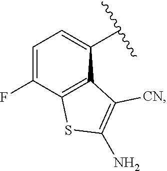

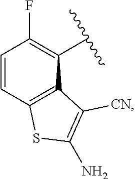

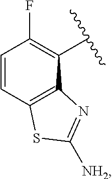

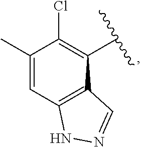

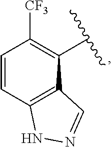

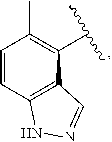

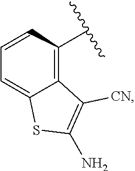

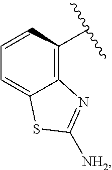

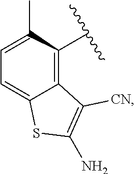

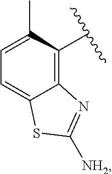

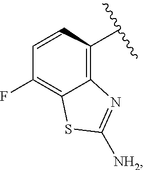

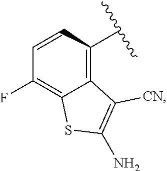

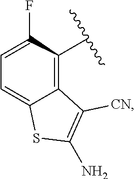

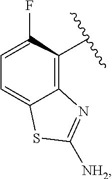

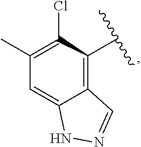

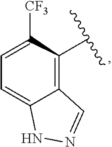

or

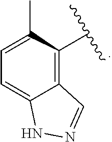

[67]. The compound of formula (I), a stereoisomer thereof, an atropisomer, a pharmaceutically acceptable salt thereof, a pharmaceutically acceptable salt of the stereoisomer, a pharmaceutically acceptable salt of the atropisomer, a prodrug thereof, a deuterated molecule thereof or a conjugated form thereof of any one of [1] to [66], wherein, the prodrug is selected from the following formula:

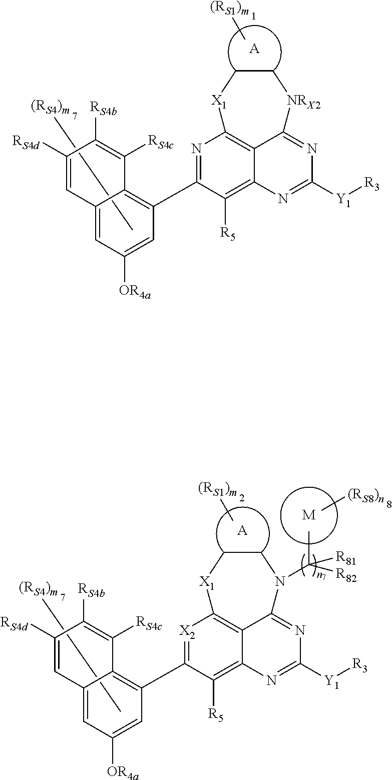

R₄ₐ is selected from

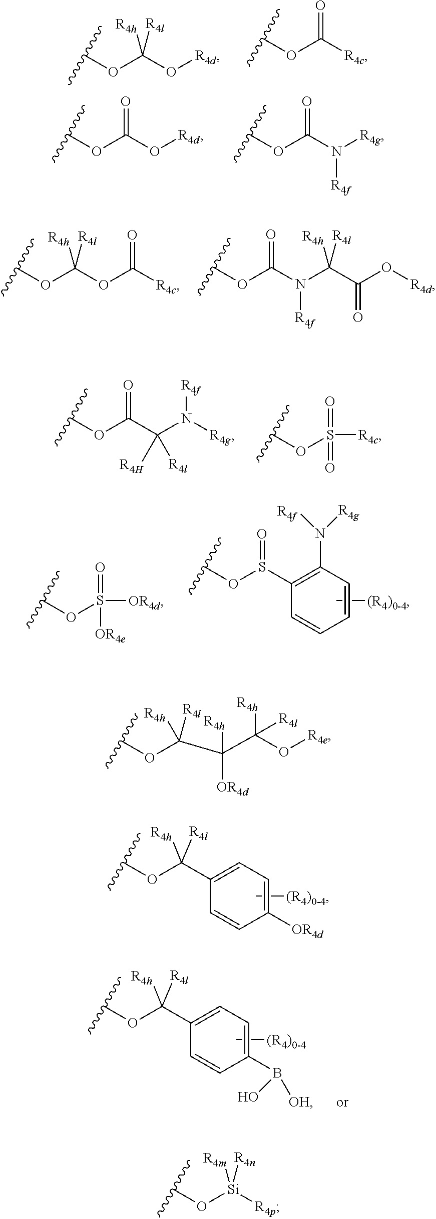

R_{4c} is selected from hydrogen, —C₁₋₃₀alkyl, —C₂₋₃₀alkenyl, —C₂₋₃₀alkynyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4d} and R₄ₑ are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R_{4f} and R_{4g} are each selected from hydrogen, —C₁₋₃₀alkyl, —C₂₋₃₀alkenyl, —C₂₋₃₀alkynyl, —C(═O)C₁₋₆alkyl, —C₀₋₆alkylene-(3-20 membered carbocyclyl), —C₀₋₆alkylene-(3-20 membered heterocyclyl), —C₀₋₆alkylene-(6-10 membered aryl) or —C₀₋₆alkylene-(5-10 membered heteroaryl), each of which is independently substituted with one or more R₄ⱼ;
R₄ₕ, R₄ᵢ, R₄ₘ, R₄ₙ and R₄ₚ are each selected from hydrogen, deuterium, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O) OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl; wherein, said —C₁₋₆alkyl, —C₂₋₆alkenyl, —C₂₋₆alkynyl, 3-10 membered cycloalkyl, 3-10 membered heterocyclyl, 6-10 membered aryl or 5-10 membered heteroaryl is optionally substituted with one or more substituents selected from deuterium, halogen, —C₁₋₆alkyl, haloC₁₋₆alkyl, haloC₁₋₆alkoxy, —C₂₋₆alkenyl, —C₂₋₆alkynyl, —CN, —NH₂, —NH(C₁₋₆alkyl), —N(C₁₋₆alkyl)₂, oxo, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)(C₁₋₆alkyl), —N(C₁₋₆alkyl)S(═O)₂(C₁₋₆alkyl), 3-10 membered cycloalkyl, 3-10 membered heterocyclyl, 6-10 membered aryl or 5-10 membered heteroaryl;

Optionally, R_{4f} and R_{4g} together with the atoms to which they are respectively attached form a 4-10 membered heterocyclyl ring, said 4-10 membered heterocyclyl ring optionally further contains 1 or 2 heteratoms selected from N, O, S, S(═O) or S(═O)₂ and optionally substituted with one or more R₄ⱼ;

Optionally, R_{4f} and R₄ₕ together with the atoms to which they are respectively attached form a 4-10 membered heterocyclyl ring, said 4-10 membered heterocyclyl ring optionally further contains 1 or 2 heteratoms selected from N, O, S, S(═O) or S(═O)₂ and optionally substituted with one or more Raj;
R₄ⱼ at each occurrence is independently selected from deuterium, halogen, —C₁₋₆alkyl, haloC₁₋₆alkyl, haloC₁₋₆alkoxy, —C₂₋₆alkenyl, —C₂₋₆alkynyl, —CN, oxo, —NO₂, —NH₂, —NH(C₁₋₆alkyl), —N(C₁₋₆alkyl)₂, —OH, —O(C₁₋₆alkyl), —SH, —S(C₁₋₆alkyl), —S(haloC₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), 3-10 membered cycloalkyl, 3-10 membered heterocyclyl, 6-10 membered aryl, or 5-10 membered heteroaryl, wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered heterocyclyl, 6-10 membered aryl or 5-10 membered heteroaryl is independently optionally substituted with 1, 2 or 3 substituents selected from halogen; —C₁₋₆alkyl; haloC₁₋₆alkyl; —CN; oxo; —OH; —NH₂; —NH(C₁₋₆alkyl); —NH(C₁₋₆alkyl)₂; —OC₁₋₆alkyl; or —C₁₋₆alkyl substituted with 1, 2 or 3 substituents selected form deuterium, halogen, haloC₁₋₆alkyl, —CN, —OH, —NH₂, —NH(C₁₋₆alkyl), —NH(C₁₋₆alkyl)₂ or —OC₁₋₆alkyl;

Other variables have the same definitions as above;

Preferably, R₄ₐ is selected from the following Table E:

TABLE E

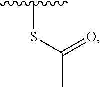 [SMILES: *SC(C)=O]

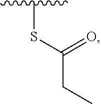 [SMILES: *SC(=O)CC]

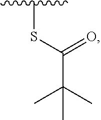 [SMILES: *SC(=O)C(C)(C)C]

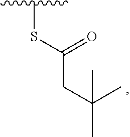 [SMILES: *SC(=O)CC(C)(C)C]

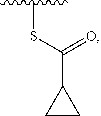 [SMILES: *SC(=O)C1CC1]

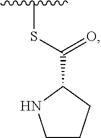 [SMILES: *SC(=O)[C@@H]1CCCN1]

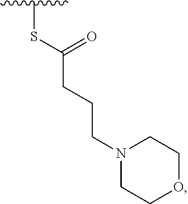 [SMILES: *SC(=O)CCCN1CCOCC1]

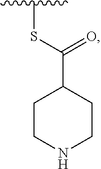 [SMILES: *SC(=O)C1CCNCC1]

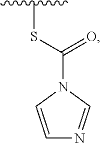 [SMILES: *SC(=O)n1ccnc1]

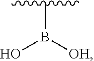 [SMILES: *B(O)O]

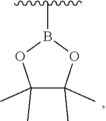

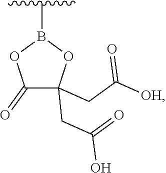 [SMILES: *B1OC(=O)C(CC(=O)O)(CC(=O)O)O1]

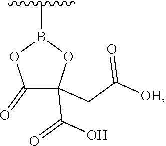 [SMILES: *B1OC(=O)C(CC(=O)O)(C(=O)O)O1]

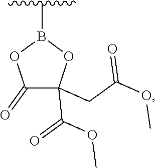 [SMILES: *B1OC(=O)C(CC(=O)OC)(C(=O)OC)O1]

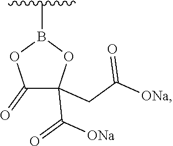

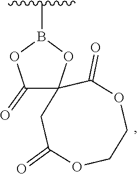 [SMILES: *B1OC(=O)C2(CC(=O)OCCOC2=O)O1]

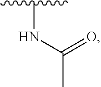 [SMILES: *NC(C)=O]

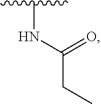 [SMILES: *NC(=O)CC]

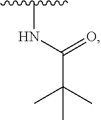 [SMILES: *NC(=O)C(C)(C)C]

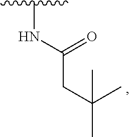 [SMILES: *NC(=O)CC(C)(C)C]

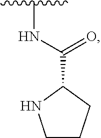 [SMILES: *NC(=O)[C@@H]1CCCN1]

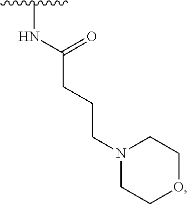 [SMILES: *NC(=O)CCCN1CCOCC1]

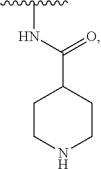 [SMILES: *NC(=O)C1CCNCC1]

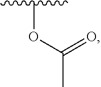 [SMILES: *OC(C)=O]

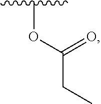 [SMILES: *OC(=O)CC]

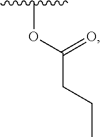 [SMILES: *OC(=O)CCC]

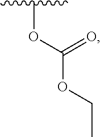 [SMILES: *OC(=O)OCC]

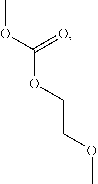

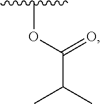 [SMILES: *OC(=O)C(C)C]

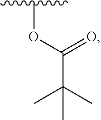 [SMILES: *OC(=O)C(C)(C)C]

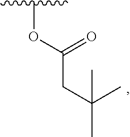 [SMILES: *OC(=O)CC(C)(C)C]

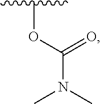 [SMILES: *OC(=O)N(C)C]

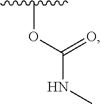 [SMILES: *OC(=O)NC]

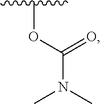 [SMILES: *OC(=O)N(C)C]

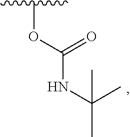 [SMILES: *OC(=O)NC(C)(C)C]

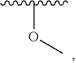 [SMILES: *OC]

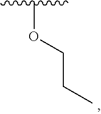 [SMILES: *OCCC]

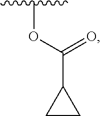 [SMILES: *OC(=O)C1CC1]

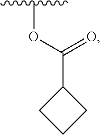 [SMILES: *OC(=O)C1CCC1]

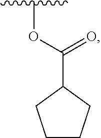 [SMILES: *OC(=O)C1CCCC1]

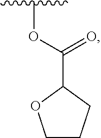 [SMILES: *OC(=O)C1CCCO1]

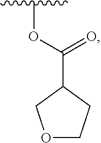 [SMILES: *OC(=O)C1CCOC1]

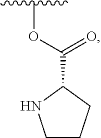 [SMILES: *OC(=O)[C@@H]1CCCN1]

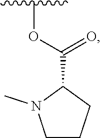 [SMILES: *OC(=O)[C@@H]1CCCN1C]

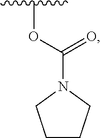 [SMILES: *OC(=O)N1CCCC1]

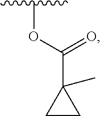 [SMILES: *OC(=O)C1(C)CC1]

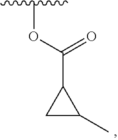 [SMILES: *OC(=O)C1CC1C]

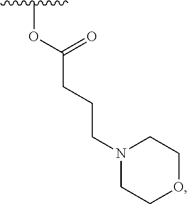 [SMILES: *OC(=O)CCCN1CCOCC1]

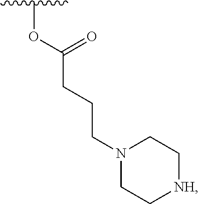 [SMILES: *OC(=O)CCCN1CCNCC1]

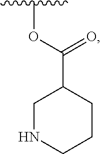 [SMILES: *OC(=O)C1CCCNC1]

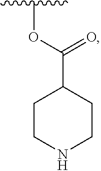

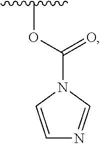 [SMILES: *OC(=O)n1ccnc1]

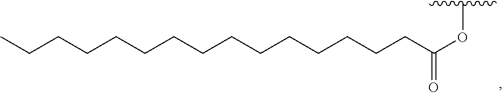

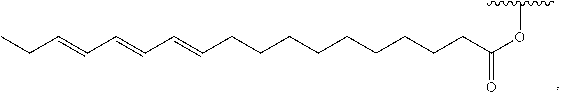

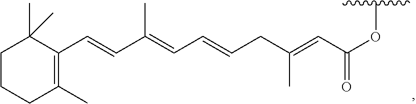

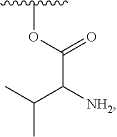 [SMILES: *OC(=O)C(N)C(C)C]

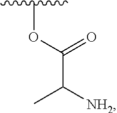 [SMILES: *OC(=O)C(C)N]

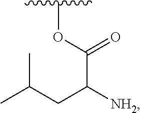 [SMILES: *OC(=O)C(N)CC(C)C]

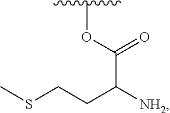 [SMILES: *OC(=O)C(N)CCSC]

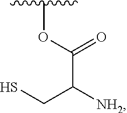 [SMILES: *OC(=O)C(N)CS]

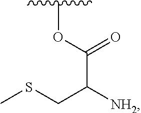 [SMILES: *OC(=O)C(N)CSC]

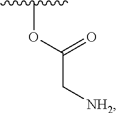 [SMILES: *OC(=O)CN]

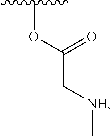 [SMILES: *OC(=O)CNC]

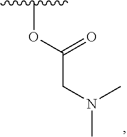

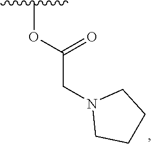

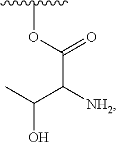 [SMILES: *OC(=O)C(N)C(C)O]

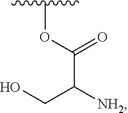 [SMILES: *OC(=O)C(N)CO]

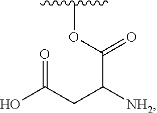 [SMILES: *OC(=O)C(N)CC(=O)O]

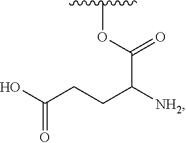 [SMILES: *OC(=O)C(N)CCC(=O)O]

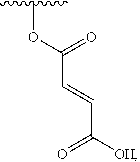 [SMILES: *OC(=O)/C=C/C(=O)O]

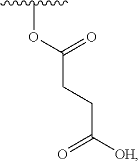 [SMILES: *OC(=O)CCC(=O)O]

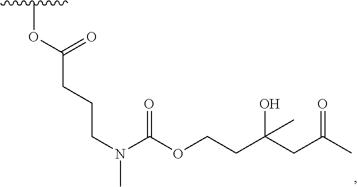

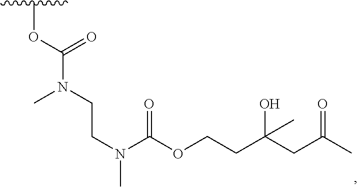

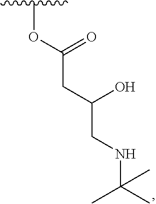

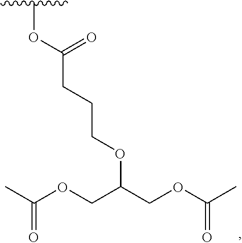

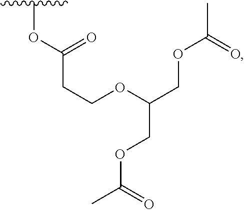 [SMILES: *OC(=O)CCOC(COC(C)=O)COC(C)=O]

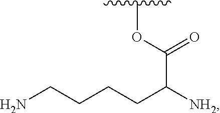 [SMILES: *OC(=O)C(N)CCCCN]

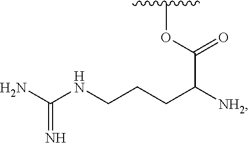

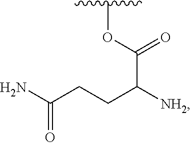 [SMILES: *OC(=O)C(N)CCC(N)=O]

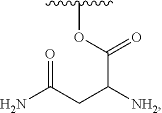 [SMILES: *OC(=O)C(N)CC(N)=O]

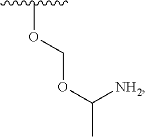 [SMILES: *OCOC(C)N]

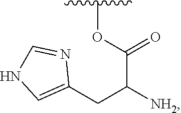 [SMILES: *OC(=O)C(N)Cc1c[nH]cn1]

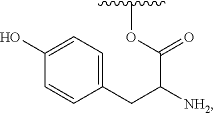 [SMILES: *OC(=O)C(N)Cc1ccc(O)cc1]

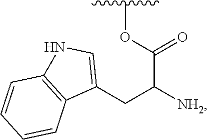 [SMILES: *OC(=O)C(N)Cc1c[nH]c2ccccc12]

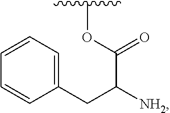 [SMILES: *OC(=O)C(N)Cc1ccccc1]

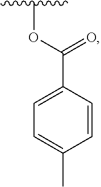 [SMILES: *OC(=O)c1ccc(C)cc1]

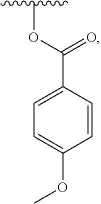 [SMILES: *OC(=O)c1ccc(OC)cc1]

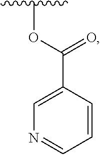 [SMILES: *OC(=O)c1cccnc1]

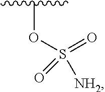 [SMILES: *OS(N)(=O)=O]

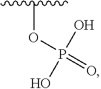 [SMILES: *OP(=O)(O)O]

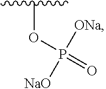

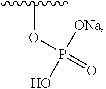

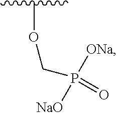

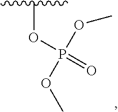

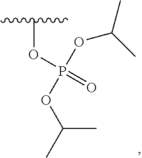

or

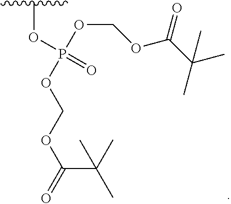

[68]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [67], wherein, R₅ is selected from halogen.
[69]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [68], wherein, R₅ is selected from F.
[70]. The compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof of any one of [1] to [69], wherein, the compound is selected from the following Table F:

Lengthy table referenced here
US20260365672A1-20261119-T00001
Please refer to the end of the specification for access instructions.

[71]. A pharmaceutical composition, comprising a therapeutically effective amount of the compound of formula (I), a stereoisomer thereof, a pharmaceutically acceptable salt thereof, a pharmaceutically acceptable salt of the stereoisomer thereof, a prodrug thereof, a deuterated molecule thereof or a PROTAC molecule thereof of any one of [1] to [81], and a pharmaceutically acceptable excipient.
[72]. A method for treating cancer in a subject comprising administering a therapeutically effective amount of a compound of formula (I), a stereoisomer thereof, a pharmaceutically acceptable salt thereof, a pharmaceutically acceptable salt of the stereoisomer thereof, a prodrug thereof, a deuterated molecule thereof or a PROTAC molecule thereof of any one of [1] to [81], or the pharmaceutical composition of [71] to a subject in need thereof.
[73]. A method for treating cancer in a subject in need thereof, the method comprising:
(a) determining whether the cancer is associated with K-Ras G12C, K-Ras G12D, K-Ras G12V, K-Ras G13D, K-Ras G12R, K-Ras G12S, K-Ras G12A, K-Ras Q61H mutation and/or K-Ras wild type amplification; and
(b) if so, administering a therapeutically effective amount of the compound of Formula (I), the stereoisomer thereof, the pharmaceutically acceptable salt thereof, the pharmaceutically acceptable salt of the stereoisomer thereof, the prodrug thereof, the deuterated molecule thereof or the PROTAC molecule thereof of any one of [1] to [81], or the pharmaceutical composition of [71] to the subject in need thereof.
[74]. The compound of formula (I), a stereoisomer thereof, a pharmaceutically acceptable salt thereof, a pharmaceutically acceptable salt of the stereoisomer thereof, a prodrug thereof, a deuterated molecule thereof or the PROTAC molecule thereof of any one of [1] to [81], or the pharmaceutical composition of [71] for use in therapy.
[75]. The compound of formula (I), a stereoisomer thereof, a pharmaceutically acceptable salt thereof, a pharmaceutically acceptable salt of the stereoisomer thereof, a prodrug thereof, a deuterated molecule thereof or a PROTAC molecule thereof of any one of [1] to [81], or the pharmaceutical composition of [71] for use as a medicament.
[76]. The compound of formula (I), a stereoisomer thereof, a pharmaceutically acceptable salt thereof, a pharmaceutically acceptable salt of the stereoisomer thereof, a prodrug thereof, a deuterated molecule thereof or a PROTAC molecule thereof of any one of [1] to [81], or the pharmaceutical composition of [71] for use in a method for the treatment of cancer.
[77]. A use of the compound of formula (I), a stereoisomer thereof, a pharmaceutically acceptable salt thereof, a pharmaceutically acceptable salt of the stereoisomer thereof, the prodrug thereof, the deuterated molecule thereof or the conjugated form thereof of any one of [1] to [81], or the pharmaceutical composition of [71] for the treatment of cancer.
[78]. A use of the compound of formula (I), a stereoisomer thereof, a pharmaceutically acceptable salt thereof, a pharmaceutically acceptable salt of the stereoisomer thereof, a prodrug thereof, a deuterated molecule thereof or a PROTAC molecule thereof of any one of [1] to [81], or the pharmaceutical composition of [71] for the manufacture of a medicament for the treatment of cancer.
[79]. The method for treating cancer of [71 ], the use in a method for the treatment of cancer of [76], the use for the treatment of cancer of [77], or the use for the manufacture of a medicament for the treatment of cancer of [78], wherein, said cancer is selected from pancreatic carcinoma, colorectal carcinoma, lung carcinoma (such as non-small cell lung cancer), breast carcinoma, large intestine carcinoma, stomach carcinoma, endometrial carcinoma, esophageal carcinoma or gastroesophageal junction carcinoma.
[80]. The method for treating cancer of [71] or [79], the use in a method for the treatment of cancer of [76] or [79], the use for the treatment of cancer of [77] or [79], or the use for the manufacture of a medicament for the treatment of cancer of [78] or [79], wherein, the cancer is associated with at least one of K-Ras G12C, K-Ras G12D, K-Ras G12V, K-Ras G13D, K-Ras G12R, K-Ras G12S, K-Ras G12A, K-Ras Q61H mutation and/or K-Ras wild type amplification.
[81]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12C associated cancer.
[82]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12D associated cancer.
[83]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12V associated cancer.
[84]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G13D associated cancer.
[85]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], the cancer a K-Ras G12R associated cancer.
[86]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12S associated cancer.
[87]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras G12A associated cancer.
[88]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras Q61H associated cancer.
[89]. The method for treating cancer of [71], [79] or [80], the use in a method for the treatment of cancer of [76], [79] or [80], the use for the treatment of cancer of [77], [79] or [80], or the use for the manufacture of a medicament for the treatment of cancer of [78], [79] or [80], wherein, the cancer a K-Ras wild type amplification associated cancer.

### DEFINITION

Unless defined otherwise, all technical and scientific terms used herein have the same meaning as is commonly understood by one of skill in the art to which this invention belongs. All patents, patent applications, and publications referred to herein are incorporated by reference.

The term “halogen” or “halo”, as used herein, unless otherwise indicated, means fluoro, chloro, bromo or iodo. The preferred halogen groups include —F, —Cl and —Br.

The term “alkyl”, as used herein, unless otherwise indicated, includes saturated monovalent hydrocarbon radicals having straight or branched. For example, —C₁₋₆alkyl radicals include methyl, ethyl, propyl, isopropyl, n-butyl, isobutyl, sec-butyl, t-butyl, n-pentyl, 3-(2-methyl)butyl, 2-pentyl, 2-methylbutyl, neopentyl, n-hexyl, 2-hexyl and 2-methylpentyl. Similary, C₁₋₃, as in C₁₋₃alkyl is defined to identify the group as having 1,2,3,4,5 or 6 carbon atoms in a linear or branched arrangement.

The term “haloalkyl” (such as —C₁₋₆haloalkyl, —C₁₋₄haloalkyl or —C₁₋₃haloalkyl) as used herein, unless otherwise indicated, an alkyl chain (such as —C₁₋₆alkyl, —C₁₋₄alkyl or —C₁₋₃alkyl) as defined herein in which one or more (such as one, two or three) hydrogen has been replaced by a halogen. Examples include trifluoromethyl, difluoromethyl and fluoromethyl.

The term “alkylene” means a difunctional group obtained by removal of a hydrogen atom from an alkyl group defined above. For example, methylene (i.e., —CH₂—), ethylene (i.e., —CH₂—CH₂— or —CH(CH₃)—) and propylene (i.e., —CH₂—CH₂—CH₂—, —CH(—CH₂—CH₃)— or —CH₂—CH(CH₃)—).

The term “alkenyl” means a straight or branch-chained hydrocarbon radical containing one or more double bonds and typically from 2 to 20 carbon atoms in length. For example, “C₂₋₆alkenyl” contains from 2 to 6 carbon atoms. Alkenyl group include, but are not limited to, for example, ethenyl, propenyl, butenyl, 2-methyl-2-buten-1-yl, hepetenyl, octenyl and the like.

The term “alkynyl” contains a straight or branch-chained hydrocarbon radical containing one or more triple bonds and typically from 2 to 20 carbon atoms in length. For example, “C₂₋₆alkynyl” contains from 2 to 6 carbon atoms. Representative alkynyl groups include, but are not limited to, for example, ethynyl, 1-propynyl, 1-butynyl, heptynyl, octynyl and the like.

The term “alkoxy” radicals are oxygen ethers formed from the previously described alkyl groups.

The term “aryl”, as used herein, unless otherwise indicated, refers to an unsubstituted or substituted mono or polycyclic aromatic ring system containing carbon ring atoms. The preferred aryls are mono cyclic or bicyclic 6-10 membered aromatic ring systems. Phenyl and naphthyl are preferred aryls.

The term “heterocyclic”, as used herein, unless otherwise indicated, refers to unsubstituted and substituted mono or polycyclic non-aromatic ring system containing one or more heteroatoms, which comprising monocyclic heterocyclic ring, bicyclic heterocyclic ring, bridged heterocyclic ring, fused heterocyclic ring of spiro heterocyclic ring. Preferred heteroatoms include N, O, and S, including N-oxides, sulfur oxides, and dioxides. Preferably, the ring is three to ten membered and is either fully saturated or has one or more degrees of unsaturation. Multiple degrees of substitution, preferably one, two or three, are included within the present definition. Examples of such heterocyclic groups include, but are not limited to azetidinyl, pyrrolidinyl, piperidinyl, piperazinyl, oxopiperazinyl, oxopiperidinyl, oxoazepinyl, azepinyl, tetrahydrofuranyl, dioxolanyl, tetrahydroimidazolyl, tetrahydrothiazolyl, tetrahydrooxazolyl, tetrahydropyranyl, morpholinyl, thiomorpholinyl, thiamorpholinylsulfoxide, thiamorpholinylsulfone and oxadiazolyl.

The term “heteroaryl”, as used herein, unless otherwise indicated, represents an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 10 hetero atoms. A polycyclic heteroaryl ring may contain fused, spiro or bridged ring junction, for example, bycyclicheteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Examples of heteroaryl groups include, but are not limited to thienyl, furanyl, imidazolyl, isoxazolyl, oxazolyl, pyrazolyl, pyrrolyl, thiazolyl, thiadiazolyl, triazolyl, pyridyl, pyridazinyl, indolyl, azaindolyl, indazolyl, benzimidazolyl, benzofuranyl, benzothienyl, benzisoxazolyl, benzoxazolyl, benzopyrazolyl, benzothiazolyl, benzothiadiazolyl, benzotriazolyladeninyl, quinolinyl or isoquinolinyl.

The term “carbocyclic” refers to a substituted or unsubstituted monocyclic ring, bicyclic ring, bridged ring, fused ring, spiro ring non-aromatic ring system only containing carbon atoms. Exemplary “cycloalkyl” groups includes but not limited to, cyclopropyl, cyclobutyl, cyclopentyl, cyclohexyl and so on.

The term “oxo” refers to oxygen atom together with the attached carbon atom forms the group

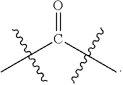 [SMILES: *C(*)=O]

The term “composition”, as used herein, is intended to encompass a product comprising the specified ingredients in the specified amounts, as well as any product which results, directly or indirectly, from combinations of the specified ingredients in the specified amounts. Accordingly, pharmaceutical compositions containing the compounds of the present invention as the active ingredient as well as methods of preparing the instan

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. When the compound of the present invention is basic, its corresponding salt can be conveniently prepared from pharmaceutically acceptable non-toxic acids, including inorganic and organic acids. Since the compounds in the present invention are intended for pharmaceutical use they are preferably provided in substantially pure form, for example at least 60% pure, more suitably at least 75% pure, especially at least 98% pure (% are on a weight for weight basis).

The present invention includes within its scope the prodrugs of the compounds of this invention. In general, such prodrugs will be functional derivatives of the compounds that are readily converted in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the subject. Conventional procedures for the selection and preparation of suitable prodrug derivatives are described, for example, in “Design of Prodrugs”, ed. H. Bundgaard, Elsevier, 1985.

It is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques know in the art as well as those methods set forth herein.

The present invention includes compounds described can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The present invention includes all stereoisomers of the compound and pharmaceutically acceptable salts thereof. Further, mixtures of stereoisomers as well as isolated specific stereoisomers are also included. During the course of the synthetic procedures used to prepare such compounds or in using racemization or epimerization procedures known to those skilled in the art, the products of such procedures can be a mixture of stereoisomers.

The term “stereoisomer” as used in the present invention refers to an isomer in which atoms or groups of atoms in the molecule are connected to each other in the same order but differ in spatial arrangement, including conformational isomers and conformational isomers. The configuration isomers include geometric isomers and optical isomers, and optical isomers mainly include enantiomers and diastereomers. The invention includes all possible stereoisomers of the compound.

Certain of the compounds provided herein may exist as atropisomers, which are conformational stereoisomers that occur when rotation about a single bond in the molecule is prevented, or greatly slowed, as a result of steric interactions with other parts of the molecule. The compounds provided herein include all atropisomers, both as pure individual atropisomer preparations, enriched preparations of each, or a non-specific mixture of each. Where the rotational barrier about the single bond is high enough, and interconversion between conformations is slow enough, separation and isolation of the isomeric species may be permitted.

The present invention is intended to include all isotopes of atoms occurring in the present compounds. Isotopes include those atoms having the same atomic number but different mass numbers. By way of general example and without limitation, isotopes of hydrogen include deuterium and tritium. The isotopes of hydrogen can be denoted as ¹H (hydrogen), ²H (deuterium) and ³H (tritium). They are also commonly denoted as D for deuterium and T for tritium. In the application, CD₃ denotes a methyl group wherein all of the hydrogen atoms are deuterium. Isotopes of carbon include ¹³C and ¹⁴C. Isotopically-labeled compounds of the invention can generally be prepared by conventional techniques known to those skilled in the art or by prcesses analogous to those described herein, using an appropriate isotopically-labeled reagent in place of the non-labeled reagent.

When a tautomer of the compound in the present invention exists, the present invention includes any possible tautomers and pharmaceutically acceptable salts thereof, and mixtures thereof, except where specifically stated otherwise.

When the compound in present invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable.

The pharmaceutical compositions of the present invention comprise a compound in present invention (or a pharmaceutically acceptable salt thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds in present invention or a prodrug or a metabolite or pharmaceutically acceptable salts thereof, of this invention can be combined as the active ingredient in intimate admixture with a pharmaceutical carrier of conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers of both. The product can then be conveniently shaped into the desired presentation.

Thus, the pharmaceutical compositions of this invention may include a pharmaceutically acceptable carrier and a compound or a pharmaceutically acceptable salt. The compounds of Formula I or pharmaceutically acceptable salts thereof, can also be included in pharmaceutical compositions in combination with one or more other therapeutically active compounds.

The pharmaceutical carrier employed can be, for example, a solid, liquid or gas. Examples of solid carriers include lactose, terra alba, sucrose, tale, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient. For example, a formulation intended for the oral administration to humans may contain from about 0.5 mg to about 5 g of active agent, compounded with an appropriate and convenient amount of carrier material which may vary from about 0.05 to about 95 percent of the total composition. Unit dosage forms will generally contain between from about 0.01 mg to about 2 g of the active ingredient, typically 0.01 mg, 0.02 mg, 1 mg, 2 mg, 3 mg, 4 mg, 5 mg, 6 mg, 7 mg, 8 mg, 9 mg, 10 mg, 25 mg, 50 mg, 100 mg, 200 mg, 300 mg, 400 mg, 500 mg, 600 mg, 800 mg, 1000 mg, 1500 mg or 2000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder or the like. Further, the compositions can be in a form suitable for use in transdermal devices. These formulations may be prepared, utilizing a compound represented by Formula I of this invention or a pharmaceutically acceptable salt thereof, via conventional processing methods. As an example, a cream or ointment is prepared by admixing hydrophilic material and water, together with about 0.05 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including antioxidants) and the like. Furthermore, other adjuvants can be included to render the formulation isotonic with the blood of the intended recipient. Compositions containing a compound described by Formula I or pharmaceutically acceptable salts thereof, may also be prepared in powder or liquid concentrate form.

Generally, dosage levels on the order of from about 0.001 mg/kg to about 150 mg/kg of body weight per day are useful in the treatment of the above-indicated conditions or alternatively about 0.05 mg to about 7 g per patient per day. For example, inflammation, cancer, psoriasis, allergy/asthma, disease and conditions of the immune system, disease and conditions of the central nervous system (CNS), may be effectively treated by the administration of from about 0.001 to 50 mg of the compound per kilogram of body weight per day or alternatively about 0.05 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

These and other aspects will become apparent from the following written description of the invention.

It is to be understood that, if any prior art publication is referred to herein; such reference does not constitute an admission that the publication forms a part of the common general knowledge in the art in any country.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and Examples should not be construed as limiting the scope of the invention.

Compounds of the present invention can be synthesized from commercially available reagents using the synthetic methods and reaction schemes described herein. The examples which outline specific synthetic route, and the generic schemes below are meant to provide guidance to the ordinarily skilled synthetic chemist, who will readily appreciate that the solvent, concentration, reagent, protecting group, order of synthetic steps, time, temperature, and the like can be modified as necessary, well within the skill and judgment of the ordinarily skilled artisan.

### EXAMPLES

The compound in the present invention could be synthesized and the activity could be tested according to the pharmacological experiments.

| | |
|---|---|
| DMF | Dimethylformamide |
| EA | Ethyl acetate |
| CH₃CN/MeCN | Acetonitrile |
| DCM | Dichloromethane |
| NaH | Sodium hydride |
| THF | Tetrahydrofuran |
| Na₂SO₄ | Sodium sulfate |
| Cs₂CO₃ | Cesium carbonate |
| TFA | 2,2,2-Trifluoroacetic acid |
| POCl₃ | Phosphoryl chloride |
| NaHCO₃ | Sodium bicarbonate |
| DMSO | Dimethyl sulfoxide |
| DIPEA/DIEA | N,N-Diisopropylethylamine |
| CsF | Cesium fluoride |
| Dabco | 1,4-Diazobicyclo(2.2.2)octane |
| sat. | Saturated |
| h/hrs | Hour/hours |

### Intermediate 1 (INT 1)

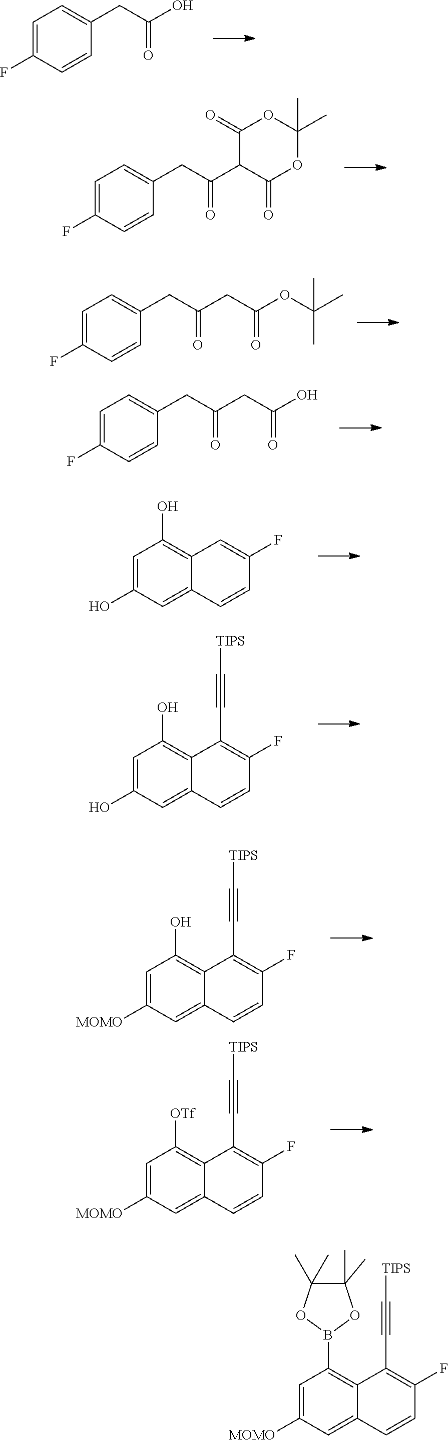

Following the procedure of WO2021041671, INT 1 was synthesized with 2-(4-fluorophenyl) acetic acid as starting material.

### Intermediate 2 (INT 2)

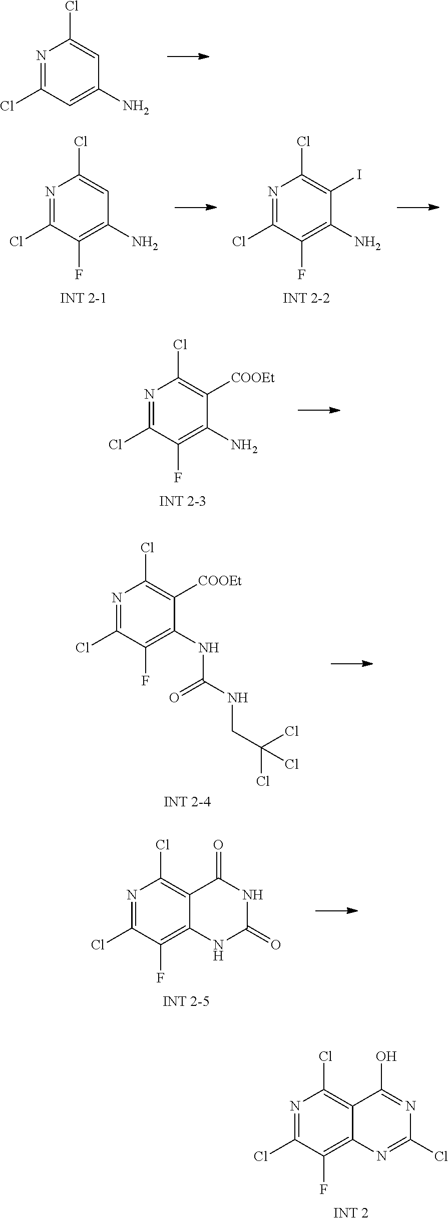

A mixture of 2,6-dichloropyridin-4-amine (35.7 g, 219.0 mmol), 1-(chloromethyl)-4-fluoro-1,4-diazoniabicyclo[2.2.2]octane ditetrafluoroborate (93.1 g, 262.8 mmol) in DMF (357 mL) and CH₃CN (357 mL) was stirred at 80 °C for 6 hours. The reaction mixture was quenched by water (400 mL), extracted with DCM (400 mL×3), the organic layers were combined, dried over anhydrous Na₂SO₄, filtered and concentrated under reduced pressure. The residue was purified by silica gel column (eluting with petroleum ether:EtOAc=30:1, v/v) to give INT 2-1 (12.6 g, purity: about 50%). MS (ESI, m/z): 181 [M+H]⁺.

A mixture of INT 2-1 (2.0 g, 11.05 mmol), NIS (2.98 g, 13.26 mmol) and p-toluenesulfonic acid monohydrate (105 mg, 0.55 mmol) in CH₃CN (8.4 mL) was stirred at 70 °C for 4 hours under nitrogen atmosphere. The reaction mixture was quenched by water (20 mL), extracted with EtOAc (20 mL×3), the organic layers were combined, dried over anhydrous Na₂SO₄, filtered and concentrated under reduced pressure. The residue was purified by silica gel column (eluting with petroleum ether; EtOAc=50:1 to 20:1, v/v) to give INT 2-2 (3.6 g). MS (ESI, m/z): 307 [M+H]⁺.

A mixture of INT 2-2 (1.0 g, 3.26 mmol), Pd(PPb₃)₂Cl₂ (229 mg, 0.33 mmol) and Et₃N (1.19 g, 11.77 mmol) in EtOH (17.0 mL) was stirred at 80 °C for 20 hours under carbon monoxide atmosphere (1.5 MPa) in a sealed tube. The reaction mixture was filtered and the filtrate was concentrated under reduced pressure. The residue was purified by silica gel column to give INT 2-3 (1.2 g). MS (ESI, m/z): 253 [M+H]⁺.

A mixture of INT 2-3 (800 mg, 3.16 mmol), trichloroacetyl isocyanate (714 mg, 3.79 mmol) in THF (8 mL) was stirred at room temperature for 1 hour. The reaction mixture was concentrated under reduced pressure. The residue was triturated with MTBE to give INT 2-4 (880 mg).

A mixture of INT 2-4 (780 mg, 1.77 mmol), NH₂/MeOH (1.26 mL, 7 M, 8.85 mmol) and MeOH (7.8 mL) was stirred at room temperature for 1 hour. The reaction mixture was concentrated under reduced pressure. The residue was triturated with MTBE to give INT 2-5 (550 mg). MS (ESI, m/z): 250 [M+H]⁺.

A mixture of INT 2-5 (375 mg, 1.50 mmol), DIPEA (595 mg, 4.60 mmol) and POCl₃ (15 mL) was stirred at 105 °C for 17 hours. The reaction mixture was concentrated under reduced pressure. The residue was diluted with 1,4-dioxane (5 mL) and the resulting solution was added dropwise to aq. K₂CO₃ (20%, 30 mL). The mixture was stirred for 2 hours at RT, adjust pH to 2~3, filtered and the filter cake was collected and dried to give INT 2 (344 mg). MS (ESI, m/z): 268 [M+H]⁺.

### Intermediate 3 (INT 3)

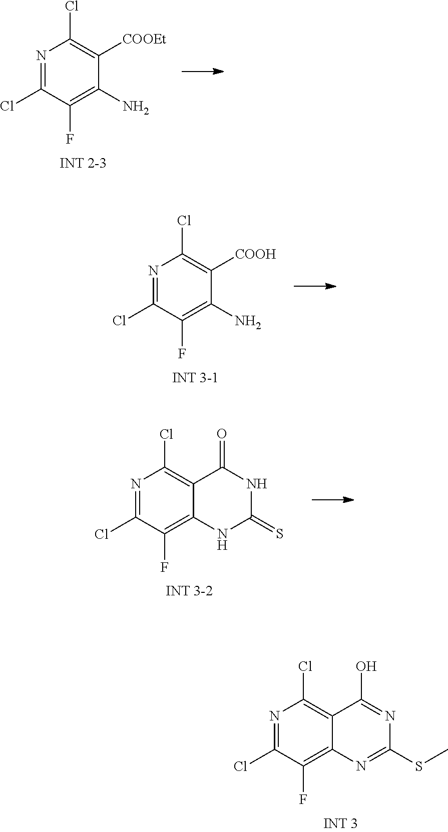 [SMILES: CCOC(=O)c1c(Cl)nc(Cl)c(F)c1N ; Nc1c(F)c(Cl)nc(Cl)c1C(=O)O ; O=c1[nH]c(=S)[nH]c2c(F)c(Cl)nc(Cl)c12 ; CSc1nc(O)c2c(Cl)nc(Cl)c(F)c2n1]

A mixture of INT 2-3 (4.53 g, 17.90 mmol), NaOH (2.99 g, 74.76 mmol) in EtOH (50 mL) and water (10 mL) was stirred for 4 hours at 50 °C. The reaction mixture was concentrated under reduced pressure. The residue was dissolved in water (40 mL), extracted with EtOAc (2×30 mL). The water phase was adjusted pH to 2, extracted with EtOAc (2× 30 mL), the organic layers were combined, dried over anhydrous Na₂SO₄, filtered and concentrated under reduced pressure to give INT 3-1 (2.68 g, 11.91 mmol, 66.54% yield). MS: m/z 225 (M+H)⁺.

A mixture of INT 3-1 (2.44 g, 10.84 mmol) and SOCl₂ (15 mL) was stirred for 2 hours at 70 °C. The mixture was concentrated under reduced pressure. A solution of the obtained residue in CH₃CN (10 mL) was added dropwise to a mixture of ammonium thiocyanate (2.22 g, 29.16 mmol) in CH₃CN (40 mL), then stirred for 1.5 hours. The reaction mixture was filtered, the filter cake was collected, dried to give INT 3-2 (2.21 g, 8.31 mmol, 76.59% yield). MS: m/z 264 (M−H)⁻.

To a mixture of INT 3-2 (2.01 g, 7.55 mmol), NaOH (aq. 0.1 M, 150 mL) and MeOH (150 mL) was added CH₃I (2.22 g, 15.64 mmol). The mixture was stirred for 0.5 hour at RT. The water phase was adjusted pH to 3 by hydrochloric acid, extracted with EtOAc (1×200 mL, 1×100 mL), the organic layers were combined and concentrated under reduced pressure. The residue was triturated with water (20 mL) to give INT 3 (1.88 g, 6.71 mmol, 88.85% yield). MS: m/z 278 (M−H)⁻.

### Intermediate 4 (INT 4)

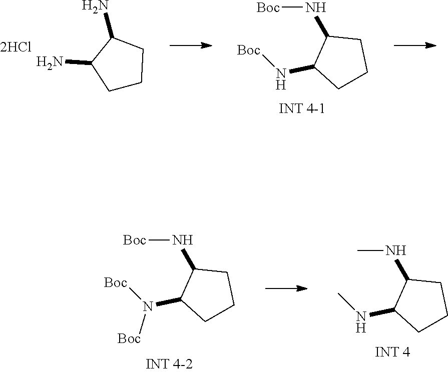

To a mixture of cis-cyclopentane-1,2-diamine (500 mg, 2.9 mmol, HCl salt) in DCM (20 mL) was added TEA (1.2 g, 11.6 mmol) at 0 °C, the resulting mixture was stirred for 15 min, then Boc₂O (1.5 g, 6.96 mmol) was added. Upon completion, the reaction mixture was diluted with DCM, washed with citric acid (aq. 10%) and brine, dried over anhydrous Na₂SO₄ and concentrated under reduced pressure. The residue was purified by silica gel chromatography (eluted with petroleum ether; EtOAc=10:1 to 5:1, v/v) to give INT 4-1 (77 mg). MS m/z: 301 [M+H]⁺.

A mixture of INT 4-1 (370 mg, 1.23 mmol) in DMF (12.3 mL) was stirred at 0 °C under nitrogen atmosphere. NaH (147.6 mg, 3.69 mmol, 60%) and CH₃I (523.8 mg, 3.69 mmol) was added, the resulting mixture was stirred for 1 hour at 0 °C. The reaction mixture was diluted with water, extracted with EtOAc, the organic layer was dried over anhydrous Na₂SO₄ and concentrated under reduced pressure. The residue was purified by silica gel chromatography (eluted with petroleum ether:EtOAc=10:1 to 5:1, v/v) to give INT 4-2 (330 mg). MS m/z: 329 [M+H]⁺.

A mixture of INT 4-2 (300 mg, 0.91 mmol) and TFA (1.5 mL) in DCM (3 mL) was stirred at RT for 2 hours. The reaction mixture was concentrated under reduced pressure to give INT 4 (480 mg, crude). MS m/z: 129 [M+H]⁺.

### Intermediate 5 (INT 5)

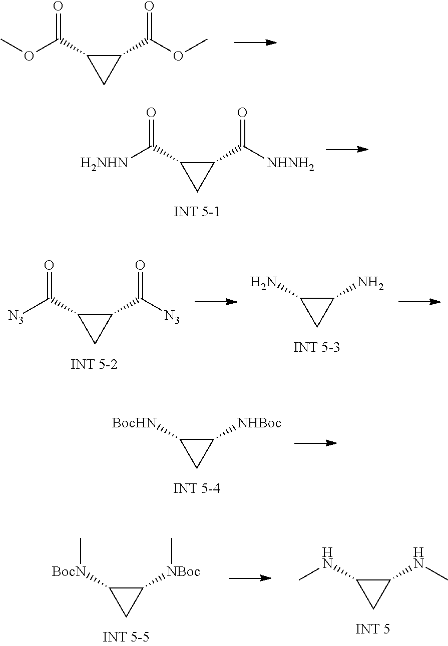

A mixture of dimethyl (1R,2S)-cyclopropane-1,2-dicarboxylate (28 g, 0.18 mol), hydrazinium hydroxide solution (35 mL) in EtOH (280 mL) was stirred at 80 °C. Upon completion, the reaction mixture was cooled to RT, filtered, the filter cake was collected and dried to give INT 5-1 (20.5 g). LCMS: 159 [M+H]⁺.

To a solution of INT 5-1 (20 g, 0.13 mol) in diethyl ether (364 mL) was added hydrochloric acid (145 mL conc hydrochloric acid diluted in 280 mL water). A solution of NaNO₂ (55 g, 0.80 mol) in water was added dropwise. The mixture was stirred for 1 hour, then the two phase was separated. The organic phase was diluted with toluene (500 mL), concentrated under reduced pressure to remove diethyl ether. The resulting solution was warmed to 85 °C, stirred, until no bubble generated. Hydrochloric acid (364 mL, warmed to 60 °C) was added to the reaction mixture, cooled, then concentrated under reduced pressure. The residue was triturated with EtOH to give INT 5-3 (5.5 g). LCMS: 181 [M+H]⁺.

A mixture of INT 5-3 (1.4 g, 19.41 mmol), NaOH (aq. 5 N, 3.9 g), 1,4-dioxane (14 mL) and Boc₂O (2.84 g, 13.01 mmol) was stirred at RT. Upon completion, the mixture was extracted with DCM, the organic layer was dried over anhydrous Na₂SO₄ and concentrated under reduced pressure. The residue was purified by silica gel chromatography (eluted with petroleum ether:EtOAc=5:1, v/v) to give INT 5-4 (2.5 g). LCMS: 273 [M+H]⁺.

A mixture of INT 5-4 (2.5 g, 9.18 mmol) in DMF (83 mL) was stirred at 0 °C under nitrogen atmosphere. NaH (1.1 g, 27.54 mmol, 60%) and CH₃I (3.9 g, 27.54 mmol) were added, the resulting mixture was stirred at 0 °C. The reaction mixture was diluted with water (100 mL), extracted with EtOAc, the organic layer was washed with brine, dried over anhydrous Na₂SO₄ and concentrated under reduced pressure. The residue was purified by silica gel chromatography (eluted with petroleum ether:EtOAc=5:1, v/v) to give INT 5-5 (750 mg). LCMS: 301 [M+H]⁺.

To a solution of INT 5-5 (566.6 mg, 1.89 mmol) in CH₃CN (10 mL) was added HCl/1,4-dioxane (3 mL, 4 M). The reaction mixture was stirred at room temperature for 4 hours. After completion, the reaction mixture was concentrated under reduced pressure to give INT 5 (466 mg, crude). LCMS: 101 [M+H]⁺.

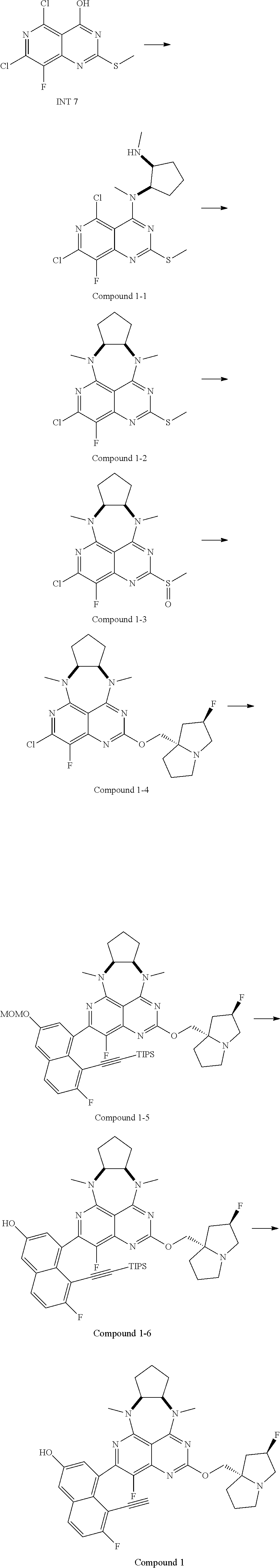

To a solution of INT 3 (209 mg, 0.75 mmol) and DIEA (0.5 mL) in toluene (10 mL) was added phosphorus oxychloride (0.5 mL) under nitrogen atmosphere and stirred at 80 °C for 4 hours. Upon completion, the mixture was concentrated under reduced pressure. The residue was diluted with DCM (5 mL), DIEA (1.5 mL) and INT 4 (386 mg, crude) were added. The reaction mixture was stirred at room temperature for 3 hours. Upon completion, the mixture was diluted with DCM (60 mL), water (30 mL) and separated. The collected organic layer was concentrated under reduced pressure to give Compound 1-1 (crude) which was used directly in the next step without purification. MS: m/z 390 [M+H]⁺.

To a solution of Compound 1-1 (crude) in CH₃CN (10 mL) was added DIEA (1 mL) and stirred at 60 °C for 6 hours. Upon completion, the mixture was concentrated under reduced pressure, the residue was purified by silica gel chromatography (eluting with Hex:EtOAc=3:1, v/v) to give Compound 1-2 (149 mg, 0.42 mmol). MS: m/z 354 [M+H]⁺.

To a solution of Compound 1-2 (131.5 mg, 0.37 mmol) in DCM (15 mL) was added 3-chlorobenzoperoxoic acid (76.7 mg, 0.44 mmol) at 0 °C and stirred for 1 hour. Upon completion, the residue was diluted with DCM (30 mL) and water (30 mL) and the mixture was adjusted to pH=8 to 9 with saturated NaHCO₃. The separated organic layer was concentrated under reduced pressure. The residue was purified by silica gel chromatography (eluting with DCM:MeOH=40:1, v/v) to give Compound 1-3 (115 mg, 0.31 mmol). MS: m/z 370 [M+H]⁺.

To a solution Compound 1-3 (103.9 mg, 0.45 mmol) and of ((2R,7aS)-2-fluorotetrahydro-1H-pyrrolizin-7a (5H)-yl) methanol (150 mg, 0.94 mmol) in THF (15 mL) was added t-BuONa (133 mg, 1.38 mmol) at 40 °C. The mixture was stirred for 30 min. The mixture was diluted with EtOAc (30 mL) and water (30 mL) and the organic layer was separated. The organic layer was washed with brine (20 mL) and dried over anhydrous Na₂SO₄, filtered and concentrated under reduced pressure to give Compound 1-4 (108 mg, crude). MS: m/z 465 [M+H]⁺.

To a solution of Compound 1-4 (108 mg, crude), INT 2 (256 mg, 0.50 mmol) and Cs₂CO₃ (346 mg, 1.06 mmol) in toluene (15 mL) and water (3 mL) was added methanesulfonato[(di(1-adamantyl)-n-butylphos-phine)-2-(2′-amino-1,1′-biphenyl)]palladium(II) (25 mg, 0.034 mmol) and the mixture was purged with nitrogen followed by stirring at 100 °C for 16 hours. Upon completion, the mixture was concentrated under reduced pressure, the residue was diluted with EtOAc (40 mL) and water (30 mL). The organic layer was separated and concentrated under reduced pressure. The residue was purified by Pre-TLC (DCM:MeOH=30:1, v/v) to give Compound 1-5 (30 mg, 0.037 mmol). MS: m/z 815 [M+1]⁺.

To a solution of Compound 1-5 (30 mg, 0.037 mmol) in CH₃CN (5 mL) was added HCl/1,4-dioxane (2 mL, 4 M). The reaction mixture was stirred at room temperature for 1 hour. After completion, the reaction mixture was concentrated under reduced pressure, the residue was diluted with EtOAc (30 mL) and water (20 mL) and adjusted to pH=8 to 9 with saturated NaHCO₃. The organic layer was separated and concentrated under reduced pressure to give Compound 1-6 (50 mg, crude). MS: m/z 771 [M+H]⁺.

To a mixture of Compound 1-6 (50 mg, crude) in DMF (5 mL) was added CsF (106 mg, 0.70 mmol). The mixture was stirred at room temperature for 6 hours. After completion, the mixture was diluted with EtOAc (40 mL) and water (30 mL) and adjusted to pH=8 to 9 with saturated NaHCO₃. The organic layer was separated and concentrated under reduced pressure. The residue was purified by Pre-HPLC (C18 column, A: 0.05% NH₃·H₂O in water, B: CH₃CN, gradient: 30% B to 85% B in 60 mins at a flow rate of 40 mL/min, 232 nm) and the product fractions were lyophilized to give Compound 1 (4.3 mg, 0.007 mmol). MS: m/z 615 [M+H]⁺.

### Example 2

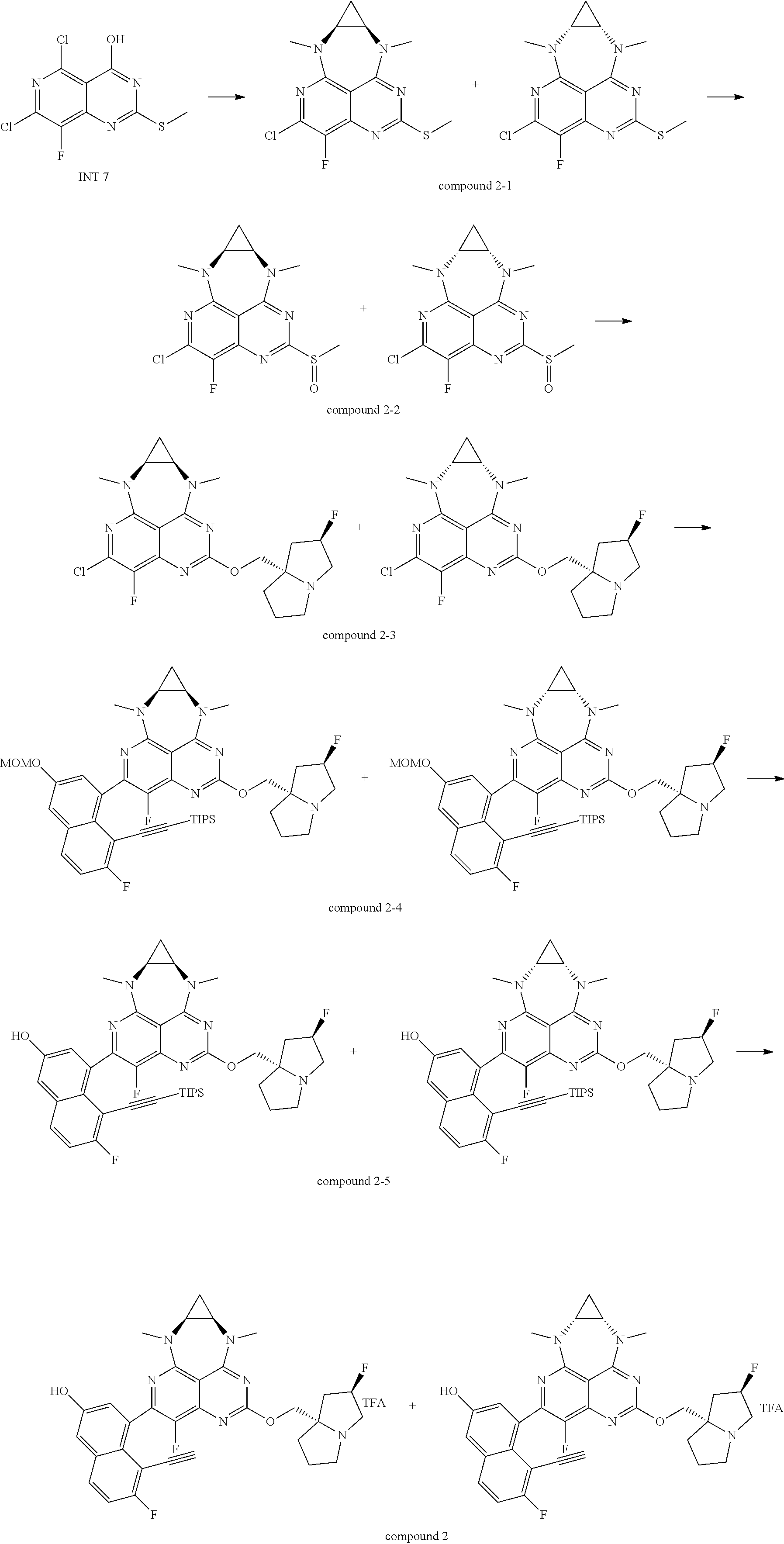

To a solution of INT 7 (512 mg, 1.83 mmol) and DIEA (0.5 mL) in toluene (15 mL) was added phosphorus oxychloride (1 mL) under nitrogen atmosphere and stirred at 80 °C for 4 hours. Upon completion, the mixture was concentrated under reduced pressure. The residue was diluted with DCM (20 mL), then DIEA (1.5 mL) and INT 10 (466 mg, crude) was added, the reaction mixture was stirred at room temperature for 16 hours. Upon completion, the residue was diluted with DCM (30 mL), water (30 mL) and separated. The collected organic layer was concentrated under reduced pressure. The residue was purified by silica gel chromatography (eluting with Hex:EtOAc=2:1, v/v) to give Compound 2-1 (276 mg, 0.85 mmol). MS: m/z 326 [M+H]⁺.

To a solution of Compound 2-1 (263.4 mg, 0.81 mmol) in DCM (15 mL) was added 3-chlorobenzoperoxoic acid (177 mg, 1.03 mmol) at 0 °C and stirred for 3 hours. Upon completion, the residue diluted with DCM (30 mL) and water (30 mL) and the mixture was adjusted to pH=8 to 9 with saturated NaHCO₃. The organic layer was separated and concentrated under reduced pressure. The residue was purified by silica gel chromatography (eluting with Hex:EtOAc=1:1, v/v) to give Compound 2-2 (171 mg, 0.50 mmol). MS: m/z 342 [M+H]+.

To a solution of Compound 2-2 (153.2 mg, 0.45 mmol) and ((2R,7aS)-2-fluorotetrahydro-1H-pyrrolizin-7a(5H)-yl) methanol (150 mg, 0.94 mmol) in THF (15 mL) was added t-BuONa (133 mg, 1.38 mmol) at room temperature. The mixture was stirred for 10 min, then diluted with EtOAc (30 mL) and water (30 mL). The organic layer was separated, washed with brine (20 mL) and dried over anhydrous Na₂SO₄, filtered and the filtrate concentrated under reduced pressure. The residue was purified by Pre-TLC (eluted with DCM:MeOH=20:1, v/v) to give Compound 2-3 (123 mg, 0.28 mmol), MS: m/z 437 [M+H]⁺.

To a mixture of Compound 2-3 (123 mg, 0.28 mmol), INT 2 (311 mg, 0.61 mmol) and Cs₂CO₃ (409 mg, 1.26 mmol) in toluene (20 mL) and water (5 mL) was added methanesulfonato[(di(1-adamantyl)-n-butylphos-phine)-2-(2′-amino-1,1′-biphenyl)]palladium(II) (31 mg, 0.043 mmol), the mixture was purged with nitrogen followed by stirring at 100 °C for 16 hours. Upon completion, the mixture was concentrated under reduced pressure, the residue was diluted with EtOAc (40 mL) and water (30 mL), the organic layer was separated and concentrated under reduced pressure. The residue was purified by Pre-TLC (eluted with DCM:MeOH=20:1, v/v) to give Compound 2-4 (107 mg, 0.14 mmol). MS: m/z 787 [M+1]⁺.

To a solution of Compound 2-4 (107 mg, 0.14 mmol) in CH₃CN (5 mL) was added HCl/1,4-dioxane (2 mL, 4M), The reaction mixture was stirred at room temperature for 1 hour. After completion, the reaction mixture was concentrated under reduced pressure, the residue was diluted with EtOAc (30 mL) and water (20 mL) and adjusted to pH=8 to 9 with saturated NaHCO₃. The organic layer was separated and concentrated under reduced pressure to give Compound 2-5 (104 mg, crude). MS: m/z 743 [M+H]⁺.

To a mixture of Compound 2-5 (104 mg, crude) in DMF (5 mL) was added CsF (319 mg, 2.10 mmol). The mixture was stirred at room temperature for 16 hours. After completion, the mixture was diluted with EtOAc (30 mL) and water (20 mL). The mixture was adjusted to pH=8 to 9 with saturated NaHCO₃. The organic layer was separated and concentrated under reduced pressure. The residue was purified by Pre-HPLC (C18 column, A: 0.1% TFA in water, B: CH₃CN, gradient: 15% B to 50% B in 47 mins at a flow rate of 60 mL/min, 285 nm) and the product fractions were lyophilized to give Compound 2 (64.5 mg, 0.092 mmol) as a TFA salt. MS: m/z 587 [M+H]⁺.

### Example 3

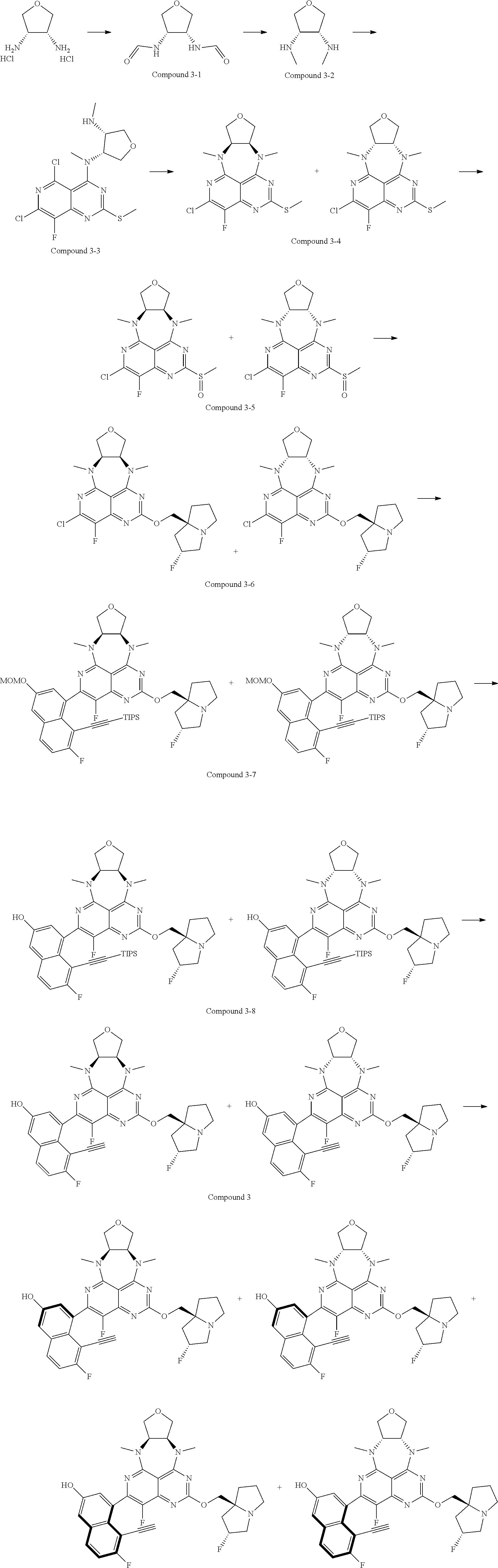

To a solution of cis-tetrahydrofuran-3,4-diamine dihydrochloride (2.04 g, 11.65 mmol) in ethyl formate (24 mL) was added DIEA (3.82 g, 29.56 mmol) at room temperature. The mixture was stirred at 57 °C overnight. The reaction mixture was filtered and the filtrate was concentrated under reduced pressure to give Compound 3-1 (4.32 g, 27.32 mmol, 234.40% yield, crude).

To a solution of Compound 3-1 (3.78 g, 23.90 mmol) in THF (30 mL) was added LiAlH₄ (1.35 g, 35.57 mmol) in portions at 0 °C under nitrogen atmosphere. The mixture was stirred at 51 °C for 7 hours. The reaction mixture was quenched with water (1.5 mL), sat. aq. NaOH (1.5 mL) and water (3 mL). The mixture was filtered and the filtrate was concentrated under reduced pressure. The residue was purified by silica gel chromatography (eluted with DCM:MeOH=50:1 to 10:1, v/v) to give Compound 3-2 (380 mg, 2.92 mmol, 12.21% yield). MS m/z: 131 [M+H]⁺.

To a solution of INT 3 (151 mg, 0.539 mmol) in toluene (5 mL) was added DIEA (0.6 mL) and POCl₃ (0.5 mL). The mixture was stirred at 81 °C for 2 hours, then concentrated under reduced pressure. A solution of the residue in DCM was added to a solution of Compound 3-2 (80 mg, 0.615 mmol) and DIEA (0.7 mL) in DCM (3 mL) at 0 °C. The mixture was stirred at room temperature for 0.5 hour. The mixture was diluted with water, extracted with DCM (20 mL×3) and dried over Na₂SO₄. The combined organic layers were concentrated under reduced pressure to give Compound 3-3 (220 mg, 0.561 mmol, 91.26% yield). MS m/z: 392 [M+H]⁺.

To a solution of Compound 3-3 (220 mg, 0.561 mmol) in acetonitrile (6 mL) was added DIEA (0.6 mL). The mixture was stirred at 81 °C for 4 hours. The reaction mixture was diluted with water, extracted with EtOAc (30 mL), the organic layer was dried over Na₂SO₄ and concentrated under reduced pressure. The residue was purified by Pre-TLC (Hex:EtOAc=2:1, v/v) to give Compound 3-4 (90 mg, 0.253 mmol, 45.10% yield). MS m/z: 356 [M+H]⁺.

To a solution of Compound 3-4 (90 mg, 0.253 mmol) in DCM (5 mL) was added m-CPBA (63 mg, 0.310 mmol, 85% wt) slowly at 0 °C under nitrogen atmosphere. The mixture was stirred at 0 °C for 2 hours. The reaction mixture was quenched with sat.aq. Na₂S₂O₃ (2 mL), the separated organic layer was washed with sat.aq. NaHCO₃ (10 mL). The organic layer was dried over Na₂SO₄ and concentrated under reduced pressure. The residue was purified by Pre-TLC (DCM:MeOH=20:1, v/v) to give Compound 3-5 (104 mg, 0.280 mmol, 110.58% yield). MS m/z: 372 [M+H]⁺.

t-BuONa (51 mg, 0.531 mmol) was added to a solution of ((2R,7aS)-2-fluorotetrahydro-1H-pyrrolizin-7a(5H)-yl)methanol (64 mg, 0.402 mmol) in THF (4 mL) at 0 °C under nitrogen atmosphere. Then a solution of Compound 3-5 (104 mg, 0.280 mmol) in THF (2 mL) was added and the mixture was stirred for 0.5 hour at room temperature. The mixture was quenched with water and extracted with EtOAc (30 mL). The organic layer was washed with brine (30 mL×2), dried over Na₂SO₄ and concentrated under reduced pressure. The residue was purified by Pre-TLC (DCM:MeOH=15:1, v/v) to give Compound 3-6 (40 mg, 0.086 mmol, 30.62% yield). MS m/z: 467 [M+H]⁺.

To a solution of Compound 3-6 (40 mg, 0.086 mmol), INT 1 (60 mg, 0.117 mmol) in toluene (10 mL) and water (2 mL) were added Cs₂CO₃ (80 mg, 0.246 mmol) and cataCXium A Pd G₃ (11 mg, 0.015 mmol). The reaction mixture was stirred at 100 °C overnight under nitrogen atmosphere. The mixture was filtered and the filtrate was concentrated under reduced pressure. The residue was purified by Pre-TLC (DCM:MeOH=20:1, v/v) to give Compound 3-7 (55 mg, 0.067 mmol, 78.57% yield). MS m/z: 817 [M+H]⁺.

A solution of Compound 3-7 (55 mg, 0.067 mmol) and HCl (0.5 mL, 4M in dioxane) in acetonitrile (5 mL) was stirred at RT for 2 hours. The solution was diluted with sat.aq. NaHCO₃ (20 mL) and extracted with EtOAc (30 mL). The organic layer was washed with brine (30 mL), dried over Na₂SO₄ and concentrated under reduced pressure to give Compound 3-8 (52 mg, 0.067 mmol, 99.93% yield). MS m/z: 773 [M+H]⁺.

To a solution of Compound 3-8 (52 mg, 0.067 mmol) in DMF (3 mL) was added CsF (0.14 g. 0.922 mmol). The reaction mixture was stirred at 40 °C for 2 hours under nitrogen atmosphere. The mixture was diluted with water (10 mL) and extracted with EtOAc (20 mL). The organic layer was dried over Na₂SO₄ and concentrated under reduced pressure. The residue was purified by Prep-HPLC (Agela Durashell C18 column, 30 mm×250 mm, 10 μm; A: 0.05% NH₃·H₂O in water, B: CH₃CN, Gradient: 25% B to 60% B in 60 min at a flow rate of 40 mL/min, 230 nm) to freeze-dried to afford Compound 3 (19.0 mg, 0.140 mmol, 45.80% yield) as an off-white solid. MS m/z: 617 [M+H]⁺.

The isomers were separated by Prep-HPLC-Gilson with the following conditions: Column, CHIRAL ART Cellulose-SA column (2 cm×25 cm, 5 μm); mobile phase, Hex (0.2% isopropylamine)/EtOH (55:45); Flowing rate: 20 mL/min. This results in Compound 3A (2.9 mg, off-white solid, the first eluting isomer, retention time: 3.802 min); Compound 3B (6.5 mg, off-white solid, a mixture of the second eluting isomer and the third eluting isomer, retention time: 4.14 min); and Compound 3C (2.6 mg, off-white solid, the fourth eluting isomer, retention time: 6.121 min).

### Example 4

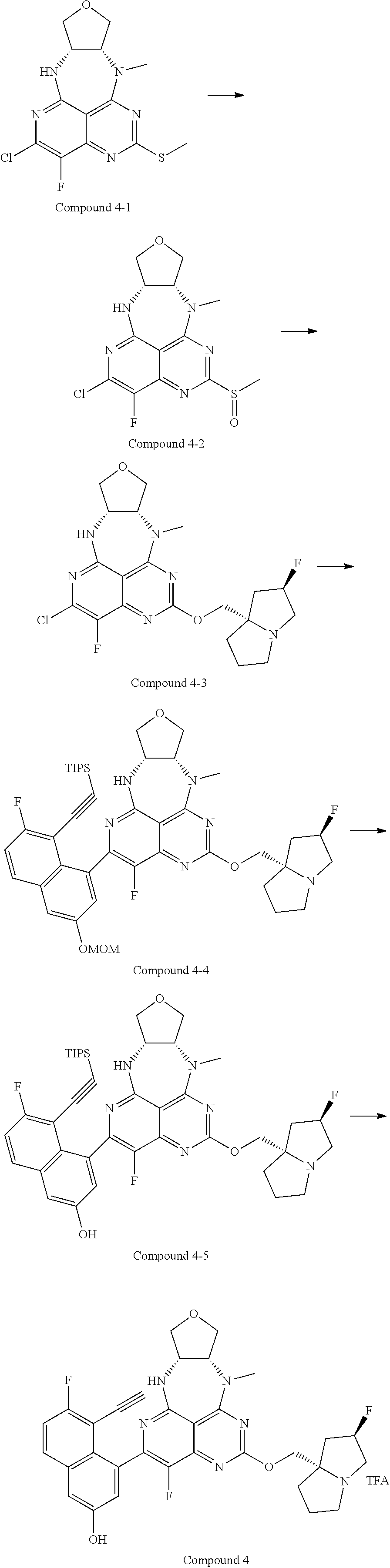

A solution of Compound 4-1 (0.096 g, 280.87 μmol) in DCM (0.006 L) was cooled down by ice ethanol bath. Then m-CPBA (62 mg, 305.39 μmol) was added and stirred for 1.5 h at the same temperature. Then the mixture was purified by Pre-TLC to afford Compound 4-2 (47 mg, 131.36 μmol, 46.7% yield). MS (ESI, m/z): 358 [M+H]⁺.

A solution of ((2R,7aS)-2-fluorotetrahydro-1H-pyrrolizin-7a (5H)-yl) methanol (0.02 g, 125.62 μmol) in THF (2 mL) was cooled down by ice ethanol bath. Then t-BuONa (0.02 g, 208.10 μmol) was added and stirred for 10 min, followed with a solution of Compound 4-2 (47 mg, 131.36 μmol) in THF (4 mL) was added. The reaction mixture was stirred for 20 min. Then the mixture was quenched with water, extracted with EA. The organic layer was dried over Na₂SO₄ and concentrated in vacuum. The residue was purified by Pre-TLC (DCM:MeOH=15:1, v/v) to afford Compound 4-3 (24 mg, 52.99 μmol, 40.3% yield). MS (ESI, m/z): 453 [M+H]⁺.

A solution of Compound 4-3 (24 mg, μmol), 52.99 ((2-fluoro-6-(methoxymethoxy)-8-(4,4,5,5-tetramethyl-1,3,2-dioxaborolan-2-yl)naphthalen-1-yl)ethynyl)triiso propylsilane (0.038 g, 74.14 μmol), cataCXium A Pd G₃ (0.02 g, 27.46 μmol), Cs₂CO₃ (0.053 g, 162.66 μmol) in toluene (4 mL) and water (1 mL) was stirred at 100 °C for 16 hours under nitrogen atmosphere. The reaction was extracted with EA. The organic layer was concentrated under vacuum. The residue was purified by Pre-TLC (DCM:MeOH=15:1, v/v) to afford Compound 4-4 (22 mg, 27.39 μmol, 51.6% yield). MS (ESI, m/z): 803 [M+H]⁺.

To a solution of Compound 4-4 (22 mg, 27.39 μmol) in CH₃CN (3 mL) was added HCl (4 M in 1,4-dioxane, 0.5 mL). The reaction mixture was stirred at room temperature for 3 hours. The mixture was quenched with aq. NaHCO₃, extracted with EA. The organic layer was concentrated under vacuum to afford Compound 4-5 (39 mg, 51.38 μmol, 187.5% yield). MS (ESI, m/z): 759 [M+H]⁺.

To a solution of Compound 4-5 (39 mg, 51.38 μmol) in DMF (3 mL) was added CsF (0.06 g, 394.98 μmol). The reaction mixture was stirred at 44 °C for 1 h. Then the mixture was extracted with EA. The organic layer was concentrated under vacuum. The residue was purified by Prep-HPLC (Agela Durashell C18, 30 mm×250 mm, 10 μm, phase A: 0.1% TFA in water, phase B: CH₃CN, Gradient: 10% B to 35% B in 37 min at a flow rate of 40 mL/min, 250 nm) to freeze-dried to afford Compound 4 (0.0091 g, 12.69 μmol, TFA salt, 24.7% yield). MS (ESI, m/z): 603 [M+H]⁺.

### Example 5

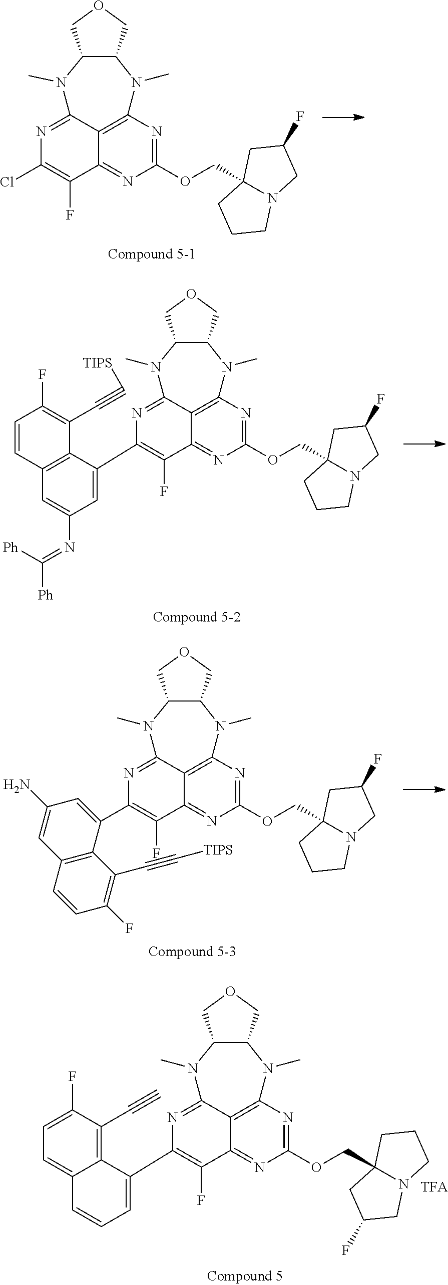

A solution of Compound 5-1 (0.053 g, 113.51 μmol), N-(6-fluoro-4-(4,4,5,5-tetramethyl-1,3,2-dioxaborolan-2-yl)-5-((triisopropylsilyl)ethynyl)naphthalen-2-yl)-1,1-diphenylmethanimine (0.098 g, 155.13 μmol), cataCXium A Pd G₃ (0.023 g, 35.58 μmol), Cs₂CO₃ (0.102 g, 313.05 μmol) in toluene (6 mL) and water (1.5 mL) was stirred at 100 °C for 16 hours under nitrogen atmosphere. The reaction was extracted with EA. The organic layer was dried over anhydrous Na₂SO₄ and concentrated in vacuum. The residue was purified by Pre-TLC (DCM:MeOH=15:1, v/v) to afford Compound 5-2 (98 mg, 104.67 μmol, 92.2% yield). MS (ESI, m/z): 936 [M+H]⁺.

To a solution of Compound 5-2 (0.098 g, 104.67 μmol) in DCM (2 mL) was added HCl (4 M in 1,4-dioxane, 0.6 mL). The reaction mixture was stirred at room temperature for 1.5 hours and added HCl (4 M in 1,4-dioxane, 0.2 mL) again, stirred at room temperature for 1.5 hours and concentrated under reduced pressure to afford Compound 5-3 (0.10 g, 129.53 μmol, 123.7% yield) as a crude. MS (ESI, m/z): 772 [M+H]⁺.

To a solution of Compound 5-3 (0.1 g, 129.53 μmol) in DMF (3 mL) was added CsF (0.24 g, 1.58 mmol). The reaction mixture was stirred at 44 °C for 1.5 h. The reaction was extracted with EA. The organic layer was dried over Na₂SO₄ and concentrated in vacuum. The residue was purified by Prep-HPLC (Daisogel-C18, 50 mm×250 mm, 10 μm, phase A: 0.1% TFA in water, phase B: CH₃CN, gradient: 10% B to 40% B in 46 min at a flow rate of 60 mL/min, 230 nm) to freeze-dried to afford Compound 5 (0.0425 g, 58.24 μmol, TFA salt, 44.9% yield). MS (ESI, m/z): 616 [M+H]⁺.

### Pharmacological Experiments

1. SOS1 catalyzed nucleotide exchange assay

GDP-loaded HIS-KRAS(G12V, aa 1-169) was pre-incubated with a compound in the presence of 10 nM GDP in a 384-well plate (Greiner) for 15 min, then purified SOS1 ExD (Flag tag, aa 564-1049), BODIPY™ FL GTP (Invitrogen) and MAb (monoclonal antibody) Anti 6HIS-Tb cryptate Gold (Cisbio) were added to the assay wells (Final concentration: 10-15 nM GDP-loaded HIS-KRAS(G12V), 5 nM GDP, 0.5 μM SOS1 ExD, 80 nM BODIPY™ FL GTP, 52.5 ng/mL MAb Anti 6HIS-Tb cryptate Gold) and incubated for 4 hours at 25 °C. Wells containing same percent of DMSO served as vehicle control, and wells without KRAS served as low control. TR-FRET signals were read on Tecan Spark multimode microplate reader. The parameters were F486: Excitation 340 nm, Emission 486 nm, Lag time 100 μs, Integration time 200 μs; F520: Excitation 340 nm, Emission 520 nm, Lag time 100 μs, Integration time 200 μs. TR-FRET ratios for each individual wells were calculated by equation: TR-FRET ratio=(Signal F520/Signal F486)*10000. The percent of activation of compounds treated wells were normalized between vehicle control and low control (% Activation=(TR-FRET ratio_{Compound treated}−TR-FRET ratio_{Low control})/(TR-FRET ratio_{Vehicle control}−TR-FRET ratio_{Low control})*100%). Then the data were analyzed either by fitting a 4-parameter logistic model or by Excel to calculate IC₅₀ values.

TABLE 1

| Compound | GDP-KRAS G12V IC₅₀ (nM) |
|---|---|
| Compound 1 | 4.38 |
| Compound 2 | 26.4 |
| Compound 3 | 0.757 |
| Compound 4 | 1.35 |
| Compound 5 | 5.58 |

LENGTHY TABLES
The patent application contains a lengthy table section. A copy of the table is available in electronic form from the USPTO web site (https://seqdata.uspto.gov/docdetail?docId=US20260365672A1). An electronic copy of the table will also be available from the USPTO upon request and payment of the fee set forth in 37 CFR 1.19(b)(3).

What is claimed is:

1. A compound of formula (I), a stereoisomer thereof, a pharmaceutically acceptable salt thereof, a pharmaceutically acceptable salt of the stereoisomer, a prodrug thereof, a deuterated molecule thereof or a PROTAC molecule thereof: 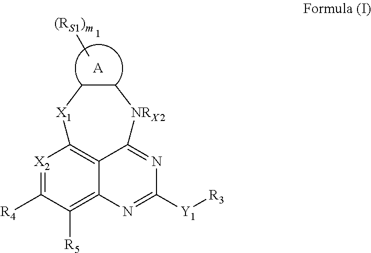
wherein,
X₁ at each occurrence is independently selected from the group consisting of a bond, —C(R_{X11})(R_{X12})—, —NR_{X13}—, —O—, —S—, —S(═O)— and —S(═O)₂—;
R_{X11} or R_{X12} is independently selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, R_{X11} and R_{X12} together with the carbon atom to which they are both attached forms 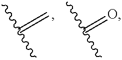
a 3-10 membered carbocyclic ring or a 3-10 heterocyclic ring; wherein, said 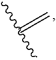
3-10 membered carbocylic ring or 3-10 heterocyclic ring is independently unsubstituted or substituted with one or more R_{SX1};
R_{X13} is selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
R_{X2} is selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
X₂ at each occurrence is independently selected from the group consisting of N and CR₁;
R₁ is selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1A})₂, —OR_{1A}, —SR_{1A}, —S(═O)R_{1B}, —S(═O)₂R_{1B}, —C(═O)R_{1B}, —C(═O)OR_{1A}, —OC(═O)R_{1B}, —C(═O)N(R_{1A})₂, —NR_{1A}C(═O)R_{1B}, —OC(═O)OR_{1A}, —NR_{1A}C(═O)OR_{1A}, —NR_{1A}C(═S)OR_{1A}, —OC(═O)N(R_{1A})₂, —NR_{1A}C(═O)N(R_{1A})₂, —S(═O)OR_{1A}, —OS(═O)R_{1B}, —S(═O)N(R_{1A})₂, —NR_{1A}S(═O)R_{1B}, —S(═O)₂OR_{1A}, —OS(═O)₂R_{1B}, —S(═O)₂N(R_{1A})₂, —NR_{1A}S(═O)₂R_{1B}, —OS(═O)₂OR_{1A}, —NR_{1A}S(═O)₂OR_{1A}, —OS(═O)₂N(R_{1A})₂, —NR_{1A}S(═O)₂N(R_{1A})₂, —P(R_{1A})₂, —P(═O)(R_{1B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{1C})₂, —OR_{1C}, —SR_{1C}, —S(═O)R_{1D}, —S(═O)₂R_{1D}, —C(═O)R_{1D}, —C(═O)OR_{1D}, —OC(═O)R_{1D}, —C(═O)N(R_{1C})₂, —NR_{1C}C(═O)R_{1D}, —OC(═O)OR_{1C}, —NR_{1C}C(═O)OR_{1C}, —NR_{1C}C(═S)OR_{1C}, —OC(═O)N(R_{1C})₂, —NR_{1C}C(═O)N(R_{1C})₂, —S(═O)OR_{1C}, —OS(═O)R_{1D}, —S(═O)N(R_{1C})₂, —NR_{1C}S(═O)R_{1D}, —S(═O)₂OR_{1C}, —OS(═O)₂R_{1D}, —S(═O)₂N(R_{1C})₂, —NR_{1C}S(═O)₂R_{1D}, —OS(═O)₂OR_{1C}, —NR_{1C}S(═O)₂OR_{1C}, —OS(═O)₂N(R_{1C})₂, —NR_{1C}S(═O)₂N(R_{1C})₂, —P(R_{1C})₂, —P(═O)(R_{1D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
ring A is selected from the group consisting of a 3-10 membered carbocyclic ring, 3-10 membered heterocyclic ring, 6-10 membered aryl ring, and 5-10 membered heteroaryl ring;
R_{S1} is independently selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1A})₂, —OR_{S1A}, —SR_{S1A}, —S(═O)R_{S1B}, —S(═O)₂R_{S1B}, —C(═O)R_{S1B}, —C(═O)OR_{S1A}, —OC(═O)R_{S1B}, —C(═O)N(R_{S1A})₂, —N_{S1A}C(═O)R_{S1B}, —OC(═O)OR_{S1A}, —N_{S1A}C(═O)OR_{S1A}, —NR_{S1A}C(═S)O_{S1A}, —OC(═O)N(R_{S1A})₂, —NR_{S1A}C(═O)N(R_{S1A})₂, —S(═O)OR_{S1A}, —OS(═O)R_{S1B}, —S(═O)N(R_{S1A})₂, —NR_{S1A}S(═O)R_{S1B}, —S(═O)₂OR_{S1A}, —OS(═O)₂R_{S1B}, —S(═O)₂N(R_{S1A})₂, —NR_{S1A}S(═O)₂R_{S1B}, —OS(═O)₂OR_{S1A}, —NR_{S1A}S(═O)₂OR_{S1A}, —OS(═O)₂N(R_{S1A})₂, —NR_{S1A}S(═O)₂N(R_{S1A})₂, —P(R_{S1A})₂, —P(═O)(R_{S1B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S1C})₂, —OR_{S1C}, —SR_{S1C}, —S(═O)R_{S1D}, —S(═O)₂R_{S1D}, —C(═O)R_{S1D}, —C(═O)OR_{S1C}, —OC(═O)R_{S1D}, —C(═O)N(R_{S1C})₂, —NR_{S1C}C(═O)R_{S1D}, —OC(═O)OR_{S1C}, —NR_{S1C}C(═O)OR_{S1C}, —NR_{S1C}C(═S)OR_{S1C}, —OC(═O)N(R_{S1C})₂, —NR_{S1C}C(═O)N(R_{S1C})₂, —S(═O)OR_{S1C}, —OS(═O)R_{S1D}, —S(═O)N(R_{S1C})₂, —NR_{S1C}S(═O)R_{S1D}, —S(═O)₂OR_{S1C}, —OS(═O)₂R_{S1D}, —S(═O)₂N(R_{S1C})₂, —NR_{S1C}S(═O)₂R_{S1D}, —OS(═O)₂OR_{S1C}, —NR_{S1C}S(═O)₂OR_{S1C}, —OS(═O)₂N(R_{S1C})₂, —NR_{S1C}S(═O)₂N(R_{S1C})₂, —P(R_{S1C})₂, —P(═O)(R_{S1D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S1} together with the carbon atom to which they are both attached form 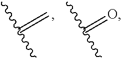
a 3-10 membered carbocyclic ring or a 3-10 heterocyclic ring; wherein, said 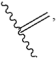
3-10 membered carbocylic ring or 3-10 heterocyclic ring is independently unsubstituted or substituted with one or more R_{S11};
optionally, two adjacent R_{S1} together with the atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S12};
optionally, two nonadjacent R_{S1} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S13};
m₁ is selected from the group consisting of 0, 1, 2, 3, 4, 5, 6, 7, 8 and 9;
Y₁ is a bond, O, S, S(═O), S(═O)₂ or NR_{Y11};
R_{Y11} is selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
R₃ is selected from the group consisting of 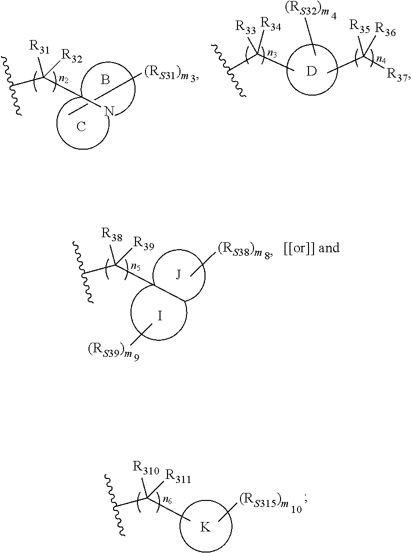
each of R₃₁, R₃₂, R₃₃, R₃₄, R₃₅, R₃₆, R₃₈, R₃₉, R₃₁₀ and R₃₁₁ is independently selected form the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and -5-10 membered heteroaryl;
optionally R₃₁ and R₃₂ together with the carbon atom to which they are both attached form 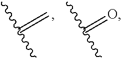
a 3-10 membered carbocyclic ring or a 3-10 heterocyclic ring; wherein, said 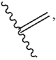
3-10 membered carbocylic ring or 3-10 heterocyclic ring is independently unsubstituted or substituted with one or more R_{S33};
optionally, R₃₃ and R₃₄ together with the carbon atom to which they are both attached form 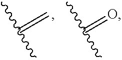
a 3-10 membered carbocyclic ring or a 3-10 heterocyclic ring; wherein, said 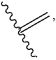
3-10 membered carbocylic ring or 3-10 heterocyclic ring is independently unsubstituted or substituted with one or more R_{S34};
optionally, R₃₅ and R₃₆ together with the carbon atom to which they are both attached form 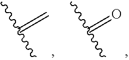
a 3-10 membered carbocyclic ring or a 3-10 heterocyclic ring; wherein, said 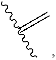
3-10 membered carbocylic ring or 3-10 heterocyclic ring is independently unsubstituted or substituted with one or more R_{S35};
optionally, R₃₈ and R₃₉ together with the carbon atom to which they are both attached form 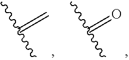
a 3-10 membered carbocyclic ring or a 3-10 heterocyclic ring; wherein, said 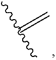
3-10 membered carbocylic ring or 3-10 heterocyclic ring is independently unsubstituted or substituted with one or more R_{S310};
optionally, R₃₁₀ and R₃₁₁ together with the carbon atom to which they are both attached form 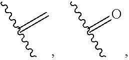
a 3-10 membered carbocyclic ring or a 3-10 heterocyclic ring; wherein, said 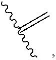
3-10 membered carbocylic ring or 3-10 heterocyclic ring is independently unsubstituted or substituted with one or more R_{S316};
n₂ is selected from the group consisting of 0, 1, 2, 3, 4, 5 and 6;
n₃ is selected from the group consisting of 0, 1, 2, 3, 4, 5 and 6;
n₄ is selected from the group consisting of 0, 1, 2, 3, 4, 5 and 6;
n₅ is selected from the group consisting of 0, 1, 2, 3, 4, 5 and 6;
n₆ is selected from the group consisting of 0, 1, 2, 3, 4, 5 and 6;
ring B is a 3-10 membered heterocyclic ring optionally further containing 1, 2, or 3 heteroatoms selected from the group consisting of N, O, S, S(═O) and S(═O)₂;
ring C is a 3-10 membered heterocyclic ring optionally further containing 1, 2, or 3 heteroatoms selected from the group consisting of N, O, S, S(═O) and S(═O)₂;
ring D is selected from the group consisting of a 3-10 membered carbocyclic ring and a 3-10 heterocyclic ring;
ring I is a 3-10 membered carbocyclic ring or a 3-10 membered heterocyclic ring containing 1, 2, or 3 heteroatoms selected from the group consisting of N, O, S, S(═O) and S(═O)₂;
ring J is a 3-10 membered carbocyclic ring or a 3-10 membered heterocyclic ring containing 1, 2, or 3 heteroatoms selected from the group consisting of N, O, S, S(═O) and S(═O)₂;
ring K is selected from the group consisting of 3-10 membered carbocyclic ring and a 3-10 membered heterocyclic ring containing 1, 2, or 3 heteroatoms selected from the group consisting of N, O, S, S(═O) and S(═O)₂;
R_{S31} is selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31A})₂, —OR_{S31A}, —SR_{S31A}, —S(═O)R_{S31B}, —S(═O)₂R_{S31B}, —C(═O)R_{S31B}, —C(═O)OR_{S31A}, —OC(═O)R_{S31B}, —C(═O)N(R_{S31A})₂, —NR_{S31A}C(═O)R_{S31B}, —OC(═O)OR_{S31A}, —NR_{S31A}C(═O)OR_{S31A}, —NR_{S31A}C(═S)OR_{S31A}, —OC(═O)N(R_{S31A})₂, —NR_{S31A}C(═O)N(R_{S31A})₂, —S(═O)OR_{S31A}, —OS(═O)R_{S31B}, —S(═O)N(R_{S31A})₂, —NR_{S31A}S(═O)R_{S31B}, —S(═O)₂OR_{S31A}, —OS(═O)₂R_{S31B}, —S(═O)₂N(R_{S31A})₂, —NR_{S31A}S(═O)₂R_{S31B}, —OS(═O)₂OR_{S31A}, —NR_{S31A}S(═O)₂OR_{S31A}, —OS(═O)₂N(R_{S31A})₂, —NR_{S31A}S(═O)₂N(R_{S31A})₂, —P(R_{S31A})₂, —P(═O)(R_{S31B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S31C})₂, —OR_{S31C}, —SR_{S31C}, —S(═O)R_{S31D}, —S(═O)₂R_{S31D}, —C(═O)R_{S31D}, —C(═O)OR_{S31C}, —OC(═O)R_{S31D}, —C(═O)N(R_{S31C})₂, —NR_{S31C}C(═O)R_{S31D}, —OC(═O)OR_{S31C}, —NR_{S31C}C(═O)OR_{S31C}, —NR_{S31C}C(═S)OR_{S31C}, —OC(═O)N(R_{S31C})₂, —NR_{S31C}C(═O)N(R_{S31C})₂, —S(═O)OR_{S31C}, —OS(═O)R_{S31D}, —S(═O)N(R_{S31C})₂, —NR_{S31C}S(═O)R_{S31D}, —S(═O)₂OR_{S3C}, —OS(═O)₂R_{S31D}, —S(═O)₂N(R_{S31C})₂, —NR_{S31C}S(═O)₂R_{S31D}, —OS(═O)₂OR_{S31C}, —NR_{S31C}S(═O)₂OR_{S31C}, —OS(═O)₂N(R_{S31C})₂, —NR_{S31C}S(═O)₂N(R_{S31C})₂, —P(R_{S31C})₂, —P(═O)(R_{S31D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S31} together with the carbon atom to which they are both attached form 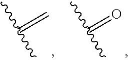
a 3-10 membered carbocyclic ring or a 3-10 membered heterocyclic ring; wherein, said 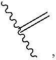
3-10 membered carbocylic ring or 3-10 heterocyclic ring is independently unsubstituted or substituted with one or more R_{S311};
optionally, two adjacent R_{S31} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S312};
optionally, two nonadjacent R_{S31} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S313};
m₃ is selected from the group consisting of 0, 1, 2, 3, 4, 5, 6, 7, 8, 9, 10, 11, and 12;
R_{S32} is selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32A})₂, —OR_{S32A}, —SR_{S32A}, —S(═O)R_{S32B}, —S(═O)₂R_{S32B}, —C(═O)R_{S32B}, —C(═O)OR_{S32A}, —OC(═O)R_{S32B}, —C(═O)N(R_{S32A})₂, —NR_{S32A}C(═O)R_{S32B}, —OC(═O)OR_{S32A}, —NR_{S32A}C(═O)OR_{S32A}, —NR_{S32A}C(═S)OR_{S32A}, —OC(═O)N(R_{S32A})₂, —NR_{S32A}C(═O)N(R_{S32A})₂, —S(═O)OR_{S32A}, —OS(═O)R_{S32B}, —S(═O)N(R_{S32A})₂, —NR_{S32A}S(═O)R_{S32B}, —S(═O)₂OR_{S32A}, —OS(═O)₂R_{S32B}, —S(═O)₂N(R_{S32A})₂, —NR_{S32A}S(═O)₂R_{S32B}, —OS(═O)₂OR_{S32A}, —NR_{S32A}S(═O)₂OR_{S32A}, —OS(═O)₂N(R_{S32A})₂, —NR_{S32A}S(═O)₂N(R_{S32A})₂, —P(R_{S32A})₂, —P(═O)(R_{S32B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S32C})₂, —OR_{S32C}, —SR_{S32C}, —S(═O)R_{S32C}, —S(═O)₂R_{S32D}, —C(═O)R_{S32D}, —C(═O)OR_{S32C}, —OC(═O)R_{S32D}, —C(═O)N(R_{S32C})₂, —NR_{S32C}C(═O)R_{S32D}, —OC(═O)OR_{S32C}, —NR_{S32C}C(═O)OR_{S32C}, —NR_{S32C}C(═S)OR_{S32C}, —OC(═O)N(R_{S32C})₂, —NR_{S32C}C(═O)N(R_{S32C})₂, —S(═O)OR_{S32C}, —OS(═O)R_{S32C}, —S(═O)N(R_{S32C})₂, —NR_{S32C}S(═O)R_{S32D}, —S(═O)₂OR_{S32C}, —OS(═O)₂R_{S32D}, —S(═O)₂N(R_{S32C})₂, —NR_{S32C}S(═O)₂R_{S32D}, —OS(═O)₂OR_{S32C}, —NR_{S32C}S(═O)₂OR_{S32C}, —OS(═O)₂N(R_{S32C})₂, —NR_{S32C}S(═O)₂N(R_{S32C})₂, —P(R_{S32C})₂, —P(═O)(R_{S32D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S32} together with the carbon atom to which they are both attached form 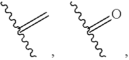
a 3-10 membered carbocyclic ring or a 3-10 heterocyclic ring; wherein, said 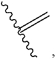
3-10 membered carbocylic ring or 3-10 heterocyclic ring is independently unsubstituted or substituted with one or more R_{S321};
optionally, two adjacent R_{S32} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S322};
optionally, two nonadjacent R_{S32} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S323};
m₄ is selected from the group consisting of 0, 1, 2, 3, 4, 5 and 6;
R₃₇ is selected from the group consisting of —N(R_{37A})₂ and 3-10 membered heterocyclyl, wherein said 3-10 membered heterocyclyl is optionally independently substituted with one or more R_{S37};
R_{S38} is selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38A})₂, —OR_{S38A}, —SR_{S38A}, —S(═O)R_{S38B}, —S(═O)₂R_{S38B}, —C(═O)R_{S38B}, —C(═O)OR_{S38A}, —OC(═O)R_{S38B}, —C(═O)N(R_{S38A})₂, —NR_{S38A}C(═O)R_{S38B}, —OC(═O)OR_{S38A}, —NR_{S38A}C(═O)OR_{S38A}, —NR_{S38A}C(═S)OR_{S38A}, —OC(═O)N(R_{S38A})₂, —NR_{S38A}C(═O)N(R_{S38A})₂, —S(═O)OR_{S38A}, —OS(═O)R_{S38B}, —S(═O)N(R_{S38A})₂, —NR_{S38A}S(═O)R_{S38B}, —S(═O)₂OR_{S38A}, —OS(═O)₂R_{S38B}, —S(═O)₂N(R_{S38A})₂, —NR_{S38A}S(═O)₂R_{S38B}, —OS(═O)₂OR_{S38A}, —NR_{S38A}S(═O)₂OR_{S38A}, —OS(═O)₂N(R_{S38A})₂, —NR_{S38A}S(═O)₂N(R_{S38A})₂, —P(R_{S38A})₂, —P(═O)(R_{S38B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S38C})₂, —OR_{S38C}, —SR_{S38C}, —S(═O)R_{S38D}, —S(═O)₂R_{S38D}, —C(═O)R_{S38D}, —C(═O)OR_{S38C}, —OC(═O)R_{S38D}, —C(═O)N(R_{S38C})₂, —NR_{S38C}C(═O)R_{S38D}, —NR_{S38C}C(═S)OR_{S38C}, —OC(═O)OR_{S38C}, —NR_{S38C}C(═O)OR_{S38C}, —OC(═O)N(R_{S38C})₂, —NR_{S38C}C(═O)N(R_{S38C})₂, —S(═O)OR_{S38C}, —OS(═O)R_{S38D}, —S(═O)N(R_{S38C})₂, —NR_{S38C}S(═O)R_{S38D}, —S(═O)₂OR_{S38C}, —OS(═O)₂R_{S38D}, —S(═O)₂N(R_{S38C})₂, —NR_{S38C}S(═O)₂R_{S38D}, —OS(═O)₂OR_{S38C}, —NR_{S38C}S(═O)₂OR_{S38C}, —OS(═O)₂N(R_{S38C})₂, —NR_{S38C}S(═O)₂N(R_{S38C})₂, —P(R_{S38C})₂, —P(═O)(R_{S38D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S38} together with the carbon atom to which they are both attached form 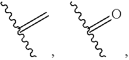
a 3-10 membered carbocyclic ring or a 3-10 membered heterocyclic ring; wherein, said 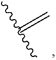
3-10 membered carbocylic ring or 3-10 heterocyclic ring is independently unsubstituted or substituted with one or more R_{S381};
optionally, two adjacent R_{S38} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S382};
optionally, two nonadjacent R_{S38} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S383};
m₈ is selected from the group consisting of 0, 1, 2, 3, 4, 5, 6, 7, 8, 9, 10, 11, and 12;
R_{S39} is selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39A})₂, —OR_{S39A}, —SR_{S39A}, —S(═O)R_{S39B}, —S(═O)₂R_{S39B}, —C(═O)R_{S39B}, —C(═O)OR_{S39A}, —OC(═O)R_{S39B}, —C(═O)N(R_{S39A})₂, —NR_{S39A}C(═O)R_{S39B}, —OC(═O)OR_{S39A}, —NR_{S39A}C(═O)OR_{S39A}, —NR_{S39A}C(═S)OR_{S39A}, —OC(═O)N(R_{S39A})₂, —NR_{S39A}C(═O)N(R_{S39A})₂, —S(═O)OR_{S39A}, —OS(═O)R_{S39B}, —S(═O)N(R_{S39A})₂, —NR_{S39A}S(═O)R_{S39B}, —S(═O)₂OR_{S39A}, —OS(═O)₂R_{S39B}, —S(═O)₂N(R_{S39A})₂, —NR_{S39A}S(═O)₂R_{S39B}, —OS(═O)₂OR_{S39A}, —NR_{S39A}S(═O)₂OR_{S39A}, —OS(═O)₂N(R_{S39A})₂, —NR_{S39A}S(═O)₂N(R_{S39A})₂, —P(R_{S39A})₂, —P(═O)(R_{S39B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S39C})₂, —OR_{S39C}, —SR_{S39C}, —S(═O)R_{S39C}, —S(═O)₂R_{S39D}, —C(═O)R_{S39D}, —C(═O)OR_{S39C}, —OC(═O)R_{S39D}, —C(═O)N(R_{S39C})₂, —NR_{S39C}C(═O)R_{S39D}, —OC(═O)OR_{S39C}, —NR_{S39C}C(═O)OR_{S39C}, —NR_{S39C}C(═S)OR_{S39C}, —OC(═O)N(R_{S39C})₂, —NR_{S39C}C(═O)N(R_{S39C})₂, —S(═O)OR_{S39C}, —OS(═O)R_{S39C}, —S(═O)N(R_{S39C})₂, —NR_{S39C}S(═O)R_{S39D}, —S(═O)₂OR_{S39C}, —OS(═O)₂R_{S39D}, —S(═O)₂N(R_{S39C})₂, —NR_{S39C}S(═O)₂R_{S39D}, —OS(═O)₂OR_{S39C}, —NR_{S39C}S(═O)₂OR_{S39C}, —OS(═O)₂N(R_{S39C})₂, —NR_{S39C}S(═O)₂N(R_{S39C})₂, —P(R_{S39C})₂, —P(═O)(R_{S39D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S39} together with the carbon atom to which they are both attached form 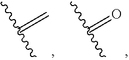
a 3-10 membered carbocyclic ring or a 3-10 heterocyclic ring; wherein, said 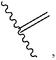
3-10 membered carbocylic ring or 3-10 heterocyclic ring is independently unsubstituted or substituted with one or more R_{S391};
optionally, two adjacent R_{S39} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S392};
optionally, two nonadjacent R_{S39} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S393};
m₉ is selected from the group consisting of 0, 1, 2, 3, 4, 5 and 6;
R_{S315} is selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315A})₂, —OR_{S315A}, —SR_{S315A}, —S(═O)R_{S315B}, —S(═O)₂R_{S315B}, —C(═O)R_{S315B}, —C(═O)OR_{S315A}, —OC(═O)R_{S315B}, —C(═O)N(R_{S315A})₂, —NR_{S315A}C(═O)R_{S315B}, —OC(═O)OR_{S315A}, —NR_{S315A}C(═O)OR_{S315A}, —NR_{S315A}C(═S)OR_{S315A}, —OC(═O)N(R_{S315A})₂, —NR_{S315A}C(═O)N(R_{S315A})₂, —S(═O)OR_{S315A}, —OS(═O)R_{S315B}, —S(═O)N(R_{S315A})₂, —NR_{S315A}S(═O)R_{S315B}, —S(═O)₂OR_{S315A}, —OS(═O)₂R_{S315B}, —S(═O)₂N(R_{S315A})₂, —NR_{S315A}S(═O)₂R_{S315B}, —OS(═O)₂OR_{S315A}, —NR_{S315A}S(═O)₂OR_{S315A}, —OS(═O)₂N(R_{S315A})₂, —NR_{S315A}S(═O)₂N(R_{S315A})₂, —P(R_{S315A})₂, —P(═O)(R_{S315B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S315C})₂, —OR_{S315C}, —SR_{S315C}, —S(═O)R_{S315C}, —S(═O)₂R_{S315D}, —C(═O)R_{S315D}, —C(═O)OR_{S315C}, —OC(═O)R_{S315D}, —C(═O)N(R_{S315C})₂, —NR_{S315C}C(═O)R_{S315D}, —OC(═O)OR_{S315C}, —NR_{S315C}C(═O)OR_{S315C}, —NR_{S315C}C(═S)OR_{S315C}, —OC(═O)N(R_{S315C})₂, —NR_{S315C}C(═O)N(R_{S315C})₂, —S(═O)OR_{S315C}, —OS(═O)R_{S315C}, —S(═O)N(R_{S315C})₂, —NR_{S315C}S(═O)R_{S315D}, —S(═O)₂OR_{S315C}, —OS(═O)₂R_{S315D}, —S(═O)₂N(R_{S315C})₂, —NR_{S315C}S(═O)₂R_{S315D}, —OS(═O)₂OR_{S315C}, —NR_{S315C}S(═O)₂OR_{S315C}, —OS(═O)₂N(R_{S315C})₂, —NR_{S315C}S(═O)₂N(R_{S315C})₂, —P(R_{S315C})₂, —P(═O)(R_{S315D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, two R_{S315} together with the carbon atom to which they are both attached form 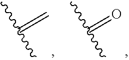
a 3-10 membered carbocyclic ring or a 3-10 heterocyclic ring; wherein, said 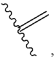
3-10 membered carbocylic ring or 3-10 heterocyclic ring is independently unsubstituted or substituted with one or more R_{S3151};
optionally, two adjacent R_{S315} together with the carbon atoms to which they are respectively attached form a 3-10 membered carbocyclic ring, a 3-10 membered heterocyclic ring, a 6-10 membered aryl ring or a 5-10 membered heteroaryl ring, wherein, each of rings is independently unsubstituted or substituted with one or more R_{S3152};
optionally, two nonadjacent R_{S315} are connected together to form a bridge containing 0, 1, 2, 3, 4, 5 or 6 carbon atoms, wherein, each of the carbon atoms in the bridge is independently not replaced or replaced by 1 or 2 heteroatoms selected from the group consisting of N, O, S, S═O and S(═O)₂; the hydrogen on the each of carbon atoms or N atoms is independently unsubstituted or substituted with R_{S3153};
m₁₀ is selected from the group consisting of 0, 1, 2, 3, 4, 5 and 6;
R₄ is selected from the group consisting of 6-10 membered aryl, 5-10 membered heteroaryl, 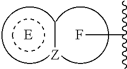
and 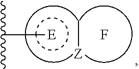
wherein said 6-10 membered aryl, 5-10 membered heteroaryl, 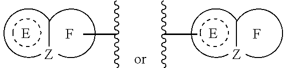
is independently unsubstituted or substituted with one or more R_{S4};
Z at each occurrence is independently selected from the group consisting of C and N;
ring E at each occurrence is independently selected from the group consisting of a 6 membered aryl ring and a 5-6 membered heteroaryl ring and ring F at each occurrence is a 3-10 membered carbocyclic ring or a 3-10 membered heterocyclic ring when Z is C;
ring E at each occurrence is a 5-6 membered heteroaryl ring and ring F at each occurrence is a 3-10 membered heterocyclic ring when Z is N;
R_{S4} is independently selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4A})₂, —OR_{S4A}, —SR_{S4A}, —S(═O)R_{S4B}, —S(═O)₂R_{S4B}, —C(═O)R_{S4B}, —C(═O)OR_{S4A}, —OC(═O)R_{S4B}, —C(═O)N(R_{S4A})₂, —NR_{S4A}C(═O)R_{S4B}, —OC(═O)OR_{S4A}, —NR_{S4A}C(═O)OR_{S4A}, —NR_{S4A}C(═S)OR_{S4A}, —OC(═O)N(R_{S4A})₂, —NR_{S4A}C(═O)N(R_{S4A})₂, —S(═O)OR_{S4A}, —OS(═O)R_{S4B}, —S(═O)N(R_{S4A})₂, —NR_{S4A}S(═O)R_{S4B}, —S(═O)₂OR_{S4A}, —OS(═O)₂R_{S4B}, —S(═O)₂N(R_{S4A})₂, —NR_{S4A}S(═O)₂R_{S4B}, —OS(═O)₂OR_{S4A}, —NR_{S4A}S(═O)₂OR_{S4A}, —OS(═O)₂N(R_{S4A})₂, —NR_{S4A}S(═O)₂N(R_{S4A})₂, —P(R_{S4A})₂, —P(═O)(R_{S4B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{S4C})₂, —OR_{S4C}, —SR_{S4C}, —S(═O)R_{S4D}, —S(═O)₂R_{S4D}, —C(═O)R_{S4D}, —C(═O)OR_{S4D}, —OC(═O)R_{S4D}, —C(═O)N(R_{S4C})₂, —NR_{S4C}C(═O)R_{S4D}, —OC(═O)OR_{S4C}, —NR_{S4C}C(═O)OR_{S4C}, —NR_{S4C}C(═S)OR_{S4C}, —OC(═O)N(R_{S4C})₂, —NR_{S4C}C(═O)N(R_{S4C})₂, —S(═O)OR_{S4C}, —OS(═O)R_{S4D}, —S(═O)N(R_{S4C})₂, —NR_{S4C}S(═O)R_{S4D}, —S(═O)₂OR_{S4C}, —OS(═O)₂R_{S4D}, —S(═O)₂N(R_{S4C})₂, —NR_{S4C}S(═O)₂R_{S4D}, —OS(═O)₂OR_{S4C}, —NR_{S4C}S(═O)₂OR_{S4C}, —OS(═O)₂N(R_{S4C})₂, —NR_{S4C}S(═O)₂N(R_{S4C})₂, —P(R_{S4C})₂, —P(═O)(R_{S4D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
R₅ is selected from the group consisting of hydrogen, deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5A})₂, —OR_{5A}, —SR_{5A}, —S(═O)R_{5B}, —S(═O)₂R_{5B}, —C(═O)R_{5B}, —C(═O)OR_{5A}, —OC(═O)R_{5B}, —C(═O)N(R_{5A})₂, —NR_{5A}C(═O)R_{5B}, —OC(═O)OR_{5A}, —NR_{5A}C(═O)OR_{5A}, —NR_{5A}C(═S)OR_{5A}, —OC(═O)N(R_{5A})₂, —NR_{5A}C(═O)N(R_{5A})₂, —S(═O)OR_{5A}, —OS(═O)R_{5B}, —S(═O)N(R_{5A})₂, —NR_{5A}S(═O)R_{5B}, —S(═O)₂OR_{5A}, —OS(═O)₂R_{5B}, —S(═O)₂N(R_{5A})₂, —NR_{5A}S(═O)₂R_{5B}, —OS(═O)₂OR_{5A}, —NR_{5A}S(═O)₂OR_{5A}, —OS(═O)₂N(R_{5A})₂, —NR_{5A}S(═O)₂N(R_{5A})₂, —P(R_{5A})₂, —P(═O)(R_{5B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —CN, —NO₂, —N₃, oxo, —N(R_{5C})₂, —OR_{5C}, —SR_{5C}, —S(═O)R_{5D}, —S(═O)₂R_{5D}, —C(═O)R_{5D}, —C(═O)OR_{5D}, —OC(═O)R_{5D}, —C(═O)N(R_{5C})₂, —NR_{5C}C(═O)R_{5D}, —OC(═O)OR_{5C}, —NR_{5C}C(═O)OR_{5C}, —NR_{5C}C(═S)OR_{5C}, —OC(═O)N(R_{5C})₂, —NR_{5C}C(═O)N(R_{5C})₂, —S(═O)OR_{5C}, —OS(═O)R_{5D}, —S(═O)N(R_{5C})₂, —NR_{5C}S(═O)R_{5D}, —S(═O)₂OR_{5C}, —OS(═O)₂R_{5D}, —S(═O)₂N(R_{5C})₂, —NR_{5C}S(═O)₂R_{5D}, —OS(═O)₂OR_{5C}, —NR_{5C}S(═O)₂OR_{5C}, —OS(═O)₂N(R_{5C})₂, —NR_{5C}S(═O)₂N(R_{5C})₂, —P(R_{5C})₂, —P(═O)(R_{5D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
each of R_{1A}, R_{1C}, R_{S1A}, R_{S1C}, R_{S31A}, R_{S31C}, R_{S32A}, R_{S32C}, R_{37A}, R_{S38A}, R_{S38C}, R_{S39A}, R_{S39C}, R_{S315A}, R_{S315C}, R_{S4A}, R_{S4C}, R_{5A} and R_{5C} is independently selected from the group consisting of hydrogen, deuterium, —C₁₋₆alkyl, haloC₁₋₆alkyl, —C₂₋₆alkenyl, —C₂₋₆alkynyl, —S(═O)R_{B}, —S(═O)₂R_{B}, —C(═O)R_{B}, —C(═O)OR_{B}, —C(═O)N(R_{B})₂, —S(═O)OR_{B}, —S(═O)N(R_{B})₂, —S(═O)₂OR_{B}, —S(═O)₂N(R_{B})₂, —P(═O)(R_{B})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
optionally, (two R_{1A}, two R_{1C}, two R_{S1A}, two R_{S1C}, two R_{S31A}, two R_{S31C}, two R_{S32A}, two R_{S32C}, two R_{37A}, two R_{S38A}, two R_{S38C}, two R_{S39A}, two R_{S39C}, two R_{S315A}, two R_{S315C}, two R_{S4A}, two R_{S4C}, two R_{5A} and two R_{5C}) together with the nitrogen atom to which they are both attached forms a 3-10 membered heterocyclic ring or a 5-10 membered heteroaryl ring, wherein, said 3-10 membered heterocyclic ring or 5-10 membered heteroaryl ring is independently unsubstituted or substituted with one or more R_{SS};
each of R_{1B}, R_{1D}, R_{S1B}, R_{S1D}, R_{S31B}, R_{S31D}, R_{S32B}, R_{S32D}, R_{S38B}, R_{S38D}, R_{S39B}, R_{S39D}, R_{S315B}, R_{S315D}, R_{S4B}, R_{S4D}, R_{5B} and R_{5D} is independently selected from the group consisting of hydrogen, deuterium, —C₁₋₆alkyl, haloC₁₋₆alkyl, haloC₁₋₆alkoxy, —C₂₋₆alkenyl, haloC₂₋₆alkenyl, —C₂₋₆alkynyl, haloC₂₋₆alkynyl, —N(R_{A})₂, —OR_{A}, —SR_{A}, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —N(R_{C})₂, —OR_{C}, —SR_{C}, —S(═O)R_{D}, —S(═O)₂R_{D}, —C(═O)R_{D}, —C(═O)OR_{C}, —OC(═O)R_{D}, —C(═O)N(R_{C})₂, —NR_{C}C(═O)R_{D}, —OC(═O)OR_{C}, —NR_{C}C(═O)OR_{D}, —OC(═O)N(R_{C})₂, —NR_{C}C(═O)N(R_{C})₂, —S(═O)OR_{C}, —OS(═O)R_{D}, —S(═O)N(R_{C})₂, —NR_{C}S(═O)R_{D}, —S(═O)₂OR_{C}, —OS(═O)₂R_{D}, —S(═O)₂N(R_{C})₂, —NR_{C}S(═O)₂R_{D}, —OS(═O)₂OR_{C}, —NR_{C}S(═O)₂OR_{C}, —OS(═O)₂NR_{C}, —NR_{C}S(═O)₂N(R_{C})₂, —P(R_{C})₂, —P(═O)(R_{D})₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
each of (R_{A}, R_{B}, R_{C} and R_{D}) is independently selected from the group consisting of hydrogen, deuterium, —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl; wherein said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl and 5-10 membered heteroaryl is independently unsubstituted or substituted with one or more R_{SA};
each of R_{SX1}, R_{S11}, R_{S12}, R_{S13}, R_{S33}, R_{S34}, R_{S35}, R_{S316}, R_{S311}, R_{S312}, R_{S313}, R_{S321}, R_{S322}, R_{S323}, R_{S37}, R_{S310}, R_{S381}, R_{S382}, R_{S383}, R_{S391}, R_{S392}, R_{S393}, R_{S3151}, R_{S3152}, R_{S3153}, R_{SS} and R_{SA} is independently selected from the group consisting of deuterium, halogen, —C₁₋₆alkyl, haloC₁₋₆alkyl, haloC₁₋₆alkoxy, —C₂₋₆alkenyl, —C₂₋₆alkynyl, —CN, —NO₂, —N₃, oxo, —NH₂, —NH(C₁₋₆alkyl), —N(C₁₋₆alkyl)₂, —OH, —O(C₁₋₆alkyl), —SH, —S(C₁₋₆alkyl), —S(═O)(C₁₋₆alkyl), —S(═O)₂(C₁₋₆alkyl), —C(═O)(C₁₋₆alkyl), —C(═O)OH, —C(═O)(OC₁₋₆alkyl), —OC(═O)(C₁₋₆alkyl), —C(═O)NH₂, —C(═O)NH(C₁₋₆alkyl), —C(═O)N(C₁₋₆alkyl)₂, —NHC(═O)(C₁₋₆alkyl), —N(C₁₋₆alkyl)C(═O)(C₁₋₆alkyl), —OC(═O)O(C₁₋₆alkyl), —NHC(═O)(OC₁₋₆alkyl), —N(C₁₋₆alkyl)C(═O)(OC₁₋₆alkyl), —OC(═O)NH(C₁₋₆alkyl), —OC(═O)N(C₁₋₆alkyl)₂, —NHC(═O)NH₂, —NHC(═O)NH(C₁₋₆alkyl), —NHC(═O)N(C₁₋₆alkyl)₂, —N(C₁₋₆alkyl)C(═O)NH₂, —N(C₁₋₆alkyl)C(═O)NH(C₁₋₆alkyl), —N(C₁₋₆alkyl)C(═O)N(C₁₋₆alkyl)₂, —S(═O)(OC₁₋₆alkyl), —OS(═O)(C₁₋₆alkyl), —S(═O)NH₂, —S(═O)NH(C₁₋₆alkyl), —S(═O)N(C₁₋₆alkyl)₂, —NHS(═O)(C₁₋₆alkyl), —N(C₁₋₆alkyl)S(═O)(C₁₋₆alkyl), —S(═O)₂(OC₁₋₆alkyl), —OS(═O)₂(C₁₋₆alkyl), —S(═O)₂NH₂, —S(═O)₂NH(C₁₋₆alkyl), —S(═O)₂N(C₁₋₆alkyl)₂, —NHS(═O)₂(C₁₋₆alkyl), —N(C₁₋₆alkyl)S(═O)₂(C₁₋₆alkyl), —OS(═O)₂O(C₁₋₆alkyl), —NHS(═O)₂O(C₁₋₆alkyl), —N(C₁₋₆alkyl)S(═O)₂O(C₁₋₆alkyl), —OS(═O)₂NH₂, —OS(═O)₂NH(C₁₋₆alkyl), —OS(═O)₂N(C₁₋₆alkyl)₂, —NHS(═O)₂NH₂, —NHS(═O)₂NH(C₁₋₆alkyl), —NHS(═O)₂N(C₁₋₆alkyl)₂, —N(C₁₋₆alkyl)S(═O)₂NH₂, —N(C₁₋₆alkyl)S(═O)₂NH(C₁₋₆alkyl), —N(C₁₋₆alkyl)S(═O)₂N(C₁₋₆alkyl)₂, —PH(C₁₋₆alkyl), —P(C₁₋₆alkyl)₂, —P(═O)H(C₁₋₆alkyl), —P(═O)(C₁₋₆alkyl)₂, 3-10 membered cycloalkyl, 3-10 membered cycloalkenyl, 3-10 membered cycloalkynyl, 3-10 membered heterocyclyl, 6-10 membered aryl and 5-10 membered heteroaryl; wherein, said —C₁₋₆alkyl, haloC₁₋₆alkyl, haloC₁₋₆alkoxy, —C₂₋₆alkenyl, —C₂₋₆alkynyl, 3-10 membered cycloalkyl, 3-10 membered cycloalkenyl, 3-10 membered cycloalkynyl, 3-10 membered heterocyclyl, 6-10 membered aryl or 5-10 membered heteroaryl is independently unsubstituted or substituted with one or more substituents selected from the group consisting of deuterium, halogen, —C₁₋₃alkyl, haloC₁₋₃alkyl, haloC₁₋₃alkoxy, —C₂₋₃alkenyl, —C₂₋₃alkynyl, —CN, —NO₂, —N₃, oxo, —NH₂, —NH(C₁₋₃alkyl), —N(C₁₋₃alkyl)₂, —OH, —O(C₁₋₃alkyl), —SH, —S(C₁₋₃alkyl), —S(═O)(C₁₋₃alkyl), —S(═O)₂(C₁₋₃alkyl), —C(═O)(C₁₋₃alkyl), —C(═O)OH, —C(═O)(OC₁₋₃alkyl), —OC(═O)(C₁₋₃alkyl), —C(═O)NH₂, —C(═O)NH(C₁₋₃alkyl), —C(═O)N(C₁₋₃alkyl)₂, —NHC(═O)(C₁₋₃alkyl), —N(C₁₋₃alkyl)C(═O)(C₁₋₃alkyl), —OC(═O)O(C₁₋₃alkyl), —NHC(═O)(OC₁₋₃alkyl), —N(C₁₋₃alkyl)C(═O)(OC₁₋₃alkyl), —OC(═O)NH(C₁₋₃alkyl), —OC(═O)N(C₁₋₃alkyl)₂, —NHC(═O)NH₂, —NHC(═O)NH(C₁₋₃alkyl), —NHC(═O)N(C₁₋₃alkyl)₂, —N(C₁₋₃alkyl)C(═O)NH₂, —N(C₁₋₃alkyl)C(═O)NH(C₁₋₃alkyl), —N(C₁₋₃alkyl)C(═O)N(C₁₋₃alkyl)₂, —S(═O)(OC₁₋₃alkyl), —OS(═O)(C₁₋₃alkyl), —S(═O)NH₂, —S(═O)NH(C₁₋₃alkyl), —S(═O)N(C₁₋₃alkyl)₂, —NHS(═O)(C₁₋₃alkyl), —N(C₁₋₃alkyl)S(═O)(C₁₋₃alkyl), —S(═O)₂(OC₁₋₃alkyl), —OS(═O)₂(C₁₋₃alkyl), —S(═O)₂NH₂, —S(═O)₂NH(C₁₋₃alkyl), —S(═O)₂N(C₁₋₃alkyl)₂, —NHS(═O)₂(C₁₋₃alkyl), —N(C₁₋₃alkyl)S(═O)₂(C₁₋₃alkyl), —OS(═O)₂O(C₁₋₃alkyl), —NHS(═O)₂O(C₁₋₃alkyl), —N(C₁₋₃alkyl)S(═O)₂O(C₁₋₃alkyl), —OS(═O)₂NH₂, —OS(═O)₂NH(C₁₋₃alkyl), —OS(═O)₂N(C₁₋₃alkyl)₂, —NHS(═O)₂NH₂, —NHS(═O)₂NH(C₁₋₃alkyl), —NHS(═O)₂N(C₁₋₃alkyl)₂, —N(C₁₋₃alkyl)S(═O)₂NH₂, —N(C₁₋₃alkyl)S(═O)₂NH(C₁₋₃alkyl), —N(C₁₋₃alkyl)S(═O)₂N(C₁₋₃alkyl)₂, —PH(C₁₋₃alkyl), —P(C₁₋₃alkyl)₂, —P(═O)H(C₁₋₃alkyl), —P(═O)(C₁₋₃alkyl)₂, 3-10 membered cycloalkyl, 3-10 membered cycloalkenyl, 3-10 membered cycloalkynyl, 3-10 membered heterocyclyl, 6-10 membered aryl and 5-10 membered heteroaryl;
each of heterocyclyl or heterocyclic at each occurrence independently contains 1, 2, 3 or 4 heteroatoms selected from the group consisting of N, O, S, S(═O) and S(═O)₂;
each of heteroaryl at each occurrence independently contains 1, 2, 3 or 4 heteroatoms selected from the group consisting of N, O, and S.

2-89. (canceled)
